(19) United States
(12) Patent Application Publication
(10) Pub. No.: US 2010/0150950 A1
(43) Pub. Date: Jun. 17, 2010

(54) HUMAN ANTIBODIES THAT BIND CD70 AND USES THEREOF

(75) Inventors: Marco A. Coccia, Scotts Valley, CA (US); Jonathan A. Terrett, Sunnyvale, CA (US); David John King, Solana Beach, CA (US); Chin Pan, Los Altos, CA (US); Josephine Cardarelli, San Carlos, CA (US); Mark Yamanaka, Pleasanton, CA (US); Ann Karla Henning, Milpitas, CA (US)

Correspondence Address: BAKER BOTTS L.L.P., 30 ROCKEFELLER PLAZA, 44th Floor, NEW YORK, NY, 10112-4498, US

(73) Assignee: Medarex, Inc., Princeton, NJ (US)

(21) Appl. No.: 12/518,838

(22) PCT Filed: Dec. 13, 2007
(86) PCT No.: PCT/US2007/087401

§ 371 Date: Dec. 18, 2009

Related U.S. Application Data
(60) Provisional application No. 60/870,091, filed on Dec. 14, 2006.
(60) Provisional application No. 60/915,314, filed on May 1, 2007.
(60) Provisional application No. 60/991,702, filed on Nov. 30, 2007.

(51) Int. Cl. A61K 39/395; C07K 16/00; A61K 38/16; A61P 35/00; A61P 29/00; A61P 31/12; A61P 37/00; C12N 5/09

(52) U.S. Cl. 424/178.1; 530/391.7; 530/391.3; 530/388.1; 530/388.23; 435/375; 530/391.9

(57) ABSTRACT

The present disclosure provides isolated monoclonal antibodies that specifically bind to CD70 with high affinity, particularly human monoclonal antibodies. Preferably, the antibodies bind human CD70. In certain embodiments, the antibodies are capable of being internalized into CD70-expressing cells or are capable of mediating antigen dependent cellular cytotoxicity. Nucleic acid molecules encoding the antibodies of this disclosure, expression vectors, host cells and methods for expressing the antibodies of this disclosure are also provided. Antibody-partner molecule conjugates, bispecific molecules and pharmaceutical compositions comprising the antibodies of this disclosure are also provided. This disclosure also provides methods for detecting CD70, as well as methods for treating cancers, such as renal cancer and lymphomas, using an anti-CD7Q antibody of this disclosure.

### RELATED APPLICATIONS

This application claims priority to U.S. Provisional Application Ser. No. 60/870,091, filed on Dec. 14, 2006, U.S. Provisional Application Ser. No. 60/915,314, filed on May 1, 2007, and U.S. Provisional Application Ser. No. 60/991,702, filed on Nov. 30, 2007, the contents of which are hereby incorporated herein by reference.

### BACKGROUND

The cytokine receptor CD27 is a member of the tumor necrosis factor receptor (TFNR) superfamily, which play a role in cell growth and differentiation, as well as apoptosis or programmed cell death. The ligand for CD27 is CD70, which belongs to the tumor necrosis factor family of ligands. CD70 is a 193 amino acid polypeptide having a 20 amino acid hydrophilic N-terminal domain and a C-terminal domain containing 2 potential N-linked glycosylation sites (Goodwin, R. G. et al. (1993) Cell 73:447-56; Bowman et al. (1994) Immunol 152:1756-61). Based on these features, CD70 was determined to be a type II transmembrane protein having an extracellular C-terminal portion.

CD70 is transiently found on activated, but not resting T and B lymphocytes and dendritic cells (Hintzen et al. (1994) J. Immunol. 152:1762-1773; Oshima et al. (1998) Int. Immunol. 10:517-26; Tesselaar et al. (2003) J. Immunol. 170:33-40). In addition to expression on normal cells, CD70 expression has been reported in different types of cancers including renal cell carcinomas, metastatic breast cancers, brain tumors, leukemias, lymphomas and nasopharyngeal carcinomas (Junker et al. (2005) J Urol. 173:2150-3; Sloan et al. (2004) Am J Pathol. 164:315-23; Held-Feindt and Mentlein (2002) Int J Cancer 98:352-6; Hishima et al. (2000) Am J Surg Pathol. 24:742-6; Lens et al. (1999) Br J Haematol. 106:491-503). Additionally, CD70 has been found to be over expressed on T cells treated with DNA methyltransferase inhibitors or ERK pathway inhibitors, possibly leading to drug-induced and idiopathic lupus (Oelke et al. (2004) Arthritis Rheum. 50:1850-60). The interaction of CD70 with CD27 has also been proposed to play a role in cell-mediated autoimmune disease and the inhibition of TNF-alpha production (Nakajima et al. (2000) J. Neuroimmunol. 109:188-96).

Accordingly, CD70 represents a valuable target for the treatment of cancer, autoimmune disorders and a variety of other diseases characterized by CD70 expression.

### SUMMARY

The present disclosure provides isolated monoclonal antibodies, in particular human monoclonal antibodies that specifically bind to CD70 and that have desirable functional properties. These properties include high affinity binding to human CD70, internalization by cells expressing CD70, the ability to mediate antibody dependent cellular cytotoxicity, the ability to bind to a renal cell carcinoma tumor cell line, and/or the ability to bind to a lymphoma cell line, e.g., a B-cell tumor cell line. The antibodies of the invention can be used, for example, to detect CD70 protein or to inhibit the growth of cells expressing CD70, such as tumor cells that express CD70.

Also provided are methods for treating a variety CD70 mediated diseases using the isolated monoclonal antibodies and compositions thereof of the instant disclosure.

In one aspect, this disclosure pertains to an isolated monoclonal antibody, or an antigen-binding portion thereof, wherein the antibody binds to CD70 and exhibits at least one of the following properties:

(a) binds to human CD70 with a K_{D} of 1×10⁻⁷ M or less; and

(b) binds to a renal cell carcinoma tumor cell line;

(c) binds to a lymphoma cell line, e.g., a B-cell tumor cell line;

(d) is internalized by CD70-expressing cells;

(e) exhibits antibody dependent cellular cytotoxicity (ADCC) against CD70-expressing cells; and

(f) inhibits growth of CD70-expressing cells in vivo when conjugated to a cytotoxin.

Preferably, the antibody exhibits at least two of properties (a), (b), (c), (d), (e), and (f). More preferably, the antibody exhibits at least three of properties (a), (b), (c), (d), (e), and (f). More preferably, the antibody exhibits four of properties (a), (b), (c), (d), (e), and (f). Even more preferably, the antibody exhibits five of properties (a), (b), (c), (d), (e), and (f). Even more preferably, the antibody exhibits all six properties (a), (b), (c), (d), (e), and (f). In yet another preferred embodiment, the antibody inhibits growth of CD70-expressing tumor cells in vivo when the antibody is conjugated to a cytotoxin.

Preferably, the antibody binds to a renal cell carcinoma tumor cell line selected from the group consisting of 786-O (ATCC Accession No. CRL-1932), A-498 (ATCC Accession No. HTB-44), ACHN (ATCC Accession No. CRL-1611), Caki-1 (ATCC Accession No. HTB-46) and Caki-2 (ATCC Accession No. HTB-47).

Preferably, the antibody binds to a B-cell tumor cell line that is selected from Daudi (ATCC Accession No. CCL-213), HuT 78 (ATCC Accession No. TIB-161), Raji (ATCC Accession No. CCL-86) or Granta-519 (DSMZ Accession No. 342) cells.

Preferably the antibody is a human antibody, although in alternative embodiments the antibody can be a murine antibody, a chimeric antibody or a humanized antibody.

In more preferred embodiments, the antibody binds to human CD70 with a K_{D} of 5.5×10⁻⁹ M or less or binds to human CD70 with a K_{D} of 3×10⁻⁹ M or less or binds to human CD70 with a K_{D} of 2×10⁻⁹ M or less or binds to human CD70 with a K_{D} of 1.5×10⁻⁹ M or less.

In another embodiment, the antibodies are internalized by 786-O renal cell carcinoma tumor cells after binding to CD70 expressed on those cells.

In another embodiment, this disclosure provides an isolated monoclonal antibody, or an antigen-binding portion thereof, wherein the antibody cross-competes for binding to an epitope on CD70 which is recognized by a reference antibody, wherein the reference antibody: (a) binds to human CD70 with a K_{D} of 1×10⁻⁷ M or less; and (b) binds to a renal cell carcinoma tumor cell line.

In various embodiments, the reference antibody comprises:

(a) a heavy chain variable region comprising the amino acid sequence of SEQ ID NO:1; and (b) a light chain variable region comprising the amino acid sequence of SEQ ID NO:7;

or the reference antibody comprises (a) a heavy chain variable region comprising the amino acid sequence of SEQ ID NO:2; and (b) a light chain variable region comprising the amino acid sequence of SEQ ID NO:8;

or the reference antibody comprises (a) a heavy chain variable region comprising the amino acid sequence of SEQ ID NO:3; and (b) a light chain variable region comprising the amino acid sequence of SEQ ID NO:9;

or the reference antibody comprises (a) a heavy chain variable region comprising the amino acid sequence of SEQ ID NO:4; and (b) a light chain variable region comprising the amino acid sequence of SEQ ID NO:10;

or the reference antibody comprises (a) a heavy chain variable region comprising the amino acid sequence of SEQ ID NO:5 or 73; and (b) a light chain variable region comprising the amino acid sequence of SEQ ID NO:11;

or the reference antibody comprises (a) a heavy chain variable region comprising the amino acid sequence of SEQ ID NO:6; and (b) a light chain variable region comprising the amino acid sequence of SEQ ID NO:12.

In another embodiment, a reference antibody of this disclosure is antibody 69A7Y. 69A7Y is the same as antibody 69A7, but contains a conservative modification in the V_{H} amino acid sequence of SEQ ID NO:5 resulting in a mutation of C (cysteine) to Y (tyrosine) at amino acid position 100. The V_{H} amino acid sequence of 69A7Y is set forth as SEQ ID NO:73. The C to Y mutation results from a single basepair substitution of G to A at nucleotide position 323 of the V_{H} nucleotide sequence of 69A7 (SEQ ID NO:53). The V_{H} nucleotide sequence of 69A7Y is set forth as SEQ ID NO:74. 69A7Y has a heavy chain variable region CDR3 comprising the amino acid sequence set forth as SEQ ID NO:75.

In another aspect, the invention pertains to an isolated monoclonal antibody, or an antigen-binding portion thereof linked to a therapeutic agent comprising a heavy chain variable region that is the product of or derived from a human V_{H} 3-30.3 gene, wherein the antibody specifically binds CD70. This disclosure also provides an isolated monoclonal antibody comprising a monoclonal antibody or an antigen-binding portion thereof linked to a therapeutic agent, wherein the antibody comprises a heavy chain variable region that is the product of or derived from a human V_{H} 3-33 gene, wherein the antibody specifically binds CD70. This disclosure also provides an isolated monoclonal antibody comprising a monoclonal antibody or an antigen-binding portion thereof linked to a therapeutic agent, wherein the antibody comprises a heavy chain variable region that is the product of or derived from a human V_{H} 4-61 gene, wherein the antibody specifically binds CD70. This disclosure also provides an isolated monoclonal antibody comprising a monoclonal antibody or an antigen-binding portion thereof linked to a therapeutic agent, wherein the antibody comprises a heavy chain variable region that is the product of or derived from a human V_{H} 3-23 gene, wherein the antibody specifically binds CD70.

This disclosure still further provides an isolated monoclonal antibody comprising a monoclonal antibody or an antigen-binding portion thereof linked to a therapeutic agent, wherein the antibody comprises a light chain variable region that is the product of or derived from a human V_{K} L6 gene, wherein the antibody specifically binds CD70. This disclosure still further provides an isolated monoclonal antibody comprising a monoclonal antibody or an antigen-binding portion thereof linked to a therapeutic agent, wherein the antibody comprises a light chain variable region that is the product of or derived from a human V_{K} L18 gene, wherein the antibody specifically binds CD70. This disclosure further provides an isolated monoclonal antibody comprising a monoclonal antibody or an antigen-binding portion thereof linked to a therapeutic agent, wherein the antibody comprises a light chain variable region that is the product of or derived from a human V_{K} L15 gene, wherein the antibody specifically binds to CD70. This disclosure further provides an isolated monoclonal antibody comprising a monoclonal antibody or an antigen-binding portion thereof linked to a therapeutic agent, wherein the antibody comprises a light chain variable region that is the product of or derived from a human V_{K} A27 gene, wherein the antibody specifically binds to CD70.

A particularly preferred antibody or antigen-binding portion thereof comprises:

(a) a heavy chain variable region CDR1 comprising SEQ ID NO:13;

(b) a heavy chain variable region CDR2 comprising SEQ ID NO:19;

(c) a heavy chain variable region CDR3 comprising SEQ ID NO:25;

(d) a light chain variable region CDR1 comprising SEQ ID NO:31;

(e) a light chain variable region CDR2 comprising SEQ ID NO:37; and

(f) a light chain variable region CDR3 comprising SEQ ID NO:43.

Another preferred combination comprises:

(a) a heavy chain variable region CDR1 comprising SEQ ID NO:14;

(b) a heavy chain variable region CDR2 comprising SEQ ID NO:20;

(c) a heavy chain variable region CDR3 comprising SEQ ID NO:26;

(d) a light chain variable region CDR1 comprising SEQ ID NO:32;

(e) a light chain variable region CDR2 comprising SEQ ID NO:38; and

(f) a light chain variable region CDR3 comprising SEQ ID NO:44.

Another preferred combination comprises:

(a) a heavy chain variable region CDR1 comprising SEQ ID NO:15;

(b) a heavy chain variable region CDR2 comprising SEQ ID NO:21;

(c) a heavy chain variable region CDR3 comprising SEQ ID NO:27;

(d) a light chain variable region CDR1 comprising SEQ ID NO:33;

(e) a light chain variable region CDR2 comprising SEQ ID NO:39; and

(f) a light chain variable region CDR3 comprising SEQ ID NO: 45.

Another preferred combination comprises:

(a) a heavy chain variable region CDR1 comprising SEQ ID NO:16;

(b) a heavy chain variable region CDR2 comprising SEQ ID NO:22;

(c) a heavy chain variable region CDR3 comprising SEQ ID NO:28;

(d) a light chain variable region CDR1 comprising SEQ ID NO:34;

(e) a light chain variable region CDR2 comprising SEQ ID NO:40; and

(f) a light chain variable region CDR3 comprising SEQ ID NO:46.

Another preferred combination comprises:

(a) a heavy chain variable region CDR1 comprising SEQ ID NO:17;

(b) a heavy chain variable region CDR2 comprising SEQ ID NO:23;

(c) a heavy chain variable region CDR3 comprising SEQ ID NO:29 or 75;

(d) a light chain variable region CDR1 comprising SEQ ID NO:35;

(e) a light chain variable region CDR2 comprising SEQ ID NO:41; and

(f) a light chain variable region CDR3 comprising SEQ ID NO:47.

Another preferred combination comprises:

(a) a heavy chain variable region CDR1 comprising SEQ ID NO:18;

(b) a heavy chain variable region CDR2 comprising SEQ ID NO:24;

(c) a heavy chain variable region CDR3 comprising SEQ ID NO:30;

(d) a light chain variable region CDR1 comprising SEQ ID NO:36;

(e) a light chain variable region CDR2 comprising SEQ ID NO:42; and

(f) a light chain variable region CDR3 comprising SEQ ID NO:48.

Other preferred antibodies of this disclosure have an antibody or antigen binding portion thereof which comprise (a) a heavy chain variable region comprising the amino acid sequence of SEQ ID NO:1; and (b) a light chain variable region comprising the amino acid sequence of SEQ ID NO:7.

Another preferred combination comprises (a) a heavy chain variable region comprising the amino acid sequence of SEQ ID NO:2; and (b) a light chain variable region comprising the amino acid sequence of SEQ ID NO:8.

Another preferred combination comprises (a) a heavy chain variable region comprising the amino acid sequence of SEQ ID NO:3; and (b) a light chain variable region comprising the amino acid sequence of SEQ ID NO:9.

Another preferred combination comprises (a) a heavy chain variable region comprising the amino acid sequence of SEQ ID NO:4; and (b) a light chain variable region comprising the amino acid sequence of SEQ ID NO:10.

Another preferred combination comprises (a) a heavy chain variable region comprising the amino acid sequence of SEQ ID NO:5 or 73; and (b) a light chain variable region comprising the amino acid sequence of SEQ ID NO:11.

Another preferred combination comprises (a) a heavy chain variable region comprising the amino acid sequence of SEQ ID NO:6; and (b) a light chain variable region comprising the amino acid sequence of SEQ ID NO:12.

In another embodiment, an antibody of this disclosure is antibody 69A7Y. 69A7Y is the same as antibody 69A7, but contains a conservative modification in the V_{H} amino acid sequence of SEQ ID NO:5 resulting in a mutation of C (cysteine) to Y (tyrosine) at amino acid position 100. The V_{H} amino acid sequence of 69A7Y is set forth as SEQ ID NO:73. The C to Y mutation results from a single basepair substitution of G to A at nucleotide position 323 of the V_{H} nucleotide sequence of 69A7 (SEQ ID NO:53). The V_{H} nucleotide sequence of 69A7Y is set forth as SEQ ID NO:74. 69A7Y has a heavy chain variable region CDR3 comprising the amino acid sequence set forth as SEQ ID NO:75.

The antibodies of this disclosure can be, for example, full-length antibodies, for example of an IgG1 or IgG4 isotype. Alternatively, the antibodies can be antibody fragments, such as Fab or Fab′₂ fragments or single chain antibodies.

This disclosure also provides an immunoconjugate comprising an antibody of this disclosure or an antigen-binding portion thereof, linked to a therapeutic agent, such as a cytotoxin or a radioactive isotope. In a particularly preferred embodiment, the invention provides an immunoconjugate comprising an antibody of this disclosure, or antigen-binding portion thereof, linked to a cytotoxin (for example, a cytotoxin described herein or in U.S. Pat. App. No. 60/882,461, filed on Dec. 28, 2006 or U.S. Pat. App. No. 60/991,300, filed on Nov. 30, 2007, which are hereby incorporated by reference in their entirety), (e.g., via a thiol linkage). In certain embodiments, the cytotoxin and linker of the immunoconjugate has the structure of N1 or N2.

For example, in various embodiments, the invention provides the following preferred immunoconjugates:

(i) an immunoconjugate comprising an antibody, or antigen-binding portion thereof, comprising:

(a) a heavy chain variable region comprising the amino acid sequence of SEQ ID NO:1 and a light chain variable region comprising the amino acid sequence of SEQ ID NO:7;

(b) a heavy chain variable region comprising the amino acid sequence of SEQ ID NO:2 and a light chain variable region comprising the amino acid sequence of SEQ ID NO:8;

(c) a heavy chain variable region comprising the amino acid sequence of SEQ ID NO:3 and a light chain variable region comprising the amino acid sequence of SEQ ID NO:9;

(d) a heavy chain variable region comprising the amino acid sequence of SEQ ID NO:4 and a light chain variable region comprising the amino acid sequence of SEQ ID NO:10;

(e) a heavy chain variable region comprising the amino acid sequence of SEQ ID NO:5 or 73 and a light chain variable region comprising the amino acid sequence of SEQ ID NO:11, and

(f) a heavy chain variable region comprising the amino acid sequence of SEQ ID NO:6 and a light chain variable region comprising the amino acid sequence of SEQ ID NO:12, where the antibody or antigen binding portion thereof is linked to a cytotoxin;

(ii) an immunoconjugate comprising an antibody, or antigen-binding portion thereof, comprising:

(a) a heavy chain variable region CDR1 comprising SEQ ID NO:13;

(b) a heavy chain variable region CDR2 comprising SEQ ID NO:19;

(c) a heavy chain variable region CDR3 comprising SEQ ID NO:25;

(d) a light chain variable region CDR1 comprising SEQ ID NO:31;

(e) a light chain variable region CDR2 comprising SEQ ID NO:37; and

(f) a light chain variable region CDR3 comprising SEQ ID NO:43;

or an antibody, or antigen-binding portion thereof, comprising:

(a) a heavy chain variable region CDR1 comprising SEQ ID NO:14;

(b) a heavy chain variable region CDR2 comprising SEQ ID NO:20;

(c) a heavy chain variable region CDR3 comprising SEQ ID NO:26;

(d) a light chain variable region CDR1 comprising SEQ ID NO:32;

(e) a light chain variable region CDR2 comprising SEQ ID NO:38; and

(f) a light chain variable region CDR3 comprising SEQ ID NO:44;

or an antibody, or antigen-binding portion thereof, comprising:

(a) a heavy chain variable region CDR1 comprising SEQ ID NO:15;

(b) a heavy chain variable region CDR2 comprising SEQ ID NO:21;

(c) a heavy chain variable region CDR3 comprising SEQ ID NO:27;

(d) a light chain variable region CDR1 comprising SEQ ID NO:33;

(e) a light chain variable region CDR2 comprising SEQ ID NO:39; and

(f) a light chain variable region CDR3 comprising SEQ ID NO: 45;

or an antibody, or antigen-binding portion thereof, comprising:

(a) a heavy chain variable region CDR1 comprising SEQ ID NO:16;

(b) a heavy chain variable region CDR2 comprising SEQ ID NO:22;

(c) a heavy chain variable region CDR3 comprising SEQ ID NO:28;

(d) a light chain variable region CDR1 comprising SEQ ID NO:34;

(e) a light chain variable region CDR2 comprising SEQ ID NO:40; and

(f) a light chain variable region CDR3 comprising SEQ ID NO:46;

or an antibody, or antigen-binding portion thereof, comprising:

(a) a heavy chain variable region CDR1 comprising SEQ ID NO:17;

(b) a heavy chain variable region CDR2 comprising SEQ ID NO:23;

(c) a heavy chain variable region CDR3 comprising SEQ ID NO:29 or 75;

(d) a light chain variable region CDR1 comprising SEQ ID NO:35;

(e) a light chain variable region CDR2 comprising SEQ ID NO:41; and

(f) a light chain variable region CDR3 comprising SEQ ID NO:47;

or an antibody, or antigen-binding portion thereof, comprising:

(a) a heavy chain variable region CDR1 comprising SEQ ID NO:18;

(b) a heavy chain variable region CDR2 comprising SEQ ID NO:24;

(c) a heavy chain variable region CDR3 comprising SEQ ID NO:30;

(d) a light chain variable region CDR1 comprising SEQ ID NO:36;

(e) a light chain variable region CDR2 comprising SEQ ID NO:42; and

(f) a light chain variable region CDR3 comprising SEQ ID NO:48, linked to a cytotoxin; and

(iii) an immunoconjugate comprising an antibody, or antigen-binding portion thereof, that binds to the same epitope that is recognized by (e.g., cross-competes for binding to human CD70 with) an antibody comprising:

(a) a heavy chain variable region comprising the amino acid sequence of SEQ ID NO:1 and a light chain variable region comprising the amino acid sequence of SEQ ID NO:7;

(b) a heavy chain variable region comprising the amino acid sequence of SEQ ID NO:2 and a light chain variable region comprising the amino acid sequence of SEQ ID NO:8;

(c) a heavy chain variable region comprising the amino acid sequence of SEQ ID NO:3 and a light chain variable region comprising the amino acid sequence of SEQ ID NO:9;

(d) a heavy chain variable region comprising the amino acid sequence of SEQ ID NO:4 and a light chain variable region comprising the amino acid sequence of SEQ ID NO:10;

(e) a heavy chain variable region comprising the amino acid sequence of SEQ ID NO:5 or 73 and a light chain variable region comprising the amino acid sequence of SEQ ID NO:11; and

(f) a heavy chain variable region comprising the amino acid sequence of SEQ ID NO:6 and a light chain variable region comprising the amino acid sequence of SEQ ID NO:12, linked to a cytotoxin.

This disclosure also provides a bispecific molecule comprising an antibody, or antigen-binding portion thereof, of this disclosure, linked to a second functional moiety having a different binding specificity than said antibody, or antigen binding portion thereof.

Compositions comprising an antibody, or antigen-binding portion thereof, or immunoconjugate or bispecific molecule of this disclosure and a pharmaceutically acceptable carrier are also provided.

Nucleic acid molecules encoding the antibodies, or antigen-binding portions thereof, of this disclosure are also encompassed by this disclosure, as well as expression vectors comprising such nucleic acids and host cells comprising such expression vectors. Methods for preparing anti-CD70 antibodies using the host cells comprising such expression vectors are also provided and may include the steps of (i) expressing the antibody in the host cell and (ii) isolating the antibody from the host cell.

In yet another aspect, the invention pertains to a method for preparing an anti-CD70 antibody. The method comprises:
(a) providing: (i) a heavy chain variable region antibody sequence comprising a CDR1 sequence selected from the group consisting of SEQ ID NOs: 13-18, a CDR2 sequence selected from the group consisting of SEQ ID NOs: 19-24, and/or a CDR3 sequence selected from the group consisting of SEQ ID NOs: 25-30 and 75; and/or (ii) a light chain variable region antibody sequence comprising a CDR1 sequence selected from the group consisting of SEQ ID NOs: 31-36, a CDR2 sequence selected from the group consisting of SEQ ID NOs:37-42, and/or a CDR3 sequence selected from the group consisting of SEQ ID NOs:43-48;
(b) altering at least one amino acid residue within the heavy chain variable region antibody sequence and/or the light chain variable region antibody sequence to create at least one altered antibody sequence; and
(c) expressing the altered antibody sequence as a protein.

The present disclosure also provides isolated anti-CD70 antibody-partner molecule conjugates that specifically bind to CD70 with high affinity, particularly those comprising human monoclonal antibodies. Certain of such antibody-partner molecule conjugates are capable of being internalized into CD70-expressing cells and are capable of mediating antibody dependent cellular cytotoxicity. This disclosure also provides methods for treating cancers, such as renal cell carcinoma cancer or lymphoma, using an anti-CD70 antibody-partner molecule conjugate disclosed herein.

Compositions comprising an antibody, or antigen-binding portion thereof, conjugated to a partner molecule of this disclosure are also provided. Partner molecules that can be advantageously conjugated to an antibody in an antibody partner molecule conjugate as disclosed herein include, but are not limited to, molecules as drugs, toxins, marker molecules (e.g., radioisotopes), proteins and therapeutic agents. Compositions comprising antibody-partner molecule conjugates and pharmaceutically acceptable carriers are also disclosed herein.

In one aspect, such antibody-partner molecule conjugates are conjugated via chemical linkers. In some embodiments, the linker is a peptidyl linker, and is depicted herein as (L⁴)ₚ-F-(L¹)ₘ. Other linkers include hydrazine and disulfide linkers, and is depicted herein as (L⁴)ₚ-H-(L¹)ₘ or (L⁴)ₚ-J-(L¹)ₘ, respectively. In addition to the linkers as being attached to the partner, the present invention also provides cleavable linker arms that are appropriate for attachment to essentially any molecular species.

In another aspect, the invention pertains to a method of inhibiting growth of a CD70-expressing tumor cell. The method comprises contacting the CD70-expressing tumor cell with an antibody-partner molecule conjugate of the disclosure such that growth of the CD70-tumor cell is inhibited. In a preferred embodiment, the partner molecule is a therapeutic agent, such as a cytotoxin. Particularly preferred CD70-expressing tumor cells are renal cancer cells and lymphoma cells.

In another aspect, the invention pertains to a method of treating cancer in a subject. The method comprises administering to the subject an antibody-partner molecule conjugate of the disclosure such that the cancer is treated in the subject. In a preferred embodiment, the partner molecule is a therapeutic agent, such as a cytotoxin. Particularly preferred cancers for treatment are renal cancer and lymphoma.

In another aspect, the invention pertains to a method of treating an autoimmune disease, inflammation, or a viral infection in a subject. The method comprises administering to the subject an antibody-partner molecule conjugate of the disclosure such that the autoimmune disorder is treated in the subject.

Other features and advantages of the instant disclosure will be apparent from the following detailed description and examples which should not be construed as limiting. The contents of all references, Genbank entries, patents and published patent applications cited throughout this application are expressly incorporated herein by reference.

### BRIEF DESCRIPTION OF THE DRAWINGS

FIG. 1A shows the nucleotide sequence (SEQ ID NO:49) and amino acid sequence (SEQ ID NO:1) of the heavy chain variable region of the 2H5 human monoclonal antibody. The CDR1 (SEQ ID NO:13), CDR2 (SEQ ID NO:19) and CDR3 (SEQ ID NO:25) regions are delineated and the V and J germline derivations are indicated.

FIG. 1B shows the nucleotide sequence (SEQ ID NO:55) and amino acid sequence (SEQ ID NO:7) of the light chain variable region of the 2H5 human monoclonal antibody. The CDR1 (SEQ ID NO:31), CDR2 (SEQ ID NO:37) and CDR3 (SEQ ID NO:43) regions are delineated and the V and J germline derivations are indicated.

FIG. 2A shows the nucleotide sequence (SEQ ID NO:50) and amino acid sequence (SEQ ID NO:2) of the heavy chain variable region of the 10B4 human monoclonal antibody. The CDR1 (SEQ ID NO:14), CDR2 (SEQ ID NO:20) and CDR3 (SEQ ID NO:26) regions are delineated and the V, D, and J germline derivations are indicated.

FIG. 2B shows the nucleotide sequence (SEQ ID NO:56) and amino acid sequence (SEQ ID NO:8) of the light chain variable region of the 10B4 human monoclonal antibody. The CDR1 (SEQ ID NO:32), CDR2 (SEQ ID NO:38) and CDR3 (SEQ ID NO:44) regions are delineated and the V and J germline derivations are indicated.

FIG. 3A shows the nucleotide sequence (SEQ ID NO: 51) and amino acid sequence (SEQ ID NO:3) of the heavy chain variable region of the 8B5 human monoclonal antibody. The CDR1 (SEQ ID NO:15), CDR2 (SEQ ID NO:21) and CDR3 (SEQ ID NO:27) regions are delineated and the V, D and J germline derivations are indicated.

FIG. 3B shows the nucleotide sequence (SEQ ID NO:57) and amino acid sequence (SEQ ID NO:9) of the light chain variable region of the 8B5 human monoclonal antibody. The CDR1 (SEQ ID NO:33), CDR2 (SEQ ID NO:39) and CDR3 (SEQ ID NO:45) regions are delineated and the V and J germline derivations are indicated.

FIG. 4A shows the nucleotide sequence (SEQ ID NO:52) and amino acid sequence (SEQ ID NO:4) of the heavy chain variable region of the 18E7 human monoclonal antibody. The CDR1 (SEQ ID NO:16), CDR2 (SEQ ID NO:22) and CDR3 (SEQ ID NO:28) regions are delineated and the V, D and J germline derivations are indicated.

FIG. 4B shows the nucleotide sequence (SEQ ID NO:58) and amino acid sequence (SEQ ID NO:10) of the light chain variable region of the 18E7 human monoclonal antibody. The CDR1 (SEQ ID NO:34), CDR2 (SEQ ID NO:40) and CDR3 (SEQ ID NO:46) regions are delineated and the V and J germline derivations are indicated.

FIG. 5A shows the nucleotide sequence (SEQ ID NO:53) and amino acid sequence (SEQ ID NO:5) of the heavy chain variable region of the 69A7 human monoclonal antibody. The CDR1 (SEQ ID NO:17), CDR2 (SEQ ID NO:23) and CDR3 (SEQ ID NO:29) regions are delineated and the V, D and J germline derivations are indicated.

FIG. 5B shows the nucleotide sequence (SEQ ID NO:59) and amino acid sequence (SEQ ID NO:11) of the light chain variable region of the 69A7 human monoclonal antibody. The CDR1 (SEQ ID NO:35), CDR2 (SEQ ID NO:41) and CDR3 (SEQ ID NO:47) regions are delineated and the V and J germline derivations are indicated.

FIG. 6A shows the nucleotide sequence (SEQ ID NO:54) and amino acid sequence (SEQ ID NO:6) of the heavy chain variable region of the 1F4 human monoclonal antibody. The CDR1 (SEQ ID NO:18), CDR2 (SEQ ID NO:24) and CDR3 (SEQ ID NO:30) regions are delineated and the V, D and J germline derivations are indicated.

FIG. 6B shows the nucleotide sequence (SEQ ID NO:60) and amino acid sequence (SEQ ID NO:12) of the light chain variable region of the 1F4 human monoclonal antibody. The CDR1 (SEQ ID NO:36), CDR2 (SEQ ID NO:42) and CDR3 (SEQ ID NO:48) regions are delineated and the V and J germline derivations are indicated.

FIG. 7 shows the alignment of the amino acid sequence of the heavy chain variable region of 2H5 and 10B4 with the human germline V_{H} 3-30.3 amino acid sequence (SEQ ID NO:61).

FIG. 8 shows the alignment of the amino acid sequence of the heavy chain variable region of 8B5 and 18E7 with the human germline V_{H} 3-33 amino acid sequence (SEQ ID NO:62).

FIG. 9 shows the alignment of the amino acid sequence of the heavy chain variable region of 69A7 with the human germline V_{H} 4-61 amino acid sequence (SEQ ID NO:63).

FIG. 10 shows the alignment of the amino acid sequence of the heavy chain variable region of 1F4 with the human germline V_{H} 3-23 amino acid sequence (SEQ ID NO:64).

FIG. 11 shows the alignment of the amino acid sequence of the light chain variable region of 2H5 with the human germline Vₖ L6 amino acid sequence (SEQ ID NO:65).

FIG. 12 shows the alignment of the amino acid sequence of the light chain variable region of 10B4 with the human germline Vₖ L18 amino acid sequence (SEQ ID NO:66).

FIG. 13 shows the alignment of the amino acid sequence of the light chain variable region of 8B5 and 18E7 with the human germline Vₖ L15 amino acid sequence (SEQ ID NO:67).

FIG. 14 shows the alignment of the amino acid sequence of the light chain variable region of 69A7 with the human germline Vₖ L6 amino acid sequence (SEQ ID NO:65).

FIG. 15 shows the alignment of the amino acid sequence of the light chain variable region of 1F4 with the human germline Vₖ A27 amino acid sequence (SEQ ID NO:68).

FIG. 16 shows the results of ELISA experiments demonstrating that human monoclonal antibodies against human CD70 specifically bind to CD70.

FIG. 17 shows the results of flow cytometry experiments demonstrating that the anti-CD70 human monoclonal antibody 2H5 binds to renal carcinoma cell lines.

FIGS. 18A and B show the results of flow cytometry experiments demonstrating that human monoclonal antibodies against human CD70 bind in a concentration dependent manner to renal cell carcinoma (RCC) cell lines. (A) 786-O RCC cell line (B) A498 RCC cell line.

FIG. 18C shows the results of flow cytometry experiments demonstrating that human monoclonal antibodies against human CD70 bind to the renal carcinoma cell line 786-O.

FIG. 18D shows the results of flow cytometry experiments demonstrating that the HuMAb 69A7 antibody against human CD70 binds in a concentration dependent manner to renal cell carcinoma (RCC) cell line 786-O.

FIG. 19 shows the results of flow cytometry experiments demonstrating that the anti-CD70 human monoclonal antibody 2H5 binds to human lymphoma cell lines.

FIGS. 20A and B show the results of flow cytometry experiments demonstrating that the anti-CD70 human monoclonal antibody 2H5 binds to human lymphoma cell lines in a concentration dependent manner. (A) Raji lymphoma cell line (B) Granta-519 lymphoma cell line.

FIG. 20C shows the results of flow cytometry experiments demonstrating that human monoclonal antibodies against human CD70 bind to the Raji lymphoma cell line.

FIG. 20D shows the results of a competition flow cytometry assay demonstrating that the HuMAbs 2H5 and 69A7 share a similar binding epitope.

FIG. 20E shows the results of flow cytometry experiments demonstrating that human monoclonal antibodies against human CD70 bind to the Daudi lymphoma cell line and 786-O renal carcinoma cell line.

FIG. 21 shows the results of Hum-Zap internalization experiments demonstrating that human monoclonal antibodies against human CD70 can internalize into CD70+ cells.

FIGS. 22A-C show the results of cell proliferation assays demonstrating that cytotoxin-conjugated human monoclonal anti-CD70 antibodies kill renal cell carcinoma cell (RCC) lines. (A) Caki-2 RCCs (B) 786-O RCCs (C) ACHN RCCs.

FIGS. 23A-D show the results of ADCC assays demonstrating that human monoclonal anti-CD70 antibodies kill human leukemia and lymphoma cell lines in an ADCC dependent manner. (A) ARH-77 leukemia cell line (B) HuT 78 lymphoma cell line (C) Raji lymphoma cell line and (D) L-540 cell line which does not express CD70.

FIG. 24 shows the results of a cell proliferation assay demonstrating that cytotoxin-conjugated human monoclonal anti-CD70 antibodies kill human lymphoma cell lines.

FIGS. 25A-B show the results of a cell proliferation assay demonstrating that cytotoxin-conjugated human monoclonal anti-CD70 antibodies show cytotoxicity to Raji cells (A) with a three-hour wash and (B) with a continuous wash.

FIGS. 26A-B show the results of an in vivo mouse tumor model study demonstrating that treatment with the cytotoxin-conjugated anti-CD70 antibody 2H5 has a direct inhibitory effect on renal cell carcinoma (RCC) tumors in vivo. (A) A-498 RCC tumors (B) ACHN RCC tumors.

FIGS. 27A-F show the results of an ADCC assay demonstrating that nonfucosylated human monoclonal anti-CD70 antibodies have increased cell cytotoxicity on human leukemia cells in an ADCC dependent manner. (A) ARH-77 cells; (B) MEC-1 cells; (C) MEC-1 cells treated with anti-CD16 antibody; (D) SU-DHL-6 cells; (E) IM-9 cells; (F) HuT 78 cells.

FIG. 28 shows the results of an ADCC assay demonstrating that human monoclonal anti-CD70 antibodies kill human leukemia cells in an ADCC concentration-dependent manner.

FIG. 29 shows the results of an antibody dependent cellular cytotoxicity (ADCC) assay demonstrating that human monoclonal anti-CD70 antibodies kill human leukemia cells in an ADCC dependent manner, but cytotoxicity is dependent upon CD16.

FIG. 30 shows the results of an ADCC assay demonstrating that human monoclonal anti-CD70 antibodies kill human activated T cells and the effect is reversed with the addition of anti-CD16 antibody.

FIG. 31 shows the results of a blocking assay demonstrating that some human monoclonal anti-CD70 antibodies block binding of CD70 to CD27 and other human monoclonal anti-CD70 antibodies do not block binding of CD70 to CD27.

FIGS. 32A-B show the results of an in vivo mouse tumor model study demonstrating that treatment with naked anti-CD70 antibody 2H5 has a direct inhibitory effect on lymphoma tumors in vivo. (A) Raji tumors; (B) ARH-77 tumors.

FIGS. 33A-C show the results of an in vivo mouse tumor model study demonstrating that treatment with the cytotoxin-conjugated anti-CD70 antibody 2H5 has a direct inhibitory effect on lymphoma tumors in vivo. (A) ARH-77 tumors; (B) Granta 519 tumors; (C) Raji tumors.

FIG. 34 shows the results of a study showing that the anti-CD70 antibody 69A7 cross-reacts with CD70 expressed on a monkey rhesus CD70+ B lymphoma cell line.

FIG. 35 shows the results of a blocking assay demonstrating that a human anti-CD70 antibody blocks the binding of a known mouse anti-human CD70 antibody.

FIGS. 36A and B show the results of treatment with either anti-CD70 antibody or the non-fucosylated form of the antibody. (A) Anti-CD70 antibodies inhibit CD70 co-stimulated cell proliferation in a dose dependent manner. (B) Anti-CD70 antibodies inhibit CD70 co-stimulated IFN-γ secretion in a dose dependent manner.

FIGS. 37A-C show the results of treatment with either anti-CD70 antibody or the non-fucosylated form of the antibody on peptide stimulated cells. (A) Anti-CD70 antibodies inhibit peptide specific CD8+ T cell expansion. (B) There was no significant reduction of total cell viability observed. (C) There was no significant reduction of total CD8+ cell numbers observed.

FIG. 38 shows that the effect of anti-CD70 antibodies on peptide specific CD8+ T cell expansion is blocked by addition of anti-CD16 antibodies.

FIGS. 39A-B show the results of an in vivo mouse tumor model study demonstrating that treatment with the cytotoxin-conjugated anti-CD70 antibody 2H5 has a direct inhibitory effect on renal carcinoma tumors in vivo. (A) 786-O tumors; (B) Caki-1 tumors.

FIG. 40 shows the in vivo efficacy of immunoconjugates anti-CD70-N1 and anti-CD70-N2 against tumor formation in a 786-O renal cell carcinoma xenograft NOD-SCID mouse model.

FIG. 41 shows the in vivo efficacy of a single dose of immunoconjugate anti-CD70-N2 against tumor formation in a 786-O renal cell carcinoma xenograft SCID mouse model.

FIG. 42 shows the in vivo efficacy of various doses of immunoconjugate anti-CD70-N2 against tumor formation in a 786-O renal cell carcinoma xenograft SCID mouse model.

FIG. 43 shows the in vivo efficacy of various doses of immunoconjugate anti-CD70-N2 against tumor formation in a Caki-1 renal cell carcinoma xenograft SCID mouse model.

FIG. 44 shows the in vivo efficacy of immunoconjugate anti-CD70-N2 against tumor formation in a Raji cell lymphoma SCID mouse model.

FIG. 45 shows the in vivo safety of immunoconjugate anti-CD70-N2 in BALB/c mice.

FIG. 46A-D shows the in vivo safety of immunoconjugate anti-CD70-N2 as compared to free drug in dogs.

FIG. 47 shows the results of an ADCC assay. hIgG1nf Neg Ctrl=human IgG1 NF negative control Ab. hIgG1 Neg Ctrl=human IgG1 negative control Ab. mIgG1 Neg Ctrl=mouse IgG1 negative control Ab (A) FACS analysis of 2H5 binding to activated B cells. (B) ADCC assay of 2H5 NF and 2H5 on activated human B cells. (C) ADCC assay with the addition of anti-CD16 Ab.

FIG. 48 depicts the capability of anti-CD70 antibodies to mediate lysis of Ag activated, CD70+ human T cells via ADCC by effector cells naturally present in stimulated human PBMC cultures.

FIG. 49 depicts binding characteristics of anti-CD70 antibodies to natively expressing CD70+ human cancer cell line 786-O cells.

FIG. 50 depicts the ability of fucosylated and non-fucosylated anti-CD70 antibodies to mediate ADCC on the CD70+ lymphoma cell line ARH77.

FIG. 51 shows the in vivo efficacy of a single dose of anti-CD70-cytotoxin E against tumor formation in a 786-O renal cell carcinoma xenograft SCID mouse model.

FIG. 52 shows the in vivo efficacy of a single dose of anti-CD70-cytotoxin E against tumor formation in a A498 renal cell carcinoma xenograft SCID mouse model.

FIG. 53 shows the in vivo efficacy of a single dose of anti-CD70-cytotoxin E against tumor formation in a Caki-1 renal cell carcinoma xenograft SCID mouse model.

FIG. 54 shows the in vivo efficacy of a single dose of anti-CD70-cytotoxin E against tumor formation in a Raji cell lymphoma SCID mouse model.

FIG. 55 shows the in vivo efficacy of a single dose of anti-CD70-cytotoxin E against tumor formation in a Daudi cell lymphoma SCID mouse model.

FIG. 56 shows the in vivo efficacy of anti-CD70-cytotoxin E against tumor formation in a Caki-1 renal cell carcinoma xenograft rat model.

FIG. 57 shows the in vivo safety of anti-CD70-cytotoxin E in BALB/c mice.

FIG. 58 shows the in vivo safety of anti-CD70-cytotoxin E in dogs.

FIG. 59 shows the in vivo safety of anti-CD70-cytotoxin E in monkeys.

FIG. 60 shows the in vivo efficacy of a single dose of anti-CD70-cytotoxin F against tumor formation in a 786-O renal cell carcinoma xenograft SCID mouse model.

FIG. 61 shows the in vivo efficacy of a single dose of anti-CD70-cytotoxin F against tumor formation in a Caki-1 renal cell carcinoma xenograft SCID mouse model.

FIG. 62 shows the in vivo efficacy of a single dose of anti-CD70-cytotoxin F against tumor formation in a Raji cell lymphoma SCID mouse model.

FIG. 63 shows the in vivo efficacy of a single dose of anti-CD70-cytotoxin G against tumor formation in a 786-O renal cell carcinoma xenograft SCID mouse model.

FIG. 64 shows the in vivo efficacy of a single dose of anti-CD70-cytotoxin G against tumor formation in a Caki-1 renal cell carcinoma xenograft SCID mouse model.

FIG. 65 shows the in vivo efficacy of a single dose of anti-CD70-cytotoxin H against tumor formation in a A498 renal cell carcinoma xenograft SCID mouse model.

FIG. 66 shows the in vivo efficacy of a single dose of anti-CD70-cytotoxin H against tumor formation in a Caki-1 renal cell carcinoma xenograft SCID mouse model.

FIG. 67 shows the in vivo efficacy of a single dose of anti-CD70-cytotoxin I against tumor formation in a 786-O renal cell carcinoma xenograft SCID mouse model.

FIG. 68 shows the in vivo efficacy of a single dose of anti-CD70-cytotoxin I against tumor formation in Caki-1 renal cell carcinoma xenograft rat model.

FIG. 69 shows the in vivo efficacy of a single dose of anti-CD70-cytotoxin J against tumor formation in a 786-O renal cell carcinoma xenograft SCID mouse model.

FIG. 70 shows anti-CD70 antibody 2H5 functional blocking of CD70 stimulated human T cell proliferation.

FIG. 71 is the structure of cytotoxin B.

FIG. 72 is the structure of cytotoxin C.

FIG. 73 is the structure of cytotoxin D.

FIG. 74 is the structure of cytotoxin E.

FIG. 75 is the structure of cytotoxin F.

FIG. 76 is the structure of cytotoxin G.

FIG. 77 is the structure of cytotoxin H.

FIG. 78 is the structure of cytotoxin I.

FIG. 79 is the structure of cytotoxin J.

### DETAILED DESCRIPTION

The present disclosure relates to isolated monoclonal antibodies, particularly human monoclonal antibodies, which bind to human CD70 and that have desirable functional properties. In certain embodiments, the antibodies of this disclosure are derived from particular heavy and light chain germline sequences and/or comprise particular structural features such as CDR regions comprising particular amino acid sequences. This disclosure provides isolated antibodies, methods of making such antibodies, antibody-partner molecule conjugates, and bispecific molecules comprising such antibodies and pharmaceutical compositions containing the antibodies, antibody-partner molecule conjugates or bispecific molecules of this disclosure. This disclosure also relates to methods of using the antibodies, such as to detect CD70 protein, as well as to methods of using the anti-CD70 antibodies of the invention to inhibit the growth of CD70-expressing cells, such as tumor cells. Accordingly, this disclosure also provides methods of using the anti-CD70 antibodies and antibody-partner molecule conjugates of this disclosure to treat various types of cancer, for example, renal cell carcinoma or lymphoma.

In order that the present disclosure may be more readily understood, certain terms are first defined. Additional definitions are set forth throughout the detailed description.

As used herein, the term “CD70” includes variants, isoforms, homologs, orthologs and paralogs. For example, antibodies specific for a human CD70 protein may, in certain cases, cross-react with a CD70 protein from a species other than human. In other embodiments, the antibodies specific for a human CD70 protein may be completely specific for the human CD70 protein and may not exhibit species or other types of cross-reactivity, or may cross-react with CD70 from certain other species but not all other species (e.g., cross-react with a primate CD70 but not mouse CD70). The term “human CD70” refers to human sequence CD70, such as the complete amino acid sequence of human CD70 having Genbank Accession Number P32970 (SEQ ID NO:76). The term “mouse CD70” refers to mouse sequence CD70, such as the complete amino acid sequence of mouse CD70 having Genbank Accession Number NP_{—}035747. The human CD70 sequence may differ from human CD70 of Genbank Accession Number P32970 by having, for example, conserved mutations or mutations in non-conserved regions and the CD70 has substantially the same biological function as the human CD70 of Genbank Accession Number P32970. For example, one biological function of human CD70 is binding to cytokine receptor CD27.

A particular human CD70 sequence will generally be at least 90% identical in amino acids sequence to human CD70 of Genbank Accession Number P32970 and contains amino acid residues that identify the amino acid sequence as being human when compared to CD70 amino acid sequences of other species (e.g., murine). In certain cases, a human CD70 may be at least 95%, or even at least 96%, 97%, 98%, or 99% identical in amino acid sequence to CD70 of Genbank Accession Number P32970. In certain embodiments, a human CD70 sequence will display no more than 10 amino acid differences from the CD70 sequence of Genbank Accession Number P32970. In certain embodiments, the human CD70 may display no more than 5, or even no more than 4, 3, 2, or 1 amino acid difference from the CD70 sequence of Genbank Accession Number P32970. Percent identity can be determined as described herein.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

A “signal transduction pathway” refers to the biochemical relationship between a variety of signal transduction molecules that play a role in the transmission of a signal from one portion of a cell to another portion of a cell. As used herein, the phrase “cell surface receptor” includes, for example, molecules and complexes of molecules capable of receiving a signal and the transmission of such a signal across the plasma membrane of a cell. An example of a “cell surface receptor” of the present disclosure is the CD70 receptor.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L} or Vₖ) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antibody fragment” and “antigen-binding portion” of an antibody (or simply “antibody portion”), as used herein, refer to one or more fragments of an antibody that retain the ability to specifically bind to an antigen (e.g., CD70). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3^{rd} ed. 1993); (iv) a Fd fragment consisting of the V_{H} and C_{H1} domains; (v) a Fv fragment consisting of the V_{L} and V_{H} domains of a single arm of an antibody, (vi) a dAb fragment (Ward et al., (1989) Nature 341:544-546), which consists of a V_{H} domain; (vii) an isolated complementarity determining region (CDR); and (viii) a nanobody, a heavy chain variable region containing a single variable domain and two constant domains. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds CD70 is substantially free of antibodies that specifically bind antigens other than CD70). An isolated antibody that specifically binds CD70 may, however, have cross-reactivity to other antigens, such as CD70 molecules from other species. In certain embodiments, an isolated antibody specifically binds to human CD70 and does not cross-react with other non-human CD70 antigens. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies may include later modifications, including natural or synthetic modifications. The human antibodies of this disclosure may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “antibody mimetic” is intended to refer to molecules capable of mimicking an antibody's ability to bind an antigen, but which are not limited to native antibody structures. Examples of such antibody mimetics include, but are not limited to, Affibodies, DARPins, Anticalins, Avimers, and Versabodies, all of which employ binding structures that, while they mimic traditional antibody binding, are generated from and function via distinct mechanisms.

As used herein, the term “partner molecule” refers to the entity which is conjugated to an antibody in an antibody-partner molecule conjugate. Examples of partner molecules include drugs, toxins, marker molecules (e.g., including, but not limited to peptide and small molecule markers such as fluorochrome markers, as well as single atom markers such as radioisotopes), proteins and therapeutic agents.

As used herein, an antibody that “specifically binds to human CD70” is intended to refer to an antibody that binds to human CD70 with a K_{D} of 5×10⁻⁸ M or less, more preferably 1×10⁻⁸ M or less, more preferably 6×10⁻⁹ M or less, more preferably 3×10⁻⁹ M or less, even more preferably 2×10⁻⁹ M or less.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore® system.

As used herein, the term “high affinity” for an IgG antibody refers to an antibody having a K_{D} of 1×10⁻⁷ M or less, more preferably 1×10⁻⁸ M or less, more preferably 1×10⁻⁹ M or less, and even more preferably 1×10⁻¹⁰ M or less for a target antigen. However, “high affinity” binding can vary for other antibody isotypes. For example, “high affinity” binding for an IgM isotype refers to an antibody having a K_{D} of 1×10⁻⁷ M or less, more preferably 1×10⁻⁸ M or less, even more preferably 1×10⁻⁹M or less.

The term “does not substantially bind” to a protein or cells, as used herein, means does not bind or does not bind with a high affinity to the protein or cells, i.e., binds to the protein or cells with a K_{D} of 1×10⁻⁶ M or more, more preferably 1×10⁻⁵ M or more, more preferably 1×10⁻⁴ M or more, more preferably 1×10⁻³ M or more, even more preferably 1×10⁻² M or more.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, fish, reptiles, etc.

The symbol “-”, whether utilized as a bond or displayed perpendicular to a bond, indicates the point at which the displayed moiety is attached to the remainder of the molecule, solid support, etc.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si, and S, and wherein the nitrogen, carbon and sulfur atoms may optionally be oxidized and the nitrogen heteroatom may optionally be quaternized. The heteroatom(s) O, N, S, and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). The terms “heteroalkyl” and “heteroalkylene” encompass poly(ethylene glycol) and its derivatives (see, for example, Shearwater Polymers Catalog, 2001). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The term “lower” in combination with the terms “alkyl” or “heteroalkyl” refers to a moiety having from 1 to 6 carbon atoms.

The terms “alkoxy,” “alkylamino,” “alkylsulfonyl,” and “alkylthio” (or thioalkoxy) are used in their conventional sense, and refer to those alkyl groups attached to the remainder of the molecule via an oxygen atom, an amino group, an SO₂ group or a sulfur atom, respectively. The term “arylsulfonyl” refers to an aryl group attached to the remainder of the molecule via an SO₂ group, and the term “sulfhydryl” refers to an SH group.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of substituted or unsubstituted “alkyl” and substituted or unsubstituted “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The heteroatoms and carbon atoms of the cyclic structures are optionally oxidized.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a substituted or unsubstituted polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen, carbon and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. “Aryl” and “heteroaryl” also encompass ring systems in which one or more non-aromatic ring systems are fused, or otherwise bound, to an aryl or heteroaryl system.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroalkyl substituents,” respectively, and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5, 6, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆) alkyl.

As used herein, the term “diphosphate” includes but is not limited to an ester of phosphoric acid containing two phosphate groups. The term “triphosphate” includes but is not limited to an ester of phosphoric acid containing three phosphate groups. For example, particular drugs having a diphosphate or a triphosphate include:

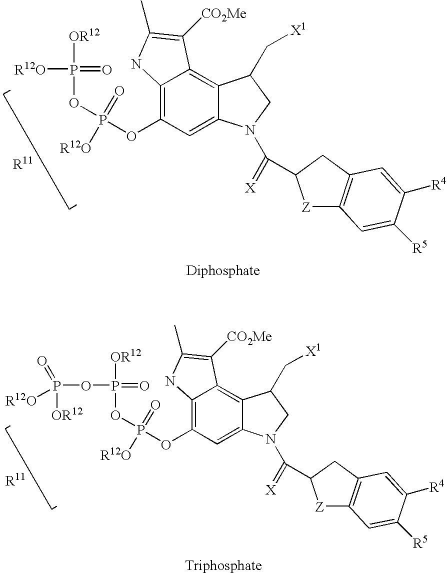

As used herein, the term “heteroatom” includes oxygen (O), nitrogen (N), sulfur (S) and silicon (Si).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocyclyl groups.

Various aspects of this disclosure are described in further detail in the following subsections.

### Anti-CD70 Antibodies Having Particular Functional Properties

The antibodies of this disclosure are characterized by particular functional features or properties of the antibodies. For example, the antibodies specifically bind to human CD70, such as human CD70 expressed on the surface of the cell. Preferably, an antibody of this disclosure binds to CD70 with high affinity, for example with a K_{D} of 1×10⁻⁷ M or less, more preferably with a K_{D} of 5×10⁻⁸ M or less and even more preferably with a K_{D} of 1×10⁻⁸ M or less. Standard assays to evaluate the binding ability of the antibodies toward CD70 are known in the art, including for example, ELISAs, Western blots and RIAs. Suitable assays are described in detail in the Examples. The binding kinetics (e.g., binding affinity) of the antibodies also can be assessed by standard assays known in the art, such as by ELISA, Scatchard and Biacore analysis. As another example, the antibodies of the present disclosure may bind to a renal carcinoma tumor cell line, for example, the 786-O, A-498, ACHN, Caki-1 or Caki-2 cell lines. As yet another example, the antibodies of the present disclosure may bind to a B-cell tumor cell line, for example, the Daudi, HuT 78, Raji or Granta-519 cell lines.

An anti-CD70 antibody of this disclosure binds to human CD70 and preferably exhibits one or more of the following properties:

(a) binds to human CD70 with a K_{D} of 1×10⁻⁷ M or less; and

(b) binds to a renal cell carcinoma tumor cell line;

(c) binds to a lymphoma cell line, e.g., a B-cell tumor cell line;

(d) is internalized by CD70-expressing cells;

(e) exhibits antibody dependent cellular cytotoxicity (ADCC) against CD70-expressing cells; and

(f) inhibits growth of CD70-expressing cells in vivo when conjugated to a cytotoxin.

Preferably, the antibody exhibits at least two of properties (a), (b), (c), (d), (e), and (f). More preferably, the antibody exhibits at least three of properties (a), (b), (c), (d), (e), and (f). More preferably, the antibody exhibits four of properties (a), (b), (c), (d), (e), and (f). Even more preferably, the antibody exhibits five of properties (a), (b), (c), (d), (e), and (f). Even more preferably, the antibody exhibits all six properties (a), (b), (c), (d), (e), and (f).

In another preferred embodiment, the antibody binds to CD70 with an affinity of 5×10⁻⁹ M or less. In yet another preferred embodiment, the antibody inhibits growth of CD70-expressing tumor cells in vivo when the antibody is conjugated to a cytotoxin.

The binding of an antibody of the invention to CD70 can be assessed using one or more techniques well established in the art. For example, in a preferred embodiment, an antibody can be tested by a flow cytometry assay in which the antibody is reacted with a cell line that expresses human CD70, such as CHO cells that have been transfected to express CD70 on their cell surface or CD70-expressing cell lines such as 786-O, A498, ACHN, Caki-1, and/or Caki-2 (see, e.g., Examples 4 and 5 for a suitable assay and further description of cell lines). Additionally or alternatively, the binding of the antibody, including the binding kinetics (e.g., K_{D} value) can be tested in BIAcore binding assays. Still other suitable binding assays include ELISA assays, for example using a recombinant CD70 protein see, e.g., Example 1 for a suitable assay).

Preferably, an antibody of this disclosure binds to a CD70 protein with a K_{D} of 5×10⁻⁸ M or less, binds to a CD70 protein with a K_{D} of 3×10⁻⁸ M or less, binds to a CD70 protein with a K_{D} of 1×10⁻⁸ M or less, binds to a CD70 protein with a K_{D} of 7×10⁻⁹ M or less, binds to a CD70 protein with a K_{D} of 6×10⁻⁹ M or less or binds to a CD70 protein with a K_{D} of 5×10⁻⁹ M or less. The binding affinity of the antibody for CD70 can be evaluated, for example, by standard BIACORE analysis.

Standard assays for evaluating internalization of anti-CD70 antibodies by CD70-expressing cells are known in the art (see e.g., the Hum-ZAP and immunofluorescence assays described in Examples 7 and 21). Standard assays for evaluating binding of CD70 to CD27, and inhibition thereof by anti-CD70 antibodies, also are known in the art (see e.g., the assay described in Example 17). Standard assays for evaluating ADCC against CD70-expressing cells also are known in the art (see, e.g., the ADCC assay described in Example 9). Standard assays for evaluating inhibition of tumor cell growth in vivo by anti-CD70 antibodies, and cytotoxin conjugates thereof, also are known in the art (see, e.g., the tumor xenograft mouse models described in Examples 18, 19, 24-31 and 36-41).

Preferred antibodies of the invention are human monoclonal antibodies. Additionally or alternatively, the antibodies can be, for example, chimeric or humanized monoclonal antibodies.

### Monoclonal Antibodies 2H5, 10B4, 8B5, 18E7, 69A7, 69A7Y and 1F4

Exemplified antibodies of this disclosure include the human monoclonal antibodies 2H5, 10B4, 8B5, 18E7, 69A7, 69A7Y and 1F4 isolated and structurally characterized as described in Examples 1 and 2. The V_{H} amino acid sequences of 2H5, 10B4, 8B5, 18E7, 69A7, 69A7Y and 1F4 are shown in SEQ ID NOs:1, 2, 3, 4, 5, 73, and 6 respectively. The V_{L} amino acid sequences of 2H5, 10B4, 8B5, 18E7, 69A7, 69A7Y and 1F4 are shown in SEQ ID NOs:7, 8, 9, 10, 11, 11, and 12, respectively (69A7 and 69A7Y both have the V_{L} amino acid sequence of SEQ ID NO:11). Given that each of these antibodies can bind to CD70, the V_{H} and V_{L} sequences can be “mixed and matched” to create other anti-CD70 binding molecules of this disclosure. CD70 binding of such “mixed and matched” antibodies can be tested using the binding assays described above and in the Examples (e.g., FACS or ELISAs). Preferably, when V_{H} and V_{L} chains are mixed and matched, a V_{H} sequence from a particular V_{H}/V_{L} pairing is replaced with a structurally similar V_{H} sequence. Likewise, preferably a V_{L} sequence from a particular V_{H}/V_{L} pairing is replaced with a structurally similar V_{L} sequence.

Accordingly, in one aspect, this disclosure provides an isolated monoclonal antibody or antigen binding portion thereof comprising:

(a) a heavy chain variable region comprising an amino acid sequence selected from the group consisting of SEQ ID NOs:1, 2, 3, 4, 5, 6, and 73; and

(b) a light chain variable region comprising an amino acid sequence selected from the group consisting of SEQ ID NOs: 7, 8, 9, 10, 11, and 12;

wherein the antibody specifically binds to CD70.

Preferred heavy and light chain combinations include:

(a) a heavy chain variable region comprising the amino acid sequence of SEQ ID NO:1; and (b) a light chain variable region comprising the amino acid sequence of SEQ ID NO:7; or

(a) a heavy chain variable region comprising the amino acid sequence of SEQ ID NO:2; and (b) a light chain variable region comprising the amino acid sequence of SEQ ID NO:8; or

(a) a heavy chain variable region comprising the amino acid sequence of SEQ ID NO:3; and (b) a light chain variable region comprising the amino acid sequence of SEQ ID NO:9; or

(a) a heavy chain variable region comprising the amino acid sequence of SEQ ID NO:4; and (b) a light chain variable region comprising the amino acid sequence of SEQ ID NO:10; or

(a) a heavy chain variable region comprising the amino acid sequence of SEQ ID NO:5 or 73; and (b) a light chain variable region comprising the amino acid sequence of SEQ ID NO:11; or

(a) a heavy chain variable region comprising the amino acid sequence of SEQ ID NO:6; and (b) a light chain variable region comprising the amino acid sequence of SEQ ID NO:12.

In another aspect, this disclosure provides antibodies that comprise the heavy chain and light chain CDR1s, CDR2s and CDR3s of 2H5, 10B4, 8B5, 18E7, 69A7, 69A7Y and 1F4 or combinations thereof. The amino acid sequences of the V_{H} CDR1s of 2H5, 10B4, 8B5, 18E7, 69A7, 69A7Y and 1F4 are shown in SEQ ID NOs:13, 14, 15, 16, 17, 17 and 18, respectively (69A7 and 69A7Y both have the V_{H} CDR1 sequence of SEQ ID NO:17). The amino acid sequences of the V_{H} CDR2s of 2H5, 10B4, 8B5, 18E7, 69A7, 69A7Y and 1F4 are shown in SEQ ID NOs:19, 20, 21, 22, 23, 23 and 24, respectively (69A7 and 69A7Y both have the V_{H} CDR2 sequence shown in SEQ ID NO:23). The amino acid sequences of the V_{H} CDR3s of 2H5, 10B4, 8B5, 18E7, 69A7, 69A7Y and 1F4 are shown in SEQ ID NOs:25, 26, 27, 28, 29, 75, and 30, respectively.

The amino acid sequences of the Vₖ CDR1s of 2H5, 10B4, 8B5, 18E7, 69A7, 69A7Y and 1F4 are shown in SEQ ID NOs:31, 32, 33, 34, 35, 35 and 36, respectively (69A7 and 69A7Y both have the Vₖ CDR1 sequence shown in SEQ ID NO:35). The amino acid sequences of the Vₖ CDR2s of 2H5, 10B4, 8B5, 18E7, 69A7, 69A7Y and 1F4 are shown in SEQ ID NOs:37, 38, 39, 40, 41, 41 and 42, respectively (69A7 and 69A7Y both have the Vₖ CDR2 sequence shown in SEQ ID NO:41). The amino acid sequences of the Vₖ CDR3s of 2H5, 10B4, 8B5, 18E7, 69A7, 69A7Y and 1F4 are shown in SEQ ID NOs:43, 44, 45, 46, 47, 47 and 48, respectively (69A7 and 69A7Y both have the Vₖ CDR3 sequence shown in SEQ ID NO:47). The CDR regions are delineated using the Kabat system (Kabat, E. A., et al. (1991) Sequences of Proteins of Immunological Interest, Fifth Edition, U.S. Department of Health and Human Services, NIH Publication No. 91-3242).

Given that each of these antibodies can bind to CD70 and that antigen-binding specificity is provided primarily by the CDR1, CDR2 and CDR3 regions, the V_{H} CDR1, CDR2 and CDR3 sequences and Vₖ CDR1, CDR2 and CDR3 sequences can be “mixed and matched” (i.e., CDRs from different antibodies can be mixed and matched, although each antibody must contain a V_{H} CDR1, CDR2 and CDR3, and a Vₖ CDR1, CDR2 and CDR3) to create other anti-CD70 binding molecules of this disclosure. CD70 binding of such “mixed and matched” antibodies can be tested using the binding assays described above and in the Examples (e.g., FACS, ELISAs, Biacore analysis). Preferably, when V_{H} CDR sequences are mixed and matched, the CDR1, CDR2 and/or CDR3 sequence from a particular V_{H} sequence is replaced with a structurally similar CDR sequence(s). Likewise, when Vₖ CDR sequences are mixed and matched, the CDR1, CDR2 and/or CDR3 sequence from a particular Vₖ sequence preferably is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences disclosed herein for monoclonal antibodies 2H5, 10B4, 8B5, 18E7, 69A7, 69A7Y and 1F4.

Accordingly, in another aspect, this disclosure provides an isolated monoclonal antibody or antigen binding portion thereof comprising:

(a) a heavy chain variable region CDR1 comprising an amino acid sequence selected from the group consisting of SEQ ID NOs:13, 14, 15, 16, 17, and 18;

(b) a heavy chain variable region CDR2 comprising an amino acid sequence selected from the group consisting of SEQ ID NOs:19, 20, 21, 22, 23, and 24;

(c) a heavy chain variable region CDR3 comprising an amino acid sequence selected from the group consisting of SEQ ID NOs:25, 26, 27, 28, 29, 30, and 75;

(d) a light chain variable region CDR1 comprising an amino acid sequence selected from the group consisting of SEQ ID NOs:31, 32, 33, 34, 35, and 36;

(e) a light chain variable region CDR2 comprising an amino acid sequence selected from the group consisting of SEQ ID NOs:37, 38, 39, 40, 41, and 42; and

(f) a light chain variable region CDR3 comprising an amino acid sequence selected from the group consisting of SEQ ID NOs:43, 44, 45, 46, 47, and 48, wherein the antibody specifically binds CD70, preferably human CD70.

In a preferred embodiment, the antibody comprises:

(a) a heavy chain variable region CDR1 comprising SEQ ID NO:13;

(b) a heavy chain variable region CDR2 comprising SEQ ID NO:19;

(c) a heavy chain variable region CDR3 comprising SEQ ID NO:25;

(d) a light chain variable region CDR1 comprising SEQ ID NO:31;

(e) a light chain variable region CDR2 comprising SEQ ID NO:37; and

(f) a light chain variable region CDR3 comprising SEQ ID NO:43.

In another preferred embodiment, the antibody comprises:

(a) a heavy chain variable region CDR1 comprising SEQ ID NO:14;

(b) a heavy chain variable region CDR2 comprising SEQ ID NO:20;

(c) a heavy chain variable region CDR3 comprising SEQ ID NO:26;

(d) a light chain variable region CDR1 comprising SEQ ID NO:32;

(e) a light chain variable region CDR2 comprising SEQ ID NO:38; and

(f) a light chain variable region CDR3 comprising SEQ ID NO:44.

In another preferred embodiment, the antibody comprises:

(a) a heavy chain variable region CDR1 comprising SEQ ID NO:15;

(b) a heavy chain variable region CDR2 comprising SEQ ID NO:21;

(c) a heavy chain variable region CDR3 comprising SEQ ID NO:27;

(d) a light chain variable region CDR1 comprising SEQ ID NO:33;

(e) a light chain variable region CDR2 comprising SEQ ID NO:39; and

(f) a light chain variable region CDR3 comprising SEQ ID NO: 45.

In another preferred embodiment, the antibody comprises:

(a) a heavy chain variable region CDR1 comprising SEQ ID NO:16;

(b) a heavy chain variable region CDR2 comprising SEQ ID NO:22;

(c) a heavy chain variable region CDR3 comprising SEQ ID NO:28;

(d) a light chain variable region CDR1 comprising SEQ ID NO:34;

(e) a light chain variable region CDR2 comprising SEQ ID NO:40; and

(f) a light chain variable region CDR3 comprising SEQ ID NO:46.

In another preferred embodiment, the antibody comprises:

(a) a heavy chain variable region CDR1 comprising SEQ ID NO:17;

(b) a heavy chain variable region CDR2 comprising SEQ ID NO:23;

(c) a heavy chain variable region CDR3 comprising SEQ ID NO:29 or 75;

(d) a light chain variable region CDR1 comprising SEQ ID NO:35;

(e) a light chain variable region CDR2 comprising SEQ ID NO:41; and

(f) a light chain variable region CDR3 comprising SEQ ID NO:47.

In another preferred embodiment, the antibody comprises:

(a) a heavy chain variable region CDR1 comprising SEQ ID NO:18;

(b) a heavy chain variable region CDR2 comprising SEQ ID NO:24;

(c) a heavy chain variable region CDR3 comprising SEQ ID NO:30;

(d) a light chain variable region CDR1 comprising SEQ ID NO:36;

(e) a light chain variable region CDR2 comprising SEQ ID NO:42; and

(f) a light chain variable region CDR3 comprising SEQ ID NO:48.

It is well known in the art that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J. of Cancer 83(2):252-260 (2000) (describing the production of a humanized anti-CD30 antibody using only the heavy chain variable domain CDR3 of murine anti-CD30 antibody Ki-4); Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin αᵥβ₃ antibodies using a heavy and light chain variable CDR3 domain of a murine anti-integrin αᵥβ₃ antibody LM609 wherein each member antibody comprises a distinct sequence outside the CDR3 domain and capable of binding the same epitope as the parent murine antibody with affinities as high or higher than the parent murine antibody); Barbas et al., J. Am. Chem. Soc. 116:2161-2162 (1994) (disclosing that the CDR3 domain provides the most significant contribution to antigen binding); Barbas et al., Proc. Natl. Acad. Sci. U.S.A. 92:2529-2533 (1995) (describing the grafting of heavy chain CDR3 sequences of three Fabs (SI-1, SI-40, and SI-32) against human placental DNA onto the heavy chain of an anti-tetanus toxoid Fab thereby replacing the existing heavy chain CDR3 and demonstrating that the CDR3 domain alone conferred binding specificity); and Ditzel et al., J. Immunol. 157:739-749 (1996) (describing grafting studies wherein transfer of only the heavy chain CDR3 of a parent polyspecific Fab LNA3 to a heavy chain of a monospecific IgG tetanus toxoid-binding Fab p313 antibody was sufficient to retain binding specificity of the parent Fab); Berezov et al., BIAjournal 8:Scientific Review 8 (2001) (describing peptide mimetics based on the CDR3 of an anti-HER2 monoclonal antibody); Igarashi et al., J. Biochem (Tokyo) 117:452-7 (1995) (describing a 12 amino acid synthetic polypeptide corresponding to the CDR3 domain of an anti-phosphatidylserine antibody); Bourgeois et al., J. Virol 72:807-10 (1998) (showing that a single peptide derived from the heavy chain CDR3 domain of an anti-respiratory syncytial virus (RSV) antibody was capable of neutralizing the virus in vitro); Levi et al., Proc. Natl. Acad. Sci. U.S.A. 90:4374-8 (1993) (describing a peptide based on the heavy chain CDR3 domain of a murine anti-HIV antibody); Polymenis and Stoller, J. Immunol. 152:5218-5329 (1994) (describing enabling binding of an scFv by grafting the heavy chain CDR3 region of a Z-DNA-binding antibody); and Xu and Davis, Immunity 13:37-45 (2000) (describing that diversity at the heavy chain CDR3 is sufficient to permit otherwise identical IgM molecules to distinguish between a variety of hapten and protein antigens). See also, U.S. Pat. Nos. 6,951,646; 6,914,128; 6,090,382; 6,818,216; 6,156,313; 6,827,925; 5,833,943; 5,762,905 and 5,760,185, describing patented antibodies defined by a single CDR domain. Each of these references is hereby incorporated by reference in its entirety.

Accordingly, the present disclosure provides monoclonal antibodies comprising one or more heavy and/or light chain CDR3 domains from an antibody derived from a human or non-human animal, wherein the monoclonal antibody is capable of specifically binding to CD70. Within certain aspects, the present disclosure provides monoclonal antibodies comprising one or more heavy and/or light chain CDR3 domains from a non-human antibody, such as a mouse or rat antibody, wherein the monoclonal antibody is capable of specifically binding to CD70. Within some embodiments, such inventive antibodies comprising one or more heavy and/or light chain CDR3 domain from a non-human antibody (a) are capable of competing for binding with; (b) retain the functional characteristics; (c) bind to the same epitope; and/or (d) have a similar binding affinity as the corresponding parental non-human antibody.

Within other aspects, the present disclosure provides monoclonal antibodies comprising one or more heavy and/or light chain CDR3 domain from a human antibody, such as, for example, a human antibody obtained from a non-human animal, wherein the human antibody is capable of specifically binding to CD70. Within other aspects, the present disclosure provides monoclonal antibodies comprising one or more heavy and/or light chain CDR3 domain from a first human antibody, such as, for example, a human antibody obtained from a non-human animal, wherein the first human antibody is capable of specifically binding to CD70 and wherein the CDR3 domain from the first human antibody replaces a CDR3 domain in a human antibody that is lacking binding specificity for CD70 to generate a second human antibody that is capable of specifically binding to CD70. Within some embodiments, such inventive antibodies comprising one or more heavy and/or light chain CDR3 domain from the first human antibody (a) are capable of competing for binding with; (b) retain the functional characteristics; (c) bind to the same epitope; and/or (d) have a similar binding affinity as the corresponding parental first human antibody.

### Antibodies Having Particular Germline Sequences

In certain embodiments, an antibody of this disclosure comprises a heavy chain variable region from a particular germline heavy chain immunoglobulin gene and/or a light chain variable region from a particular germline light chain immunoglobulin gene.

For example, in a preferred embodiment, this disclosure provides an isolated monoclonal antibody or an antigen-binding portion thereof, comprising a heavy chain variable region that is the product of or derived from a human V_{H} 3-30.3 gene, wherein the antibody specifically binds CD70. In another preferred embodiment, this disclosure provides an isolated monoclonal antibody or an antigen-binding portion thereof, comprising a heavy chain variable region that is the product of or derived from a human V_{H} 3-33 gene, wherein the antibody specifically binds CD70. In another preferred embodiment, this disclosure provides an isolated monoclonal antibody or an antigen-binding portion thereof, comprising a heavy chain variable region that is the product of or derived from a human V_{H} 4-61 gene, wherein the antibody specifically binds CD70. In another preferred embodiment, this disclosure provides an isolated monoclonal antibody or an antigen-binding portion thereof, comprising a heavy chain variable region that is the product of or derived from a human V_{H} 3-23 gene, wherein the antibody specifically binds CD70.

In another preferred embodiment, this disclosure provides an isolated monoclonal antibody or an antigen-binding portion thereof, comprising a light chain variable region that is the product of or derived from a human V_{K} L6 gene, wherein the antibody specifically binds CD70. In another preferred embodiment, this disclosure provides an isolated monoclonal antibody or an antigen-binding portion thereof, comprising a light chain variable region that is the product of or derived from a human V_{K} L18 gene, wherein the antibody specifically binds CD70. In another preferred embodiment, this disclosure provides an isolated monoclonal antibody or an antigen-binding portion thereof, comprising a light chain variable region that is the product of or derived from a human V_{K} L15 gene, wherein the antibody specifically binds CD70. In another preferred embodiment, this disclosure provides an isolated monoclonal antibody or an antigen-binding portion thereof, comprising a light chain variable region that is the product of or derived from a human V_{K} A27 gene, wherein the antibody specifically binds CD70.

In yet another preferred embodiment, this disclosure provides an isolated monoclonal antibody or antigen-binding portion thereof, wherein the antibody:

(a) comprises a heavy chain variable region that is the product of or derived from a human V_{H} 3-30.3, 3-33, 4-61, or 3-23 gene (which genes encode the amino acid sequences set forth in SEQ ID NOs:61, 62, 63, and 64, respectively);

(b) comprises a light chain variable region that is the product of or derived from a human V_{K} L6, L18, L15, or A27 gene (which genes encode the amino acid sequences set forth in SEQ ID NOs:65, 66, 67, and 68, respectively); and

(c) the antibody specifically binds to CD70.

Such antibodies also may possess one or more of the functional characteristics described in detail above, such as high affinity binding to human CD70, internalization by CD70-expressing cells, the ability to mediate ADCC against CD70-expressing cells and/or the ability to inhibit tumor growth of CD70-expressing tumor cells in vivo when conjugated to a cytotoxin.

An example of an antibody having V_{H} and V_{K} of V_{H} 3-30.3 and V_{K} L6, respectively, is 2H5. An example of an antibody having V_{H} and V_{K} of V_{H} 3-30.3 and V_{K} L18, respectively, is 10B4. Examples of antibodies having V_{H} and V_{K} of V_{H} 3-33 and V_{K} L15, respectively, are 8B5 and 18E7. An example of an antibody having V_{H} and V_{K} of V_{H} 4-61 and V_{K} L6, respectively, is 69A7 and 69A7Y. An example of an antibody having V_{H} and V_{K} of V_{H} 3-23 and V_{K} A27, respectively, is 1F4.

Such antibodies also may possess one or more of the functional characteristics described in detail above, such as high affinity binding to human CD70, internalization by CD70-expressing cells, binding to a renal cell carcinoma tumor cell line, binding to a lymphoma cell line, the ability to mediate ADCC against CD70-expressing cells, and/or the ability to inhibit tumor growth of CD70-expressing tumor cells in vivo when conjugated to a cytotoxin.

As used herein, a human antibody comprises heavy or light chain variable regions that is “the product of or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95% or even at least 96%, 97%, 98% or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5 or even no more than 4, 3, 2 or 1 amino acid difference from the amino acid sequence encoded by the germline immunoglobulin gene.

### Homologous Antibodies

In yet another embodiment, an antibody of this disclosure comprises heavy and light chain variable regions comprising amino acid sequences that are homologous to the amino acid sequences of the preferred antibodies described herein and wherein the antibodies retain the desired functional properties of the anti-CD70 antibodies of this disclosure.

For example, this disclosure provides an isolated monoclonal antibody or antigen binding portion thereof, comprising a heavy chain variable region and a light chain variable region, wherein:

(a) the heavy chain variable region comprises an amino acid sequence that is at least 80% homologous to an amino acid sequence selected from the group consisting of SEQ ID NOs:1, 2, 3, 4, 5, 6 and 73;

(b) the light chain variable region comprises an amino acid sequence that is at least 80% homologous to an amino acid sequence selected from the group consisting of SEQ ID NOs:7, 8, 9, 10, 11, and 12; and

(c) the antibody specifically binds to CD70.

Additionally or alternatively, the antibody may possess one or more of the following functional properties discussed above, such as high affinity binding to human CD70, internalization by CD70-expressing cells, binding to a renal cell carcinoma tumor cell line, binding to a lymphoma cell line, the ability to mediate ADCC against CD70-expressing cells, and/or the ability to inhibit tumor growth of CD70-expressing tumor cells in vivo when conjugated to a cytotoxin.

In various embodiments, the antibody can be, for example, a human antibody, a humanized antibody or a chimeric antibody.

In other embodiments, the V_{H} and/or V_{L} amino acid sequences may be 85%, 90%, 95%, 96%, 97%, 98% or 99% homologous to the sequences set forth above. An antibody having V_{H} and V_{L} regions having high (i.e., 80% or greater) homology to the V_{H} and V_{L} regions of the sequences set forth above, can be obtained by mutagenesis (e.g., site-directed or PCR-mediated mutagenesis) of nucleic acid molecules encoding SEQ ID NOs:1-12 and 73, followed by testing of the encoded altered antibody for retained function (i.e., the functions set forth above) using the functional assays described herein.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix and a gap weight of 16, 14, 12, 10, 8, 6 or 4 and a length weight of 1, 2, 3, 4, 5 or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of this disclosure. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) are useful. See www.ncbi.nlm.nih.gov.

### Antibodies With Conservative Modifications

In certain embodiments, an antibody of this disclosure comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on known anti-CD70 antibodies or conservative modifications thereof and wherein the antibodies retain the desired functional properties of the anti-CD70 antibodies of this disclosure. It is understood in the art that certain conservative sequence modification can be made which do not remove antigen binding. See, for example, Brummell et al. (1993) Biochem 32:1180-8 (describing mutational analysis in the CDR3 heavy chain domain of antibodies specific for Salmonella); de Wildt et al. (1997) Prot. Eng. 10:835-41 (describing mutation studies in anti-UA1 antibodies); Komissarov et al. (1997) J. Biol. Chem. 272:26864-26870 (showing that mutations in the middle of HCDR3 led to either abolished or diminished affinity); Hall et al. (1992) J. Immunol. 149:1605-12 (describing that a single amino acid change in the CDR3 region abolished binding activity); Kelley and O'Connell (1993) Biochem. 32:6862-35 (describing the contribution of Tyr residues in antigen binding); Adib-Conquy et al. (1998) Int. Immunol. 10:341-6 (describing the effect of hydrophobicity in binding) and Beers et al. (2000) Clin. Can. Res. 6:2835-43 (describing HCDR3 amino acid mutants). Accordingly, this disclosure provides an isolated monoclonal antibody or antigen binding portion thereof, comprising a heavy chain variable region comprising CDR1, CDR2 and CDR3 sequences and a light chain variable region comprising CDR1, CDR2 and CDR3 sequences, wherein:

(a) the heavy chain variable region CDR3 sequence comprises an amino acid sequence selected from the group consisting of amino acid sequences of SEQ ID NOs:25, 26, 27, 28, 29, 30, and 75 and conservative modifications thereof;

(b) the light chain variable region CDR3 sequence comprises an amino acid sequence selected from the group consisting of amino acid sequence of SEQ ID NOs: 43, 44, 45, 46, 47, and 48 and conservative modifications thereof; and

(c) the antibody specifically binds to CD70.

Additionally or alternatively, the antibody may possess one or more of the following functional properties described above, such as high affinity binding to human CD70, internalization by CD70-expressing cells, binding to a renal cell carcinoma tumor cell line, binding to a lymphoma cell line, the ability to mediate ADCC against CD70-expressing cells, and/or the ability to inhibit tumor growth of CD70-expressing tumor cells in vivo when conjugated to a cytotoxin.

In a preferred embodiment, the heavy chain variable region CDR2 sequence comprises an amino acid sequence selected from the group consisting of amino acid sequences of SEQ ID NOs:19, 20, 21, 22, 23, and 24 and conservative modifications thereof; and the light chain variable region CDR2 sequence comprises an amino acid sequence selected from the group consisting of amino acid sequences of SEQ ID NOs:37, 38, 39, 40, 41, and 42 and conservative modifications thereof. In another preferred embodiment, the heavy chain variable region CDR1 sequence comprises an amino acid sequence selected from the group consisting of amino acid sequences of SEQ ID NOs:13, 14, 15, 16, 17, and 18 and conservative modifications thereof; and the light chain variable region CDR1 sequence comprises an amino acid sequence selected from the group consisting of amino acid sequences of SEQ ID NOs:31, 32, 33, 34, 35, and 36 and conservative modifications thereof.

In various embodiments, the antibody can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of this disclosure by standard techniques known in the art, such as site-directed mutagenesis and PCR-mediated mutagenesis. Conservative amino acid substitutions are the ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of this disclosure can be replaced with other amino acid residues from the same side chain family and the altered antibody can be tested for retained function (i.e., the functions set forth above) using the functional assays described herein.

### Antibodies That Bind to the Same Epitope as Anti-CD70 Antibodies of This Disclosure

In another embodiment, this disclosure provides antibodies that bind an epitope on human CD70 as recognized by any of the CD70 monoclonal antibodies of this disclosure (i.e., antibodies that have the ability to cross-compete for binding to CD70 with any of the monoclonal antibodies of this disclosure). In preferred embodiments, the reference antibody for cross-competition studies can be the monoclonal antibody 2H5 (having V_{H} and V_{L} sequences as shown in SEQ ID NOs:1 and 7, respectively) or the monoclonal antibody 10B4 (having V_{H} and V_{L} sequences as shown in SEQ ID NOs:2 and 8, respectively) or the monoclonal antibody 8B5 (having V_{H} and V_{L} sequences as shown in SEQ ID NOs:3 and 9, respectively) or the monoclonal antibody 18E7 (having V_{H} and V_{L} sequences as shown in SEQ ID NOs:4 and 10, respectively) or the monoclonal antibody 69A7 (having V_{H} and V_{L} sequences as shown in SEQ ID NOs:5 and 11, respectively) or the monoclonal antibody 69A7Y (having V_{H} and V_{L} sequences as shown in SEQ ID NOs:73 and 11, respectively) or the monoclonal antibody 1F4 (having V_{H} and V_{L} sequences as shown in SEQ ID NOs:6 and 12, respectively).

Such cross-competing antibodies can be identified based on their ability to cross-compete with 2H5, 10B4, 8B5, 18E7, 69A7, 69A7Y or 1F4 in standard CD70 binding assays. For example, standard ELISA assays can be used in which a recombinant human CD70 protein is immobilized on the plate, one of the antibodies is fluorescently labeled and the ability of non-labeled antibodies to compete off the binding of the labeled antibody is evaluated. Additionally or alternatively, BIAcore analysis can be used to assess the ability of the antibodies to cross-compete. For example, epitope binding experiments using BIAcore demonstrated that the 2H5, 10B4, 8B5, 18E7, 69A7, 69A7Y or 1F4 antibodies bind to distinct epitopes on CD70. The ability of a test antibody to inhibit the binding of, for example, 2H5, 10B4, 8B5, 18E7, 69A7, 69A7Y or 1F4, to human CD70 demonstrates that the test antibody can compete with 2H5, 10B4, 8B5, 18E7, 69A7, 69A7Y or 1 F4 for binding to human CD70 and thus binds to the same epitope on human CD70 as is recognized by 2H5 (having V_{H} and V_{L} sequences as shown in SEQ ID NOs: 1 and 7, respectively), 10B4 (having V_{H} and V_{L} sequences as shown in SEQ ID NOs: 2 and 8, respectively), 8B5 (having V_{H} and V_{L} sequences as shown in SEQ ID NOs: 3 and 9, respectively), 18E7 (having V_{H} and V_{L} sequences as shown in SEQ ID NOs: 4 and 10, respectively), 69A7 (having V_{H} and V_{L} sequences as shown in SEQ ID NOs: 5 and 11, respectively), 69A7Y (having V_{H} and V_{L} sequences as shown in SEQ ID NOs: 73 and 11, respectively), or 1F4 (having V_{H} and V_{L} sequences as shown in SEQ ID NOs: 6 and 12, respectively).

In a preferred embodiment, the antibody that binds to the same epitope on human CD70 as is recognized by 2H5, 10B4, 8B5, 18E7, 69A7, 69A7Y or 1F4 is a human monoclonal antibody. Such human monoclonal antibodies can be prepared and isolated as described in the Examples.

### Engineered and Modified Antibodies

An antibody of this disclosure further can be prepared using an antibody having one or more of the V_{H} and/or V_{L} sequences disclosed herein as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

In certain embodiments, CDR grafting can be used to engineer variable regions of the antibodies. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter and U.S. Pat. Nos. 5,530,101; 5,585,089; 5,693,762 and 6,180,370 to Queen et al.)

Accordingly, another embodiment of this disclosure pertains to an isolated monoclonal antibody or antigen binding portion thereof, comprising a heavy chain variable region comprising CDR1, CDR2 and CDR3 sequences comprising an amino acid sequence selected from the group consisting of SEQ ID NOs:13, 14, 15, 16, 17, and 18, SEQ ID NOs:19, 20, 21, 22, 23, and 24 and SEQ ID NOs:25, 26, 27, 28, 29, 75 and 30, respectively and a light chain variable region comprising CDR1, CDR2 and CDR3 sequences comprising an amino acid sequence selected from the group consisting of SEQ ID NOs:31, 32, 33, 34, 35, and 36, SEQ ID NOs:37, 38, 39, 40, 41, and 42, and SEQ ID NOs:43, 44, 45, 46, 47, and 48, respectively. Thus, such antibodies contain the V_{H} and V_{L} CDR sequences of monoclonal antibodies 2H5, 10B4, 8B5, 18E7, 69A7, 69A7Y, or 1F4 yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Eur. J. Immunol. 24:827-836; the contents of each of which are expressly incorporated herein by reference. As another example, the germline DNA sequences for human heavy and light chain variable region genes can be found in the Genbank database. For example, the following heavy chain germline sequences found in the HCo7 HuMAb mouse are available in the accompanying Genbank accession numbers: 1-69 (NG_{—}0010109, NT_{—}024637 and BC070333), 3-33 (NG_{—}0010109 and NT_{—}024637) and 3-7 (NG_{—}0010109 and NT_{—}024637). As another example, the following heavy chain germline sequences found in the HCo12 HuMAb mouse are available in the accompanying Genbank accession numbers: 1-69 (NG_{—}0010109, NT_{—}024637 and BC070333), 5-51 (NG_{—}0010109 and NT_{—}024637), 4-34 (NG_{—}0010109 and NT_{—}024637), 3-30.3 (CAJ556644) and 3-23 (AJ406678). Yet another source of human heavy and light chain germline sequences is the database of human immunoglobulin genes available from IMGT (http://imgt.cines.fr).

Antibody protein sequences are compared against a compiled protein sequence database using one of the sequence similarity searching methods called the Gapped BLAST (Altschul et al. (1997) Nucleic Acids Research 25:3389-3402), which is well known to those skilled in the art. BLAST is a heuristic algorithm in that a statistically significant alignment between the antibody sequence and the database sequence is likely to contain high-scoring segment pairs (HSP) of aligned words. Segment pairs whose scores cannot be improved by extension or trimming is called a hit. Briefly, the nucleotide sequences of VBASE origin (http://vbase.mrc-cpe.cam.ac.uk/vbase1/list2.php) are translated and the region between and including FR1 through FR3 framework region is retained. The database sequences have an average length of 98 residues. Duplicate sequences which are exact matches over the entire length of the protein are removed. A BLAST search for proteins using the program blastp with default, standard parameters except the low complexity filter, which is turned off, and the substitution matrix of BLOSUM62, filters for top 5 hits yielding sequence matches. The nucleotide sequences are translated in all six frames and the frame with no stop codons in the matching segment of the database sequence is considered the potential hit. This is in turn confirmed using the BLAST program tblastx, which translates the antibody sequence in all six frames and compares those translations to the VBASE nucleotide sequences dynamically translated in all six frames. Other human germline sequence databases, such as that available from IMGT (http://imgt.cines.fr), can be searched similarly to VBASE as described above.

The identities are exact amino acid matches between the antibody sequence and the protein database over the entire length of the sequence. The positives (identities+substitution match) are not identical but amino acid substitutions are guided by the BLOSUM62 substitution matrix. If the antibody sequence matches two of the database sequences with same identity, the hit with most positives would be decided to be the matching sequence hit.

Preferred framework sequences for use in the antibodies of this disclosure are those that are structurally similar to the framework sequences used by selected antibodies of this disclosure, e.g., similar to the V_{H} 3-30.3 framework sequences (SEQ ID NO:61) and/or the V_{H} 3-33 framework sequences (SEQ ID NO:62) and/or the V_{H} 4-61 framework sequences (SEQ ID NO:63) and/or the V_{H} 3-23 framework sequences (SEQ ID NO:64) and/or the V_{K} L6 framework sequences (SEQ ID NO:65) and/or the V_{K} L18 framework sequences (SEQ ID NO:66) and/or the V_{K} L15 framework sequences (SEQ ID NO:67) and/or the V_{K} A27 framework sequences (SEQ ID NO:68) used by preferred monoclonal antibodies of this disclosure.

The V_{H} CDR1, CDR2 and CDR3 sequences and the V_{K} CDR1, CDR2 and CDR3 sequences, can be grafted onto framework regions that have the identical sequence as that found in the germline immunoglobulin gene from which the framework sequence derive or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{K} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding or other functional property of interest, can be evaluated in in vitro or in vivo assays as described herein and provided in the Examples. Preferably conservative modifications (as discussed above) are introduced. The mutations may be amino acid substitutions, additions or deletions, but are preferably substitutions. Moreover, typically no more than one, two, three, four or five residues within a CDR region are altered.

Accordingly, in another embodiment, this disclosure provides isolated anti-CD70 monoclonal antibodies or antigen binding portions thereof, comprising a heavy chain variable region comprising: (a) a V_{H} CDR1 region comprising an amino acid sequence selected from the group consisting of SEQ ID NOs:13, 14, 15, 16, 17, and 18 or an amino acid sequence having one, two, three, four or five amino acid substitutions, deletions or additions as compared to SEQ ID NOs: 13, 14, 15, 16, 17, and 18; (b) a V_{H} CDR2 region comprising an amino acid sequence selected from the group consisting of SEQ ID NOs:19, 20, 21, 22, 23, and 24 or an amino acid sequence having one, two, three, four or five amino acid substitutions, deletions or additions as compared to SEQ ID NOs: 19, 20, 21, 22, 23, and 24; (c) a V_{H} CDR3 region comprising an amino acid sequence selected from the group consisting of SEQ ID NOs:25, 26, 27, 28, 29, 75 and 30 or an amino acid sequence having one, two, three, four or five amino acid substitutions, deletions or additions as compared to SEQ ID NOs: 25, 26, 27, 28, 29, 75 and 30; (d) a V_{K} CDR1 region comprising an amino acid sequence selected from the group consisting of SEQ ID NOs:31, 32, 33, 34, 35, and 36 or an amino acid sequence having one, two, three, four or five amino acid substitutions, deletions or additions as compared to SEQ ID NOs: 31, 32, 33, 34, 35, and 36; (e) a V_{K} CDR2 region comprising an amino acid sequence selected from the group consisting of SEQ ID NOs:37, 38, 39, 40, 41, and 42 or an amino acid sequence having one, two, three, four or five amino acid substitutions, deletions or additions as compared to SEQ ID NOs:37, 38, 39, 40, 41, and 42; and (f) a V_{K} CDR3 region comprising an amino acid sequence selected from the group consisting of SEQ ID NOs:43, 44, 45, 46, 47, and 48 or an amino acid sequence having one, two, three, four or five amino acid substitutions, deletions or additions as compared to SEQ ID NOs:43, 44, 45, 46, 47, and 48.

Engineered antibodies of this disclosure include those in which modifications have been made to framework residues within V_{H} and/or V_{K}, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. Such “backmutated” antibodies are also intended to be encompassed by this disclosure. For example, for 10B4, amino acid residue #2 (within FR1) of V_{H} is an isoleucine whereas this residue in the corresponding V_{H} 3-30.3 germline sequence is a valine. To return the framework region sequences to their germline configuration, the somatic mutations can be “backmutated” to the germline sequence by, for example, site-directed mutagenesis or PCR-mediated mutagenesis (e.g., residue 2 of FR1 of the V_{H} of 10B4 can be “backmutated” from isoleucine to valine).

As another example, for 10B4, amino acid residue #30 (within FR1) of V_{H} is a glycine whereas this residue in the corresponding V_{H} 3-30.3 germline sequence is a serine. To return the framework region sequences to their germline configuration, for example, residue 30 of FR1 of the V_{H} of 10B4 can be “backmutated” from glycine to serine.

As another example, for 8B5, amino acid residue #24 (within FR1) of V_{H} is a threonine whereas this residue in the corresponding V_{H} 3-33 germline sequence is an alanine. To return the framework region sequences to their germline configuration, for example, residue 24 of FR1 of the V_{H} of 8B5 can be “backmutated” from threonine to alanine.

As another example, for 8B5, amino acid residue #77 (within FR3) of V_{H} is a lysine whereas this residue in the corresponding V_{H} 3-33 germline sequence is an asparagine. To return the framework region sequences to their germline configuration, for example, residue 11 of FR3 of the V_{H} of 8B5 can be “backmutated” from lysine to asparagine.

As another example, for 8B5, amino acid residue #80 (within FR3) of V_{H} is a serine whereas this residue in the corresponding V_{H} 3-33 germline sequence is a tyrosine. To return the framework region sequences to their germline configuration, for example, residue 14 of FR3 of the V_{H} of 8B5 can be “backmutated” from serine to tyrosine.

As another example, for 69A7, amino acid residue #50 (within FR2) of V_{H} is a leucine whereas this residue in the corresponding V_{H} 4-61 germline sequence is an isoleucine. To return the framework region sequences to their germline configuration, for example, residue 13 of FR2 of the V_{H} of 69A7 can be “backmutated” from leucine to isoleucine.

As another example, for 69A7, amino acid residue #85 (within FR3) of V_{H} is an arginine whereas this residue in the corresponding V_{H} 4-61 germline sequence is a serine. To return the framework region sequences to their germline configuration, for example, residue 18 of FR3 of the V_{H} of 69A7 can be “backmutated” from arginine to serine.

As another example, for 69A7, amino acid residue #89 (within FR3) of V_{H} is a threonine whereas this residue in the corresponding V_{H} 4-61 germline sequence is an alanine. To return the framework region sequences to their germline configuration, for example, residue 22 of FR3 of the V_{H} of 69A7 can be “backmutated” from threonine to alanine.

As another example, for 10B4, amino acid residue #46 (within FR2) of V_{L} is a phenylalanine whereas this residue in the corresponding V_{L} L18 germline sequence is a leucine. To return the framework region sequences to their germline configuration, for example, residue 12 of FR2 of the V_{L} of 10B4 can be “backmutated” from phenylalanine to leucine.

As another example, for 69A7, amino acid residue #49 (within FR2) of V_{L} is a phenylalanine whereas this residue in the corresponding V_{L} L6 germline sequence is a tyrosine. To return the framework region sequences to their germline configuration, for example, residue 15 of FR2 of the V_{L} of 69A7 can be “backmutated” from phenylalanine to tyrosine.

Another type of framework modification involves mutating one or more residues within the framework region or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

Engineered antibodies of this disclosure also include those in which modifications have been made to amino acid residues to increase or decrease immunogenic responses through amino acid modifications that alter interaction of a T-cell epitope on the antibody (see e.g., U.S. Pat. Nos. 6,835,550; 6,897,049 and 6,936249).

In addition or alternative to modifications made within the framework or CDR regions, antibodies of this disclosure may be engineered to include modifications within the Fc region, typically to alter one or more functional properties of the antibody, such as serum half-life, complement fixation, Fc receptor binding and/or antigen-dependent cellular cytotoxicity. Furthermore, an antibody of this disclosure may be chemically modified (e.g., one or more chemical moieties can be attached to the antibody) or be modified to alter its glycosylation, again to alter one or more functional properties of the antibody. Each of these embodiments is described in further detail below. The numbering of residues in the Fe region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fe region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Pc receptor or the Cl component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered Clq binding and/or reduced or abolished complement dependent cytotoxicity (CDC). This approach is described in further detail in U.S. Pat. Nos. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate ADCC and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In still another embodiment, the C-terminal end of an antibody of the present invention is modified by the introduction of a cysteine residue as is described in U.S. Provisional Application Ser. No. 60/957,271, which is hereby incorporated by reference in its entirety. Such modifications include, but are not limited to, the replacement of an existing amino acid residue at or near the C-terminus of a full-length heavy chain sequence, as well as the introduction of a cysteine-containing extension to the c-terminus of a full-length heavy chain sequence. In preferred embodiments, the cysteine-containing extension comprises the sequence alanine-alanine-cysteine (from N-terminal to C-terminal).

In preferred embodiments the presence of such C-terminal cysteine modifications provide a location for conjugation of a partner molecule, such as a therapeutic agent or a marker molecule. In particular, the presence of a reactive thiol group, due to the C-terminal cysteine modification, can be used to conjugate a partner molecule employing the disulfide linkers described in detail below. Conjugation of the antibody to a partner molecule in this manner allows for increased control over the specific site of attachment. Furthermore, by introducing the site of attachment at or near the C-terminus, conjugation can be optimized such that it reduces or eliminates interference with the antibody's functional properties, and allows for simplified analysis and quality control of conjugate preparations.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 to Co et al. Additional approaches for altering glycosylation are described in further detail in U.S. Pat. No. 7,214,775 to Hanai et al., U.S. Pat. No. 6,737,056 to Presta, U.S. Pub No. 20070020260 to Presta, PCT Publication No. WO/2007/084926 to Dickey et al., PCT Publication No. WO/2006/089294 to Zhu et al., and PCT Publication No. WO/2007/055916 to Ravetch et al., each of which is hereby incorporated by reference in its entirety.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of this disclosure to thereby produce an antibody with altered glycosylation. For example, the cell lines Ms704, Ms705 and Ms709 lack the fucosyltransferase gene, FUT8 (alpha (1,6) fucosyltransferase), such that antibodies expressed in the Ms704, Ms705 and Ms709 cell lines lack fucose on their carbohydrates. The Ms704, Ms705 and Ms709 FUT8^{-/-} cell lines were created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase alpha-L-fucosidase removes fucosyl residues from antibodies (Tarentino, A. L. et al. (1975) Biochem. 14:5516-23).

Additionally or alternatively, an antibody can be made that has an altered type of glycosylation, wherein that alteration relates to the level of sialyation of the antibody. Such alterations are described in PCT Publication No. WO/2007/084926 to Dickey et al , and PCT Publication No. WO/2007/055916 to Ravetch et al., both of which are incorporated by reference in their entirety. For example, one may employ an enzymatic reaction with sialidase, such as, for example, Arthrobacter ureafacens sialidase. The conditions of such a reaction are generally described in the U.S. Pat. No. 5,831,077, which is hereby incorporated by reference in its entirety. Other non-limiting examples of suitable enzymes are neuraminidase and N-Glycosidase F, as described in Schloemer et al., J. Virology, 15(4), 882-893 (1975) and in Leibiger et al ., Biochem J., 338, 529-538 (1999), respectively. Desialylated antibodies may be further purified by using affinity chromatography. Alternatively, one may employ methods to increase the level of sialyation, such as by employing sialytransferase enzymes. Conditions of such a reaction are generally described in Basset et al., Scandinavian Journal of Immunology, 51(3), 307-311 (2000).

Another modification of the antibodies herein that is contemplated by this disclosure is pegylation. An antibody can be pegylated to, for example, increase the biological (e.g., serum) half life of the antibody. To pegylate an antibody, the antibody or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of this disclosure. See for example, EP 0 154 316 by Nishimura et al. and EP 0 401 384 by Ishikawa et al.

### Antibody Fragments and Antibody Mimetics

The instant invention is not limited to traditional antibodies and may be practiced through the use of antibody fragments and antibody mimetics. As detailed below, a wide variety of antibody fragment and antibody mimetic technologies have now been developed and are widely known in the art. While a number of these technologies, such as domain antibodies, Nanobodies, and UniBodies make use of fragments of, or other modifications to, traditional antibody structures, there are also alternative technologies, such as Affibodies, DARPins, Anticalins, Avimers, and Versabodies that employ binding structures that, while they mimic traditional antibody binding, are generated from and function via distinct mechanisms.

Domain Antibodies (dAbs) are the smallest functional binding units of antibodies, corresponding to the variable regions of either the heavy (VH) or light (VL) chains of human antibodies. Domain Antibodies have a molecular weight of approximately 13 kDa. Domantis has developed a series of large and highly functional libraries of fully human VH and VL dAbs (more than ten billion different sequences in each library), and uses these libraries to select dAbs that are specific to therapeutic targets. In contrast to many conventional antibodies, Domain Antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; U.S. Serial No. 2004/0110941; European patent application No. 1433846 and European Patents 0368684 & 0616640; WO05/035572, WO04/101790, WO04/081026, WO04/058821, WO04/003019 and WO03/002609, each of which is herein incorporated by reference in its entirety.

Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a perfectly stable polypeptide harboring the full antigen-binding capacity of the original heavy-chain antibody. Nanobodies have a high homology with the VH domains of human antibodies and can be further humanized without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see, e.g., WO 04/041867, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The Nanoclone method (see, e.g., WO 06/079372, which is herein incorporated by reference in its entirety) is a proprietary method for generating Nanobodies against a desired target, based on automated high-throughout selection of B-cells and could be used in the context of the instant invention.

UniBodies are another antibody fragment technology, however this one is based upon the removal of the hinge region of IgG4 antibodies. The deletion of the hinge region results in a molecule that is essentially half the size of traditional IgG4 antibodies and has a univalent binding region rather than the bivalent binding region of IgG4 antibodies. It is also well known that IgG4 antibodies are inert and thus do not interact with the immune system, which may be advantageous for the treatment of diseases where an immune response is not desired, and this advantage is passed onto UniBodies. For example, UniBodies may function to inhibit or silence, but not kill, the cells to which they are bound. Additionally, UniBody binding to cancer cells do not stimulate them to proliferate. Furthermore, because UniBodies are about half the size of traditional IgG4 antibodies, they may show better distribution over larger solid tumors with potentially advantageous efficacy. UniBodies are cleared from the body at a similar rate to whole IgG4 antibodies and are able to bind with a similar affinity for their antigens as whole antibodies. Further details of UniBodies may be obtained by reference to patent application WO2007/059782, which is herein incorporated by reference in its entirety.

Affibody molecules represent a new class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55). The simple, robust structure of Affibody molecules in combination with their low molecular weight (6 kDa), make them suitable for a wide variety of applications, for instance, as detection reagents (Ronmark J, Hansson M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). Further details of Affibodies and methods of production thereof may be obtained by reference to U.S. Pat. No. 5,831,012 which is herein incorporated by reference in its entirety.

Labeled Affibodies may also be useful in imaging applications for determining abundance of Isoforms.

DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules, which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains.

DARPins can be produced in bacterial expression systems at very high yields and they belong to the most stable proteins known. Highly specific, high-affinity DARPins to a broad range of target proteins, including human receptors, cytokines, kinases, human proteases, viruses and membrane proteins, have been selected. DARPins having affinities in the single-digit nanomolar to picomolar range can be obtained.

DARPins have been used in a wide range of applications, including ELISA, sandwich ELISA, flow cytometric analysis (FACS), immunohistochemistry (IHC), chip applications, affinity purification or Western blotting. DARPins also proved to be highly active in the intracellular compartment for example as intracellular marker proteins fused to green fluorescent protein (GFP). DARPins were further used to inhibit viral entry with IC50 in the pM range. DARPins are not only ideal to block protein-protein interactions, but also to inhibit enzymes. Proteases, kinases and transporters have been successfully inhibited, most often an allosteric inhibition mode. Very fast and specific enrichments on the tumor and very favorable tumor to blood ratios make DARPins well suited for in vivo diagnostics or therapeutic approaches.

Additional information regarding DARPins and other DRP technologies can be found in U.S. Patent Application Publication No. 2004/0132028 and International Patent Application Publication No. WO 02/20565, both of which are hereby incorporated by reference in their entirety.

Anticalins are an additional antibody mimetic technology, however in this case the binding specificity is derived from lipocalins, a family of low molecular weight proteins that are naturally and abundantly expressed in human tissues and body fluids. Lipocalins have evolved to perform a range of functions in vivo associated with the physiological transport and storage of chemically sensitive or insoluble compounds. Lipocalins have a robust intrinsic structure comprising a highly conserved β-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved β-sheet framework is reminiscent of immunoglobulins, lipocalins differ considerably from antibodies in terms of size, being composed of a single polypeptide chain of 160-180 amino acids which is marginally larger than a single immunoglobulin domain.

Lipocalins are cloned and their loops are subjected to engineering in order to create Anticalins. Libraries of structurally diverse Anticalins have been generated and Anticalin display allows the selection and screening of binding function, followed by the expression and production of soluble protein for further analysis in prokaryotic or eukaryotic systems. Studies have successfully demonstrated that Anticalins can be developed that are specific for virtually any human target protein can be isolated and binding affinities in the nanomolar or higher range can be obtained.

Anticalins can also be formatted as dual targeting proteins, so-called Duocalins. A Duocalin binds two separate therapeutic targets in one easily produced monomeric protein using standard manufacturing processes while retaining target specificity and affinity regardless of the structural orientation of its two binding domains.

Modulation of multiple targets through a single molecule is particularly advantageous in diseases known to involve more than a single causative factor. Moreover, bi- or multivalent binding formats such as Duocalins have significant potential in targeting cell surface molecules in disease, mediating agonistic effects on signal transduction pathways or inducing enhanced internalization effects via binding and clustering of cell surface receptors. Furthermore, the high intrinsic stability of Duocalins is comparable to monomeric Anticalins, offering flexible formulation and delivery potential for Duocalins.

Additional information regarding Anticalins can be found in U.S. Pat. No. 7,250,297 and International Patent Application Publication No. WO 99/16873, both of which are hereby incorporated by reference in their entirety.

Another antibody mimetic technology useful in the context of the instant invention are Avimers. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets.

Additional information regarding Avimers can be found in U.S. Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

Versabodies are another antibody mimetic technology that could be used in the context of the instant invention. Versabodies are small proteins of 3-5 kDa with >15% cysteines, which form a high disulfide density scaffold, replacing the hydrophobic core that typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

The inspiration for Versabodies comes from the natural injectable biopharmaceuticals produced by leeches, snakes, spiders, scorpions, snails, and anemones, which are known to exhibit unexpectedly low immunogenicity. Starting with selected natural protein families, by design and by screening the size, hydrophobicity, proteolytic antigen processing, and epitope density are minimized to levels far below the average for natural injectable proteins.

Given the structure of Versabodies, these antibody mimetics offer a versatile format that includes multi-valency, multi-specificity, a diversity of half-life mechanisms, tissue targeting modules and the absence of the antibody Fc region. Furthermore, Versabodies are manufactured in E. coli at high yields, and because of their hydrophilicity and small size, Versabodies are highly soluble and can be formulated to high concentrations. Versabodies are exceptionally heat stable (they can be boiled) and offer extended shelf-life.

Additional information regarding Versabodies can be found in U.S. Patent Application Publication No. 2007/0191272 which is hereby incorporated by reference in its entirety.

The detailed description of antibody fragment and antibody mimetic technologies provided above is not intended to be a comprehensive list of all technologies that could be used in the context of the instant specification. For example, and also not by way of limitation, a variety of additional technologies including alternative polypeptide-based technologies, such as fusions of complimentary determining regions as outlined in Qui et al., Nature Biotechnology, 25(8) 921-929 (2007), which is hereby incorporated by reference in its entirety, as well as nucleic acid-based technologies, such as the RNA aptamer technologies described in U.S. Pat. Nos. 5,789,157, 5,864,026, 5,712,375, 5,763,566, 6,013,443, 6,376,474, 6,613,526, 6,114,120, 6,261,774, and 6,387,620, all of which are hereby incorporated by reference, could be used in the context of the instant invention.

### Antibody Physical Properties

The antibodies of the present disclosure may be further characterized by the various physical properties of the anti-CD70 antibodies. Various assays may be used to detect and/or differentiate different classes of antibodies based on these physical properties.

In some embodiments, antibodies of the present disclosure may contain one or more glycosylation sites in either the light or heavy chain variable region. The presence of one or more glycosylation sites in the variable region may result in increased immunogenicity of the antibody or an alteration of the pK of the antibody due to altered antigen binding (Marshall et al (1972) Annu Rev Biochem 41:673-702; Gala F A and Morrison S L (2004) J Immunol 172:5489-94; Wallick et al (1988) J Exp Med 168:1099-109; Spiro R G (2002) Glycobiology 12:43R-56R; Parekh et al (1985) Nature 316:452-7; Mimura et al. (2000) Mol Immunol 37:697-706). Glycosylation has been known to occur at motifs containing an N—X—S/T sequence. Variable region glycosylation may be tested using a Glycoblot assay, which cleaves the antibody to produce a Fab, and then tests for glycosylation using an assay that measures periodate oxidation and Schiff base formation. Alternatively, variable region glycosylation may be tested using Dionex light chromatography (Dionex-LC), which cleaves saccharides from a Fab into monosaccharides and analyzes the individual saccharide content. In some instances, it is preferred to have an anti-CD70 antibody that does not contain variable region glycosylation. This can be achieved either by selecting antibodies that do not contain the glycosylation motif in the variable region or by mutating residues within the glycosylation motif using standard techniques well known in the art.

In a preferred embodiment, the antibodies of the present disclosure do not contain asparagine isomerism sites. A deamidation or isoaspartic acid effect may occur on N-G or D-G sequences, respectively. The deamidation or isoaspartic acid effect results in the creation of isoaspartic acid which decreases the stability of an antibody by creating a kinked structure off a side chain carboxy terminus rather than the main chain. The creation of isoaspartic acid can be measured using an iso-quant assay, which uses a reverse-phase HPLC to test for isoaspartic acid.

Each antibody will have a unique isoelectric point (pI), but generally antibodies will fall in the pH range of between 6 and 9.5. The pI for an IgG1 antibody typically falls within the pH range of 7-9.5 and the pI for an IgG4 antibody typically falls within the pH range of 6-8. Antibodies may have a pI that is outside this range. Although the effects are generally unknown, there is speculation that antibodies with a pI outside the normal range may have some unfolding and instability under in vivo conditions. The isoelectric point may be tested using a capillary isoelectric focusing assay, which creates a pH gradient and may utilize laser focusing for increased accuracy (Janini et at (2002) Electrophoresis 23:1605-11; Ma et al. (2001) Chromatographia 53:S75-89; Hunt et at (1998) J Chromatogr A 800:355-67). In some instances, it is preferred to have an anti-CD70 antibody that contains a pI value that falls in the normal range. This can be achieved either by selecting antibodies with a pI in the normal range, or by mutating charged surface residues using standard techniques well known in the art.

Each antibody will have a melting temperature that is indicative of thermal stability (Krishnamurthy R and Manning M C (2002) Curr Pharm Biotechnol 3:361-71). A higher thermal stability indicates greater overall antibody stability in vivo. The melting point of an antibody may be measured using techniques such as differential scanning calorimetry (Chen et al (2003) Pharm Res 20:1952-60; Ghirlando et al (1999) Immunol Lett 68:47-52). T_{M1} indicates the temperature of the initial unfolding of the antibody. T_{M2} indicates the temperature of complete unfolding of the antibody. Generally, it is preferred that the T_{M1} of an antibody of the present disclosure is greater than 60° C., preferably greater than 65° C., even more preferably greater than 70° C. Alternatively, the thermal stability of an antibody may be measured using circular dichroism (Murray et al. (2002) J. Chromatogr Sci 40:343-9).

In a preferred embodiment, antibodies that do not rapidly degrade are selected. Fragmentation of an anti-CD70 antibody may be measured using capillary electrophoresis (CE) and MALDI-MS, as is well understood in the art (Alexander A J and Hughes D E (1995) Anal Chem 67:3626-32).

In another preferred embodiment, antibodies that have minimal aggregation effects are selected. Aggregation may lead to triggering of an unwanted immune response and/or altered or unfavorable pharmacokinetic properties. Generally, antibodies are acceptable with aggregation of 25% or less, preferably 20% or less, even more preferably 15% or less, even more preferably 10% or less and even more preferably 5% or less. Aggregation may be measured by several techniques well known in the art, including size-exclusion column (SEC) high performance liquid chromatography (HPLC), and light scattering to identify monomers, dimers, trimers or multimers.

### Methods of Engineering Antibodies

As discussed above, the anti-CD70 antibodies having V_{H} and V_{K} sequences disclosed herein can be used to create new anti-CD70 antibodies by modifying the V_{H} and/or V_{K} sequences or the constant region(s) attached thereto. Thus, in another aspect of this disclosure, the structural features of an anti-CD70 antibody of this disclosure, e.g. 2H5, 10B4, 8B5, 18E7, 69A7, 69A7Y or 1F4, are used to create structurally related anti-CD70 antibodies that retain at least one functional property of the antibodies of this disclosure, such as binding to human CD70. For example, one or more CDR regions of 2H5, 10B4, 8B5, 18E7, 69A7, 69A7Y or 1F4 or mutations thereof, can be combined recombinantly with known framework regions and/or other CDRs to create additional, recombinantly-engineered, anti-CD70 antibodies of this disclosure, as discussed above. Other types of modifications include those described in the previous section. The starting material for the engineering method is one or more of the V_{H} and/or V_{K} sequences provided herein or one or more CDR regions thereof. To create the engineered antibody, it is not necessary to actually prepare (i.e., express as a protein) an antibody having one or more of the V_{H} and/or V_{K} sequences provided herein or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, this disclosure provides a method for preparing an anti-CD70 antibody comprising:

(a) providing: (i) a heavy chain variable region antibody sequence comprising a CDR1 sequence selected from the group consisting of SEQ ID NOs:13, 14, 15, 16, 17, and 18, a CDR2 sequence selected from the group consisting of SEQ ID NOs:19, 20, 21, 22, 23, and 24 and/or a CDR3 sequence selected from the group consisting of SEQ ID NOs:25, 26, 27, 28, 29, 75, and 30; and/or (ii) a light chain variable region antibody sequence comprising a CDR1 sequence selected from the group consisting of SEQ ID NOs:31, 32, 33, 34, 35, and 36, a CDR2 sequence selected from the group consisting of SEQ ID NOs:37, 38, 39, 40, 41, and 42 and/or a CDR3 sequence selected from the group consisting of SEQ ID NOs:43, 44, 45, 46, 47, and 48;

(b) altering at least one amino acid residue within the heavy chain variable region antibody sequence and/or the light chain variable region antibody sequence to create at least one altered antibody sequence; and

(c) expressing the altered antibody sequence as a protein.

Standard molecular biology techniques can be used to prepare and express the altered antibody sequence.

Preferably, the antibody encoded by the altered antibody sequence(s) is one that retains one, some or all of the functional properties of the anti-CD70 antibodies described herein, which functional properties include, but are not limited to

(a) binds to human CD70 with a K_{D} of 1×10⁻⁷ M or less; and

(b) binds to a renal cell carcinoma tumor cell line;

(c) binds to a lymphoma cell line, e.g., a B-cell tumor cell line;

(d) is internalized by CD70-expressing cells;

(e) exhibits antibody dependent cellular cytotoxicity (ADCC) against CD70-expressing cells; and

(f) inhibits growth of CD70-expressing cells in vivo when conjugated to a cytotoxin.

The functional properties of the altered antibodies can be assessed using standard assays available in the art and/or described herein, such as those set forth in the Examples (e.g., flow cytometry, binding assays).

In certain embodiments of the methods of engineering antibodies of this disclosure, mutations can be introduced randomly or selectively along all or part of an anti-CD70 antibody coding sequence and the resulting modified anti-CD70 antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly or a combination thereof. Alternatively, PCT Publication WO 03/074679 by Lazar et al. describes methods of using computational screening methods to optimize physiochemical properties of antibodies.

Nucleic Acid Molecules Encoding Antibodies of This disclosure

Another aspect of this disclosure pertains to nucleic acid molecules that encode the antibodies of this disclosure. The nucleic acids may be present in whole cells, in a cell lysate or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of this disclosure can be, for example, DNA or RNA and may or may not contain intronic sequences. In a preferred embodiment, the nucleic acid is a cDNA molecule.

Nucleic acids of this disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), a nucleic acid encoding such antibodies can be recovered from the gene library.

Preferred nucleic acids molecules of this disclosure are those encoding the VH and VL sequences of the 2H5, 10B4, 8B5, 18E7, 69A7, 69A7Y or 1F4 monoclonal antibodies. DNA sequences encoding the VH sequences of 2H5, 10B4, 8B5, 18E7, 69A7, 69A7Y and 1F4 are shown in SEQ ID NOs:49, 50, 51, 52, 53, 74 and 54, respectively. DNA sequences encoding the VL sequences of 2H5, 10B4, 8B5, 18E7, 69A7, 69A7Y and 1F4 are shown in SEQ ID NOs:55, 56, 57, 58, 59, 59 and 60, respectively (69A7 and 69A7Y have the same DNA sequences encoding the VL sequence as shown in SEQ ID NO:59).

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1, IgG2, IgG3 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. In preferred embodiments, the light chain constant region can be a kappa or lambda constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

### Production of Monoclonal Antibodies of This Disclosure

Monoclonal antibodies (mAbs) of the present disclosure can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present disclosure can be prepared based on the sequence of a non-human monoclonal antibody prepared as described above. DNA encoding the heavy and light chain immunoglobulins can be obtained from the non-human hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, murine CDR regions can be inserted into a human framework using methods known in the art (see e.g., U.S. Pat. No. 5,225,539 to Winter and U.S. Pat. Nos. 5,530,101; 5,585,089; 5,693,762 and 6,180,370 to Queen et al.).

In a preferred embodiment, the antibodies of this disclosure are human monoclonal antibodies. Such human monoclonal antibodies directed against CD70 can be generated using transgenic or transchromosomic mice carrying parts of the human immune system rather than the mouse system. These transgenic and transchromosomic mice include mice referred to herein as the HuMAb Mouse® and KM Mouse®, respectively and are collectively referred to herein as “human Ig mice.”

The HuMAb Mouse® (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368(6474): 856-859). Accordingly, the mice exhibit reduced expression of mouse IgM or κ and in response to immunization, the introduced human heavy and light chain transgenes undergo class switching and somatic mutation to generate high affinity human IgGκ monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93 and Harding, F. and Lonberg, N. (1995) Ann. N.Y. Acad. Sci. 764:536-546). Preparation and use of the HuMab Mouse® and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al. Transgenic mice carrying human lambda light chain genes also can be used, such as those described in PCT Publication No. WO 00/26373 by Bruggemann. For example, a mouse carrying a human lambda light chain transgene can be crossbred with a mouse carrying a human heavy chain transgene (e.g., HCo7), and optionally also carrying a human kappa light chain transgene (e.g., KCo5) to create a mouse carrying both human heavy and light chain transgenes (see e.g., Example 1).

In another embodiment, human antibodies of this disclosure can be raised using a mouse that carries human immunoglobulin sequences on transgenes and transchomosomes, such as a mouse that carries a human heavy chain transgene and a human light chain transchromosome. This mouse is referred to herein as the “KM Mouse®”, and is described in detail in PCT Publication WO 02/43478 to Ishida et al.

Still further, alternative transgenic animal systems expressing human immunoglobulin genes are available in the art and can be used to raise anti-CD70 antibodies of this disclosure. For example, an alternative transgenic system referred to as the Xenomouse (Abgenix, Inc.) can be used; such mice are described in, for example, U.S. Pat. Nos. 5,939,598; 6,075,181; 6,114,598; 6, 150,584 and 6,162,963 to Kucherlapati et al.

Moreover, alternative transchromosomic animal systems expressing human immunoglobulin genes are available in the art and can be used to raise anti-CD70 antibodies of this disclosure. For example, mice carrying both a human heavy chain transchromosome and a human light chain transchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894 and PCT application No. WO 2002/092812) and can be used to raise anti-CD70 antibodies of this disclosure.

Human monoclonal antibodies of this disclosure can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of this disclosure can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In another embodiment, human anti-CD70 antibodies are prepared using a combination of human Ig mouse and phage display techniques, as described in U.S. Pat. No. 6,794,132 by Buechler et al. More specifically, the method first involves raising an anti-CD70 antibody response in a human Ig mouse (such as a HuMab mouse or KM mouse as described above) by immunizing the mouse with one or more CD70 antigens, followed by isolating nucleic acids encoding human antibody chains from lymphatic cells of the mouse and introducing these nucleic acids into a display vector (e.g., phage) to provide a library of display packages. Thus, each library member comprises a nucleic acid encoding a human antibody chain and each antibody chain is displayed from the display package. The library then is screened with CD70 protein to isolate library members that specifically bind to CD70. Nucleic acid inserts of the selected library members then are isolated and sequenced by standard methods to determine the light and heavy chain variable sequences of the selected CD70 binders. The variable regions can be converted to full-length antibody chains by standard recombinant DNA techniques, such as cloning of the variable regions into an expression vector that carries the human heavy and light chain constant regions such that the V_{H} region is operatively linked to the C_{H} region and the V_{L} region is operatively linked to the C_{L} region.

### Immunization of Human Ig Mice

When human Ig mice are used to raise human antibodies of this disclosure, such mice can be immunized with a CD70-expressing cell line, a purified or enriched preparation of CD70 antigen and/or recombinant CD70 or an CD70 fusion protein, as described by Lonberg, N. et al. (1994) Nature 368(6474): 856-859; Fishwild, D. et al. (1996) Nature Biotechnology 14: 845-851; and PCT Publication WO 98/24884 and WO 01/14424. Preferably, the mice will be 6-16 weeks of age upon the first infusion. For example, a purified or recombinant preparation (5-50 μg) of CD70 antigen can be used to immunize the human Ig mice intraperitoneally and/or subcutaneously.

Detailed procedures to generate fully human monoclonal antibodies that bind CD70 are described in Example 1 below. Cumulative experience with various antigens has shown that the transgenic mice respond when initially immunized intraperitoneally (IP) with antigen in complete Freund's adjuvant, followed by every other week IP immunizations up to a total of 6) with antigen in incomplete Freund's adjuvant. However, adjuvants other than Freund's are also found to be effective (e.g., RIBI adjuvant). In addition, whole cells in the absence of adjuvant are found to be highly immunogenic. The immune response can be monitored over the course of the immunization protocol with plasma samples being obtained by retroorbital bleeds. The plasma can be screened by ELISA (as described below) and mice with sufficient titers of anti-CD70 human immunoglobulin can be used for fusions. Mice can be boosted intravenously with antigen, for example, 3 days before sacrifice and removal of the spleen. It is expected that 2-3 fusions for each immunization may need to be performed. Between 6 and 24 mice are typically immunized for each antigen. Usually both HCo7 and HCo12 strains are used. Generation of HCo7 and HCo12 mouse strains are described in U.S. Pat. No. 5,770,429 and Example 2 of PCT Publication WO 01/09187, respectively. In addition, both HCo7 and HCo12 transgene can be bred together into a single mouse having two different human heavy chain transgenes (HCo7/HCo12). Alternatively or additionally, the KM Mouse® strain can be used, as described in PCT Publication WO 02/43478.

Generation of Hybridomas Producing Human Monoclonal Antibodies of this Disclosure

To generate hybridomas producing human monoclonal antibodies of this disclosure, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspension of splenic lymphocytes from immunized mice can be fused to one-sixth the number of P3X63-Ag8.653 nonsecreting mouse myeloma cells (ATCC, CRL 1580) with 50% PEG. Alternatively, the single cell suspension of splenic lymphocytes from immunized mice can be fused using an electric field based electrofusion method, using a CytoPulse large chamber cell fusion electroporator (CytoPulse Sciences, Inc., Glen Burnie, Md.). Cells are plated at approximately 2×10⁵ in flat bottom microtiter plate, followed by a one week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin, and 1× Hypoxanthine-aminopterin-thymidine (HAT) media (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS and the concentration can be determined by OD₂₈₀ using 1.43 extinction coefficient. The monoclonal antibodies can be aliquoted and stored at −80° C.

Generation of Transfectomas Producing Monoclonal Antibodies of this Disclosure

Antibodies of this disclosure also can be produced in a host cell transfectoma using, for example, a combination of recombinant DNA techniques and gene transfection methods as is well known in the art (e.g., Morrison, S. (1985) Science 229:1202).

For example, to express the antibodies or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{K} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of this disclosure carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors of this disclosure may carry additional sequences, such as sequences that regulate replication of the vector in host cells (e.g. origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr- host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of this disclosure in either prokaryotic or eukaryotic host cells, expression of antibodies in eukaryotic cells and most preferably mammalian host cells, is the most preferred because such eukaryotic cells and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of this disclosure include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Characterization of Antibody Binding to Antigen

Antibodies of this disclosure can be tested for binding to CD70 by, for example, flow cytometry. Briefly, CD70-expressing cells are freshly harvested from tissue culture flasks and a single cell suspension prepared. CD70-expressing cell suspensions are either stained with primary antibody directly or after fixation with 1% paraformaldehyde in PBS. Approximately one million cells are resuspended in PBS containing 0.5% BSA and 50-200 μg/ml of primary antibody and incubated on ice for 30 minutes. The cells are washed twice with PBS containing 0.1% BSA, 0.01% NaN₃, resuspended in 100 μl of 1:100 diluted FITC-conjugated goat-anti-human IgG (Jackson ImmunoResearch, West Grove, Pa.) and incubated on ice for an additional 30 minutes. The cells are again washed twice, resuspended in 0.5 ml of wash buffer and analyzed for fluorescent staining on a FACSCalibur cytometer (Becton-Dickinson, San Jose, Calif.).

Alternatively, antibodies of this disclosure can be tested for binding to CD70 by standard ELISA. Briefly, microtiter plates are coated with purified CD70 at 0.25 μg/ml in PBS and then blocked with 5% bovine serum albumin in PBS. Dilutions of antibody (e.g., dilutions of plasma from CD70-immunized mice) are added to each well and incubated for 1-2 hours at 37° C. The plates are washed with PBS/Tween and then incubated with secondary reagent (e.g., for human antibodies, a goat-anti-human IgG Fc-specific polyclonal reagent) conjugated to alkaline phosphatase for 1 hour at 37° C. After washing, the plates are developed with pNPP substrate (1 mg/ml) and analyzed at OD of 405-650. Preferably, mice which develop the highest titers will be used for fusions.

An ELISA assay as described above can also be used to screen for hybridomas that show positive reactivity with CD70 immunogen. Hybridomas that bind with high avidity to CD70 are subcloned and further characterized. One clone from each hybridoma, which retains the reactivity of the parent cells (by ELISA), can be chosen for making a 5-10 vial cell bank stored at −140 ° C. and for antibody purification.

To purify anti-CD70 antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS and the concentration can be determined by OD₂₈₀ using 1.43 extinction coefficient. The monoclonal antibodies can be aliquoted and stored at −80 ° C.

To determine if the selected anti-CD70 monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using CD70 coated-ELISA plates as described above. Biotinylated mAb binding can be detected with a strep-avidin-alkaline phosphatase probe. Alternatively, competition studies can be performed using radiolabelled antibody and unlabelled competing antibodies can be detected in a Scatchard analysis, as further described in the Examples below.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-CD70 human IgGs can be further tested for reactivity with CD70 antigen by Western blotting. Briefly, CD70 can be prepared and subjected to sodium dodecyl sulfate polyacrylamide gel electrophoresis. After electrophoresis, the separated antigens are transferred to nitrocellulose membranes, blocked with 10% fetal calf serum and probed with the monoclonal antibodies to be tested. Human IgG binding can be detected using anti-human IgG alkaline phosphatase and developed with BCIP/NBT substrate tablets (Sigma Chem. Co., St. Louis, Mo.).

The binding specificity of an antibody of this disclosure may also be determined by monitoring binding of the antibody to cells expressing a CD70 protein, for example by flow cytometry. Cells or cell lines that naturally expresses CD70 protein, such 786-O, A498, ACHN, Caki-1, and/or Caki-2 cells (described further in Examples 4 and 5), may be used or a cell line, such as a CHO cell line, may be transfected with an expression vector encoding CD70 such that CD70 is expressed on the surface of the cells. The transfected protein may comprise a tag, such as a myc-tag or a his-tag, preferably at the N-terminus, for detection using an antibody to the tag. Binding of an antibody of this disclosure to a CD70 protein may be determined by incubating the transfected cells with the antibody, and detecting bound antibody. Binding of an antibody to the tag on the transfected protein may be used as a positive control.

### Bispecific Molecules

In another aspect, the present disclosure features bispecific molecules comprising an anti-CD70 antibody or a fragment thereof, of this disclosure. An antibody of this disclosure or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody of this disclosure may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule of this disclosure, an antibody of this disclosure can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present disclosure includes bispecific molecules comprising at least one first binding specificity for CD70 and a second binding specificity for a second target epitope. In a particular embodiment of this disclosure, the second target epitope is an Fc receptor, e.g., human FcγRI (CD64) or a human Fcα receptor (CD89). Therefore, this disclosure includes bispecific molecules capable of binding both to FcγR or FcαR expressing effector cells (e.g., monocytes, macrophages or polymorphonuclear cells (PMNs)) and to target cells expressing CD70. These bispecific molecules target CD70 expressing cells to effector cell and trigger Fc receptor-mediated effector cell activities, such as phagocytosis of an CD70 expressing cells, antibody dependent cell-mediated cytotoxicity (ADCC), cytokine release or generation of superoxide anion.

In an embodiment of this disclosure in which the bispecific molecule is multispecific, the molecule can further include a third binding specificity, in addition to an anti-Fc binding specificity and an anti-CD70 binding specificity. In one embodiment, the third binding specificity is an anti-enhancement factor (EF) portion, e.g., a molecule which binds to a surface protein involved in cytotoxic activity and thereby increases the immune response against the target cell. The “anti-enhancement factor portion” can be an antibody, functional antibody fragment or a ligand that binds to a given molecule, e.g., an antigen or a receptor and thereby results in an enhancement of the effect of the binding determinants for the F_{c} receptor or target cell antigen. The “anti-enhancement factor portion” can bind an F_{c} receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g. via CD2, CD3, CD8, CD28, CD4, CD40, ICAM-1 or other immune cell that results in an increased immune response against the target cell).

In one embodiment, the bispecific molecules of this disclosure comprise as a binding specificity at least one antibody or an antibody fragment thereof, including, e.g., an Fab, Fab′, F(ab′)₂, Fv, Fd, dAb or a single chain Fv. The antibody may also be a light chain or heavy chain dimer or any minimal fragment thereof such as a Fv or a single chain construct as described in Ladner et al. U.S. Pat. No. 4,946,778 to Ladner et al., the contents of which is expressly incorporated by reference.

In one embodiment, the binding specificity for an Fey receptor is provided by a monoclonal antibody, the binding of which is not blocked by human immunoglobulin G (IgG). As used herein, the term “IgG receptor” refers to any of the eight γ-chain genes located on chromosome 1. These genes encode a total of twelve transmembrane or soluble receptor isoforms which are grouped into three Fcγ receptor classes: FcγRI (CD64), FcγRII(CD32) and FcγRIII (CD16). In one preferred embodiment, the Fey receptor a human high affinity FcγRI. The human FcγRI is a 72 kDa molecule, which shows high affinity for monomeric IgG (10⁸-10⁹M⁻¹).

The production and characterization of certain preferred anti-Fey monoclonal antibodies are described by Fanger et al. in PCT Publication WO 88/00052 and in U.S. Pat. No. 4,954,617, the teachings of which are fully incorporated by reference herein. These antibodies bind to an epitope of FcγRI, FcγRII or FcγRIII at a site which is distinct from the Fey binding site of the receptor and, thus, their binding is not blocked substantially by physiological levels of IgG. Specific anti-FcγRI antibodies useful in this disclosure are mAb 22, mAb 32, mAb 44, mAb 62 and mAb 197. The hybridoma producing mAb 32 is available from the American Type Culture Collection, ATCC Accession No. HB9469. In other embodiments, the anti-Fcγ receptor antibody is a humanized form of monoclonal antibody 22 (H22). The production and characterization of the H22 antibody is described in Graziano, R. F. et al. (1995) J. Immunol 155 (10): 4996-5002 and PCT Publication WO 94/10332. The H22 antibody producing cell line was deposited at the American Type Culture Collection under the designation HA022CL1 and has the accession no. CRL11177.

In still other preferred embodiments, the binding specificity for an Fc receptor is provided by an antibody that binds to a human IgA receptor, e.g., an Fe-alpha receptor (FcαRI (CD89)), the binding of which is preferably not blocked by human immunoglobulin A (IgA). The term “IgA receptor” is intended to include the gene product of one α-gene (FcαRI) located on chromosome 19. This gene is known to encode several alternatively spliced transmembrane isoforms of 55 to 110 kDa. FcαRI (CD89) is constitutively expressed on monocytes/macrophages, eosinophilic and neutrophilic granulocytes, but not on non-effector cell populations. FcαRI has medium affinity (≈5×10⁷ M⁻¹) for both IgA1 and IgA2, which is increased upon exposure to cytokines such as G-CSF or GM-CSF (Morton, H. C. et al. (1996) Critical Reviews in Immunology 16:423-440). Four FcαRI-specific monoclonal antibodies, identified as A3, A59, A62 and A77, which bind FcαRI outside the IgA ligand binding domain, have been described (Monteiro, R. C. et al. (1992) J. Immunol. 148:1764).

FcαRI and FcγRI are preferred trigger receptors for use in the bispecific molecules of this disclosure because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); (4) mediate enhanced antigen presentation of antigens, including self-antigens, targeted to them.

While human monoclonal antibodies are preferred, other antibodies which can be employed in the bispecific molecules of this disclosure are murine, chimeric and humanized monoclonal antibodies.

The bispecific molecules of the present disclosure can be prepared by conjugating the constituent binding specificities, e.g., the anti-FcR and anti-CD70 binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP) and sulfosuccinimidyl 4-(N-maleimidomethyl)cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83) and Glennie et al. (1987) J. Immunol. 139: 2367-2375). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand×Fab fusion protein. A bispecific molecule of this disclosure can be a single chain molecule comprising one single chain antibody and a binding determinant or a single chain bispecific molecule comprising two binding determinants. Bispecific molecules may comprise at least two single chain molecules. Methods for preparing bispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858, all of which are expressly incorporated herein by reference.

Binding of the bispecific molecules to their specific targets can be confumed by, for example, enzyme-linked immunosorbent assay (ELISA), radioimmunoassay (RIA), FACS analysis, bioassay (e.g., growth inhibition) or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a y counter or a scintillation counter or by autoradiography.

### Linkers

The present invention provides for antibody-partner conjugates where the antibody is linked to the partner through a chemical linker. In some embodiments, the linker is a peptidyl linker, and is depicted herein as (L⁴)ₚ-F-(L¹)ₘ. Other linkers include hydrazine and disulfide linkers, and is depicted herein as (L⁴)ₚ-H-(L¹)ₘ or (L⁴)ₚ-J-(L₁)ₘ, respectively. In addition to the linkers as being attached to the partner, the present invention also provides cleavable linker arms that are appropriate for attachment to essentially any molecular species. The linker arm aspect of the invention is exemplified herein by reference to their attachment to a therapeutic moiety. It will, however, be readily apparent to those of skill in the art that the linkers can be attached to diverse species including, but not limited to, diagnostic agents, analytical agents, biomolecules, targeting agents, detectable labels and the like.

The use of peptidyl and other linkers in antibody-partner conjugates is described in U.S. Provisional Patent Applications Ser. Nos. 60/295,196; 60/295,259; 60/295342; 60/304,908; 60/572,667; 60/661,174; 60/669,871; 60/720,499; 60/730,804; and 60/735,657 and U.S. patent applications Ser. Nos. 10/160,972; 10/161,234; 11/134,685; 11/134,826; and 11/398,854 and U.S. Pat. No. 6,989,452 and PCT Patent Application No. PCT/US2006/37793, all of which are incorporated herein by reference.

Additional linkers are described in U.S. Pat. No. 6,214,345 (Bristol-Myers Squibb), U.S. Pat. Appl. 2003/0096743 and U.S. Pat. Appl. 2003/0130189 (both to Seattle Genetics), de Groot et al., J. Med. Chem. 42, 5277 (1999); de Groot et al. J. Org. Chem. 43, 3093 (2000); de Groot et al., J. Med. Chem. 66, 8815, (2001); WO 02/083180 (Syntarga); Carl et al., J. Med. Chem. Lett. 24, 479, (1981); Dubowchik et al., Bioorg & Med. Chem. Lett. 8, 3347 (1998); and 60/891,028 (filed on February 21, 2007).

In one aspect, the present invention relates to linkers that are useful to attach targeting groups to therapeutic agents and markers. In another aspect, the invention provides linkers that impart stability to compounds, reduce their in vivo toxicity, or otherwise favorably affect their pharmacokinetics, bioavailability and/or pharmacodynamics. It is generally preferred that in such embodiments, the linker is cleaved, releasing the active drug, once the drug is delivered to its site of action. Thus, in one embodiment of the invention, the linkers of the invention are traceless, such that once removed from the therapeutic agent or marker (such as during activation), no trace of the linker's presence remains.

In another embodiment of the invention, the linkers are characterized by their ability to be cleaved at a site in or near the target cell such as at the site of therapeutic action or marker activity. Such cleavage can be enzymatic in nature. This feature aids in reducing systemic activation of the therapeutic agent or marker, reducing toxicity and systemic side effects. Preferred cleavable groups for enzymatic cleavage include peptide bonds, ester linkages, and disulfide linkages. In other embodiments, the linkers are sensitive to pH and are cleaved through changes in pH.

An important aspect of the current invention is the ability to control the speed with which the linkers cleave. Often a linker that cleaves quickly is desired. In some embodiments, however, a linker that cleaves more slowly may be preferred. For example, in a sustained release formulation or in a formulation with both a quick release and a slow release component, it may be useful to provide a linker which cleaves more slowly. WO 02/096910 provides several specific ligand-drug complexes having a hydrazine linker. However, there is no way to “tune” the linker composition dependent upon the rate of cyclization required, and the particular compounds described cleave the ligand from the drug at a slower rate than is preferred for many drug-linker conjugates. In contrast, the hydrazine linkers of the current invention provide for a range of cyclization rates, from very fast to very slow, thereby allowing for the selection of a particular hydrazine linker based on the desired rate of cyclization.

For example, very fast cyclization can be achieved with hydrazine linkers that produce a single 5-membered ring upon cleavage. Preferred cyclization rates for targeted delivery of a cytotoxic agent to cells are achieved using hydrazine linkers that produce, upon cleavage, either two 5-membered rings or a single 6-membered ring resulting from a linker having two methyls at the geminal position. The gem-dimethyl effect has been shown to accelerate the rate of the cyclization reaction as compared to a single 6-membered ring without the two methyls at the geminal position. This results from the strain being relieved in the ring. Sometimes, however, substitutents may slow down the reaction instead of making it faster. Often the reasons for the retardation can be traced to steric hindrance. For example, the gem dimethyl substitution allows for a much faster cyclization reaction to occur compared to when the geminal carbon is a CH₂.

It is important to note, however, that in some embodiments, a linker that cleaves more slowly may be preferred. For example, in a sustained release formulation or in a formulation with both a quick release and a slow release component, it may be useful to provide a linker which cleaves more slowly. In certain embodiments, a slow rate of cyclization is achieved using a hydrazine linker that produces, upon cleavage, either a single 6-membered ring, without the gem-dimethyl substitution, or a single 7-membered ring.

The linkers also serve to stabilize the therapeutic agent or marker against degradation while in circulation. This feature provides a significant benefit since such stabilization results in prolonging the circulation half-life of the attached agent or marker. The linker also serves to attenuate the activity of the attached agent or marker so that the conjugate is relatively benign while in circulation and has the desired effect, for example is toxic, after activation at the desired site of action. For therapeutic agent conjugates, this feature of the linker serves to improve the therapeutic index of the agent.

The stabilizing groups are preferably selected to limit clearance and metabolism of the therapeutic agent or marker by enzymes that may be present in blood or non-target tissue and are further selected to limit transport of the agent or marker into the cells. The stabilizing groups serve to block degradation of the agent or marker and may also act in providing other physical characteristics of the agent or marker. The stabilizing group may also improve the agent or marker's stability during storage in either a formulated or non-formulated form.

Ideally, the stabilizing group is useful to stabilize a therapeutic agent or marker if it serves to protect the agent or marker from degradation when tested by storage of the agent or marker in human blood at 37° C. for 2 hours and results in less than 20%, preferably less than 10%, more preferably less than 5% and even more preferably less than 2%, cleavage of the agent or marker by the enzymes present in the human blood under the given assay conditions.

The present invention also relates to conjugates containing these linkers. More particularly, the invention relates to the use of prodrugs that may be used for the treatment of disease, especially for cancer chemotherapy. Specifically, use of the linkers described herein provide for prodrugs that display a high specificity of action, a reduced toxicity, and an improved stability in blood relative to prodrugs of similar structure.

The linkers of the present invention as described herein may be present at a variety of positions within the partner molecule.

Thus, there is provided a linker that may contain any of a variety of groups as part of its chain that will cleave in vivo, e.g., in the blood stream, at a rate which is enhanced relative to that of constructs that lack such groups. Also provided are conjugates of the linker arms with therapeutic and diagnostic agents. The linkers are useful to form prodrug analogs of therapeutic agents and to reversibly link a therapeutic or diagnostic agent to a targeting agent, a detectable label, or a solid support. The linkers may be incorporated into complexes that include cytotoxins.

The attachment of a prodrug to an antibody may give additional safety advantages over conventional antibody conjugates of cytotoxic drugs. Activation of a prodrug may be achieved by an esterase, both within tumor cells and in several normal tissues, including plasma. The level of relevant esterase activity in humans has been shown to be very similar to that observed in rats and non-human primates, although less than that observed in mice. Activation of a prodrug may also be achieved by cleavage by glucuronidase.

In addition to the cleavable peptide, hydrazine, or disulfide group, one or more self-immolative linker groups L¹ are optionally introduced between the cytotoxin and the targeting agent. These linker groups may also be described as spacer groups and contain at least two reactive functional groups. Typically, one chemical functionality of the spacer group bonds to a chemical functionality of the therapeutic agent, e.g., cytotoxin, while the other chemical functionality of the spacer group is used to bond to a chemical functionality of the targeting agent or the cleavable linker. Examples of chemical functionalities of spacer groups include hydroxy, mercapto, carbonyl, carboxy, amino, ketone, and mercapto groups.

The self-immolative linkers, represented by L¹, are generally a substituted or unsubstituted alkyl, substituted or unsubstituted aryl, substituted or unsubstituted heteroaryl or substituted or unsubstituted heteroalkyl group. In one embodiment, the alkyl or aryl groups may comprise between 1 and 20 carbon atoms. They may also comprise a polyethylene glycol moiety.

Exemplary spacer groups include, for example, 6-aminohexanol, 6-mercaptohexanol, 10-hydroxydecanoic acid, glycine and other amino acids, 1,6-hexanediol, β-alanine, 2-aminoethanol, cysteamine (2-aminoethanethiol), 5-aminopentanoic acid, 6-aminohexanoic acid, 3-maleimidobenzoic acid, phthalide, α-substituted phthalides, the carbonyl group, animal esters, nucleic acids, peptides and the like.

The spacer can serve to introduce additional molecular mass and chemical functionality into the cytotoxin-targeting agent complex. Generally, the additional mass and functionality will affect the serum half-life and other properties of the complex. Thus, through careful selection of spacer groups, cytotoxin complexes with a range of serum half-lives can be produced.

The spacer(s) located directly adjacent to the drug moiety is also denoted as (L¹)ₘ, wherein m is an integer selected from 0, 1, 2, 3, 4, 5, and 6. When multiple L¹ spacers are present, either identical or different spacers may be used. L¹ may be any self-immolative group.

L⁴ is a linker moiety that preferably imparts increased solubility or decreased aggregation properties to conjugates utilizing a linker that contains the moiety or modifies the hydrolysis rate of the conjugate. The L⁴ linker does not have to be self immolative. In one embodiment, the L⁴ moiety is substituted alkyl, unsubstituted alkyl, substituted aryl, unsubstituted aryl, substituted heteroalkyl, or unsubstituted heteroalkyl, any of which may be straight, branched, or cyclic. The substitutions may be, for example, a lower (C¹-C⁶) alkyl, alkoxy, aklylthio, alkylamino, or dialkylamino. In certain embodiments, L⁴ comprises a non-cyclic moiety. In another embodiment, L⁴ comprises any positively or negatively charged amino acid polymer, such as polylysine or polyargenine. L⁴ can comprise a polymer such as a polyethylene glycol moiety. Additionally the L⁴ linker can comprise, for example, both a polymer component and a small chemical moiety.

In a preferred embodiment, L⁴ comprises a polyethylene glycol (PEG) moiety. The PEG portion of L⁴ may be between 1 and 50 units long. Preferably, the PEG will have 1-12 repeat units, more preferably 3-12 repeat units, more preferably 2-6 repeat units, or even more preferably 3-5 repeat units and most preferably 4 repeat units. L⁴ may consist solely of the PEG moiety, or it may also contain an additional substituted or unsubstituted alkyl or heteroalkyl. It is useful to combine PEG as part of the L⁴ moiety to enhance the water solubility of the complex. Additionally, the PEG moiety reduces the degree of aggregation that may occur during the conjugation of the drug to the antibody.

In some embodiments, L⁴ comprises

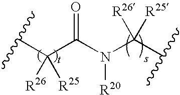

directly attached to the N-terminus of (AA¹)_{c}. R²⁰ is a member selected from H, substituted or unsubstituted alkyl, substituted or unsubstituted heteroalkyl, and acyl. Each R²⁵, R^{25′}, R^{26′}, and R^{26′} is independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl; and s and t are independently integers from 1 to 6. Preferably, R²° , R²⁵, R^{25′}, R²⁶ and R^{26′} are hydrophobic. In some embodiments, R ²⁰ is H or alkyl (preferably, unsubstituted lower alkyl). In some embodiments, R²⁵, R^{25′}, R²⁶ and R^{26′} are independently H or alkyl (preferably, unsubstituted C¹ to C⁴ alkyl). In some embodiments, R²⁵, R^{25′}, R²⁶ and R^{26′}are all H. In some embodiments, t is 1 and s is 1 or 2.

Peptide Linkers (F)

As discussed above, the peptidyl linkers of the invention can be represented by the general formula: (L⁴)ₚ-F-(L¹)ₘ, wherein F represents the linker portion comprising the peptidyl moiety. In one embodiment, the F portion comprises an optional additional self-immolative linker(s), L², and a carbonyl group. In another embodiment, the F portion comprises an amino group and an optional spacer group(s), L³.

Accordingly, in one embodiment, the conjugate comprising the peptidyl linker comprises a structure of the following formula (a):

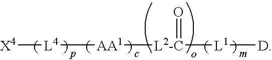

In this embodiment, L¹ is a self-immolative linker, as described above, and L⁴ is a moiety that preferably imparts increased solubility, or decreased aggregation properties, or modifies the hydrolysis rate, as described above. L² represents a self-immolative linker(s). In addition, m is 0, 1, 2, 3, 4, 5, or 6; and o and p are independently 0 or 1. AA¹ represents one or more natural amino acids, and/or unnatural α-amino acids; c is an integer from 1 and 20. In some embodiments, c is in the range of 2 to 5 or c is 2 or 3.

In the peptide linkers of the invention of the above formula (a), AA¹ is linked, at its amino terminus, either directly to L⁴ or, when L⁴ is absent, directly to the X⁴ group (i.e., the targeting agent, detectable label, protected reactive functional group or unprotected reactive functional group). In some embodiments, when L⁴ is present, L⁴ does not comprise a carboxylic acyl group directly attached to the N-terminus of (AA¹)_{c}. Thus, it is not necessary in these embodiments for there to be a carboxylic acyl unit directly between either L⁴ or X⁴ and AA¹, as is necessary in the peptidic linkers of U.S. Pat. No. 6,214,345.

In another embodiment, the conjugate comprising the peptidyl linker comprises a structure of the following formula (b):

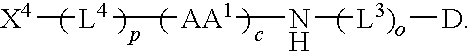

In this embodiment, L⁴ is a moiety that preferably imparts increased solubility, or decreased aggregation properties, or modifies the hydrolysis rate, as described above; L³ is a spacer group comprising a primary or secondary amine or a carboxyl functional group, and either the amine of L³ forms an amide bond with a pendant carboxyl functional group of D or the carboxyl of L³ fauns an amide bond with a pendant amine functional group of D; and o and p are independently 0 or 1. AA¹ represents one or more natural amino acids, and/or unnatural α-amino acids; c is an integer from 1 and 20. In this embodiment, L¹ is absent (i.e., m is 0 in the general formula).

In the peptide linkers of the invention of the above formula (b), AA¹ is linked, at its amino terminus, either directly to L⁴ or, when L⁴ is absent, directly to the X⁴ group (i.e., the targeting agent, detectable label, protected reactive functional group or unprotected reactive functional group). In some embodiments, when L⁴ is present, L⁴ does not comprise a carboxylic acyl group directly attached to the N-terminus of (AA¹)_{c}. Thus, it is not necessary in these embodiments for there to be a carboxylic acyl unit directly between either L⁴ or X⁴ and AA¹, as is necessary in the peptidic linkers of U.S. Pat. No. 6,214,345.

The SelfImmolative Linker L²

The self-immolative linker L² is a bifunctional chemical moiety which is capable of covalently linking together two spaced chemical moieties into a noiuially stable tripartate molecule, releasing one of said spaced chemical moieties from the tripartate molecule by means of enzymatic cleavage; and following said enzymatic cleavage, spontaneously cleaving from the remainder of the molecule to release the other of said spaced chemical moieties. In accordance with the present invention, the self-immolative spacer is covalently linked at one of its ends to the peptide moiety and covalently linked at its other end to the chemically reactive site of the drug moiety whose derivatization inhibits pharmacological activity, so as to space and covalently link together the peptide moiety and the drug moiety into a tripartate molecule which is stable and pharmacologically inactive in the absence of the target enzyme, but which is enzymatically cleavable by such target enzyme at the bond covalently linking the spacer moiety and the peptide moiety to thereby affect release of the peptide moiety from the tripartate molecule. Such enzymatic cleavage, in turn, will activate the self-immolating character of the spacer moiety and initiate spontaneous cleavage of the bond covalently linking the spacer moiety to the drug moiety, to thereby affect release of the drug in pharmacologically active form.

The self-immolative linker L² may be any self-immolative group. Preferably L² is a substituted alkyl, unsubstituted alkyl, substituted heteroalkyl, unsubstituted heteroalkyl, unsubstituted heterocycloalkyl, substituted heterocycloalkyl, substituted and unsubstituted aryl, and substituted and unsubstituted heteroaryl.

One particularly preferred self-immolative spacer L² may be represented by the formula (c):

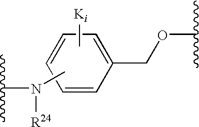

The aromatic ring of the aminobenzyl group may be substituted with one or more “K” groups. A “K” group is a substituent on the aromatic ring that replaces a hydrogen otherwise attached to one of the four non-substituted carbons that are part of the ring structure. The “K” group may be a single atom, such as a halogen, or may be a multi-atom group, such as alkyl, heteroalkyl, amino, nitro, hydroxy, alkoxy, haloalkyl, and cyano. Each K is independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², NR²¹COR²², OCONR²¹R²², OCOR²¹, and OR²¹, wherein R²¹ and R²² are independently selected from the group consisting of H, substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl and unsubstituted heterocycloalkyl. Exemplary K substituents include, but are not limited to, F, Cl, Br, I, NO₂, OH, OCH₃, NHCOCH₃, N(CH₃)₂, NHCOCF₃ and methyl. For “Kᵢ”, i is an integer of 0, 1, 2, 3, or 4. In one preferred embodiment, i is 0.

The ether oxygen atom of the structure shown above is connected to a carbonyl group. The line from the NR²⁴ functionality into the aromatic ring indicates that the amine functionality may be bonded to any of the five carbons that both form the ring and are not substituted by the —CH₂—O— group. Preferably, the NR²⁴ functionality of X is covalently bound to the aromatic ring at the para position relative to the —CH₂—O— group. R²⁴ is a member selected from the group consisting of H, substituted alkyl, unsubstituted alkyl, substituted heteroalkyl, and unsubstituted heteroalkyl. In a specific embodiment, R²⁴ is hydrogen.

In one embodiment, the invention provides a peptide linker of formula (a) above, wherein F comprises the structure:

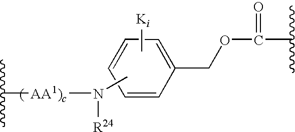

where R²⁴ is selected from the group consisting of H, substituted alkyl, unsubstituted alkyl, substituted heteroalkyl, and unsubstituted heteroalkyl. Each K is a member independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², NR²¹COR²², OCONR²¹R²², OCOR²¹, and OR²¹ where R²¹ and R²² are independent selected from the group consisting of H, substituted alkyl, ubsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl; and i is an integer of 0,1, 2, 3, or 4.

In another embodiment, the peptide linker of formula (a) above comprises a -F-(L¹)ₘ- that comprises the structure:

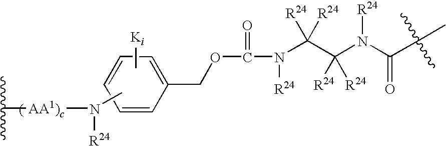

where each R²⁴ is a member independently selected from the group consisting of H, substituted alkyl, unsubstituted alkyl, substituted heteroalkyl, and unsubstituted heteroalkyl.

In some embodiments, the self-immolative spacer L¹ or L² includes

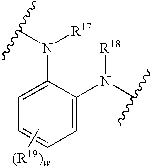

where each R¹⁷, R¹⁸, and R¹⁹ is independently selected from H, substituted or unsubstituted alkyl, substituted or unsubstituted heteroalkyl and substituted or unsubstituted aryl, and w is an integer from 0 to 4. In some embodiments, R¹⁷ and R¹⁸ are independently H or alkyl (preferably, unsubstituted C1-4 alkyl). Preferably, R¹⁷ and R¹⁸ are C1-4 alkyl, such as methyl or ethyl. In some embodiments, w is 0. While not wishing to be bound to any particular theory, it has been found experimentally that this particular self-immolative spacer cyclizes relatively quickly.

In some embodiments, L¹ or L² includes

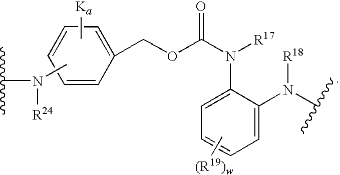

The Spacer Group L³

The spacer group L³ is characterized in that it comprises a primary or secondary amine or a carboxyl functional group, and either the amine of the L³ group forms an amide bond with a pendant carboxyl functional group of D or the carboxyl of L³ fauns an amide bond with a pendant amine functional group of D. L³ can be selected from the group consisting of substituted or unsubstituted alkyl, substituted or unsubstituted heteroalkyl, substituted or unsubstituted aryl, substituted or unsubstituted hteroaryl, or substituted or unsubstituted heterocycloalkyl. In a preferred embodiment, L³ comprises an aromatic group. More preferably, L³ comprises a benzoic acid group, an aniline group or indole group. Non-limiting examples of structures that can serve as an -L³-NH— spacer include the following structures:

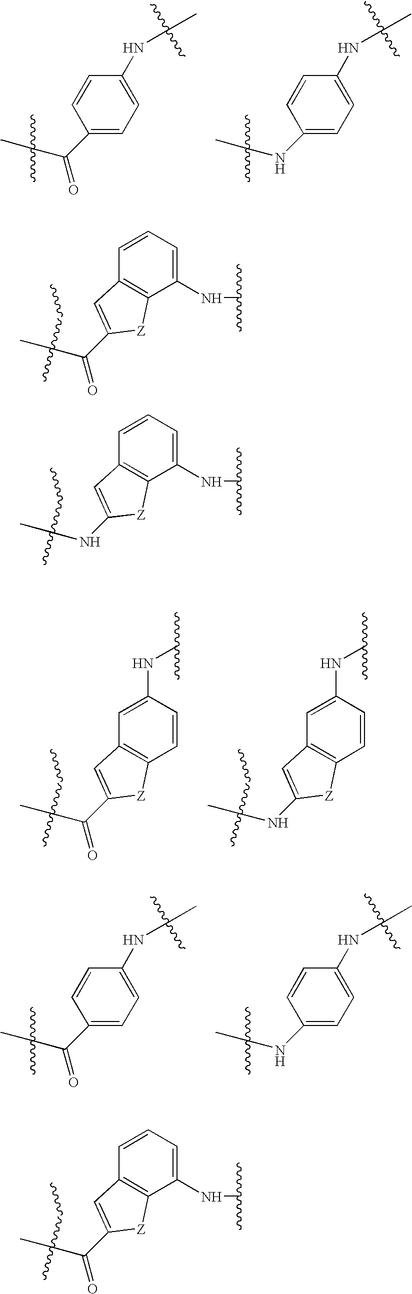

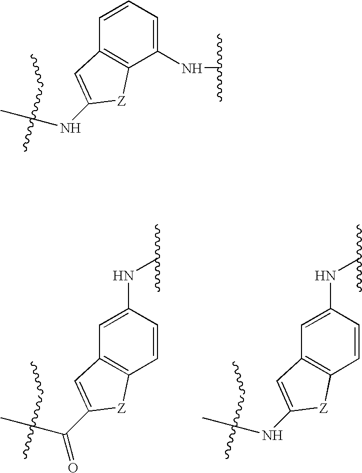

where Z is a member selected from O, S and NR²³, and where R²³ is a member selected from H, substituted or unsubstituted alkyl, substituted or unsubstituted heteroalkyl, and acyl.

Upon cleavage of the linker of the invention containing L³, the L³ moiety remains attached to the drug, D. Accordingly, the L³ moiety is chosen such that its presence attached to D does not significantly alter the activity of D. In another embodiment, a portion of the drug D itself functions as the L³ spacer. For example, in one embodiment, the drug, D, is a duocarmycin derivative in which a portion of the drug functions as the L³ spacer. Non-limiting examples of such embodiments include those in which NH₂-(L³)-D has a structure selected from the group consisting of:

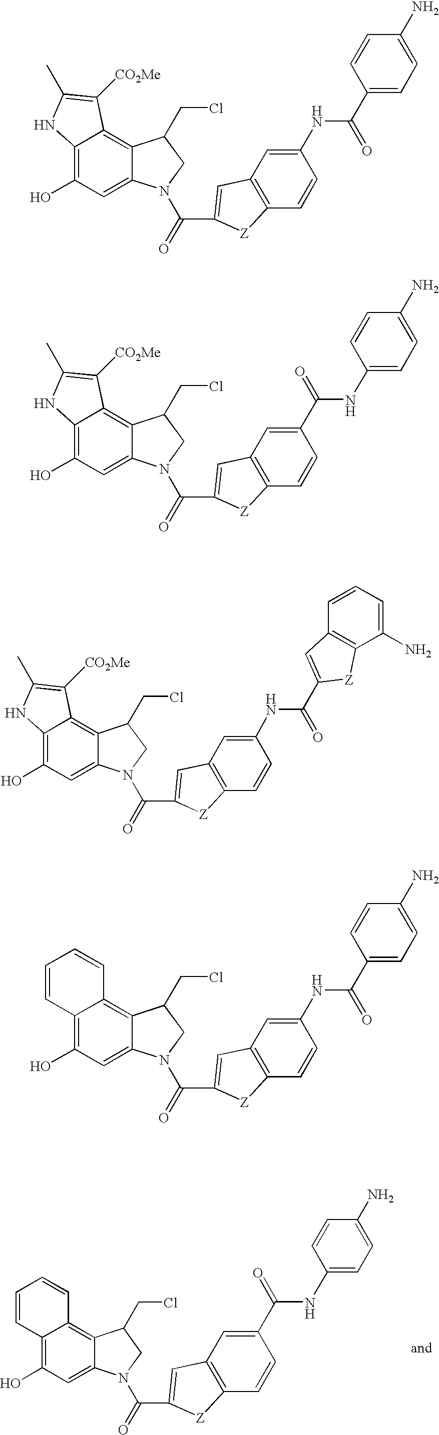

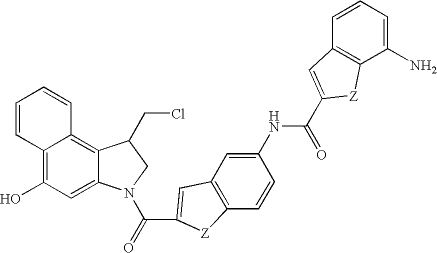

where Z is a member selected from O, S and NR²³, where R²³ is a member selected from H, substituted or unsubstituted alkyl, substituted or unsubstituted heteroalkyl, and acyl; and where the NH₂ group on each structure reacts with (AA¹)_{c}-NH—.

The Peptide Sequence AA¹

The group AA¹ represents a single amino acid or a plurality of amino acids that are joined together by amide bonds. The amino acids may be natural amino acids and/or unnatural α-amino acids.

The peptide sequence (AA¹), is functionally the amidification residue of a single amino acid (when c=1) or a plurality of amino acids joined together by amide bonds. The peptide of the current invention is selected for directing enzyme-catalyzed cleavage of the peptide by an enzyme in a location of interest in a biological system. For example, for conjugates that are targeted to a cell using a targeting agent, but not internalized by that cell, a peptide is chosen that is cleaved by one or more proteases that may exist in the extracellular matrix, e.g., due to release of the cellular contents of nearby dying cells, such that the peptide is cleaved extracellularly. The number of amino acids within the peptide can range from 1 to 20; but more preferably there will be 1-8 amino acids, 1-6 amino acids or 1, 2, 3 or 4 amino acids comprising (AA¹)_{c}. Peptide sequences that are susceptible to cleavage by specific enzymes or classes of enzymes are well known in the art.

Many peptide sequences that are cleaved by enzymes in the serum, liver, gut, etc. are known in the art. An exemplary peptide sequence of the invention includes a peptide sequence that is cleaved by a protease. The focus of the discussion that follows on the use of a protease-sensitive sequence is for clarity of illustration and does not serve to limit the scope of the present invention.

When the enzyme that cleaves the peptide is a protease, the linker generally includes a peptide containing a cleavage recognition sequence for the protease. A cleavage recognition sequence for a protease is a specific amino acid sequence recognized by the protease during proteolytic cleavage. Many protease cleavage sites are known in the art, and these and other cleavage sites can be included in the linker moiety. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); Bouvier et al. Meth. Enzymol. 248: 614 (1995), Hardy et al., in Amyloid Protein Precursor in Development, Aging, and Alzheimer's Disease, ed. Masters et al. pp. 190-198 (1994).

The amino acids of the peptide sequence (AA¹)_{c} are chosen based on their suitability for selective enzymatic cleavage by particular molecules such as tumor-associated protease. The amino acids used may be natural or unnatural amino acids. They may be in the L or the D configuration. In one embodiment, at least three different amino acids are used. In another embodiment, only two amino acids are used.

In a preferred embodiment, the peptide sequence (AA¹)_{c} is chosen based on its ability to be cleaved by a lysosomal proteases, non-limiting examples of which include cathepsins B, C, D, H, L and S. Preferably, the peptide sequence (AA¹)_{c}, is capable of being cleaved by cathepsin B in vitro, which can be tested using in vitro protease cleavage assays known in the art.

In another embodiment, the peptide sequence (AA¹)_{c} is chosen based on its ability to be cleaved by a tumor-associated protease, such as a protease that is found extracellularly in the vicinity of tumor cells, non-limiting examples of which include thimet oligopeptidase (TOP) and CD10. The ability of a peptide to be cleaved by TOP or CD10 can be tested using in vitro protease cleavage assays known in the art.

Suitable, but non-limiting, examples of peptide sequences suitable for use in the conjugates of the invention include Val-Cit, Cit-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO:77), β-Ala-Leu-Ala-Leu (SEQ ID NO:78), Gly-Phe-Leu-Gly (SEQ: ID NO:79), Val-Ala, Leu-Leu-Gly-Leu (SEQ ID NO: 91), Leu-Asn-Ala, and Lys-Leu-Val. Preferred peptides sequences are Val-Cit and Val-Lys.

In another embodiment, the amino acid located the closest to the drug moiety is selected from the group consisting of Ala, Asn, Asp, Cit, Cys, Gln, Glu, Gly, Ile, Leu, Lys, Met, Phe, Pro, Ser, Thr, Trp, Tyr, and Val. In yet another embodiment, the amino acid located the closest to the drug moiety is selected from the group consisting of: Ala, Asn, Asp, Cys, Gln, Glu, Gly, Ile, Leu, Met, Phe, Pro, Ser, Thr, Tip, Tyr, and Val.

Proteases have been implicated in cancer metastasis. Increased synthesis of the protease urokinase was correlated with an increased ability to metastasize in many cancers. Urokinase activates plasmin from plasminogen, which is ubiquitously located in the extracellular space and its activation can cause the degradation of the proteins in the extracellular matrix through which the metastasizing tumor cells invade. Plasmin can also activate the collagenases thus promoting the degradation of the collagen in the basement membrane surrounding the capillaries and lymph system thereby allowing tumor cells to invade into the target tissues (Dano, et al. Adv. Cancer. Res., 44:139 (1985)). Thus, it is within the scope of the present invention to utilize as a linker a peptide sequence that is cleaved by urokinase.

The invention also provides the use of peptide sequences that are sensitive to cleavage by tryptases. Human mast cells express at least four distinct tryptases, designated α βI, βII, and βIII. These enzymes are not controlled by blood plasma proteinase inhibitors and only cleave a few physiological substrates in vitro. The tryptase family of serine proteases has been implicated in a variety of allergic and inflammatory diseases involving mast cells because of elevated tryptase levels found in biological fluids from patients with these disorders. However, the exact role of tryptase in the pathophysiology of disease remains to be delineated. The scope of biological functions and corresponding physiological consequences of tryptase are substantially defined by their substrate specificity.

Tryptase is a potent activator of pro-urokinase plasminogen activator (uPA), the zymogen form of a protease associated with tumor metastasis and invasion. Activation of the plasminogen cascade, resulting in the destruction of extracellular matrix for cellular extravasation and migration, may be a function of tryptase activation of pro-urokinase plasminogen activator at the P4-P 1 sequence of Pro-Arg-Phe-Lys (SEQ ID NO:80) (Stack, et al., Journal of Biological Chemistry 269 (13): 9416-9419 (1994)). Vasoactive intestinal peptide, a neuropeptide that is implicated in the regulation of vascular permeability, is also cleaved by tryptase, primarily at the Thr-Arg-Leu-Arg (SEQ ID NO:81) sequence (Tam, et al., Am. J. Respir. Cell Mol. Biol. 3: 27-32 (1990)). The G-protein coupled receptor PAR-2 can be cleaved and activated by tryptase at the Ser-Lys-Gly-Arg (SEQ ID NO:82) sequence to drive fibroblast proliferation, whereas the thrombin activated receptor PAR-1 is inactivated by tryptase at the Pro-Asn-Asp-Lys (SEQ ID NO: 83) sequence (Molino et al., Journal of Biological Chemistry 272(7): 4043-4049 (1997)). Taken together, this evidence suggests a central role for tryptase in tissue remodeling as a consequence of disease. This is consistent with the profound changes observed in several mast cell-mediated disorders. One hallmark of chronic asthma and other long-term respiratory diseases is fibrosis and thickening of the underlying tissues that could be the result of tryptase activation of its physiological targets. Similarly, a series of reports have shown angiogenesis to be associated with mast cell density, tryptase activity and poor prognosis in a variety of cancers (Coussens et al., Genes and Development 13(11): 1382-97 (1999)); Takanami et al., Cancer 88(12): 2686-92 (2000); Toth-Jakatics et al., Human Pathology 31(8): 955-960 (2000); Ribatti et al., International Journal of Cancer 85(2): 171-5 (2000)).

Methods are known in the art for evaluating whether a particular protease cleaves a selected peptide sequence. For example, the use of 7-amino-4-methyl coumarin (AMC) fluorogenic peptide substrates is a well-established method for the determination of protease specificity (Zimmerman, M., et al., (1977) Analytical Biochemistry 78:47-51). Specific cleavage of the anilide bond liberates the fluorogenic AMC leaving group allowing for the simple determination of cleavage rates for individual substrates. More recently, arrays (Lee, D., et al., (1999) Bioorganic and Medicinal Chemistry Letters 9:1667-72) and positional-scanning libraries (Rano, T. A., et al., (1997) Chemistry and Biology 4:149-55) of AMC peptide substrate libraries have been employed to rapidly profile the N-terminal specificity of proteases by sampling a wide range of substrates in a single experiment. Thus, one of skill in the art may readily evaluate an array of peptide sequences to determine their utility in the present invention without resort to undue experimentation.

The antibody-partner conjugate of the current invention may optionally contain two or more linkers. These linkers may be the same or different. For example, a peptidyl linker may be used to connect the drug to the ligand and a second peptidyl linker may attach a diagnostic agent to the complex. Other uses for additional linkers include linking analytical agents, biomolecules, targeting agents, and detectable labels to the antibody-partner complex.

Also within the scope of the present invention are compounds of the invention that are poly- or multi-valent species, including, for example, species such as dimers, trimers, tetramers and higher homologs of the compounds of the invention or reactive analogues thereof. The poly- and multi-valent species can be assembled from a single species or more than one species of the invention. For example, a dimeric construct can be “homo-dimeric” or “heterodimeric.” Moreover, poly- and multi-valent constructs in which a compound of the invention or a reactive analogue thereof, is attached to an oligomeric or polymeric framework (e.g., polylysine, dextran, hydroxyethyl starch and the like) are within the scope of the present invention. The framework is preferably polyfunctional (i.e. having an array of reactive sites for attaching compounds of the invention). Moreover, the framework can be derivatized with a single species of the invention or more than one species of the invention.

Moreover, the present invention includes compounds that are functionalized to afford compounds having water-solubility that is enhanced relative to analogous compounds that are not similarly functionalized. Thus, any of the substituents set forth herein can be replaced with analogous radicals that have enhanced water solubility. For example, it is within the scope of the invention to, for example, replace a hydroxyl group with a diol, or an amine with a quaternary amine, hydroxy amine or similar more water-soluble moiety. In a preferred embodiment, additional water solubility is imparted by substitution at a site not essential for the activity towards the ion channel of the compounds set forth herein with a moiety that enhances the water solubility of the parent compounds. Methods of enhancing the water-solubility of organic compounds are known in the art. Such methods include, but are not limited to, functionalizing an organic nucleus with a permanently charged moiety, e.g., quaternary ammonium, or a group that is charged at a physiologically relevant pH, e.g. carboxylic acid, amine. Other methods include, appending to the organic nucleus hydroxyl- or amine-containing groups, e.g. alcohols, polyols, polyethers, and the like. Representative examples include, but are not limited to, polylysine, polyethyleneimine, poly(ethyleneglycol) and poly(propyleneglycol). Suitable functionalization chemistries and strategies for these compounds are known in the art. See, for example, Dunn, R. L., et al., Eds. Polymeric Drugs and Drug Delivery Systems, ACS Symposium Series Vol. 469, American Chemical Society, Washington, D.C. 1991.

Hydrazine Linkers (H)

In a second embodiment, the conjugate of the invention comprises a hydrazine self-immolative linker, wherein the conjugate has the structure:

X⁴-(L⁴)ₚ-H-(L¹)ₘ-D

wherein D, L¹, L⁴, and X⁴ are as defined above and described further herein, and H is a linker comprising the structure:

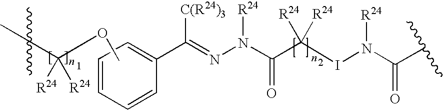

wherein n₁ is an integer from 1-10; n₂ is 0, 1, or 2; each R²⁴ is a member independently selected from the group consisting of H, substituted alkyl, unsubstituted alkyl, substituted heteroalkyl, and unsubstituted heteroalkyl; and I is either a bond (i.e., the bond between the carbon of the backbone and the adjacent nitrogen) or:

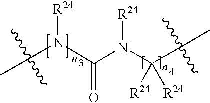

wherein n₃ is 0 or 1, with the proviso that when n₃ is 0, n₂ is not 0; and n₄ is 1, 2, or 3, wherein when I is a bond, n₁ is 3 and n₂ is 1, D can not be

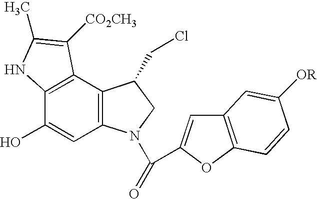

where R is Me or CH₂—CH₂—NMe₂.

In one embodiment, the substitution on the phenyl ring is a para substitution. In preferred embodiments, n₁ is 2, 3, or 4 or n₁ is 3. In preferred embodiments, n₂ is 1. In preferred embodiments, I is a bond (i.e., the bond between the carbon of the backbone and the adjacent nitrogen). In one aspect, the hydrazine linker, H, can form a 6-membered self immolative linker upon cleavage, for example, when n₃ is 0 and n₄ is 2. In another aspect, the hydrazine linker, H, can form two 5-membered self immolative linkers upon cleavage. In yet other aspects, H forms a 5-membered self immolative linker, H forms a 7-membered self immolative linker, or H forms a 5-membered self immolative linker and a 6-membered self immolative linker, upon cleavage. The rate of cleavage is affected by the size of the ring formed upon cleavage. Thus, depending upon the rate of cleavage desired, an appropriate size ring to be formed upon cleavage can be selected.

Five Membered Hydrazine Linkers

In one embodiment, the hydrazine linker comprises a 5-membered hydrazine linker, wherein H comprises the structure:

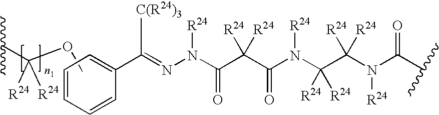

In a preferred embodiment, n₁ is 2, 3, or 4. In another preferred embodiment, n₁ is 3.

In the above structure, each R²⁴ is a member independently selected from the group consisting of H, substituted alkyl, unsubstituted alkyl, substituted heteroalkyl, and unsubstituted heteroalkyl. In one embodiment, each R²⁴ is independently H or a C₁-C₆ alkyl. In another embodiment, each R²⁴ is independently H or a C₁-C₃ alkyl, more preferably H or CH₃. In another embodiment, at least one R²⁴ is a methyl group. In another embodiment, each R₂₄ is H. Each R²⁴ is selected to tailor the compounds steric effects and for altering solubility.

The 5-membered hydrazine linkers can undergo one or more cyclization reactions that separate the drug from the linker, and can be described, for example, by:

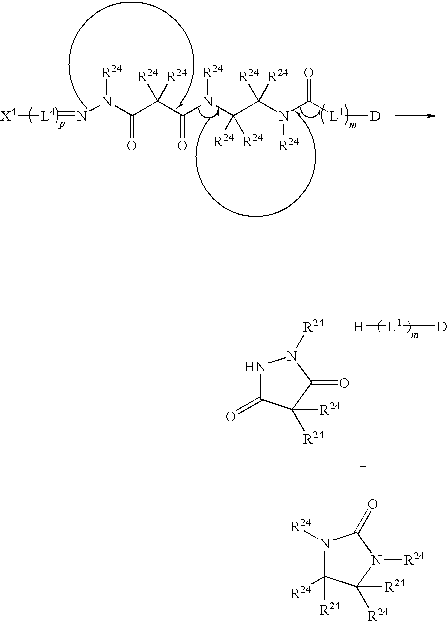

An exemplary synthetic route for preparing a five membered linker of the invention is:

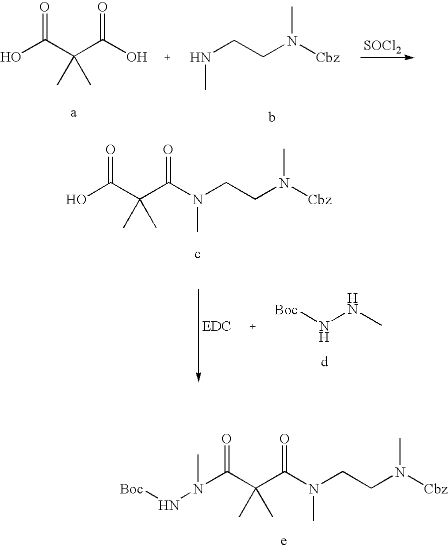

The Cbz-protected DMDA b is reacted with 2,2-Dimethyl-malonic acid a in solution with thionyl chloride to form a Cbz-DMDA-2,2-dimethylmalonic acid c. Compound c is reacted with Boc-N-methyl hydrazine d in the presence of EDC to form DMDA-2,2-dimetylmalonic-Boc-N-methylhydrazine e.

Six Membered Hydrazine Linkers

In another embodiment, the hydrazine linker comprises a 6-membered hydrazine linker, wherein H comprises the structure:

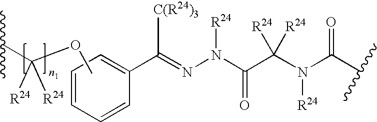

In a preferred embodiment, n₁ is 3. In the above structure, each R²⁴ is a member independently selected from the group consisting of H, substituted alkyl, unsubstituted alkyl, substituted heteroalkyl, and unsubstituted heteroalkyl. In one embodiment, each R²⁴ is independently H or a C₁-C₆ alkyl. In another embodiment, each R²⁴ is independently H or a C₁-C₃ alkyl, more preferably H or CH₃. In another embodiment, at least one R²⁴ is a methyl group. In another embodiment, each R₂₄ is H. Each R²⁴ is selected to tailor the compounds steric effects and for altering solubility. In a preferred embodiment, H comprises the structure:

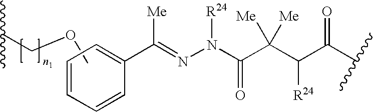

In one embodiment, H comprises a geminal dimethyl substitution. In one embodiment of the above structure, each R²⁴ is independently an H or a substituted or unsubstituted alkyl.

The 6-membered hydrazine linkers will undergo a cyclization reaction that separates the drug from the linker, and can be described as:

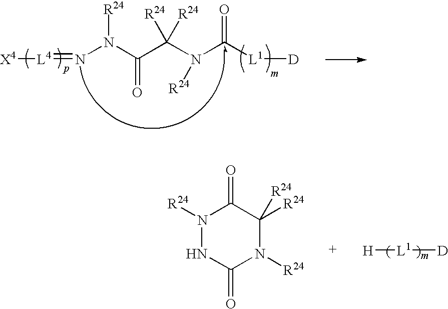

An exemplary synthetic route for preparing a six membered linker of the invention is:

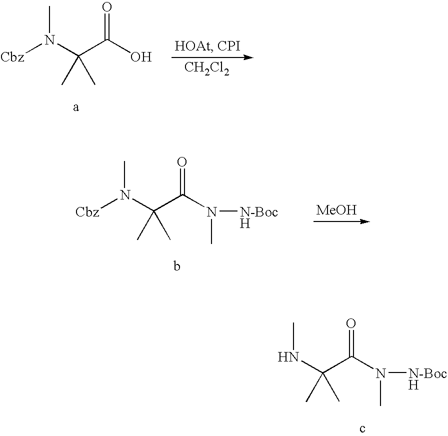

The Cbz-protected dimethyl alanine a in solution with dichlormethane, was reacted with HOAt, and CPI to form a Cbz-protected dimethylalanine hydrazine b. The hydrazine b is deprotected by the action of methanol, forming compound c.

Other Hydrazine Linkers

It is contemplated that the invention comprises a linker having seven members. This linker would likely not cyclize as quickly as the five or six membered linkers, but this may be preferred for some antibody-partner conjugates. Similarly, the hydrazine linker may comprise two six membered rings or a hydrazine linker having one six and one five membered cyclization products. A five and seven membered linker as well as a six and seven membered linker are also contemplated.

Another hydrazine structure, H, has the formula:

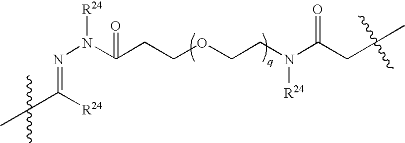

where q is 0, 1,2, 3, 4, 5, or 6; and

each R²⁴ is a member independently selected from the group consisting of H, substituted alkyl, unsubstituted alkyl, substituted heteroalkyl, and unsubstituted heteroalkyl. This hydrazine structure can also faun five-, six-, or seven-membered rings and additional components can be added to form multiple rings.

Disulfide Linkers (J)

In yet another embodiment, the linker comprises an enzymatically cleavable disulfide group. In one embodiment, the invention provides a cytotoxic antibody-partner compound having a structure according to Formula (d):

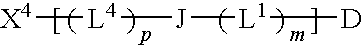

wherein D, L¹, L⁴, and X⁴ are as defined above and described further herein, and J is a disulfide linker comprising a group having the structure:

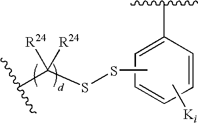

wherein each R²⁴ is a member independently selected from the group consisting of H, substituted alkyl, unsubstituted alkyl, substituted heteroalkyl, and unsubstituted heteroalkyl; each K is a member independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², NR²¹COR²², OCONR²¹, R²², OCOR²¹, and OR²¹ and R²² are independently selected from the group consisting of H, substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl and unsubstituted heterocycloalkyl; i is an integer of 0,1, 2, 3, or 4; and d is an integer of 0, 1, 2, 3, 4, 5, or 6.

The aromatic ring of the disulfides linker may be substituted with one or more “K” groups. A “K” group is a substituent on the aromatic ring that replaces a hydrogen otherwise attached to one of the four non-substituted carbons that are part of the ring structure. The “K” group may be a single atom, such as a halogen, or may be a multi-atom group, such as alkyl, heteroalkyl, amino, nitro, hydroxy, alkoxy, haloalkyl, and cyano. Exemplary K substituents independently include, but are not limited to, F, Cl, Br, I, NO₂, OH, OCH₃, NHCOCH₃, N(CH₃)₂, NHCOCF₃ and methyl. For “Kᵢ”, i is an integer of 0, 1, 2, 3, or 4. In a specific embodiment, i is 0.

In a preferred embodiment, the linker comprises an enzymatically cleavable disulfide group of the following formula:

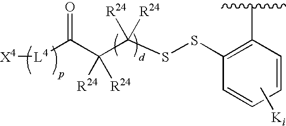

In this embodiment, the identities of L⁴, X⁴, p, and R²⁴ are as described above, and d is 0, 1, 2, 3, 4, 5, or 6. In a particular embodiment, d is 1 or 2.

A more specific disulfide linker is shown in the formula below:

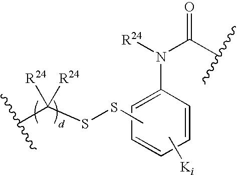

A specific example of this embodiment is as follows:

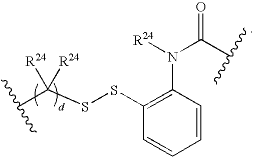

Preferably, d is 1 or 2.

Another disulfide linker is shown in the formula below:

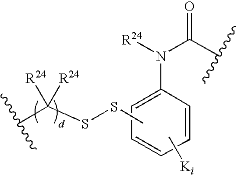

A specific example of this embodiment is as follows:

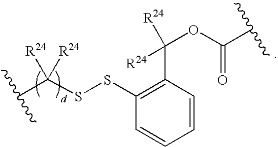

Preferably, d is 1 or 2.

In various embodiments, the disulfides are ortho to the amine. In another specific embodiment, a is 0. In preferred embodiments, R²⁴ is independently selected from H and CH₃.

An exemplary synthetic route for preparing a disulfide linker of the invention is as follows:

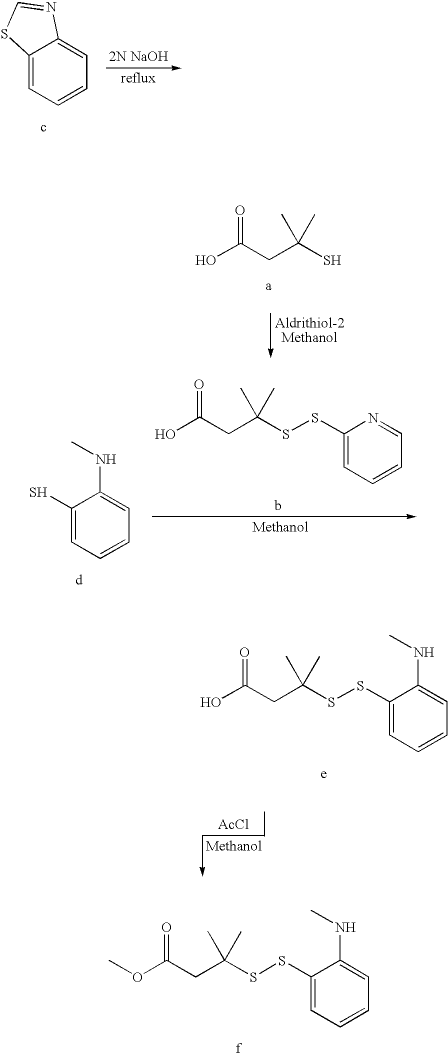

A solution of 3-mercaptopropionic acid a is reacted with aldrithiol-2 to form 3-methyl benzothiazolium iodide b. 3-methylbenzothiazolium iodide c is reacted with sodium hydroxide to form compound d. A solution of compound d with methanol is further reacted with compound b to form compound e. Compound e deprotected by the action of acetyl chloride and methanol forms compound f.

For further discussion of types of cytotoxins, linkers and other methods for conjugating therapeutic agents to antibodies, see also PCT Publication WO 2007/059404 to Gangwar et al. and entitled “Cytotoxic Compounds And Conjugates,” Saito, G. et al. (2003) Adv. Drug Deliv. Rev. 55:199-215; Trail, P. A. et al. (2003) Cancer Immunol Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264, each of which is hereby incorporated by reference in their entirety.

Partner Molecules

The present invention features an antibody conjugated to a partner molecule, such as a cytotoxin, a drug (e.g., an immunosuppressant) or a radiotoxin. Such conjugates are also referred to herein as “immunoconjugates.” Immnnoconjugates that include one or more cytotoxins are referred to as “immunotoxins.” A cytotoxin or cytotoxic agent includes any agent that is detrimental to (e.g., kills) cells.

Examples of partner molecules of the present invention include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Examples of partner molecules also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other preferred examples of partner molecules that can be conjugated to an antibody of the invention include duocarmycins, calicheamicins, maytansines and auristatins, and derivatives thereof. An example of a calicheamicin antibody conjugate is commercially available (Mylotarg®; American Home Products).

Preferred examples of partner molecule are CC-1065 and the duocannycins. CC-1065 was first isolated from Streptomyces zelensis in 1981 by the Upjohn Company (Hanka et al., J. Antibiot. 31: 1211 (1978); Martin et al., J. Antibiot. 33: 902 (1980); Martin et al., J. Antibiot. 34: 1119 (1981)) and was found to have potent antitumor and antimicrobial activity both in vitro and in experimental animals (Li et al., Cancer Res. 42: 999 (1982)). CC-1065 binds to double-stranded B-DNA within the minor groove (Swenson et al., Cancer Res. 42: 2821 (1982)) with the sequence preference of 5′-d(A/GNTTA)-3′ and 5′-d(AAAAA)-3′ and alkylates the N3 position of the 3′-adenine by its CPI left-hand unit present in the molecule (Hurley et al., Science 226: 843 (1984)). Despite its potent and broad antitumor activity, CC-1065 cannot be used in humans because it causes delayed death in experimental animals.

Many analogues and derivatives of CC-1065 and the duocarmycins are known in the art. The research into the structure, synthesis and properties of many of the compounds has been reviewed. See, for example, Boger et al., Angew. Chem. Int. Ed. Engl. 35: 1438 (1996); and Boger et al., Chem. Rev. 97: 787 (1997).

A group at Kyowa Hakko Kogya Co., Ltd. has prepared a number of CC-1065 derivatives. See, for example, U.S. Pat. Nos. 5,101,038; 5,641,780; 5,187,186; 5,070,092; 5,703,080; 5,070,092; 5,641,780; 5,101,038; and 5,084,468; and published PCT application, WO 96/10405 and published European application 0 537 575 A1.

The Upjohn Company (Pharmacia Upjohn) has also been active in preparing derivatives of CC-1065. See, for example, U.S. Pat. Nos. 5,739,350; 4,978,757, 5,332, 837 and 4,912,227.

A particularly preferred aspect of the current invention provides a cytotoxic compound having a structure according to the following formula (e):

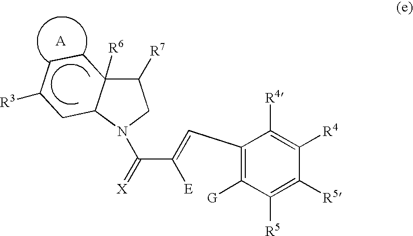

in which ring system A is a member selected from substituted or unsubstituted aryl substituted or unsubstituted heteroaryl and substituted or unsubstituted heterocycloalkyl groups. Exemplary ring systems include phenyl and pyrrole.

The symbols E and G are independently selected from H, substituted or unsubstituted alkyl, substituted or unsubstituted heteroalkyl, a heteroatom, a single bond or E and G are optionally joined to form a ring system selected from substituted or unsubstituted aryl, substituted or unsubstituted heteroaryl and substituted or unsubstituted heterocycloalkyl.

The symbol X represents a member selected from O, S and NR²³. R²³ is a member selected from H, substituted or unsubstituted alkyl, substituted or unsubstituted heteroalkyl, and acyl.

The symbol R³ represents a member selected from (═O), SR¹¹, NHR¹¹ and OR¹¹, in which R¹¹ is H, substituted or unsubstituted alkyl, substituted or unsubstituted heteroalkyl, monophosphates, diphosphates, triphosphates, sulfonates, acyl, C(O)R¹²R¹³, C(O)OR¹², C(O)NR¹²R¹³P(O)(OR¹²)₂, C(O)CHR¹²R¹³, SR¹² or SiR¹²R¹³R¹⁴. The symbols R¹², R¹³, and R¹⁴ independently represent H, substituted or unsubstituted alkyl, substituted or unsubstituted heteroalkyl and substituted or unsubstituted aryl, where R¹² and R¹³ together with the nitrogen or carbon atom to which they are attached are optionally joined to form a substituted or unsubstituted heterocycloalkyl ring system having from 4 to 6 members, optionally containing two or more heteroatoms. One or more of R¹², R¹³, or R¹⁴ can include a cleavable group within its structure.

R⁴, R^{4,}, R⁵ and R^{5,} are members independently selected from H, substituted or unsubstituted alkyl, substituted or unsubstituted aryl, substituted or unsubstituted heteroaryl, substituted or unsubstituted heterocycloalkyl, halogen, NO₂, NR¹⁵R¹⁶, NC(O)R¹⁵, OC(O)NR¹⁵R¹⁶, OC(O)OR^{15, C(O)R}¹⁵, SR¹⁵, OR¹⁵, CR¹⁵═NR¹⁶, and O(CH₂)ₙN(CH₃)₂, where n is an integer from 1 to 20, or any adjacent pair of R⁴, R^{4,} , R⁵ and R^{5,}, together with the carbon atoms to which they are attached, are joined to form a substituted or unsubstituted cycloalkyl or heterocycloalkyl ring system having from 4 to 6 members. R¹⁵ and R¹⁶ independently represent H, substituted or unsubstituted alkyl, substituted or unsubstituted heteroalkyl, substituted or unsubstituted aryl, substituted or unsubstituted heteroaryl, substituted or unsubstituted heterocycloalkyl and substituted or unsubstituted peptidyl, where R¹⁵ and R¹⁶ together with the nitrogen atom to which they are attached are optionally joined to form a substituted or unsubstituted heterocycloalkyl ring system having from 4 to 6 members, optionally containing two or more heteroatoms. One exemplary structure is aniline.

R⁴, R^{4,}, R⁵, R^{5,}, R¹¹, R¹², R¹³, R¹⁵ and R¹⁶ optionally contain one or more cleavable groups within their structure, such as a cleavable linker or cleavable substrate. Exemplary cleavable groups include, but are not limited to peptides, amino acids, hydrazines, disulfides, and cephalosporin derivatives.

In some embodiments, at least one of R⁴, R^{4,}, R⁵, R^{5,}, R¹¹, R¹², R¹³, R¹⁵ and R¹⁶ is used to join the drug to a linker or enzyme cleavable substrate of the present invention, as described herein, for example to L¹, if present or to F, H, J, or X², or J.

In a still further exemplary embodiment, at least one of R⁴, R^{4,}, R⁵, R^{5,}, R¹¹, R¹², R¹³, R¹⁵ and R¹⁶ bears a reactive group appropriate for conjugating the compound. In a further exemplary embodiment, R⁴, R^{4,}, R⁵, R^{5,}, R¹¹, R¹², R¹³, R¹⁵ and R¹⁶ are independently selected from H, substituted alkyl and substituted heteroalkyl and have a reactive functional group at the free terminus of the alkyl or heteroalkyl moiety. One or more of R⁴, R^{4,}, R⁵, R^{5,}, R¹¹, R¹², R¹³, R¹⁵ and R¹⁶ may be conjugated to another species, e.g., targeting agent, detectable label, solid support, etc.

R⁶ is a single bond which is either present or absent. When R⁶ is present, R⁶ and R⁷ are joined to form a cyclopropyl ring. R⁷ is CH₂—X¹ or —CH₂—. When R⁷ is —CH₂—it is a component of the cyclopropane ring. The symbol X¹ represents a leaving group such as a halogen, for example Cl, Br or F. The combinations of R⁶ and R⁷ are interpreted in a manner that does not violate the principles of chemical valence.

X¹ may be any leaving group. Useful leaving groups include, but are not limited to, halogens, azides, sulfonic esters (e.g., alkylsulfonyl, arylsulfonyl), oxonium ions, alkyl perchlorates, ammonioalkanesulfonate esters, alkylfluorosulfonates and fluorinated compounds (e.g., triflates, nonaflates, tresylates) and the like. Particular halogens useful as leaving groups are F, Cl and Br. The choice of these and other leaving groups appropriate for a particular set of reaction conditions is within the abilities of those of skill in the art (see, for example, March J, Advanced Organic Chemistry, 2nd Edition, John Wiley and Sons, 1992; Sandler S R, Karo W, Organic Functional Group Preparations, 2nd Edition, Academic Press, Inc., 1983; and Wade L G, Compendium of Organic Synthetic Methods, John Wiley and Sons, 1980).

The curved line within the six-membered ring indicates that the ring may have one or more degrees of unsaturation, and it may be aromatic. Thus, ring structures such as those set forth below, and related structures, are within the scope of Formula (f):

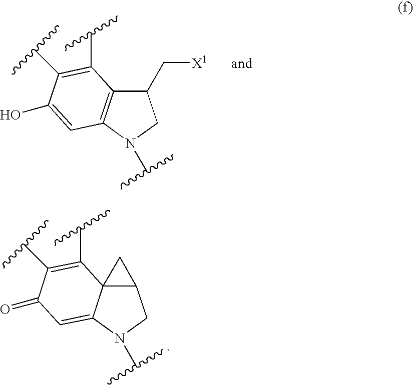

In some embodiments, at least one of R⁴, R^{4,}, R⁵, and R^{5,} links said drug to L¹, if present, or to F, H, J, or X², and includes

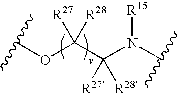

where v is an integer from 1 to 6; and each R²⁷, R^{27′}, R²⁸, and R^{28′} is independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl. In some embodiments, R²⁷, R^{27′}, R²⁸, and R^{28′} are all H. In some embodiments, v is an integer from 1 to 3 (preferably, 1). This unit can be used to separate aryl substituents from the drug and thereby resist or avoid generating compounds that are substrates for multi-drug resistance.

In one embodiment, R¹¹ includes a moiety, X⁵, that does not self-cyclize and links the drug to L^{I}, if present, or to F, H, J, or X². The moiety, X⁵, is preferably cleavable using an enzyme and, when cleaved, provides the active drug. As an example, R¹¹ can have the following structure (with the right side coupling to the remainder of the drug):

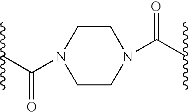

In an exemplary embodiment, ring system A of formula (e) is a substituted or unsubstituted phenyl ring. Ring system A may be substituted with one or more aryl group substituents as set forth in the definitions section herein. In some embodiments, the phenyl ring is substituted with a CN or methoxy moiety.

In some embodiments, at least one of R⁴, R^{4,}, R⁵, and R^{5,} links said drug to L¹, if present, or to F, H, J, or X², and R³ is selected from SR¹¹, NHR¹¹ and OR¹¹. R¹¹ is selected from —SO(OH)₂, —PO(OH)₂, -AAₙ, —Si(CH₃)₂C(CH₃)₃, —C(O)OPhNH(AA)ₘ,

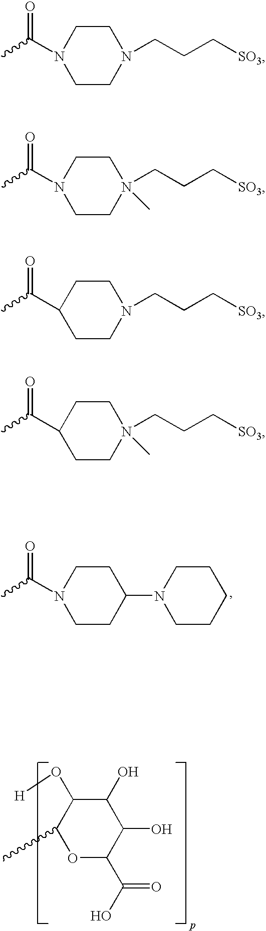

or any other sugar or combination of sugars,

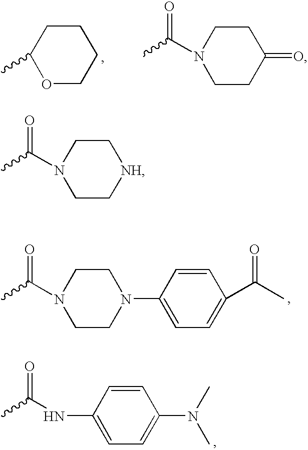

and pharmaceutically acceptable salts thereof, where n is any integer in the range of 1 to 10, m is any integer in the range of 1 to 4, p is any integer in the range of 1 to 6, and AA is any natural or non-natural amino acid. In some embodiments, AAₙ or AAₘ is selected from the same amino acid sequences described above for the peptide linkers (F) and optionally is the same as the amino acid sequence used in the linker portion of R⁴, R^{4,}, R⁵, or R^{5,}. In at least some embodiments, R³ is cleavable in vivo to provide an active drug compound. In at least some embodiments, R³ increases in vivo solubility of the compound. In some embodiments, the rate of decrease of the concentration of the active drug in the blood is substantially faster than the rate of cleavage of R³ to provide the active drug. This may be particularly useful where the toxicity of the active drug is substantially higher than that of the prodrug form. In other embodiments, the rate of cleavage of R³ to provide the active drug is faster than the rate of decrease of concentration of the active drug in the blood.

In another exemplary embodiment, the invention provides a compound having a structure according to Formula (g):

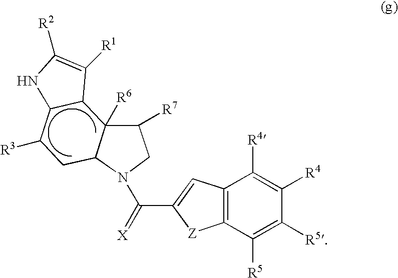

In this embodiment, the identities of the substituents R³, R⁴, R^{4,} , R⁵, R^{5,}, R⁶, R⁷ and X are substantially as described above for Formula (a), as well as preferences for particular embodiments. The symbol Z is a member independently selected from O, S and NR²³. The symbol R²³ represents a member selected from H, substituted or unsubstituted alkyl, substituted or unsubstituted heteroalkyl, and acyl. Each R²³ is independently selected. The symbol R¹ represents H, substituted or unsubstituted lower alkyl, or C(O)R⁸ or CO₂R⁸. R⁸ is a member selected from substituted alkyl, unsubstituted alkyl, NR⁹R¹⁰, NR⁹NHR¹⁰ and OR⁹. R⁹ and R¹⁰ are independently selected from H, substituted or unsubstituted alkyl and substituted or unsubstituted heteroalkyl. R² is H, or substituted or unsubstituted lower alkyl. It is generally preferred that when R² is substituted alkyl, it is other than a perfluoroalkyl, e.g., CF₃. In one embodiment, R² is a substituted alkyl wherein the substitution is not a halogen. In another embodiment, R² is an unsubstituted alkyl.

In some embodiments R¹ is an ester moiety, such as CO₂CH₃. In some embodiments, R² is a lower alkyl group, which may be substituted or unsubstituted. A presently preferred lower alkyl group is CH₃. In some preferred embodiments, R¹ is CO₂CH₃ and R² is CH₃. In some embodiments, R⁴, R^{4,}, R⁵, and R^{5,} are members independently selected from H, halogen, NH₂, OMe, O(CH₂)₂N(R²⁹)₂ and NO₂. Each R²⁹ is independently H or lower alkyl (e.g., methyl).

In some embodiments, the drug is selected such that the leaving group X¹ is a member selected from the group consisting of halogen, alkylsulfonyl, arylsulfonyl, and azide. In some embodiments, X¹ is F, Cl, or Br.

In some embodiments, Z is O or NH. In some embodiments, X is O.

In yet another exemplary embodiment, the invention provides compounds having a structure according to Formula (h) or (i):

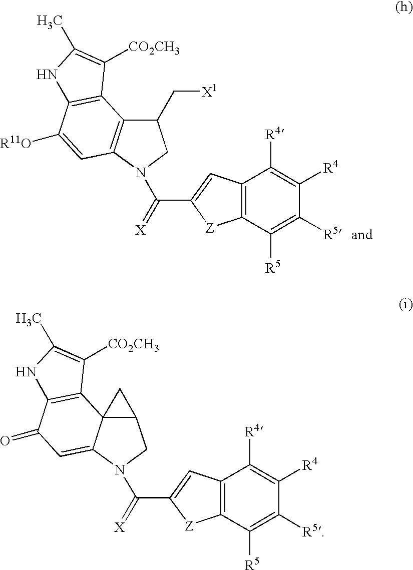

Another preferred structure of the duocarmycin analog of Formula (e) is a structure in which the ring system A is an unsubstituted or substituted phenyl ring. The preferred substituents on the drug molecule described hereinabove for the structure of Formula 7 when the ring system A is a pyrrole are also preferred substituents when the ring system A is an unsubstituted or substituted phenyl ring.

For example, in a preferred embodiment, the drug (D) comprises a structure (j):

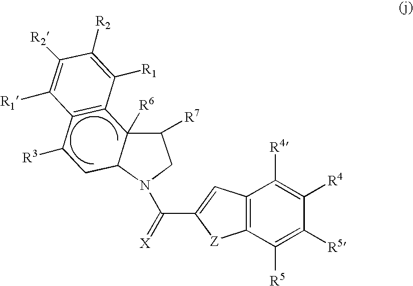

In this structure, R³, R⁶, R⁷, X are as described above for Formula (e). Furthermore, Z is a member selected from O, S and NR²³, wherein R²³ is a member selected from H, substituted or unsubstituted alkyl, substituted or unsubstituted heteroalkyl, and acyl;

R¹ is H, substituted or unsubstituted lower alkyl, C(O)R⁸, or CO₂R⁸, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl;

R^{1′} is H, substituted or unsubstituted lower alkyl, or C(O)R⁸, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl;

R² is H, or substituted or unsubstituted lower alkyl or unsubstituted heteroalkyl or cyano or alkoxy; and R^{2′} is H, or substituted or unsubstituted lower alkyl or unsubstituted heteroalkyl. At least one of R⁴, R^{4,}, R⁵, R^{5,}, R¹¹, R¹², R¹³, R¹⁵ or R¹⁶ links the drug to L¹, if present, or to F, H, J, or X².

Another embodiment of the drug (D) comprises a structure (k) where R⁴ and R^{4′} have been joined to from a heterocycloalkyl:

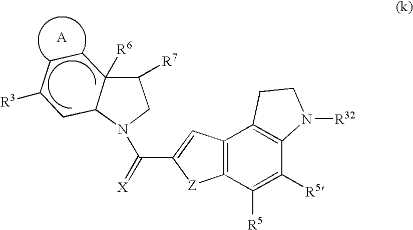

In this structure, R³, R⁵, R^{5′}, R⁶, R⁷, X are as described above for Formula (e). Furthermore, Z is a member selected from O, S and NR²³, wherein R²³ is a member selected from H, substituted or unsubstituted alkyl, substituted or unsubstituted heteroalkyl, and acyl;

R³² is selected from H, substituted or unsubstituted alkyl, substituted or unsubstituted aryl, substituted or unsubstituted heteroaryl, substituted or unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, where n is an integer from 1 to 20. R¹⁵ and R¹⁶ independently represent H, substituted or unsubstituted alkyl, substituted or unsubstituted heteroalkyl, substituted or unsubstituted aryl, substituted or unsubstituted heteroaryl, substituted or unsubstituted heterocycloalkyl and substituted or unsubstituted peptidyl, where R¹⁵ and R¹⁶ together with the nitrogen atom to which they are attached are optionally joined to form a substituted or unsubstituted heterocycloalkyl ring system having from 4 to 6 members, optionally containing two or more heteroatoms. R³² optionally contains one or more cleavable groups within its structure, such as a cleavable linker or cleavable substrate. Exemplary cleavable groups include, but are not limited to, peptides, amino acids, hydrazines, disulfides, and cephalosporin derivatives. Moreover, any selection of substituents described herein for R⁴, R^{4′}, R⁵, R^{5,}, R¹⁵, and R¹⁶ is also applicable to R³².

At least one of R⁵, R^{5,}, R^{11,}R¹², R¹³, R¹⁵, R¹⁶, or R³² links the drug to L¹if present, or to F, H, J, or X². In at least some embodiments, R³² links the drug to L¹, if present, or to F, H, J, or X².

One preferred embodiment of this compound is:

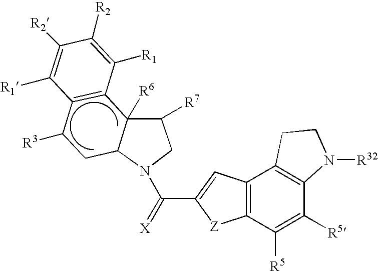

R¹ is H, substituted or unsubstituted lower alkyl, C(O)R⁸, or CO₂R⁸, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl;

R^{1′} is H, substituted or unsubstituted lower alkyl, or C(O)R⁸, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl;

R² is H, or substituted or unsubstituted lower alkyl or unsubstituted heteroalkyl or cyano or alkoxy; and R^{2′} is H, or substituted or unsubstituted lower alkyl or unsubstituted heteroalkyl.

A further embodiment has the formula:

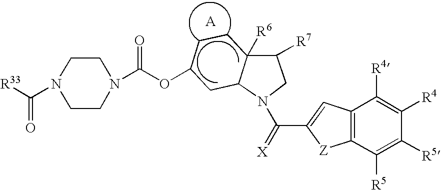

In this structure, A, R⁶, R⁷, X, R⁴, R^{4′}, R⁵, and R^{5′} are as described above for Formula (e). Furthermore, Z is a member selected from O, S and NR²³, where R²³ is a member selected from H, substituted or unsubstituted alkyl, substituted or unsubstituted heteroalkyl, and acyl;

R³³ is selected from H, substituted or unsubstituted alkyl, substituted or unsubstituted aryl, substituted or unsubstituted heteroaryl, substituted or unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR^{15═NR}¹⁶, and O(CH₂)ₙN(CH₃)₂, where n is an integer from 1 to 20. R¹⁵ and R¹⁶ independently represent H, substituted or unsubstituted alkyl, substituted or unsubstituted heteroalkyl, substituted or unsubstituted aryl, substituted or unsubstituted heteroaryl, substituted or unsubstituted heterocycloalkyl and substituted or unsubstituted peptidyl, where R¹⁵ and R¹⁶ together with the nitrogen atom to which they are attached are optionally joined to form a substituted or unsubstituted heterocycloalkyl ring system having from 4 to 6 members, optionally containing two or more heteroatoms. R³³ links the drug to L¹, if present, or to F, H, J, or X².

Preferably, A is substituted or unsubstituted phenyl or substituted or unsubstituted pyrrole. Moreover, any selection of substituents described herein for R¹¹ is also applicable to R³³.

Ligands

X⁴ represents a ligand selected from the group consisting of protected reactive functional groups, unprotected reactive functional groups, detectable labels, and targeting agents. Preferred ligands are targeting agents, such as antibodies and fragments thereof.

In some embodiments, the group X⁴ can be described as a member selected from R²⁹, COOR²⁹, C(O)NR²⁹, and C(O)NNR²⁹ wherein R²⁹ is a member selected from substituted or unsubstituted alkyl, substituted or unsubstituted heteroalkyl and substituted or unsubstituted heteroaryl. In yet another exemplary embodiment, R²⁹ is a thiol reactive member. In a further exemplary embodiment, R²⁹ is a thiol reactive member selected from haloacetyl and alkyl halide derivatives, maleimides, aziridines, and acryloyl derivatives. The above thiol reactive members can act as reactive protective groups that can be reacted with, for example, a side chain of an amino acid of a targeting agent, such as an antibody, to thereby link the targeting agent to the linker-drug moiety.

Detectable Labels

The particular label or detectable group used in conjunction with the compounds and methods of the invention is generally not a critical aspect of the invention, as long as it does not significantly interfere with the activity or utility of the compound of the invention. The detectable group can be any material having a detectable physical or chemical property. Such detectable labels have been well developed in the field of immunoassays and, in general, any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to a compound of the invention according to methods well known in the art. As indicated above, a wide variety of labels may be used, with the choice of label depending on sensitivity required, ease of conjugation with the compound, stability requirements, available instrumentation, and disposal provisions.

When the compound of the invention is conjugated to a detectable label, the label is preferably a member selected from the group consisting of radioactive isotopes, fluorescent agents, fluorescent agent precursors, chromophores, enzymes and combinations thereof. Methods for conjugating various groups to antibodies are well known in the art. For example, a detectable label that is frequently conjugated to an antibody is an enzyme, such as horseradish peroxidase, alkaline phosphatase, β-galactosidase, and glucose oxidase.

Non-radioactive labels are often attached by indirect means. Generally, a ligand molecule (e.g., biotin) is covalently bound to a component of the conjugate. The ligand then binds to another molecules (e.g., streptavidin) molecule, which is either inherently detectable or covalently bound to a signal system, such as a detectable enzyme, a fluorescent compound, or a chemiluminescent compound.

Components of the conjugates of the invention can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see, U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Fluorescent labels are presently preferred as they have the advantage of requiring few precautions in handling, and being amenable to high-throughput visualization techniques (optical analysis including digitization of the image for analysis in an integrated system comprising a computer). Preferred labels are typically characterized by one or more of the following: high sensitivity, high stability, low background, low environmental sensitivity and high specificity in labeling. Many fluorescent labels are commercially available from the SIGMA chemical company (Saint Louis, Mo.), Molecular Probes (Eugene, Oreg.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica- Biochemika Analytika (Fluka Chemie AG, Buchs, Switzerland), and Applied Biosystems (Foster City, Calif.), as well as many other commercial sources known to one of skill. Furthermore, those of skill in the art will recognize how to select an appropriate fluorophore for a particular application and, if it is not readily available commercially, will be able to synthesize the necessary fluorophore de novo or synthetically modify commercially available fluorescent compounds to arrive at the desired fluorescent label.

In addition to small molecule fluorophores, naturally occurring fluorescent proteins and engineered analogues of such proteins are useful in the present invention. Such proteins include, for example, green fluorescent proteins of cnidarians (Ward et al., Photochem. Photobiol. 35:803-808 (1982); Levine et al., Comp. Biochem. Physiol., 72B:77-85 (1982)), yellow fluorescent protein from Vibrio fischeri strain (Baldwin et al., Biochemistry 29:5509-15 (1990)), Peridinin-chlorophyll from the dinoflagellate Symbiodinium sp. (Morris et al., Plant Molecular Biology 24:673:77 (1994)), phycobiliproteins from marine cyanobacteria, such as Synechococcus, e.g., phycoerythrin and phycocyanin (Wilbanks et al., J. Biol. Chem. 268:1226-35 (1993)), and the like.

Generally, prior to forming the linkage between the cytotoxin and the targeting (or other) agent, and optionally, the spacer group, at least one of the chemical functionalities will be activated. One skilled in the art will appreciate that a variety of chemical functionalities, including hydroxy, amino, and carboxy groups, can be activated using a variety of standard methods and conditions. For example, a hydroxyl group of the cytotoxin or targeting agent can be activated through treatment with phosgene to form the corresponding chioroformate, or p-nitrophenylchloroformate to form the corresponding carbonate.

In an exemplary embodiment, the invention makes use of a targeting agent that includes a carboxyl functionality. Carboxyl groups may be activated by, for example, conversion to the corresponding acyl halide or active ester. This reaction may be performed under a variety of conditions as illustrated in March, supra pp. 388-89. In an exemplary embodiment, the acyl halide is prepared through the reaction of the carboxyl-containing group with oxalyl chloride. The activated agent is reacted with a cytotoxin or cytotoxin-linker arm combination to form a conjugate of the invention. Those of skill in the art will appreciate that the use of carboxyl-containing targeting agents is merely illustrative, and that agents having many other functional groups can be conjugated to the linkers of the invention.

Reactive Functional Groups

For clarity of illustration the succeeding discussion focuses on the conjugation of a cytotoxin to a targeting agent. The focus exemplifies one embodiment of the invention from which, others are readily inferred by one of skill in the art. No limitation of the invention is implied, by focusing the discussion on a single embodiment.

Exemplary compounds of the invention bear a reactive functional group, which is generally located on a substituted or unsubstituted alkyl or heteroalkyl chain, allowing their facile attachment to another species. A convenient location for the reactive group is the terminal position of the chain.

Reactive groups and classes of reactions useful in practicing the present invention are generally those that are well known in the art of bioconjugate chemistry. The reactive functional group may be protected or unprotected, and the protected nature of the group may be changed by methods known in the art of organic synthesis. Preferred classes of reactions available with reactive cytotoxin analogues are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al., Modification of Proteins; Advances in Chemistry Series, Vol. 198, American Chemical Society, Washington, D.C., 1982.

Exemplary reaction types include the reaction of carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters. Hydroxyl groups can be converted to esters, ethers, aldehydes, etc. Haloalkyl groups are converted to new species by reaction with, for example, an amine, a carboxylate anion, thiol anion, carbanion, or an alkoxide ion. Dienophile (e.g., maleimide) groups participate in Diels-Alder. Aldehyde or ketone groups can be converted to imines, hydrazones, semicarbazones or oximes, or via such mechanisms as Grignard addition or alkyllithium addition. Sulfonyl halides react readily with amines, for example, to form sulfonamides. Amine or sulfhydryl groups are, for example, acylated, alkylated or oxidized. Alkenes, can be converted to an array of new species using cycloadditions, acylation, Michael addition, etc. Epoxides react readily with amines and hydroxyl compounds.

One skilled in the art will readily appreciate that many of these linkages may be produced in a variety of ways and using a variety of conditions. For the preparation of esters, see, e.g., March supra at 1157; for thioesters, see, March, supra at 362-363, 491, 720-722, 829, 941, and 1172; for carbonates, see, March, supra at 346-347; for carbamates, see, March, supra at 1156-57; for amides, see, March supra at 1152; for ureas and thioureas, see, March supra at 1174; for acetals and ketals, see, Greene et al. supra 178-210 and March supra at 1146; for acyloxyalkyl derivatives, see, Prodrugs: Topical and Ocular Drug Delivery, K. B. Sloan, ed., Marcel Dekker, Inc., New York, 1992; for enol esters, see, March supra at 1160; for N-sulfonylimidates, see, Bundgaard et al., J. Med. Chem., 31:2066 (1988); for anhydrides, see, March supra at 355-56, 636-37, 990-91, and 1154; for N-acylamides, see, March supra at 379; for N-Mannich bases, see, March supra at 800-02, and 828; for hydroxymethyl ketone esters, see, Petracek et al. Annals NY Acad. Sci., 507:353-54 (1987); for disulfides, see, March supra at 1160; and for phosphonate esters and phosphonamidates.

The reactive functional groups can be unprotected and chosen such that they do not participate in, or interfere with, the reactions. Alternatively, a reactive functional group can be protected from participating in the reaction by the presence of a protecting group. Those of skill in the art will understand how to protect a particular functional group from interfering with a chosen set of reaction conditions. For examples of useful protecting groups, See Greene et al., Protective Groups in Organic Synthesis, John Wiley & Sons, New York, 1991.

Typically, the targeting agent is linked covalently to a cytotoxin using standard chemical techniques through their respective chemical functionalities. Optionally, the linker or agent is coupled to the agent through one or more spacer groups. The spacer groups can be equivalent or different when used in combination.

Generally, prior to forming the linkage between the cytotoxin and the reactive functional group, and optionally, the spacer group, at least one of the chemical functionalities will be activated. One skilled in the art will appreciate that a variety of chemical functionalities, including hydroxy, amino, and carboxy groups, can be activated using a variety of standard methods and conditions. In an exemplary embodiment, the invention comprises a carboxyl functionality as a reactive functional group. Carboxyl groups may be activated as described hereinabove.

Cleavable Substrate

The cleavable substrates of the current invention are depicted as “X²”. Preferably, the cleavable substrate is a cleavable enzyme substrate that can be cleaved by an enzyme. Preferably, the enzyme is preferentially associated, directly or indirectly, with the tumor or other target cells to be treated. The enzyme may be generated by the tumor or other target cells to be treated. For example, the cleavable substrate can be a peptide that is preferentially cleavable by an enzyme found around or in a tumor or other target cell. Additionally or alternatively, the enzyme can be attached to a targeting agent that binds specifically to tumor cells, such as an antibody specific for a tumor antigen.

As examples of enzyme cleavable substrates suitable for coupling to the drugs described above, PCT Patent Applications Publication Nos. WO 00/33888, WO 01/95943, WO 01/95945, WO 02/00263, and WO 02/100353, all of which are incorporated herein by reference, disclose attachment of a cleavable peptide to a drug. The peptide is cleavable by an enzyme, such as a trouase (such as thimet oligopeptidase), CD10 (neprilysin), a matrix metalloprotease (such as MMP2 or MMP9), a type II transmembrane serine protease (such as Hepsin, testisin, TMPRSS4, or matriptase/MT-SP1), or a cathepsin, associated with a tumor. In this embodiment, a prodrug includes the drug as described above, a peptide, a stabilizing group, and optionally a linking group between the drug and the peptide. The stabilizing group is attached to the end of the peptide to protect the prodrug from degradation before arriving at the tumor or other target cell. Examples of suitable stabilizing groups include non-amino acids, such as succinic acid, diglycolic acid, maleic acid, polyethylene glycol, pyroglutamic acid, acetic acid, naphthylcarboxylic acid, terephthalic acid, and glutaric acid derivatives; as well as non-genetically-coded amino acids or aspartic acid or glutamic acid attached to the N-terminus of the peptide at the β-carboxy group of aspartic acid or the γ-carboxyl group of glutamic acid.

The peptide typically includes 3-12 (or more) amino acids. The selection of particular amino acids will depend, at least in part, on the enzyme to be used for cleaving the peptide, as well as, the stability of the peptide in vivo. One example of a suitable cleavable peptide is β̅AlaLeuAlaLeu (SEQ ID NO:92). This can be combined with a stabilizing group to form succinyl-βAlaLeuAlaLeu (SEQ ID NO:92). Other examples of suitable cleavable peptides are provided in the references cited above.

As one illustrative example, CD 10, also known as neprilysin, neutral endopeptidase (NEP), and common acute lymphoblastic leukemia antigen (CALLA), is a type II cell-surface zinc-dependent metalloprotease. Cleavable substrates suitable for use with CD10 include LeuAlaLeu and IleAlaLeu. Other known substrates for CD10 include peptides of up to 50 amino acids in length, although catalytic efficiency often declines as the substrate gets larger.

Another illustrative example is based on matrix metalloproteases (MMP). Probably the best characterized proteolytic enzymes associated with tumors, there is a clear correlation of activation of MMPs within tumor microenvironments. In particular, the soluble matrix enzymes MMP2 (gelatinase A) and MMP9 (gelatinase B), have been intensively studied, and shown to be selectively activated during tissue remodeling including tumor growth. Peptide sequences designed to be cleaved by MMP2 and MMP9 have been designed and tested for conjugates of dextran and methotrexate (Chau et al., Bioconjugate Chem. 15:931-941 (2004)); PEG (polyethylene glycol) and doxorubicin (Bae et al., Drugs Exp. Clin. Res. 29:15-23 (2004)); and albumin and doxorubicin (Kratz et al., Bioorg. Med. Chem. Lett. 11:2001-2006 (2001)). Examples of suitable sequences for use with MMPs include, but are not limited to, ProValGlyLeuIleGly (SEQ ID NO:84), GlyProLeuGlyVal (SEQ ID NO:85), GlyProLeuGlylleAlaGlyGln (SEQ ID NO:86), ProLeuGlyLeu (SEQ ID NO:87), GlyProLeuGlyMetLeuSerGln (SEQ ID NO:88), and GlyProLeuGlyLeuTrpAlaGln (SEQ ID NO:89). (See, e.g., the previously cited references as well as Kline et al., Mol. Pharmaceut. 1:9-22 (2004) and Liu et al., Cancer Res. 60:6061-6067 (2000).) Other cleavable substrates can also be used.

Yet another example is type II transmembrane serine proteases. This group of enzymes includes, for example, hepsin, testisin, and TMPRSS4. G1nAlaArg is one substrate sequence that is useful with matriptase/MT-SP 1 (which is over-expressed in breast and ovarian cancers) and LeuSerArg is useful with hepsin (over-expressed in prostate and some other tumor types). (See, e.g., Lee et. al., J. Biol. Chem. 275:36720-36725 and Kurachi and Yamamoto, Handbook of Proeolytic Enzymes Vol. 2, 2^{nd} edition (Barrett AJ, Rawlings N D & Woessner J F, eds) pp. 1699-1702 (2004).) Other cleavable substrates can also be used.

Another type of cleavable substrate arrangement includes preparing a separate enzyme capable of cleaving the cleavable substrate that becomes associated with the tumor or cells. For example, an enzyme can be coupled to a tumor-specific antibody (or other entity that is preferentially attracted to the tumor or other target cell such as a receptor ligand) and then the enzyme-antibody conjugate can be provided to the patient. The enzyme-antibody conjugate is directed to, and binds to, antigen associated with the tumor. Subsequently, the drug-cleavable substrate conjugate is provided to the patient as a prodrug. The drug is only released in the vicinity of the tumor when the drug-cleavable substrate conjugate interacts with the enzyme that has become associated with the tumor so that the cleavable substrate is cleaved and the drug is freed. For example, U.S. Pat. Nos. 4,975,278; 5,587,161; 5,660,829; 5,773,435; and 6,132,722, all of which are incorporated herein by reference, disclose such an arrangement. Examples of suitable enzymes and substrates include, but are not limited to, β-lactamase and cephalosporin derivatives, carboxypeptidase G2 and glutamic and aspartic folate derivatives. In one embodiment, the enzyme-antibody conjugate includes an antibody, or antibody fragment, that is selected based on its specificity for an antigen expressed on a target cell, or at a target site, of interest. A discussion of antibodies is provided hereinabove. One example of a suitable cephalosporin-cleavable substrate is

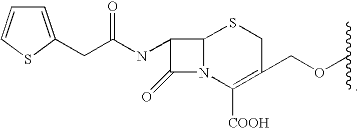

Examples of Conjugates

The linkers and cleavable substrates of the invention can be used in conjugates containing a variety of partner molecules. Examples of conjugates of the invention are described in further detail below. Unless otherwise indicated, substituents are defined as set forth above in the sections regarding cytotoxins, linkers, and cleavable substrates.

A. Linker Conjugates

One example of a suitable conjugate is a compound of the formula:

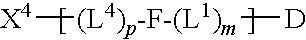

wherein L¹ is a self-immolative linker; m is an integer 0, 1, 2, 3, 4, 5, or 6; F is a linker comprising the structure:

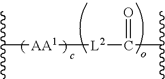

wherein AA¹ is one or more members independently selected from the group consisting of natural amino acids and unnatural α-amino acids; c is an integer from 1 to 20; L² is a self-immolative linker and comprises

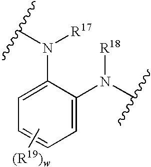

wherein each R¹⁷, R¹⁸, and R¹⁹ is independently selected from H, substituted or unsubstituted alkyl, substituted or unsubstituted heteroalkyl and substituted or unsubstituted aryl, and w is an integer from 0 to 4; o is 1; L⁴ is a linker member; p is 0 or 1; X⁴ is a member selected from the group consisting of protected reactive functional groups, unprotected reactive functional groups, detectable labels, and targeting agents; and D comprises a structure:

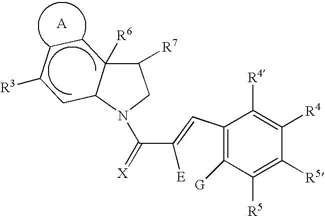

wherein the ring system A is a member selected from substituted or unsubstituted aryl, substituted or unsubstituted heteroaryl and substituted or unsubstituted heterocycloalkyl groups; E and G are members independently selected from H, substituted or unsubstituted alkyl, substituted or unsubstituted heteroalkyl, a heteroatom, a single bond, or E and G are joined to foim a ring system selected from substituted or unsubstituted aryl, substituted or unsubstituted heteroaryl and substituted or unsubstituted heterocycloalkyl; X is a member selected from O, S and NR²³; R²³ is a member selected from H, substituted or unsubstituted alkyl, substituted or unsubstituted heteroalkyl, and acyl; R³ is OR¹¹, wherein R¹¹ is a member selected from the group consisting of H, substituted alkyl, unsubstituted alkyl, substituted heteroalkyl, unsubstituted heteroalkyl, monophosphates, diphosphates, triphosphates, sulfonates, acyl, C(O)R¹²R¹³, C(O)OR¹², C(O)NR¹²R¹³, P(O)(OR¹²)₂, C(O)CHR¹²R¹³, SR¹² and SiR¹²R¹³R¹⁴, R⁴, R^{4,}, R⁵ and members independently selected from the group consisting of H, substituted alkyl, unsubstituted alkyl, substituted aryl, unsubstituted aryl, substituted heteroaryl, unsubstituted heteroaryl, substituted heterocycloalkyl, unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, C¹⁵═NR¹⁶, and O(CH₂)ₙN(CH₃)₂, or any adjacent pair of R⁴, R^{4,}, R⁵ and R^{5,}, together with the carbon atoms to which they are attached, are joined to form a substituted or unsubstituted cycloalkyl or heterocycloalkyl ring system having from 4 to 6 members; wherein n is an integer from 1 to 20; R¹⁵ and R¹⁶ are independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl, and substituted or unsubstituted peptidyl, wherein R¹⁵ and R¹⁶ together with the nitrogen atom to which they are attached are optionally joined to form a substituted or unsubstituted heterocycloalkyl ring system having from 4 to 6 members, optionally containing two or more heteroatoms; R⁶ is a single bond which is either present or absent and when present R⁶ and R⁷ are joined to form a cyclopropyl ring; and R⁷ is CH₂—X¹ or —CH₂— joined in said cyclopropyl ring with R⁶, wherein X¹ is a leaving group, wherein R¹¹ links said drug to L¹, if present, or to F.

In some embodiments, the drug has structure (c) or (f) above. One specific example of a compound suitable for use as a conjugate is

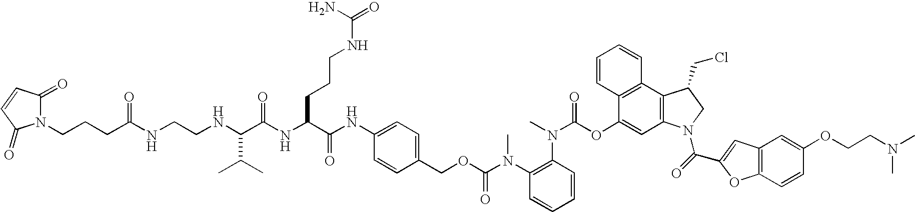

Another example of a type of conjugate is a compound of the formula

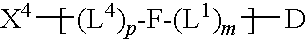

wherein L¹ is a self-immolative linker; m is an integer 0, 1, 2, 3, 4, 5, or 6; F is a linker comprising the structure:

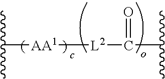

wherein AA¹ is one or more members independently selected from the group consisting of natural amino acids and unnatural α-amino acids; c is an integer from 1 to 20; L² is a self-imrnolative linker; o is 0 or 1; L⁴ is a linker member; p is 0 or 1; X⁴ is a member selected from the group consisting of protected reactive functional groups, unprotected reactive functional groups, detectable labels, and targeting agents; and D comprises a structure:

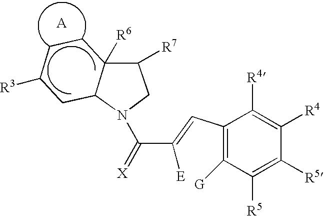

wherein the ring system A is a member selected from substituted or unsubstituted aryl, substituted or unsubstituted heteroaryl and substituted or unsubstituted heterocycloalkyl groups; E and G are members independently selected from H, substituted or unsubstituted alkyl, substituted or unsubstituted heteroalkyl, a heteroatom, a single bond, or E and G are joined to form a ring system selected from substituted or unsubstituted aryl, substituted or unsubstituted heteroaryl and substituted or unsubstituted heterocycloalkyl; X is a member selected from O, S and NR²³; R²³ is a member selected from H, substituted or unsubstituted alkyl, substituted or unsubstituted heteroalkyl, and acyl; R³ is a member selected from the group consisting of (═O), SR¹¹, NHR¹¹ and OR¹¹, wherein R¹¹ is a member selected from the group consisting of H, substituted alkyl, unsubstituted alkyl, substituted heteroalkyl, unsubstituted heteroalkyl, monophosphates, diphosphates, triphosphates, sulfonates, acyl, C(O)R¹²R¹³, C(O)OR¹², C(O)NR¹²R¹³, P(O)(OR¹²)₂, C(O)CHR¹²R¹³, SR¹² and SiR¹²R¹³R¹⁴, in which R¹², R¹³, and R¹⁴ are members independently selected from H, substituted or unsubstituted alkyl, substituted or unsubstituted heteroalkyl and substituted or unsubstituted aryl, wherein R¹² and R¹³ together with the nitrogen or carbon atom to which they are attached are optionally joined to form a substituted or unsubstituted heterocycloalkyl ring system having from 4 to 6 members, optionally containing two or more heteroatoms; R⁴ , R^{4,}, R⁵ and R^{5,} are members independently selected from the group consisting of H, substituted alkyl, unsubstituted alkyl, substituted aryl, unsubstituted aryl, substituted heteroaryl, unsubstituted heteroaryl, substituted heterocycloalkyl, unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, or any adjacent pair of R⁴, R^{4,}, R⁵ and R^{5,}, together with the carbon atoms to which they are attached, are joined to form a substituted or unsubstituted cycloalkyl or heterocycloalkyl ring system having from 4 to 6 members, wherein
n is an integer from 1 to 20; R¹⁵ and R¹⁶ are independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl, and substituted or unsubstituted peptidyl, wherein R¹⁵ and R¹⁶ together with the nitrogen atom to which they are attached are optionally joined to form a substituted or unsubstituted heterocycloalkyl ring system having from 4 to 6 members, optionally containing two or more heteroatoms; wherein at least one of R⁴ , R^{4,}, R⁵ and R^{5,} links said drug to L¹, if present, or to F, and comprises

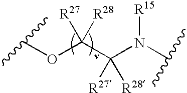

wherein v is an integer from 1 to 6; and each R²⁷, R^{27′}, R²⁸, and R^{28′} is independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl; R⁶ is a single bond which is either present or absent and when present R⁶ and R⁷ are joined to form a cyclopropyl ring; and R⁷ is CH₂—X¹ or —CH₂— joined in said cyclopropyl ring with R⁶, wherein X¹ is a leaving group.

In some embodiment, the drug has structure (c) or (f) above. One specific example of a compound suitable for use as a conjugate is

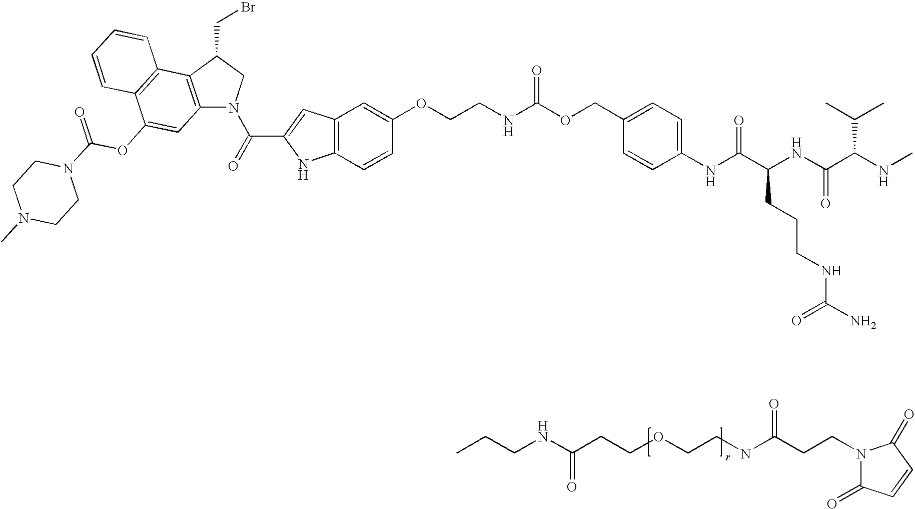

where r is an integer in the range from 0 to 24.

Another example of a suitable conjugate is a compound of the formula

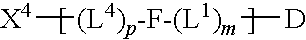

wherein L¹ is a self-immolative linker; m is an integer 0, 1, 2, 3, 4, 5, or 6; F is a linker comprising the structure:

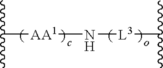

wherein AA¹ is one or more members independently selected from the group consisting of natural amino acids and unnatural α-amino acids; c is an integer from 1 to 20; L³ is a spacer group comprising a primary or secondary amine or a carboxyl functional group; wherein if L³ is present, m is 0 and either the amine of L³ forms an amide bond with a pendant carboxyl functional group of D or the carboxyl of L³ forms an amide bond with a pendant amine functional group of D; o is 0 or 1; L⁴ is a linker member, wherein L⁴ comprises

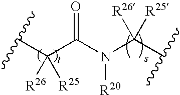

directly attached to the N-terminus of (AA¹)_{c}, wherein R²⁰ is a member selected from H, substituted or unsubstituted alkyl, substituted or unsubstituted heteroalkyl, and acyl, each R²⁵, R^{25′}, R²⁶, and R^{26′} is independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl; and s and t are independently integers from 1 to 6; p is 1; X⁴ is a member selected from the group consisting of protected reactive functional groups, unprotected reactive functional groups, detectable labels, and targeting agents; and D comprises a structure:

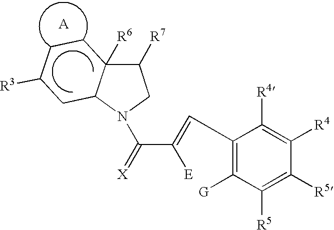

wherein the ring system A is a member selected from substituted or unsubstituted aryl, substituted or unsubstituted heteroaryl and substituted or unsubstituted heterocycloalkyl groups; E and G are members independently selected from H, substituted or unsubstituted alkyl, substituted or unsubstituted heteroalkyl, a heteroatom, a single bond, or E and G are joined to form a ring system selected from substituted or unsubstituted aryl, substituted or unsubstituted heteroaryl and substituted or unsubstituted heterocycloalkyl; X is a member selected from O, S and NR²³; R²³ is a member selected from H, substituted or unsubstituted alkyl, substituted or unsubstituted heteroalkyl, and acyl; R³ is a member selected from the group consisting of (═O), SR¹¹, NHR¹¹ and OR¹¹, wherein R¹¹ is a member selected from the group consisting of H, substituted alkyl, unsubstituted alkyl, substituted heteroalkyl, unsubstituted heteroalkyl, monophosphates, diphosphates, triphosphates, sulfonates, acyl, C(O)R¹²R¹³, C(O)OR¹², C(O)NR¹²R¹³, P(O)(OR¹²)₂, C(O)CHR¹²R¹³, SR¹²and SiR¹²R¹³R¹⁴, in which R¹², R¹³, and R¹⁴ are members independently selected from H, substituted or unsubstituted alkyl, substituted or unsubstituted heteroalkyl and substituted or unsubstituted aryl, wherein R¹² and R¹³ together with the nitrogen or carbon atom to which they are attached are optionally joined to form a substituted or unsubstituted heterocycloalkyl ring system having from 4 to 6 members, optionally containing two or more heteroatoms; R⁴, R^{4,}, R⁵ and R^{5,} are members independently selected from the group consisting of H, substituted alkyl, unsubstituted alkyl, substituted aryl, unsubstituted aryl, substituted heteroaryl, unsubstituted heteroaryl, substituted heterocycloalkyl, unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, or any adjacent pair of R⁴, R^{4,}, R⁵ and R^{5,}, together with the carbon atoms to which they are attached, are joined to form a substituted or unsubstituted cycloalkyl or heterocycloalkyl ring system having from 4 to 6 members, wherein n is an integer from 1 to 20; R¹⁵ and R¹⁶ are independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl, and substituted or unsubstituted peptidyl, wherein R¹⁵ and R¹⁶ together with the nitrogen atom to which they are attached are optionally joined to form a substituted or unsubstituted heterocycloalkyl ring system having from 4 to 6 members, optionally containing two or more heteroatoms; R⁶ is a single bond which is either present or absent and when present R⁶ and R⁷ are joined to form a cyclopropyl ring; and R⁷ is CH₂—X¹ or —CH₂— joined in said cyclopropyl ring with R⁶, wherein X¹ is a leaving group, wherein at least one of R⁴ , R^{4,}, R⁵, R^{5,}, R¹⁵ or R¹⁶ links said drug to L¹, if present or to F.

In some embodiment, the drug has structure (c) or (f) above. One specific example of a compound suitable for use as conjugate is

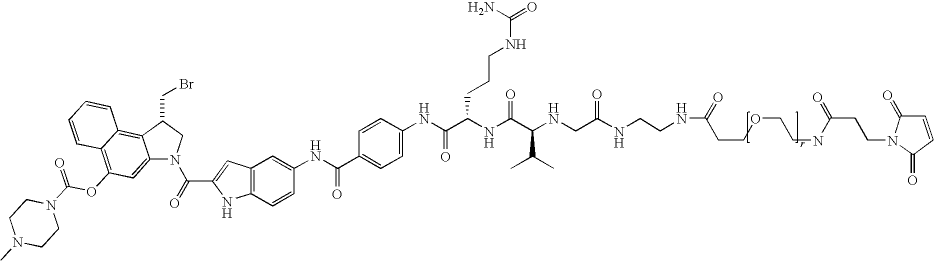

where r is an integer in the range from 0 to 24.

Other examples of suitable compounds for use as conjugates include:

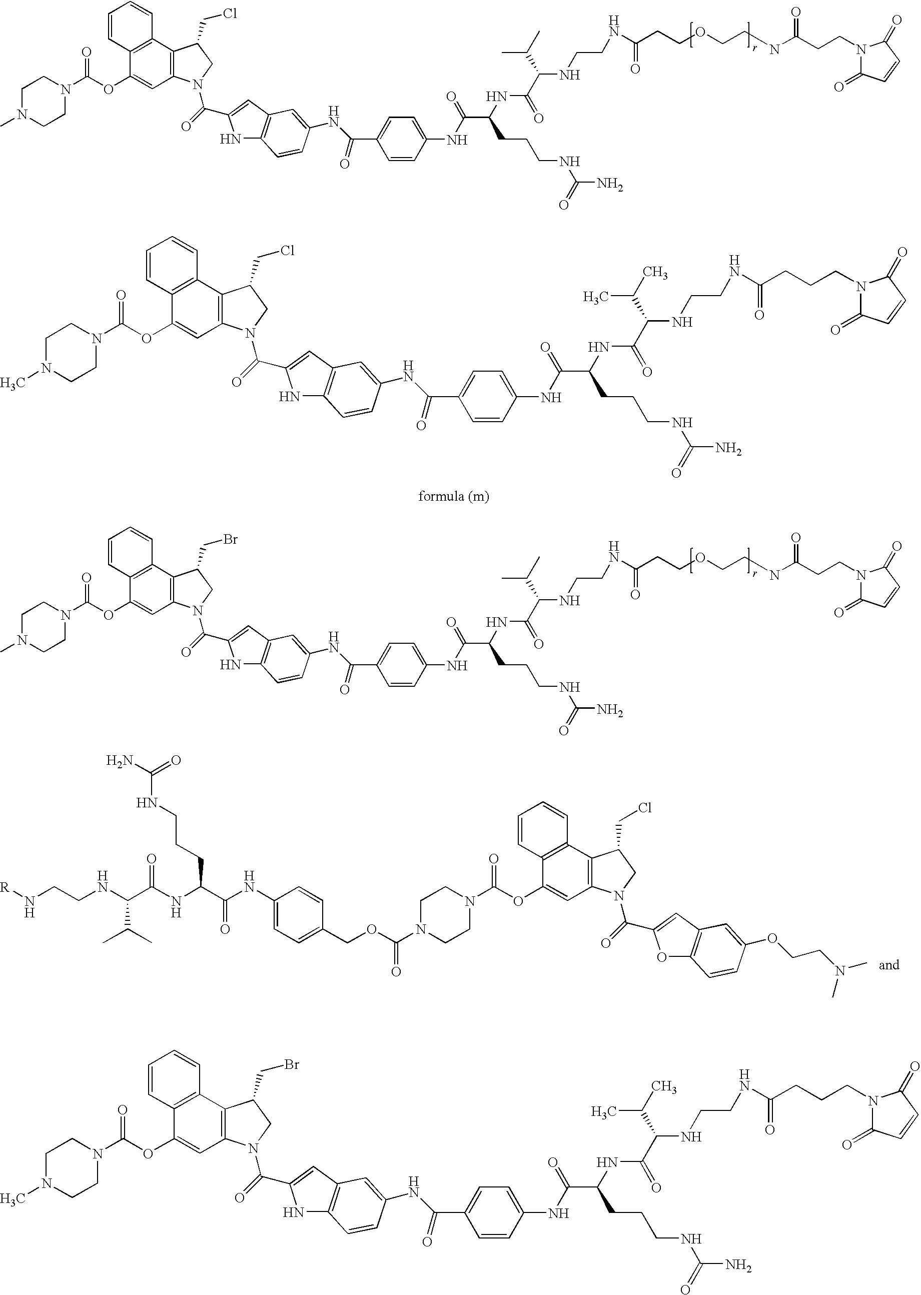

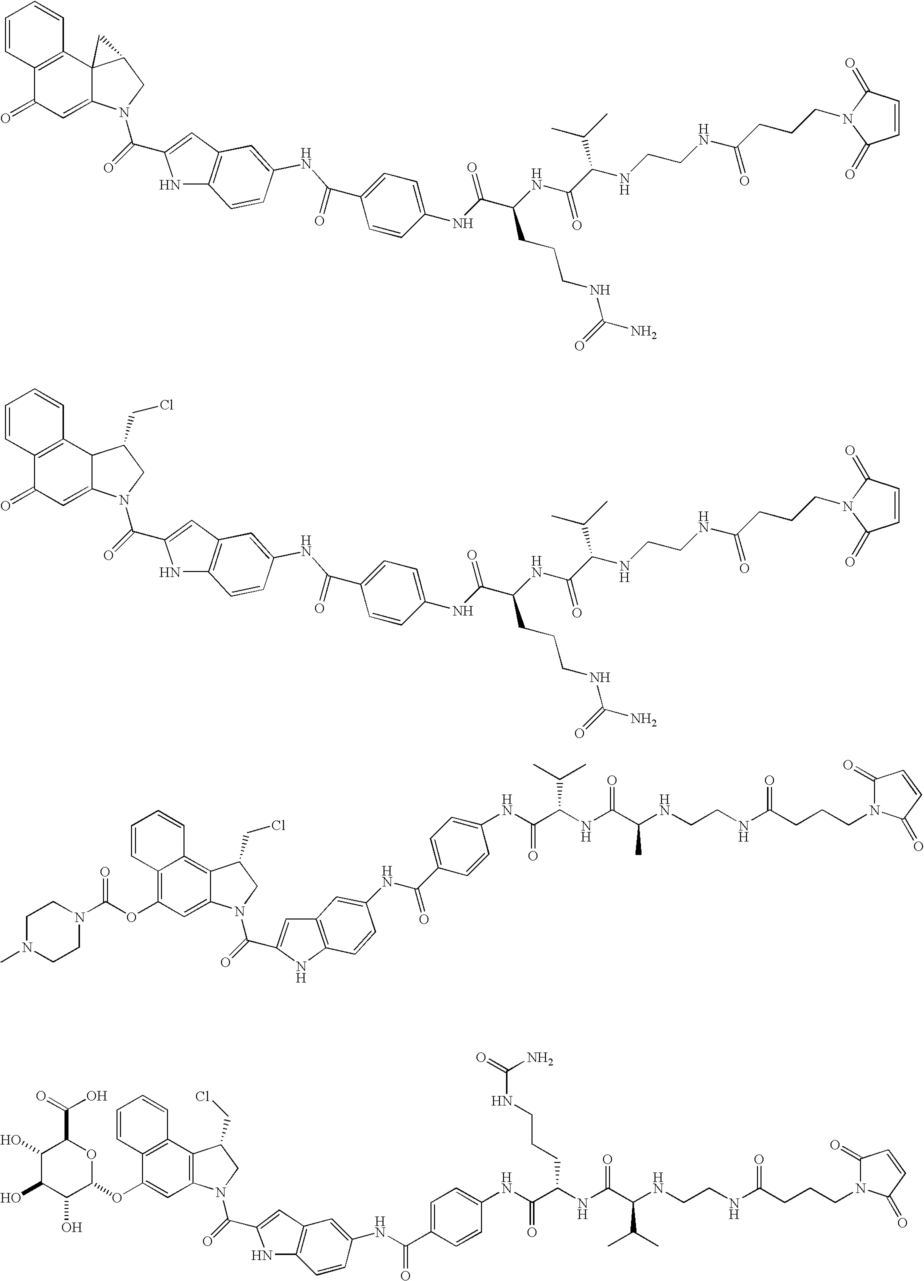

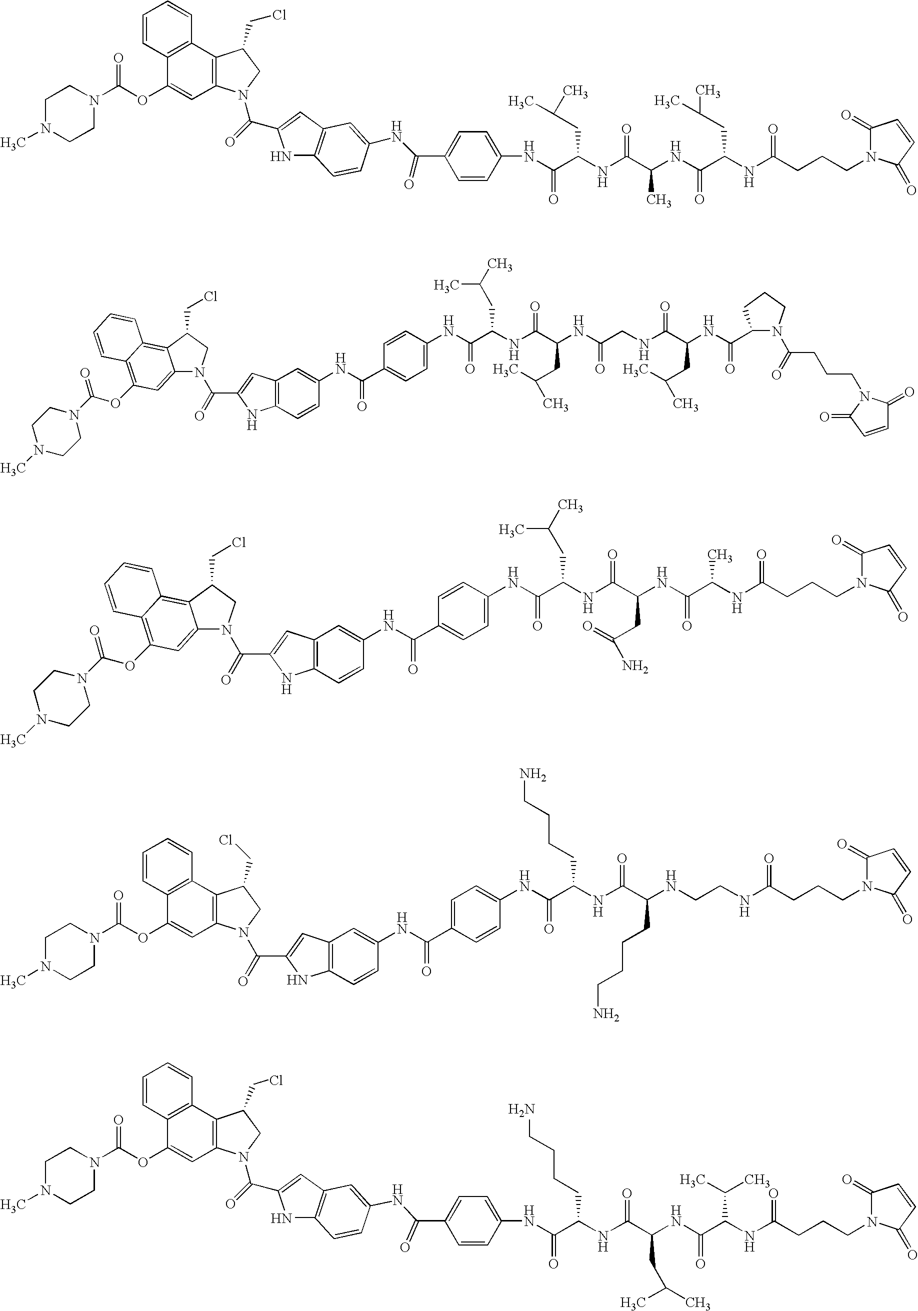

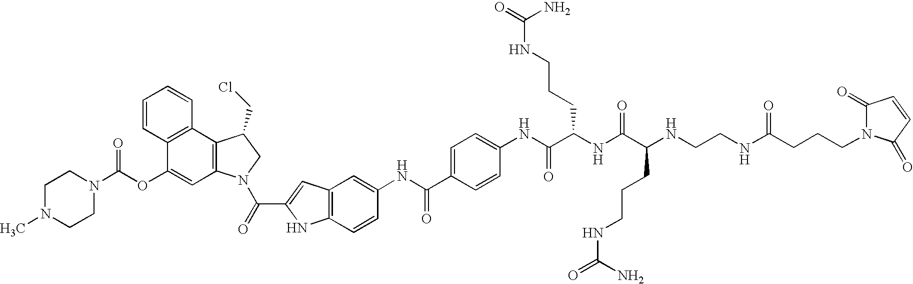

where R is

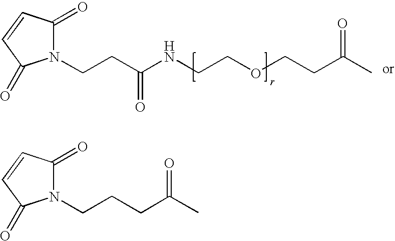

and r is an integer in the range from 0 to 24.

Conjugates can also be formed using the drugs having structure (g), such as the following compounds:

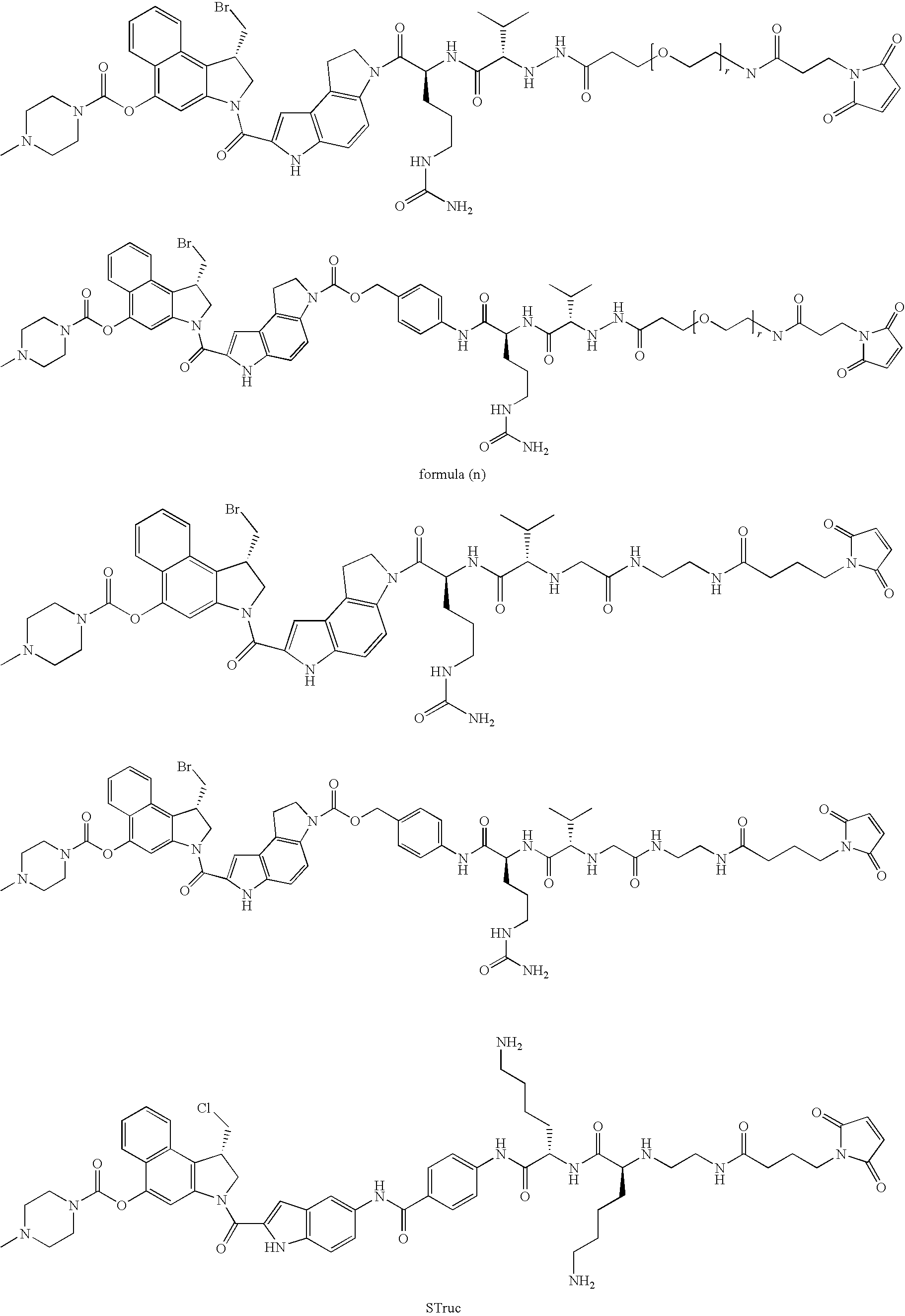

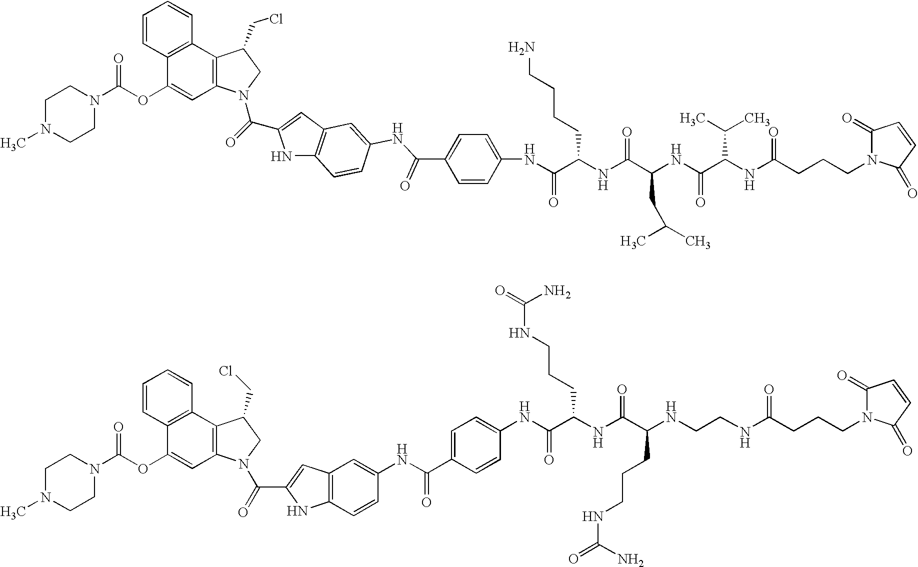

(where r is an integer in the range from 0 to 24).

Conjugates can also be formed using the drugs having the following structures:

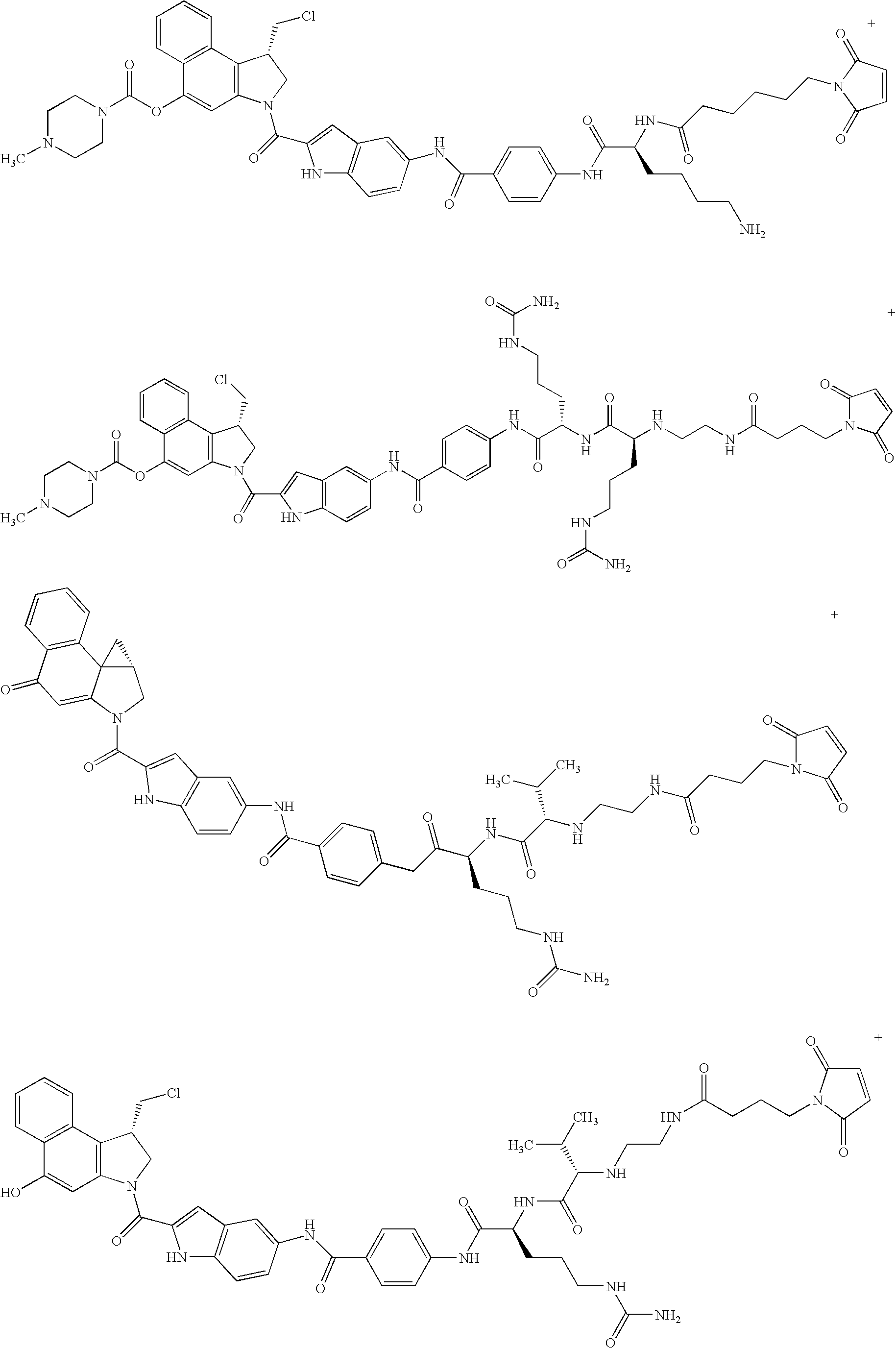

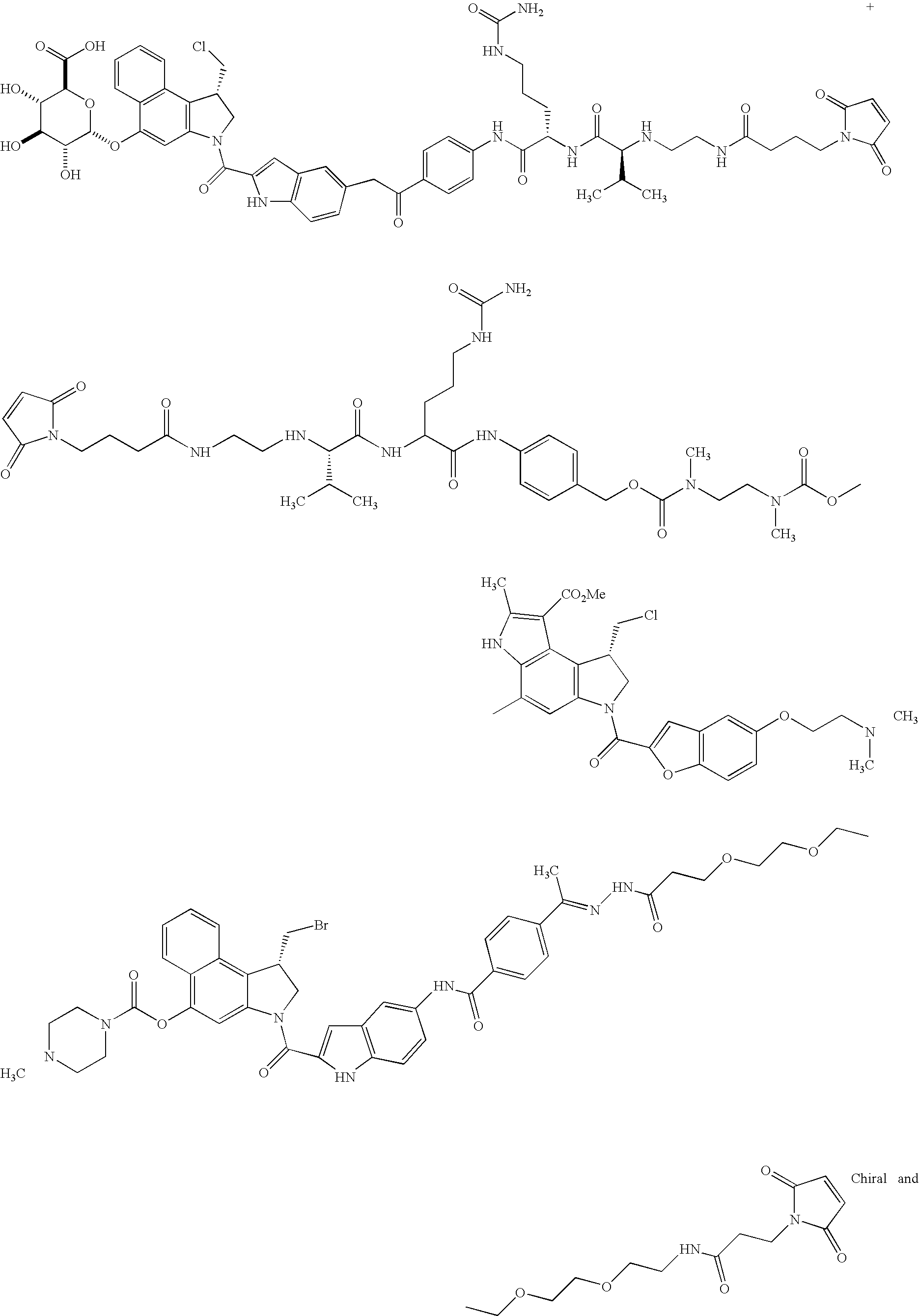

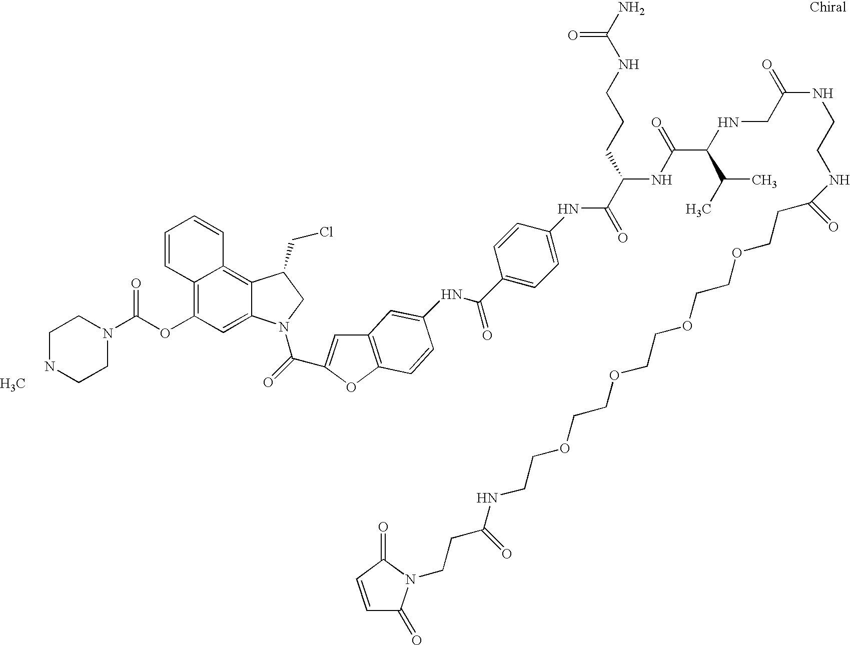

Synthesis of such toxins, as well as details regarding their linkage to antibodies is disclosed in U.S. Patent Application having U.S. Ser. No. 60/991,300, filed on Nov. 30, 2007.

In certain embodiments, the anti-CD70 is conjugated to the linker and therapeutic agent of structure N1:

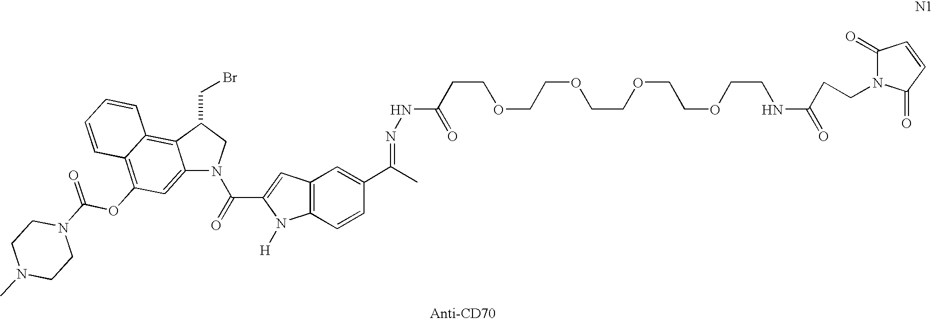

In certain embodiments, the anti-CD70 is conjugated to the linker and therapeutic agent of structure N2:

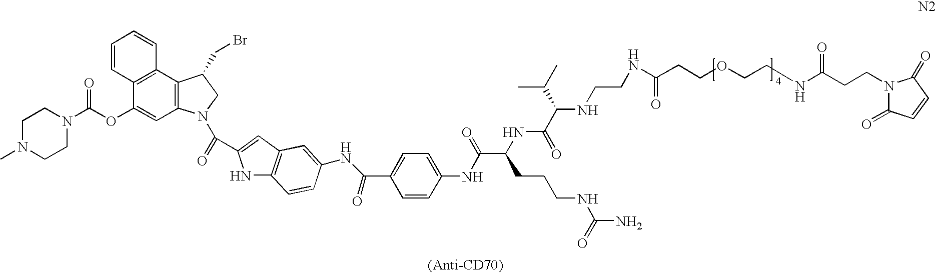

B. Cleavable Linker Conjugates

One example of a suitable conjugate is a compound having the following structure:

X²L¹ₘ-D

wherein L¹ is a self-immolative spacer; m is an integer of 0, 1, 2, 3, 4, 5, or 6; X² is a cleavable substrate; and D comprises a structure:

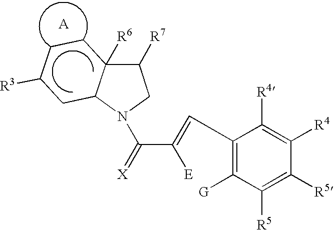

wherein the ring system A is a member selected from substituted or unsubstituted aryl, substituted or unsubstituted heteroaryl and substituted or unsubstituted heterocycloalkyl groups; E and G are members independently selected from H, substituted or unsubstituted alkyl, substituted or unsubstituted heteroalkyl, a heteroatom, a single bond, or E and G are joined to form a ring system selected from substituted or unsubstituted aryl, substituted or unsubstituted heteroaryl and substituted or unsubstituted heterocycloalkyl; X is a member selected from O, S and NR²³; R²³ is a member selected from H, substituted or unsubstituted alkyl, substituted or unsubstituted heteroalkyl, and acyl; R³ is a member selected from the group consisting of (═O), SR¹¹, NHR¹¹ and OR¹¹, wherein R¹¹ is a member selected from the group consisting of H, substituted alkyl, unsubstituted alkyl, substituted heteroalkyl, unsubstituted heteroalkyl, monophosphates, diphosphates, triphosphates, sulfonates, acyl, C(O)R¹²R¹³, C(O)OR¹², C(O)NR¹²R¹³, P(O)(OR¹²)₂, C(O)CHR¹²R¹³, SR¹² and SiR¹²R¹³R¹⁴, in which R¹², R¹³, and R¹⁴ are members independently selected from H, substituted or unsubstituted alkyl, substituted or unsubstituted heteroalkyl and substituted or unsubstituted aryl, wherein R¹² and R¹³ together with the nitrogen or carbon atom to which they are attached are optionally joined to form a substituted or unsubstituted heterocycloalkyl ring system having from 4 to 6 members, optionally containing two or more heteroatoms; R⁶ is a single bond which is either present or absent and when present R⁶ and R⁷ are joined to form a cyclopropyl ring; and R⁷ is CH₂—X¹ or —CH₂— joined in said cyclopropyl ring with R⁶, wherein X¹ is a leaving group, R⁴ , R⁴′, R⁵ and R⁵′ are members independently selected from the group consisting of H, substituted alkyl, unsubstituted alkyl, substituted aryl, unsubstituted aryl, substituted heteroaryl, unsubstituted heteroaryl, substituted heterocycloalkyl, unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, or any adjacent pair of R⁴, R⁴′, R⁵ and R⁵′, together with the carbon atoms to which they are attached, are joined to form a substituted or unsubstituted cycloalkyl or heterocycloalkyl ring system having from 4 to 6 members, wherein n is an integer from 1 to 20; R¹⁵ and R¹⁶ are independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl, and substituted or unsubstituted peptidyl, wherein R¹⁵ and R¹⁶ together with the nitrogen atom to which they are attached are optionally joined to form a substituted or unsubstituted heterocycloalkyl ring system having from 4 to 6 members, optionally containing two or more heteroatoms; wherein at least one of members R⁴, R⁴′, R⁵ and R⁵′ links said drug to L¹, if present, or to X², and is selected from the group consisting of

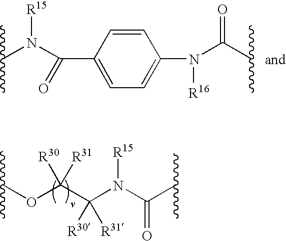

wherein R³⁰, R^{30′}, R³¹, and R^{31′} are independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl; and v is an integer from 1 to 6.

Examples of suitable cleavable linkers include β-AlaLeuAlaLeu (SEQ ID NO:92) and

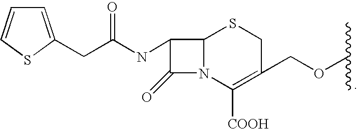

### Pharmaceutical Compositions

In another aspect, the present disclosure provides a composition, e.g., a pharmaceutical composition, containing one or a combination of monoclonal antibodies or antigen-binding portion(s) thereof, of the present disclosure, formulated together with a pharmaceutically acceptable carrier. Such compositions may include one or a combination of (e.g., two or more different) antibodies or immunoconjugates or bispecific molecules of this disclosure. For example, a pharmaceutical composition of this disclosure can comprise a combination of antibodies (or immunoconjugates or bispecifics) that bind to different epitopes on the target antigen or that have complementary activities.

Pharmaceutical compositions of this disclosure also can be administered in combination therapy, i.e., combined with other agents. For example, the combination therapy can include an anti-CD70 antibody of the present disclosure combined with at least one other anti-cancer, anti-inflammatory or immunosuppressant agent. Examples of therapeutic agents that can be used in combination therapy are described in greater detail below in the section on uses of the antibodies of this disclosure.

As used herein, “pharmaceutically acceptable carrier” includes any and all solvents, dispersion media, coatings, antibacterial and antifungal agents, isotonic and absorption delaying agents and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjuage or bispecific molecule, may be coated in a material to protect the compound from the action of acids and other natural conditions that may inactivate the compound.

The pharmaceutical compounds of this disclosure may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of this disclosure also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol and the like; and (3) metal chelating agents, such as citric acid, ethylenediamine tetraacetic acid (EDTA), sorbitol, tartaric acid, phosphoric acid and the like.

Examples of suitable aqueous and nonaqueous carriers that may be employed in the pharmaceutical compositions of this disclosure include water, ethanol, polyols (such as glycerol, propylene glycol, polyethylene glycol and the like) and suitable mixtures thereof, vegetable oils, such as olive oil and injectable organic esters, such as ethyl oleate. Proper fluidity can be maintained, for example, by the use of coating materials, such as lecithin, by the maintenance of the required particle size in the case of dispersions and by the use of surfactants.

These compositions may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol sorbic acid and the like. It may also be desirable to include isotonic agents, such as sugars, sodium chloride and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of this disclosure is contemplated. Supplementary active compounds can also be incorporated into the compositions.

Therapeutic compositions typically must be sterile and stable under the conditions of manufacture and storage. The composition can be formulated as a solution, microemulsion, liposome or other ordered structure suitable to high drug concentration. The carrier can be a solvent or dispersion medium containing, for example, water, ethanol, polyol (for example, glycerol, propylene glycol and liquid polyethylene glycol and the like) and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated and the particular mode of administration. The amount of active ingredient which can be combined with a carrier material to produce a single dosage form will generally be that amount of the composition which produces a therapeutic effect. Generally, out of one hundred per cent, this amount will range from about 0.01 per cent to about ninety-nine percent of active ingredient, preferably from about 0.1 per cent to about 70 per cent, most preferably from about 1 per cent to about 30 per cent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of this disclosure are dictated by and directly dependent on (a) the unique characteristics of the active compound and the particular therapeutic effect to be achieved and (b) the limitations inherent in the art of compounding such an active compound for the treatment of sensitivity in individuals.

For administration of the antibody, the dosage ranges from about 0.0001 to 100 mg/kg and more usually 0.01 to 25 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. Higher dosages, e.g., 15 mg/kg body weight, 20 mg/kg body weight or 25 mg/kg body weight can be used as needed. An exemplary treatment regime entails administration once per week, once every two weeks, once every three weeks, once every four weeks, once a month, once every 3 months or once every three to 6 months. Particular dosage regimens for an anti-CD70 antibody of this disclosure include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more anti-CD70 monoclonal antibodies of this disclosure with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated and preferably until the patient shows partial or complete amelioration of symptoms of disease. Thereafter, the patient can be administered a prophylactic regime.

For use in the prophylaxis and/or treatment of diseases related to abnormal cellular proliferation, a circulating concentration of administered compound of about 0.001 μM to 20 μM is preferred, with about 0.01 μM to 5 μM being preferred.

Patient doses for oral administration of the compounds described herein, typically range from about 1 mg/day to about 10,000 mg/day, more typically from about 10 mg/day to about 1,000 mg/day, and most typically from about 50 mg/day to about 500 mg/day. Stated in terms of patient body weight, typical dosages range from about 0.01 to about 150 mg/kg/day, more typically from about 0.1 to about 15 mg/kg/day, and most typically from about 1 to about 10 mg/kg/day, for example 5 mg/kg/day or 3 mg/kg/day.

In at least some embodiments, patient doses that retard or inhibit tumor growth can be 1 μmol/kg/day or less. For example, the patient doses can be 0.9, 0.8, 0.7, 0.6, 0.5, 0.45, 0.3, 0.2, 0.15, 0.1, 0.09, 0.08, 0.07, 0.06, 0.05, 0.04, 0.03, 0.02, 0.01, or 0.005 μmol/kg or less (referring to moles of the drug). Preferably, the antibody-drug conjugate retards growth of the tumor when administered in the daily dosage amount over a period of at least five days. In at least some embodiments, the tumor is a human-type tumor in a SCID mouse. As an example, the SCID mouse can be a CB17.SCID mouse (available from Taconic, Germantown, N.Y.).

Actual dosage levels of the active ingredients in the pharmaceutical compositions of the present disclosure may be varied so as to obtain an amount of the active ingredient which is effective to achieve the desired therapeutic response for a particular patient, composition and mode of administration, without being toxic to the patient. The selected dosage level will depend upon a variety of pharmacokinetic factors including the activity of the particular compositions of the present disclosure employed or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated and like factors well known in the medical arts.

A “therapeutically effective dosage” of an anti-CD70 antibody of this disclosure preferably results in a decrease in severity of disease symptoms, an increase in frequency and duration of disease symptom-free periods or a prevention of impairment or disability due to the disease affliction. For example, for the treatment of CD70+ tumors, a “therapeutically effective dosage” preferably inhibits cell growth or tumor growth by at least about 20%, more preferably by at least about 40%, even more preferably by at least about 60% and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit cell growth, such inhibition can be measured in vitro by assays known to the skilled practitioner. A therapeutically effective amount of a therapeutic compound can decrease tumor size or otherwise ameliorate symptoms in a subject. One of ordinary skill in the art would be able to determine such amounts based on such factors as the subject's size, the severity of the subject's symptoms and the particular composition or route of administration selected.

A composition of the present disclosure can be administered via one or more routes of administration using one or more of a variety of methods known in the art. As will be appreciated by the skilled artisan, the route and/or mode of administration will vary depending upon the desired results. Preferred routes of administration for antibodies of this disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody of this disclosure can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches and microencapsulated delivery systems. Biodegradable, biocompatible polymers can be used, such as ethylene vinyl acetate, polyanhydrides, polyglycolic acid, collagen, polyorthoesters and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition of this disclosure can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion primp for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems and modules are known to those skilled in the art.

In certain embodiments, the human monoclonal antibodies of this disclosure can be formulated to ensure proper distribution in vivo. For example, the blood-brain barrier (BBB) excludes many highly hydrophilic compounds. To ensure that the therapeutic compounds of this disclosure cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p 120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

Uses and Methods of this Disclosure

The antibodies, particularly the human antibodies, antibody compositions, antibody-partner molecule conjugate compositions and methods of the present disclosure have numerous in vitro and in vivo diagnostic and therapeutic utilities involving the diagnosis and treatment of CD70 mediated disorders. For example, these molecules can be administered to cells in culture, in vitro or ex vivo or to human subjects, e.g., in vivo, to treat, prevent and to diagnose a variety of disorders. As used herein, the term “subject” is intended to include human and non-human animals. “Non-human animals” include all vertebrates, e.g., mammals and non-mammals, such as non-human primates, sheep, dogs, cats, cows, horses, chickens, amphibians and reptiles. Preferred subjects include human patients having disorders mediated by CD70 activity. The methods are particularly suitable for treating human patients having a disorder associated with aberrant CD70 expression. When antibody-partner molecule conjugates to CD70 are administered together with another agent, the two can be administered in either order or simultaneously.

Given the specific binding of the antibodies of this disclosure for CD70, the antibodies of this disclosure can be used to specifically detect CD70 expression on the surface of cells and, moreover, can be used to purify CD70 via immunoaffinity purification.

CD70 is expressed in a variety of human cancers, including renal cell carcinomas, metastatic breast cancers, brain tumors, leukemias, lymphomas and nasopharangeal carcinomas (Junker et al. (2005) J Urol. 173:2150-3; Sloan et al. (2004) Am J Pathol. 164:315-23; Held-Feindt and Mentlein (2002) Int J Cancer 98:352-6; Hishima et al. (2000) Am J Surg Pathol. 24:742-6; Lens et al. (1999) Br J Haematol. 106:491-503). An anti-CD70 antibody may be used alone to inhibit the growth of cancerous tumors. Alternatively, an anti-CD70 antibody may be used in conjunction with other immunogenic agents, standard cancer treatments or other antibodies, as described below.

Preferred cancers whose growth may be inhibited using the antibodies of this disclosure include cancers typically responsive to immunotherapy. Non-limiting examples of preferred cancers for treatment include renal cancer (e.g., renal cell carcinoma), breast cancer, brain tumors, chronic or acute leukemias including acute myeloid leukemia, chronic myeloid leukemia, acute lymphoblastic leukemia, chronic lymphocytic leukemia, lymphomas (e.g., Hodgkin's and non-Hodgkin's lymphoma, lymphocytic lymphoma, primary CNS lymphoma, T-cell lymphoma) and nasopharangeal carcinomas. Examples of other cancers that may be treated using the methods of this disclosure include melanoma (e.g., metastatic malignant melanoma), prostate cancer, colon cancer, lung cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, Kaposi's sarcoma, epidermoid cancer, squamous cell cancer, environmentally induced cancers including those induced by asbestos, e.g., mesothelioma and combinations of said cancers.

Furthermore, given the expression of CD70 on various tumor cells, the human antibodies, antibody compositions and methods of the present disclosure can be used to treat a subject with a tumorigenic disorder, e.g., a disorder characterized by the presence of tumor cells expressing CD70 including, for example, renal cell carcinomas (RCC), such as clear cell RCC, glioblastoma, breast cancer, brain tumors, nasopharangeal carcinomas, non-Hodgkin's lymphoma (NHL), acute lymphocytic leukemia (ALL), chronic lymphocytic leukemia (CLL), Burkitt's lymphoma, anaplastic large-cell lymphomas (ALCL), multiple myeloma, cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), entroblastic/centrocytic (cb/cc) follicular lymphomas cancers, diffuse large cell lymphomas of B lineage, angioimmunoblastic lymphadenopathy (AILD)-like T cell lymphoma, HIV associated body cavity based lymphomas, embryonal carcinomas, undifferentiated carcinomas of the rhino-pharynx (e.g., Schmincke's tumor), Castleman's disease, Kaposi's Sarcoma, multiple myeloma, Waldenstrom's macroglobulinemia and other B-cell lymphomas.

Accordingly, in one embodiment, this disclosure provides a method of inhibiting growth of tumor cells in a subject, comprising administering to the subject a therapeutically effective amount of an anti-CD70 antibody or antigen-binding portion thereof Preferably, the antibody is a human anti-CD70 antibody (such as any of the human anti-human CD70 antibodies described herein). Additionally or alternatively, the antibody may be a chimeric or humanized anti-CD70 antibody.

Additionally, the interaction of CD70 with CD27 has also been proposed to play a role in cell-mediated autoimmune diseases, such as experimental autoimmune encephalomyelitis (EAE) (Nakajima et al. (2000) J. Neuroimmunol. 109:188-96). This effect was thought to be mediated in part by an inhibition of TNF-alpha production. Furthermore, blocking of CD70 signaling inhibits CD40-mediated clonal expansion of CD8+ T-cells and reduces the generation of CD8+ memory T-cells (Taraban et al. (2004) J. Immunol. 173:6542-6). As such, the human antibodies, antibody compositions and methods of the present disclosure can be used to treat a subject with an autoimmune disorder, e.g., a disorder characterized by the presence of B-cells expressing CD70 including, for example, experimental autoimmune encephalomyelitis. Additional autoimmune disorders in which the antibodies of this disclosure can be used include, but are not limited to systemic lupus erythematosus (SLE), insulin dependent diabetes mellitus (IDDM), inflammatory bowel disease (IBD) (including Crohn's Disease, ulcerative colitis and Celiac disease), multiple sclerosis (MS), psoriasis, autoimmune thyroiditis, rheumatoid arthritis (RA) and glomerulonephritis. Furthermore, the antibody compositions of this disclosure can be used for inhibiting or preventing transplant rejection or in the treatment of graft versus host disease (GVHD).

Additionally, the interaction of CD70 with CD27 has also been proposed to play a role in signaling on CD4+ T cells. Some viruses have been shown to signal the CD27 pathway, leading to destruction of neutralizing antibody responses (Matter et al. (2006) J Exp Med 203:2145-55). As such, the human antibodies, antibody compositions and methods of the present disclosure can be used to treat a subject with a viral infection including, for example, infections from human immunodeficiency virus (HIV), Hepatitis (A, B, & C), Herpesvirus, (e.g., VZV, HSV-1, HAV-6, HSV-II and CMV, Epstein Barr virus), adenovirus, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus and lymphocytic choriomeningitis virus (LCMV) or in the treatment of HIV infection/AIDS. Additionally, the human antibodies, antibody compositions and methods of the present disclosure can be used to inhibit TNF-alpha production.

In one embodiment, the antibodies (e.g., human monoclonal antibodies, multispecific and bispecific molecules and compositions) of this disclosure can be used to detect levels of CD70 or levels of cells which contain CD70 on their membrane surface, which levels can then be linked to certain disease symptoms. Alternatively, the antibodies can be used to inhibit or block CD70 function which, in turn, can be linked to the prevention or amelioration of certain disease symptoms, thereby implicating CD70 as a mediator of the disease. This can be achieved by contacting an experimental sample and a control sample with the anti-CD70 antibody under conditions that allow for the formation of a complex between the antibody and CD70. Any complexes formed between the antibody and CD70 are detected and compared in the experimental sample and the control.

In another embodiment, the antibodies (e.g., human antibodies, multispecific and bispecific molecules and compositions) of this disclosure can be initially tested for binding activity associated with therapeutic or diagnostic use in vitro. For example, compositions of this disclosure can be tested using the flow cytometric assays described in the Examples below.

The antibodies (e.g., human antibodies, multispecific and bispecific molecules, immunoconjugates and compositions) of this disclosure have additional utility in therapy and diagnosis of CD70-related diseases. For example, the human monoclonal antibodies, the multispecific or bispecific molecules and the immunoconjugates can be used to elicit in vivo or in vitro one or more of the following biological activities: to inhibit the growth of and/or kill a cell expressing CD70; to mediate phagocytosis or ADCC of a cell expressing CD70 in the presence of human effector cells; or to block CD70 ligand binding to CD70.

In a particular embodiment, the antibodies (e.g., human antibodies, multispecific and bispecific molecules and compositions) are used in vivo to treat, prevent or diagnose a variety of CD70-related diseases. Examples of CD70-related diseases include, among others, autoimmune disorders, experimental autoimmune encephalomyelitis (EAE), cancer, renal cell carcinomas (RCC), such as clear cell RCC, glioblastoma, breast cancer, brain tumors, nasopharangeal carcinomas, non-Hodgkin's lymphoma, acute lymphocytic leukemia (ALL), chronic lymphocytic leukemia (CLL), Burkitt's lymphoma, anaplastic large-cell lymphomas (ALCL), multiple myeloma, cutaneous T-cell lymphomas, nodular small cleaved-cell lymphomas, lymphocytic lymphomas, peripheral T-cell lymphomas, Lennert's lymphomas, immnnoblastic lymphomas, T-cell leukemia/lymphomas (ATLL), adult T-cell leukemia (T-ALL), entroblastic/centrocytic (cb/cc) follicular lymphomas cancers, diffuse large cell lymphomas of B lineage, angioimmunoblastic lymphadenopathy (AILD)-like T cell lymphoma, HIV associated body cavity based lymphomas, Embryonal Carcinomas, undifferentiated carcinomas of the rhino-pharynx (e.g., Schmincke's tumor), Castleman's disease, Kaposi's Sarcoma, Multiple Myeloma, Waldenstrom's macroglobulinemia, and other B-cell lymphomas.

Suitable routes of administering the antibody compositions (e.g., human monoclonal antibodies, multispecific and bispecific molecules and immunoconjugates) of this disclosure in vivo and in vitro are well known in the art and can be selected by those of ordinary skill. For example, the antibody compositions can be administered by injection (e.g., intravenous or subcutaneous). Suitable dosages of the molecules used will depend on the age and weight of the subject and the concentration and/or formulation of the antibody composition.

As previously described, human anti-CD70 antibodies of this disclosure can be co-administered with one or more therapeutic agents, e.g., a cytotoxic agent, a radiotoxic agent or an immunosuppressive agent. The antibody can be linked to the agent (as an immunocomplex) or can be administered separately from the agent. In the latter case (separate administration), the antibody can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil and cyclophosphamide hydroxyurea which, by themselves, are only effective at levels which are toxic or subtoxic to a patient. Cisplatin is intravenously administered as a 100 mg/dose once every four weeks and adriamycin is intravenously administered as a 60-75 mg/ml dose once every 21 days. Co-administration of the human anti-CD70 antibodies or antigen binding fragments thereof, of the present disclosure with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

Target-specific effector cells, e.g., effector cells linked to compositions (e.g., human antibodies, multispecific and bispecific molecules) of this disclosure can also be used as therapeutic agents. Effector cells for targeting can be human leukocytes such as macrophages, neutrophils or monocytes. Other cells include eosinophils, natural killer cells and other IgG- or IgA-receptor bearing cells. If desired, effector cells can be obtained from the subject to be treated. The target-specific effector cells can be administered as a suspension of cells in a physiologically acceptable solution. The number of cells administered can be in the order of 10⁸-10⁹ but will vary depending on the therapeutic purpose. In general, the amount will be sufficient to obtain localization at the target cell, e.g., a tumor cell expressing CD70 and to effect cell killing by, e.g., phagocytosis. Routes of administration can also vary.

Therapy with target-specific effector cells can be performed in conjunction with other techniques for removal of targeted cells. For example, anti-tumor therapy using the compositions (e.g., human antibodies, multispecific and bispecific molecules) of this disclosure and/or effector cells armed with these compositions can be used in conjunction with chemotherapy. Additionally, combination immunotherapy may be used to direct two distinct cytotoxic effector populations toward tumor cell rejection. For example, anti-CD70 antibodies linked to anti-Fc-gamma RI or anti-CD3 may be used in conjunction with IgG- or IgA-receptor specific binding agents.

Bispecific and multispecific molecules of this disclosure can also be used to modulate FcγR or FcγR levels on effector cells, such as by capping and elimination of receptors on the cell surface. Mixtures of anti-Fc receptors can also be used for this purpose.

The compositions (e.g., human antibodies, multispecific and bispecific molecules and immunoconjugates) of this disclosure which have complement binding sites, such as portions from IgG1, -2 or -3 or IgM which bind complement, can also be used in the presence of complement. In one embodiment, ex vivo treatment of a population of cells comprising target cells with a binding agent of this disclosure and appropriate effector cells can be supplemented by the addition of complement or serum containing complement. Phagocytosis of target cells coated with a binding agent of this disclosure can be improved by binding of complement proteins. In another embodiment target cells coated with the compositions (e.g., human antibodies, multispecific and bispecific molecules) of this disclosure can also be lysed by complement. In yet another embodiment, the compositions of this disclosure do not activate complement.

The compositions (e.g., human antibodies, multispecific and bispecific molecules and immunoconjugates) of this disclosure can also be administered together with complement. Accordingly, within the scope of this disclosure are compositions comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules of this disclosure and the complement or serum can be administered separately.

Also within the scope of the present disclosure are kits comprising the antibody compositions of this disclosure (e.g., human antibodies, bispecific or multispecific molecules or immunoconjugates) and instructions for use. The kit can further contain one or more additional reagents, such as an immunosuppressive reagent, a cytotoxic agent or a radiotoxic agent or one or more additional human antibodies of this disclosure (e.g., a human antibody having a complementary activity which binds to an epitope in the CD70 antigen distinct from the first human antibody).

Accordingly, patients treated with antibody compositions of this disclosure can be additionally administered (prior to, simultaneously with or following administration of a human antibody of this disclosure) with another therapeutic agent, such as a cytotoxic or radiotoxic agent, which enhances or augments the therapeutic effect of the human antibodies.

In other embodiments, the subject can be additionally treated with an agent that modulates, e.g., enhances or inhibits, the expression or activity of Fcγ or Fcγ receptors by, for example, treating the subject with a cytokine. Preferred cytokines for administration during treatment with the multispecific molecule include of granulocyte colony-stimulating factor (G-CSF), granulocyte-macrophage colony-stimulating factor (GM-CSF), interferon-γ (IFN-γ) and tumor necrosis factor (TNF).

The compositions (e.g., human antibodies, multispecific and bispecific molecules) of this disclosure can also be used to target cells expressing FcγR or CD70, for example for labeling such cells. For such use, the binding agent can be linked to a molecule that can be detected. Thus, this disclosure provides methods for localizing ex vivo or in vitro cells expressing Fc receptors, such as FcγR or CD70. The detectable label can be, e.g., a radioisotope, a fluorescent compound, an enzyme or an enzyme co-factor.

In a particular embodiment, this disclosure provides methods for detecting the presence of CD70 antigen in a sample or measuring the amount of CD70 antigen, comprising contacting the sample and a control sample, with a human monoclonal antibody or an antigen binding portion thereof, which specifically binds to CD70, under conditions that allow for formation of a complex between the antibody or portion thereof and CD70. The formation of a complex is then detected, wherein a difference complex formation between the sample compared to the control sample is indicative the presence of CD70 antigen in the sample.

In yet another embodiment, immunoconjugates of this disclosure can be used to target compounds (e.g., therapeutic agents, labels, cytotoxins, radiotoxoins immunosuppressants, etc.) to cells which have CD70 cell surface receptors by linking such compounds to the antibody. For example, an anti-CD70 antibody can be conjugated to any of the cytotoxin compounds described in U.S. Pat. Nos. 6,281,354 and 6,548,530, U.S. Ser. No. 60/991,300, US patent publication Nos. 20030050331, 20030064984, 20030073852 and 20040087497 or published in WO 03/022806, which are hereby incorporated by reference in their entireties. Thus, this disclosure also provides methods for localizing ex vivo or in vivo cells expressing CD70 (e.g., with a detectable label, such as a radioisotope, a fluorescent compound, an enzyme or an enzyme co-factor). Alternatively, the immunoconjugates can be used to kill cells which have CD70 cell surface receptors by targeting cytotoxins or radiotoxins to CD70.

The present disclosure is further illustrated by the following Examples which should not be construed as further limiting. The contents of all figures and all references, Genbank sequences, patents and published patent applications cited throughout this application are expressly incorporated herein by reference in their entirety.

### Examples

### Example 1

### Generation of Human Monoclonal Antibodies Against CD70

### Antigen

Immunization protocols utilized as antigen recombinant human CD70 fused with a dual myc-His tag. Alternatively, whole cell immunization using the renal carcinoma cell line 786-O (ATCC Accession No. CRL-1932) and boosted with the renal carcinoma cell line A-498 (ATCC Accession No. HTB-44) was used in some immunizations.

### Transgenic HuMAb Mouse® and KM Mouse®

Fully human monoclonal antibodies to CD70 were prepared using the HCo7, HCo12 and HCo17 strains of HuMab transgenic mice and the KM strain of transgenic transchromosomic mice, each of which express human antibody genes. In these mouse strains, the endogenous mouse kappa light chain gene has been homozygously disrupted as described in Chen et al. (1993) EMBO J. 12:811-820 and the endogenous mouse heavy chain gene has been homozygously disrupted as described in Example 1 of PCT Publication WO 01/09187. Furthermore, this mouse strain carries a human kappa light chain transgene, KCo5, as described in Fishwild et al. (1996) Nature Biotechnology 14:845-851 and a human heavy chain transgene, HCo7, HCo12 or HCo17 as described in Example 2 of PCT Publication WO 01/09187. The KM Mouse® strain contains the SC20 transchromosome as described in PCT Publication WO 02/43478.

### HuMab and KM Immunizations:

To generate fully human monoclonal antibodies to CD70, mice of the HuMAb Mouse® and KM Mouse® were immunized with recombinant human CD70 as antigen or whole cells expressing CD70 on the cell surface. General immunization schemes for

HuMab mice are described in Lonberg, N. et al (1994) Nature 368(6474): 856-859; Fishwild, D. et al. (1996) Nature Biotechnology 14: 845-851 and PCT Publication WO 98/24884. The mice were 6-16 weeks of age upon the first infusion of antigen. 5-10×10⁶ cells were used to immunize the HuMab mice intraperitonealy (IP), subcutaneously (Sc) or via footpad injection.

Transgenic mice were immunized twice with antigen in complete Freund's adjuvant or Ribi adjuvant IP, followed by 3-21 days IP (up to a total of 11 immunizations) with the antigen in incomplete Freund's or Ribi adjuvant. The immune response was monitored by retroorbital bleeds. The plasma was screened by ELISA and FACS (as described below) and mice with sufficient titers of anti-CD70 human immunogolobulin were used for fusions. Mice were boosted intravenously with antigen 3 days before sacrifice and removal of the spleen. Typically, 10-35 fusions for each antigen were performed. Several dozen mice were immunized for each antigen.

### Selection of a HuMab Mouse® or KM Mouse® Producing Anti-CD70 Antibodies:

To select a HuMab Mouse® or KM Mouse® producing antibodies that bound CD70, sera from immunized mice were screened by flow cytometry for binding to a cell line expressing recombinant human CD70, but not to a control cell line that does not express CD70. In addition, the sera were screened by flow cytometry for binding to 786-O or A-498 cells. Briefly, the binding of anti-CD70 antibodies was assessed by incubating CD70-expressing CHO cells, 786-0 cells or A498 cells with the anti-CD70 antibody at 1:20 dilution. The cells were washed and binding was detected with a FITC-labeled anti-human IgG Ab. Flow cytometric analyses were performed using a FACSCalibur flow cytometry (Becton Dickinson, San Jose, Calif.). Antibodies that bound to the CD70 expressing CHO cells but not the non-CD70 expressing parental CHO cells were further tested for binding to CD70 by ELISA, as described by Fishwild, D. et al. (1996). Briefly, microtiter plates were coated with purified recombinant CD70 fusion protein from transfected CHO cells at 1-2 μg /ml in PBS, 100 μl/wells incubated 4° C. overnight then blocked with 200 μl/well of 5% chicken serum in PBS/Tween (0.05%). Dilutions of sera from CD70-immunized mice were added to each well and incubated for 1-2 hours at ambient temperature. The plates were washed with PBS/Tween and then incubated with a goat-anti-human IgG polyclonal antibody conjugated with horseradish peroxidase (HRP) for 1 hour at room temperature. After washing, the plates were developed with ABTS substrate (Sigma, A-1888, 0.22 mg/ml) and analyzed by spectrophotometer at OD 415-495. Mice that developed the highest titers of anti-CD70 antibodies were used for fusions. Fusions were performed as described below and hybridoma supernatants were tested for anti-CD70 activity by ELISA.

### Generation of Hybridomas Producing Human Monoclonal Antibodies to CD70:

The mouse splenocytes, isolated from a HuMab mouse® and/or a KM mouse®, were fused to a mouse myeloma cell line either using PEG based upon standard protocols or electric field based electrofusion using a Cyto Pulse large chamber cell fusion electroporator (Cyto Pulse Sciences, Inc., Glen Burnie, Md.). The resulting hybridomas were then screened for the production of antigen-specific antibodies. Single cell suspensions of splenocytes from immunized mice were fused to one-fourth the number of SP2/0 nonsecreting mouse myeloma cells (ATCC, CRL 1581) with 50% PEG (Sigma). Cells were plated at approximately 1×10⁵/well in flat bottom microtiter plate, followed by a one week incubation in DMEM high glucose medium with L-glutamine and sodium pyruvate (Mediatech, Inc., Herndon, Va.) and further containing 10% fetal Bovine Serum (Hyclone, Logan, Utah), 18% P388DI conditional media, 5% Origen Hybridoma cloning factor (BioVeris, Gaithersburg, Va.), 4 mM L-glutamine, 5 mM HEPES, 0.055 mM β-mercaptoethanol, 50 units/ml penicillin, 50 mg/ml streptomycin and 1× Hypoxanthine-aminopterin-thymidine (HAT) media (Sigma; the HAT is added 24 hours after the fusion). After one week, cells cultured in medium in which HAT was used was replaced with HT. Individual wells were then screened by FACS or ELISA (described above) for human anti-CD70 monoclonal IgG antibodies. Once extensive hybridoma growth occurred, medium was monitored usually after 10-14 days. The antibody-secreting hybridomas were replated, screened again and, if still positive for human IgG, anti-CD70 monoclonal antibodies were subcloned at least twice by limiting dilution. The stable subclones were then cultured in vitro to generate small amounts of antibody in tissue culture medium for further characterization.

Hybridoma clones 2H5, 10B4, 8B5, 18E7 and 69A7, were selected for further analysis.

### Example 2

### Structural Characterization of Human Monoclonal Antibodies 2H5, 10B4, 8B5, 18E7, 69A7 and 1F4

The cDNA sequences encoding the heavy and light chain variable regions of the 2H5, 10B4, 8B5, 18E7, 69A7 and 1F4 monoclonal antibodies were obtained from the 2H5, 10B4, 8B5, 18E7, 69A7 and 1F4 hybridomas, respectively, using standard PCR techniques and were sequenced using standard DNA sequencing techniques.

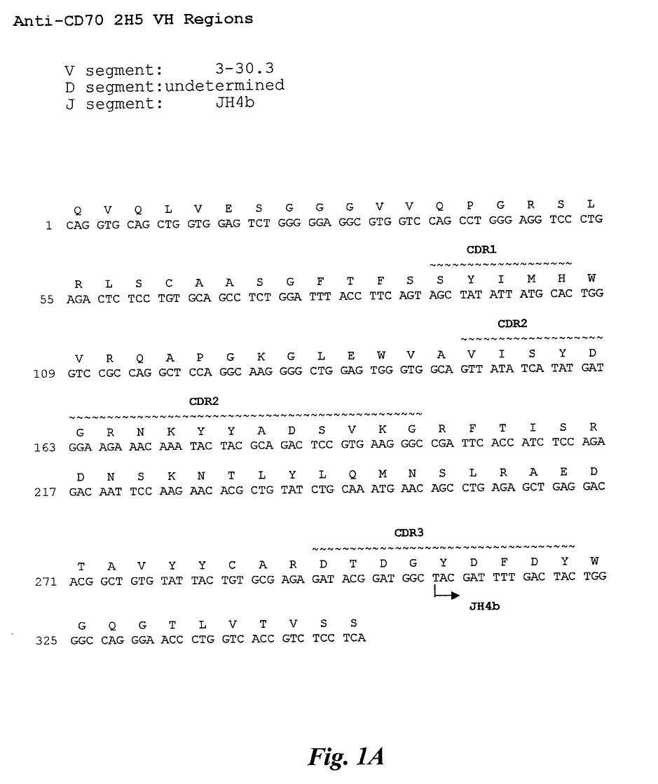

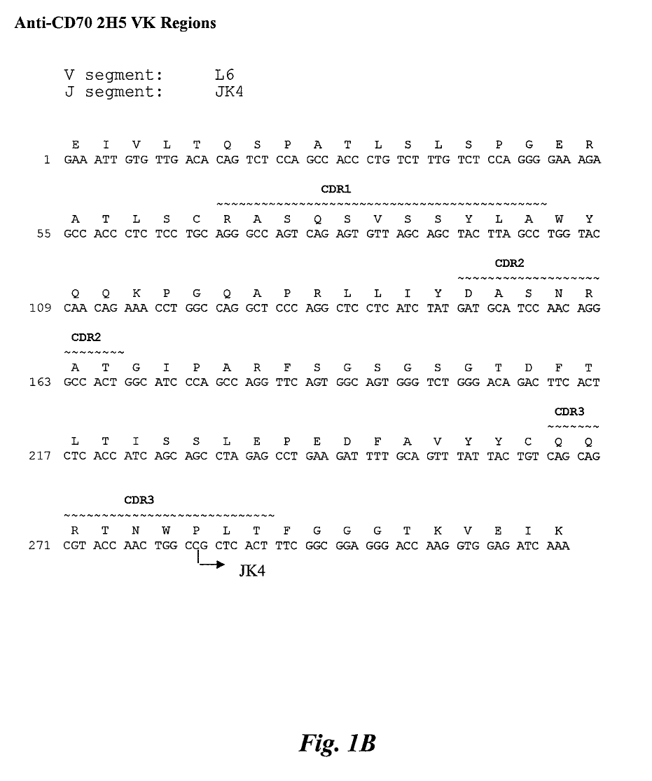

The nucleotide and amino acid sequences of the heavy chain variable region of 2H5 are shown in FIG. 1A and in SEQ ID NO:49 and 1, respectively.

The nucleotide and amino acid sequences of the light chain variable region of 2H5 are shown in FIG. 1B and in SEQ ID NO:55 and 7, respectively.

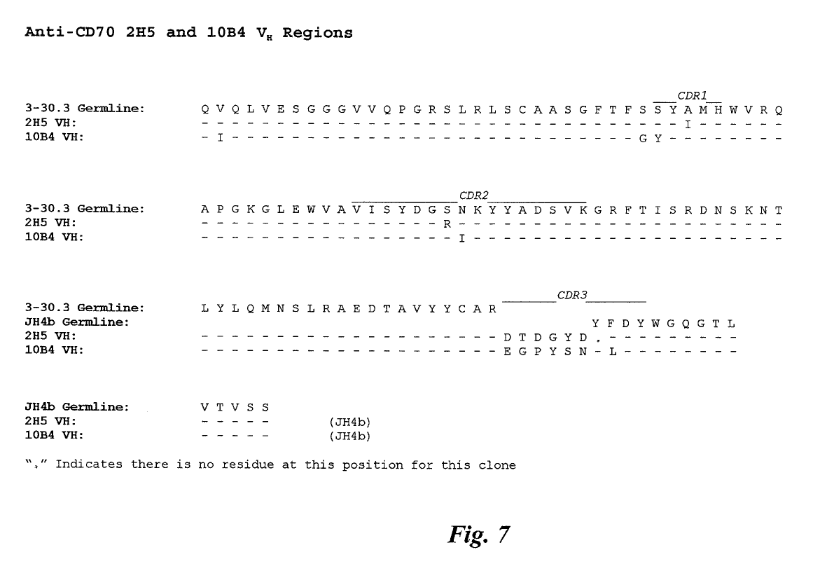

Comparison of the 2H5 heavy chain immunoglobulin sequence to the known human germline immunoglobulin heavy chain sequences demonstrated that the 2H5 heavy chain utilizes a VH segment from human germline VH 3-30.3, an undetermined D segment and a JH segment from human germline JH 4b. The alignment of the 2H5 VH sequence to the germline VH 3-30.3 sequence is shown in FIG. 7. Further analysis of the 2H5 VH sequence using the Kabat system of CDR region determination led to the delineation of the heavy chain CDR1, CDR2 and CDR3 regions as shown in FIGS. 1A and 7 and in SEQ ID NOs:13, 19 and 25, respectively.

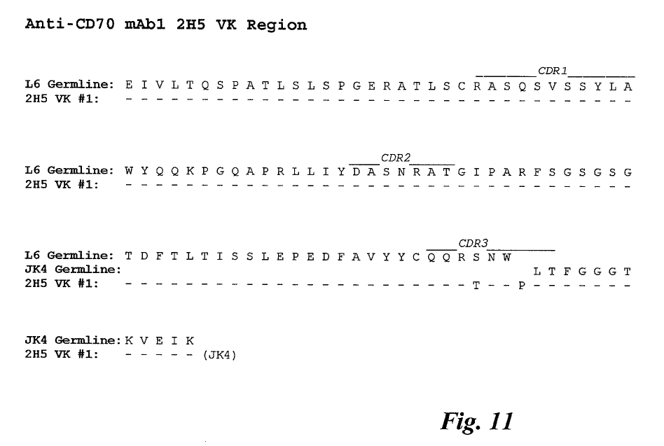

Comparison of the 2H5 light chain immunoglobulin sequence to the known human germline immunoglobulin light chain sequences demonstrated that the 2H5 light chain utilizes a VL segment from human germline VK L6 and a JK segment from human germline JK 4. The alignment of the 2H5 VL sequence to the germline VK L6 sequence is shown in FIG. 11. Further analysis of the 2H5 VL sequence using the Kabat system of CDR region determination led to the delineation of the light chain CDR1, CDR2 and CDR3 regions as shown in FIGS. 1B and 11 and in SEQ ID NOs:31, 37, and 43 respectively.

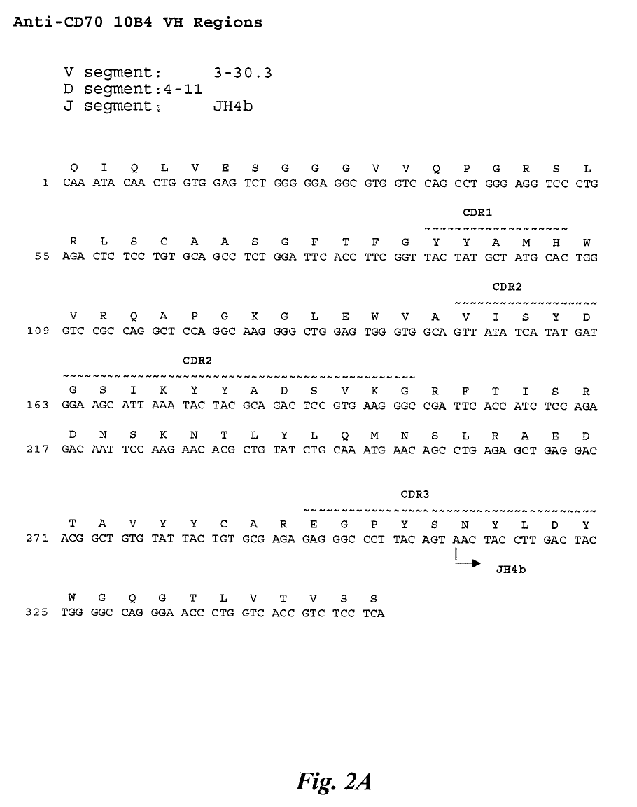

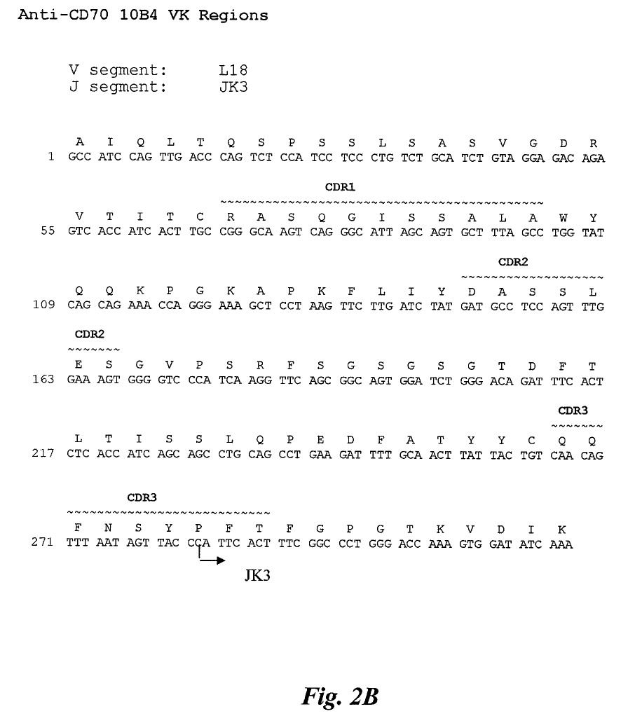

The nucleotide and amino acid sequences of the heavy chain variable region of 10B4 are shown in FIG. 2A and in SEQ ID NO:50 and 2, respectively.

The nucleotide and amino acid sequences of the light chain variable region of 10B4 are shown in FIG. 2B and in SEQ ID NO:56 and 8, respectively.

Comparison of the 10B4 heavy chain immunoglobulin sequence to the known human germline immunoglobulin heavy chain sequences demonstrated that the 10B4 heavy chain utilizes a VH segment from human germline VH 3-30.3, a D segment from human germline 4-11 and a JH segment from human germline JH 4b. The alignment of the 10B4 VH sequence to the germline VH 3-30.3 sequence is shown in FIG. 7. Further analysis of the 10B4 VH sequence using the Kabat system of CDR region determination led to the delineation of the heavy chain CDR1, CDR2 and CDR3 regions as shown in FIGS. 2A and 7 and in SEQ ID NOs:14, 20, and 26, respectively.

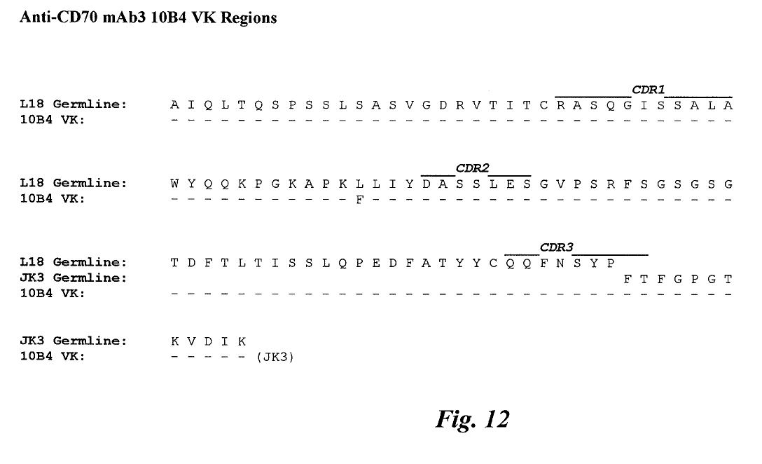

Comparison of the 10B4 light chain immunoglobulin sequence to the known human germline immunoglobulin light chain sequences demonstrated that the 10B4 light chain utilizes a VL segment from human germline VK L18 and a JK segment from human germline JK 3. The alignment of the 10B4 VL sequence to the germline VK L18 sequence is shown in FIG. 12. Further analysis of the 10B4 VL sequence using the Kabat system of CDR region determination led to the delineation of the light chain CDR1, CDR2 and CDR3 regions as shown in FIGS. 2B and 12 and in SEQ ID NOs:32, 38, and 44, respectively.

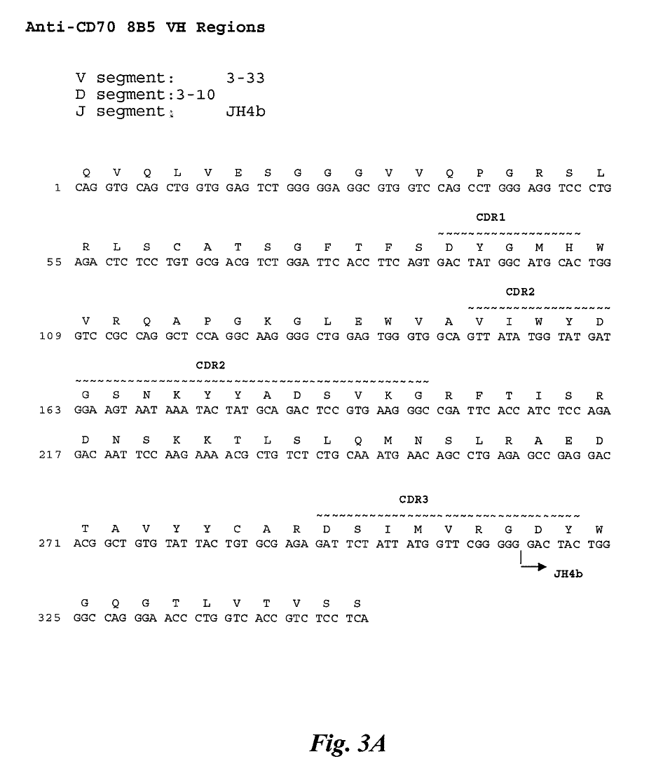

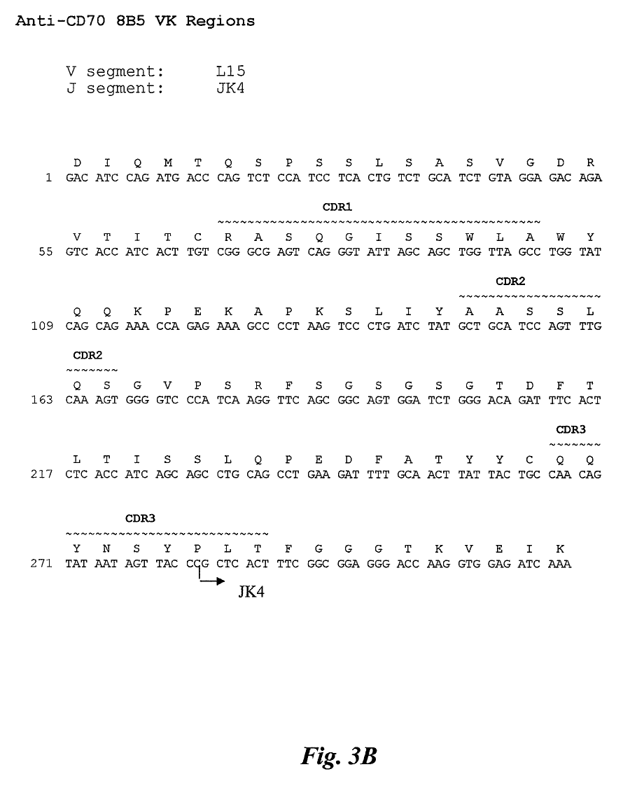

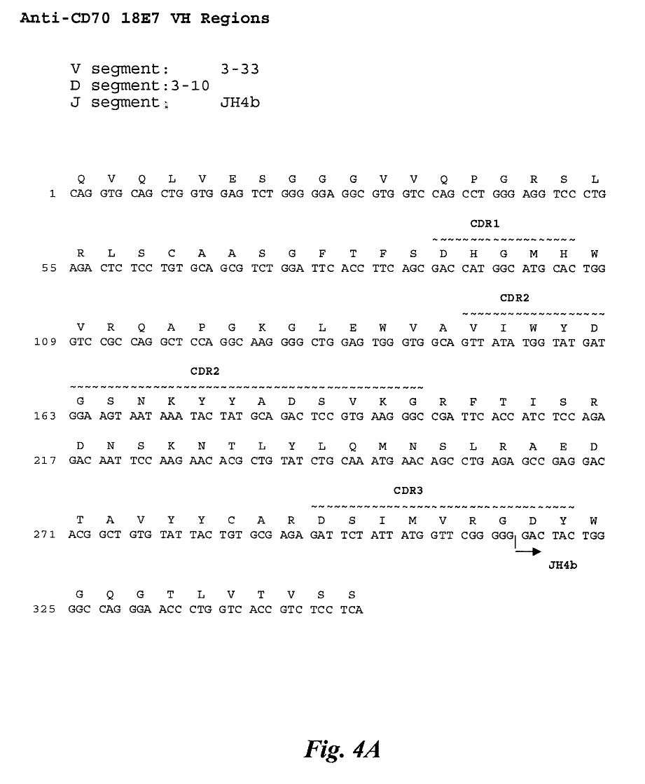

The nucleotide and amino acid sequences of the heavy chain variable region of 8B5 are shown in FIG. 3A and in SEQ ID NO:51 and 3, respectively.

The nucleotide and amino acid sequences of the light chain variable region of 8B5 are shown in FIG. 3B and in SEQ ID NO:57 and 9, respectively.

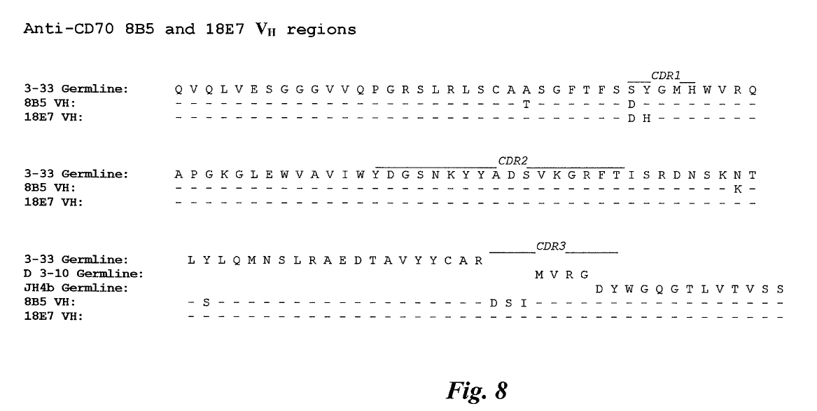

Comparison of the 8B5 heavy chain immunoglobulin sequence to the known human germline immunoglobulin heavy chain sequences demonstrated that the 8B5 heavy chain utilizes a VH segment from human germline VH 3-33, a D segment from human germline 3-10 and a JH segment from human germline JH 4b. The alignment of the 8B5 VH sequence to the germline VH 3-33 sequence is shown in FIG. 8. Further analysis of the 8B5 VH sequence using the Kabat system of CDR region determination led to the delineation of the heavy chain CDR1, CDR2 and CDR3 regions as shown in FIGS. 3A and 8 and in SEQ ID NOs:15, 21, and 27, respectively.

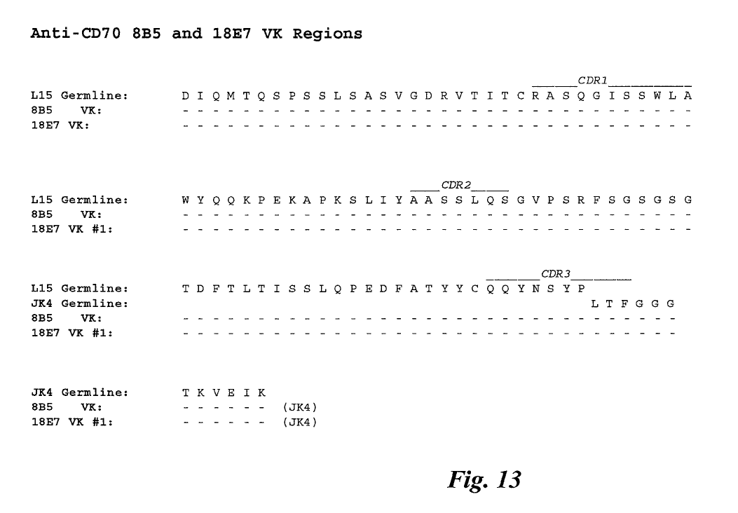

Comparison of the 8B5 light chain immunoglobulin sequence to the known human germline immunoglobulin light chain sequences demonstrated that the 8B5 light chain utilizes a VL segment from human germline VK L15 and a JK segment from human germline JK 4. The alignment of the 8B5 VL sequence to the germline VK L15 sequence is shown in FIG. 13. Further analysis of the 8B5 VL sequence using the Kabat system of CDR region determination led to the delineation of the light chain CDR1, CDR2 and CDR3 regions as shown in FIGS. 3B and 13 and in SEQ ID NOs:33, 39, and 45, respectively.

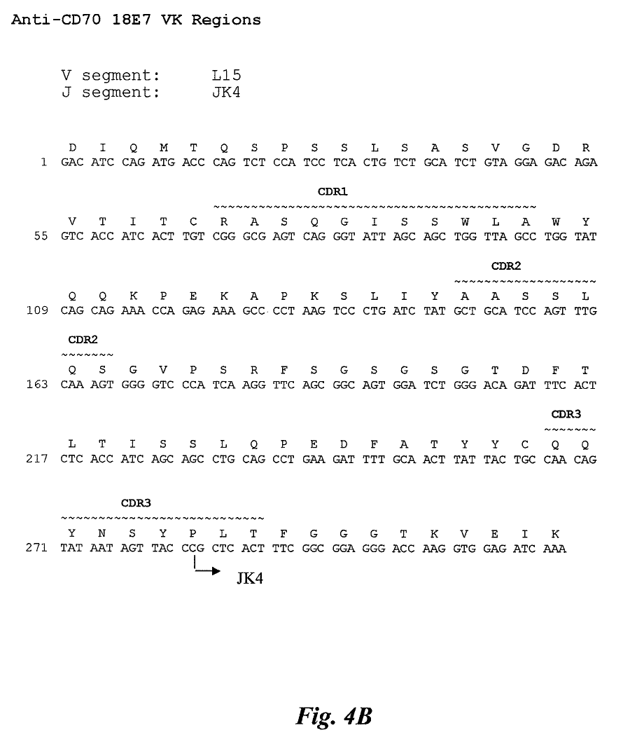

The nucleotide and amino acid sequences of the heavy chain variable region of 18E7 are shown in FIG. 4A and in SEQ ID NO:52 and 4, respectively.

The nucleotide and amino acid sequences of the light chain variable region of 18E7 are shown in FIG. 4B and in SEQ ID NO:58 and 10, respectively.

Comparison of the 18E7 heavy chain immunoglobulin sequence to the known human germline immunoglobulin heavy chain sequences demonstrated that the 18E7 heavy chain utilizes a VH segment from human germline VH 3-33, a D segment from human germline 3-10 and a JH segment from human germline JH 4b. The alignment of the 18E7 VH sequence to the germline VH 3-33 sequence is shown in FIG. 8. Further analysis of the 18E7 VH sequence using the Kabat system of CDR region determination led to the delineation of the heavy chain CDR1, CDR2 and CDR3 regions as shown in FIGS. 4A and 8 and in SEQ ID NOs:16, 22, and 28, respectively.

Comparison of the 18E7 light chain immunoglobulin sequence to the known human germline immunoglobulin light chain sequences demonstrated that the 18E7 light chain utilizes a VL segment from human germline VK L15 and a JK segment from human germline JK 4. The alignment of the 18E7 VL sequence to the germline VK L15 sequence is shown in FIG. 13. Further analysis of the 18E7 VL sequence using the Kabat system of CDR region determination led to the delineation of the light chain CDR1, CDR2 and CDR3 regions as shown in FIGS. 4B and 13 and in SEQ ID NOs:34, 40, and 46, respectively.

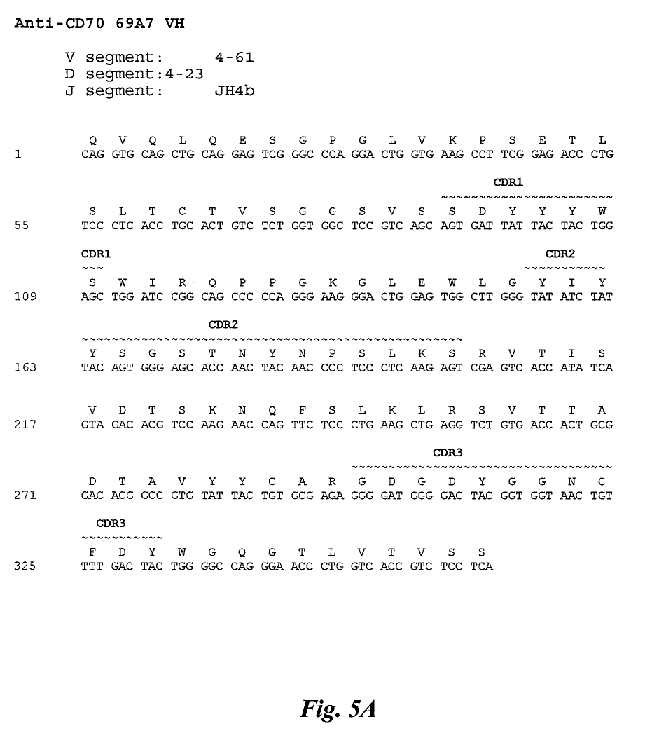

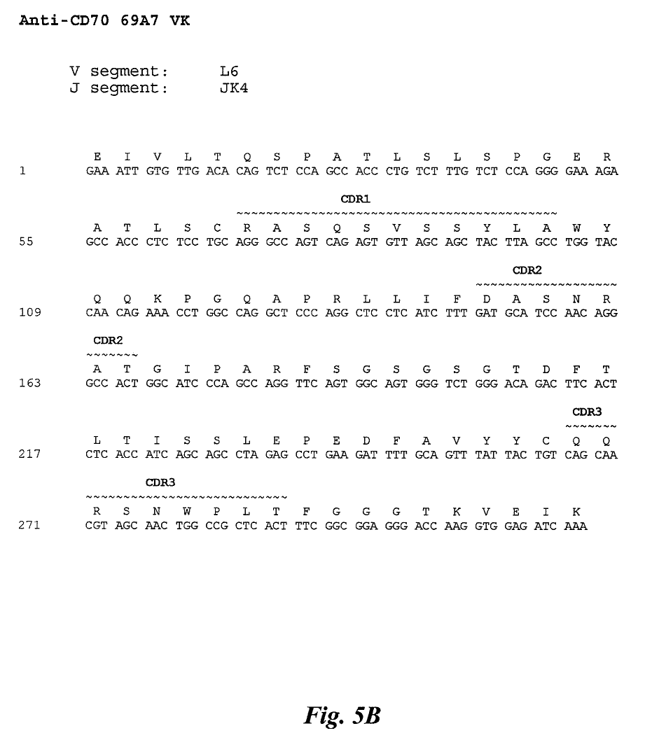

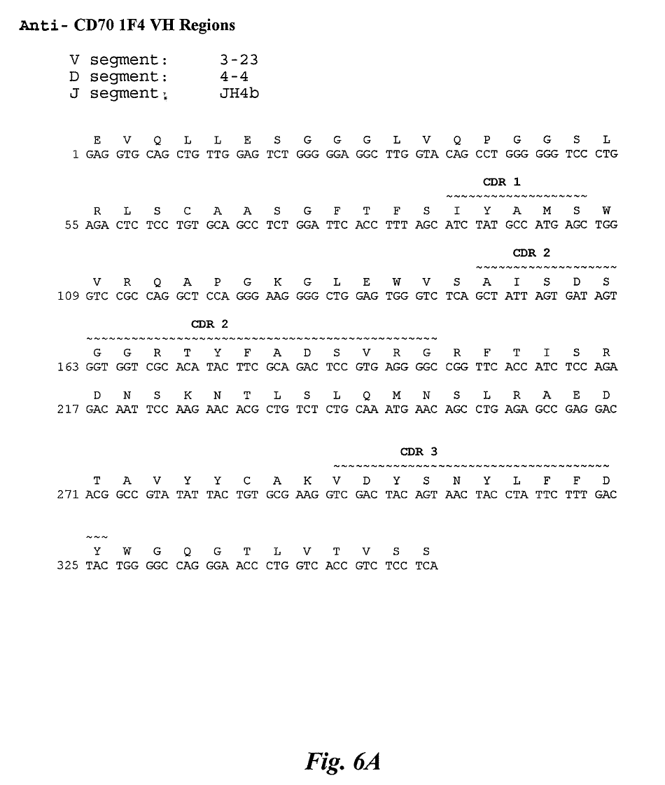

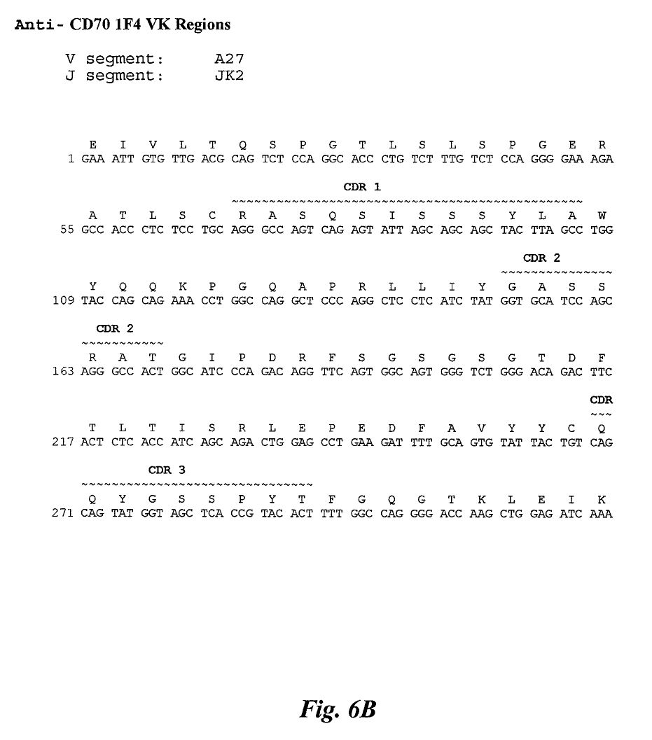

The nucleotide and amino acid sequences of the heavy chain variable region of 69A7 are shown in FIG. 5A and in SEQ ID NO:53 and 5, respectively.

The nucleotide and amino acid sequences of the light chain variable region of 69A7 are shown in FIG. 5B and in SEQ ID NO:59 and 11, respectively.

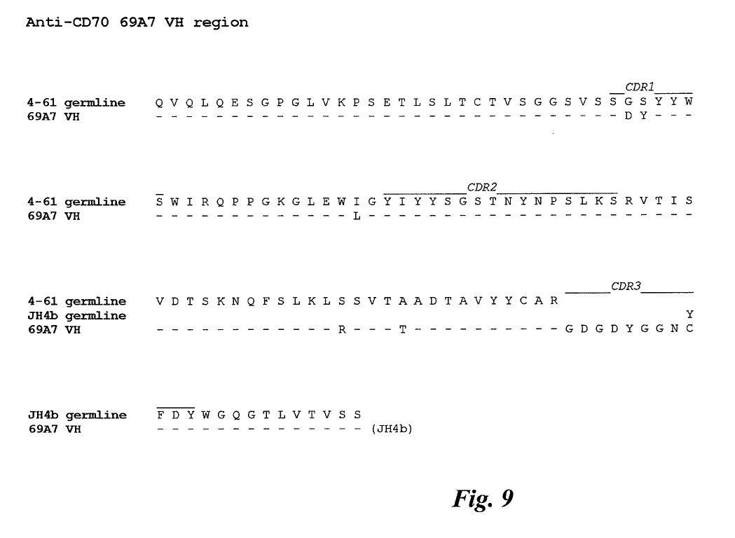

Comparison of the 69A7 heavy chain immunoglobulin sequence to the known human germline immunoglobulin heavy chain sequences demonstrated that the 69A7 heavy chain utilizes a VH segment from human germline VH 4-61, a D segment from human germline 4-23 and a JH segment from human germline JH 4b. The alignment of the 69A7 VH sequence to the germline VH 4-61 sequence is shown in FIG. 9. Further analysis of the 69A7 VH sequence using the Kabat system of CDR region determination led to the delineation of the heavy chain CDR1, CDR2 and CDR3 regions as shown in FIGS. 5A and 9 and in SEQ ID NOs:17, 23, and 29, respectively.

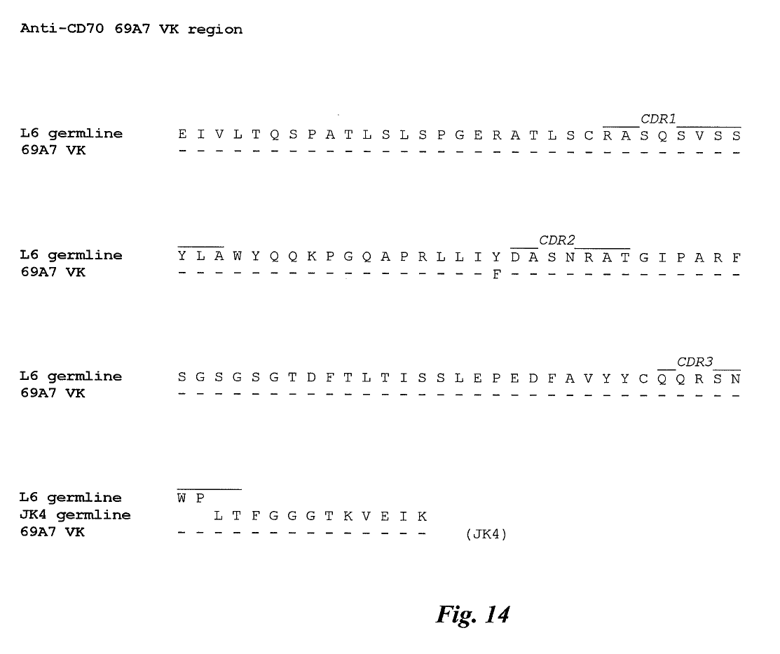

Comparison of the 69A7 light chain immunoglobulin sequence to the known human germline immunoglobulin light chain sequences demonstrated that the 69A7 light chain utilizes a VL segment from human germline VK L6 and a JK segment from human germline JK 4. The alignment of the 69A7 VL sequence to the germline VK L6 sequence is shown in FIG. 14. Further analysis of the 69A7 VL sequence using the Kabat system of CDR region determination led to the delineation of the light chain CDR1, CDR2 and CDR3 regions as shown in FIGS. 5B and 14 and in SEQ ID NOs:35, 41, and 47, respectively.

The nucleotide and amino acid sequences of the heavy chain variable region of 1F4 are shown in FIG. 5A and in SEQ ID NO:54 and 6, respectively.

The nucleotide and amino acid sequences of the light chain variable region of 1F4 are shown in FIG. 5B and in SEQ ID NO:60 and 12, respectively.

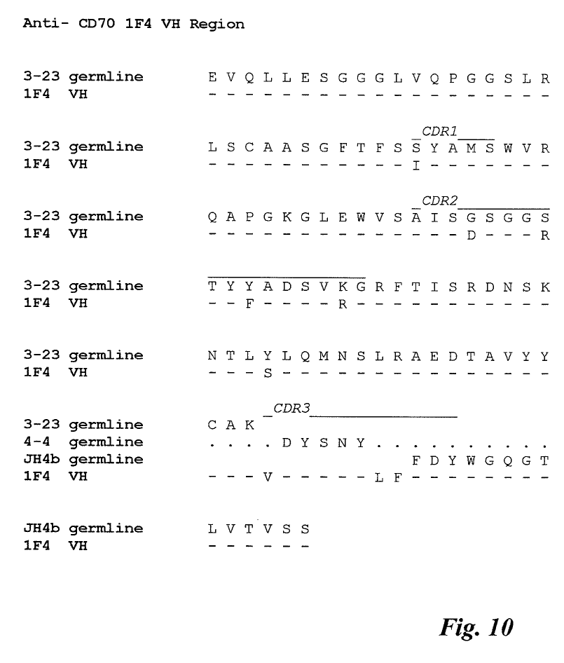

Comparison of the 1F4 heavy chain immunoglobulin sequence to the known human germline immunoglobulin heavy chain sequences demonstrated that the 1F4 heavy chain utilizes a VH segment from human germline VH 3-23, a D segment from human germline 4-4 and a JH segment from human germline JH 4b. The alignment of the 1F4 VH sequence to the germline VH 3-23 sequence is shown in FIG. 10. Further analysis of the 1 F4 VH sequence using the Kabat system of CDR region determination led to the delineation of the heavy chain CDR1, CDR2 and CDR3 regions as shown in FIGS. 5A and 10 and in SEQ ID NOs:18, 24, and 30, respectively.

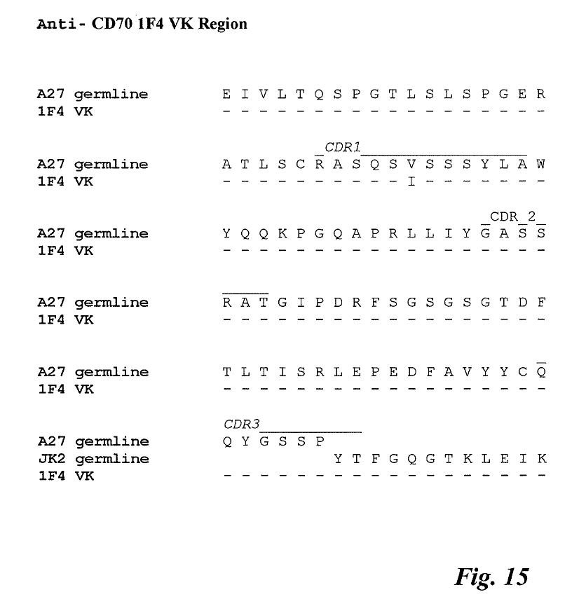

Comparison of the 1F4 light chain immunoglobulin sequence to the known human germline immunoglobulin light chain sequences demonstrated that the 1F4 light chain utilizes a VL segment from human germline VK A27 and a JK segment from human germline JK 2. The alignment of the 1F4 VL sequence to the germline VK A27 sequence is shown in FIG. 15. Further analysis of the 1F4 VL sequence using the Kabat system of CDR region determination led to the delineation of the light chain CDR1, CDR2 and CDR3 regions as shown in FIGS. 5B and 15 and in SEQ ID NOs:36, 42, and 48, respectively.

### Example 3

### Characterization of Binding Specificity of Anti-CD70 Human Monoclonal Antibodies

A comparison of anti-CD70 antibodies on binding to immunopurified CD70 was performed by standard ELISA to examine the specificity of binding for CD70.

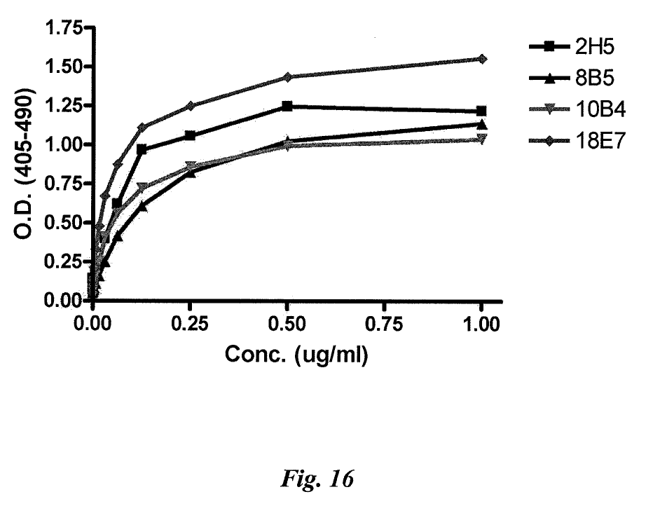

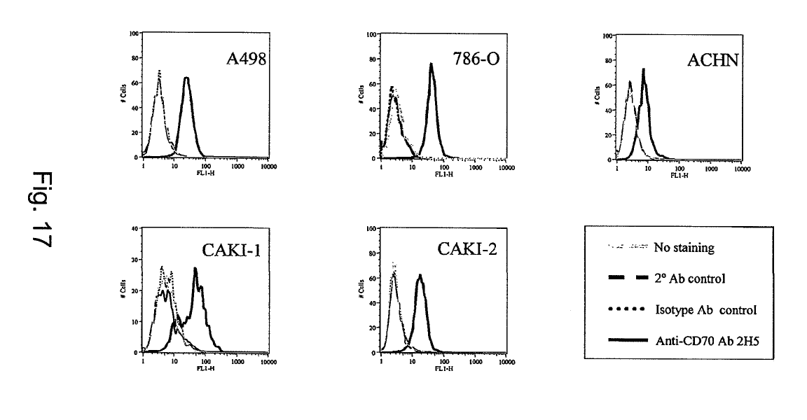

Recombinant myc-tagged CD70 was coated on a plate overnight, then tested for binding against the anti-CD70 human monoclonal antibodies 2H5, 10B4, 8B5, and 18E7. Standard ELISA procedures were performed. The anti-CD70 human monoclonal antibodies were added at a concentration of 1 μg/ml and titrated down at 1:2 serial dilutions. Goat-anti-human IgG (Fc or kappa chain-specific) polyclonal antibody conjugated with horseradish peroxidase (HRP) was used as secondary antibody. The results are shown in FIG. 16. The anti-CD70 human monoclonal antibodies 2H5, 10B4, 8B5 and 18E7 bound with high specificity to CD70.

### Example 4

### Characterization of Anti-CD70 Antibody Binding to CD70 Expressed on the Surface of Renal Cancer Carcinoma Cell Lines

Anti-CD70 antibodies were tested for binding to renal cell carcinoma cells expressing CD70 on their cell surface by flow cytometry.

The renal cell carcinoma cell lines A-498 (ATCC Accession No. HTB-44), 786-O (ATCC Accession No. CRL-1932), ACHN (ATCC Accession No. CRL-1611), Caki-1 (ATCC Accession No. HTB-46) and Caki-2 (ATCC Accession No. HTB-47) were each tested for antibody binding. Binding of the HuMAb 2H5 anti-CD70 human monoclonal antibody was assessed by incubating 1×10⁵ cells with 2H5 at a concentration of 1 μg/ml. The cells were washed and binding was detected with a FITC-labeled anti-human IgG Ab. Flow cytometric analyses were performed using a FACSCalibur flow cytometry (Becton Dickinson, San Jose, Calif.). The results are shown in FIG. 17. The anti-CD70 monoclonal antibody 2H5 bound to the renal carcinoma cell lines A-498, 786-O, ACHN, Caki-1 and Caki-2.

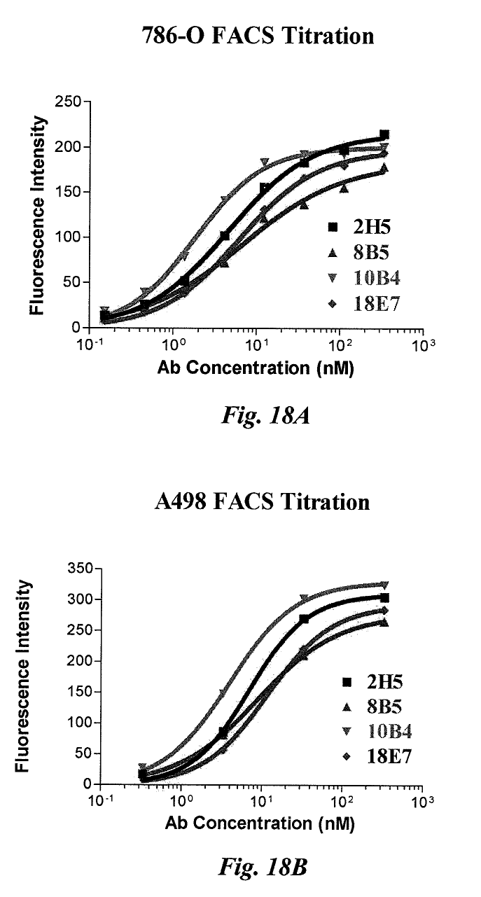

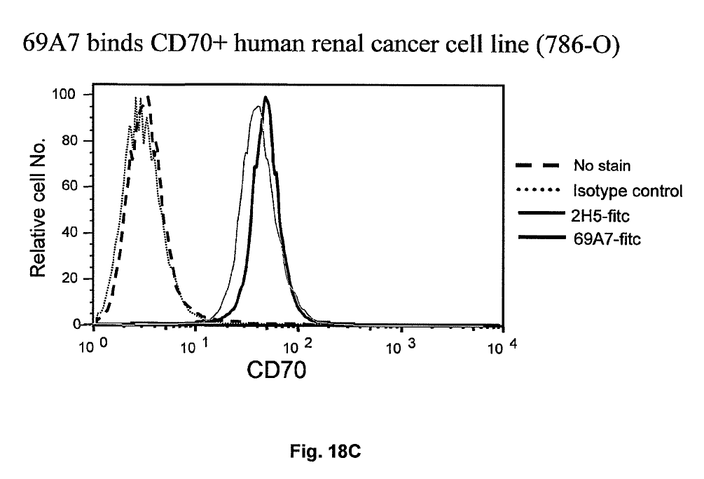

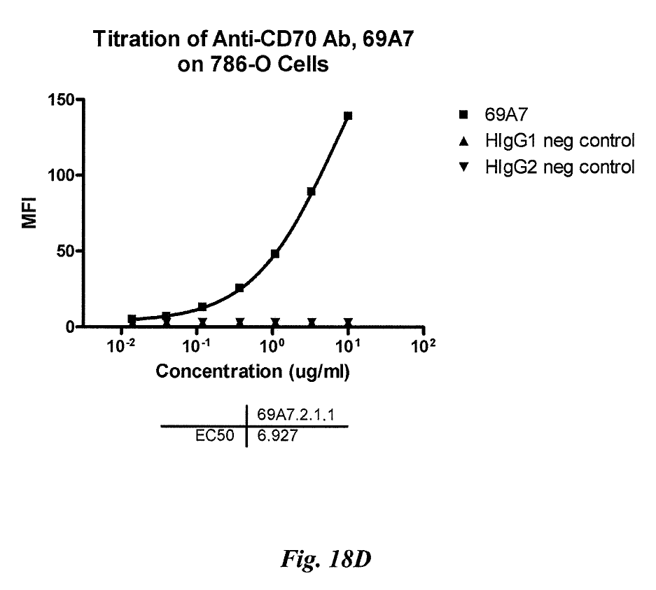

The renal cell carcinoma cell lines 786-O and A-498 were tested for binding of the HuMAb anti-CD70 human monoclonal antibodies 2H5, 8B5, 10B4 and 18E7 at different concentrations. Binding of the anti-CD70 human monoclonal antibodies was assessed by incubating 5×10⁵ cells with antibody at a starting concentration of 50 μg/ml and serially diluting the antibody at a 1:3 dilution. The cells were washed and binding was detected with a PE-labeled anti-human IgG Ab. Flow cytometric analyses were performed using a FACSCalibur flow cytometry (Becton Dickinson, San Jose, Calif.). The results are shown in FIG. 18A (786-O) and FIG. 18B (A-498). The anti-CD70 monoclonal antibodies 2H5, 8B5, 10B4 and 18E7 bound to the renal carcinoma cell lines 786-O and A-498 in a concentration dependent manner, as measured by the mean fluorescent intensity (MFI) of staining. The EC₅₀ values for the anti-CD70 monoclonal antibodies ranged from 1.844 nM to 6.669 nM for the 786-O cell line and 3.984 nM to 11.84 nM for the A-498 cell line.

Binding of the HuMAb 2H5 and 69A7 anti-CD70 human monoclonal antibodies to the renal cell carcinoma cell line 786-O was assessed by incubating 2×10⁵ cells with either 2H5 or 69A7 at a concentration of 10 μg/ml. An isotype control antibody was used as a negative control. The cells were washed and binding was detected with a FITC-labeled anti-human IgG Ab. Flow cytometric analyses were performed using a FACSCalibur flow cytometry (Becton Dickinson, San Jose, Calif.). The results are shown in FIG. 18C. Both anti-CD70 monoclonal antibodies bound to the renal carcinoma cell line 786-O.

The renal cell carcinoma cell line 786-O was tested for binding of the HuMAb anti-CD70 human monoclonal antibody 69A7 at different concentrations. Binding of the anti-CD70 human monoclonal antibodies was assessed by incubating 5×10⁵ cells with antibody at a starting concentration of 10 μg/ml and serially diluting the antibody at a 1:3 dilution. The cells were washed and binding was detected with a PE-labeled anti-human IgG Ab. Flow cytometric analyses were performed using a FACSCalibur flow cytometry (Becton Dickinson, San Jose, Calif.). The results are shown in FIG. 18D. The anti-CD70 monoclonal antibody 69A7 bound to the renal carcinoma cell line 786-O in a concentration dependent manner, as measured by the mean fluorescent intensity (MFI) of staining. The EC₅₀ value for the anti-CD70 monoclonal antibody 69A7 binding to 786-O cells was 6.927 nM.

These data demonstrate that the anti-CD70 HuMAbs bind to renal cell carcinoma cell lines.

### Example 5

### Characterization of Anti-CD70 Antibody binding to CD70 Expressed on the Surface of Lymphoma Cell Lines

Anti-CD70 antibodies were tested for binding to lymphoma cells expressing CD70 on their cell surface by flow cytometry.

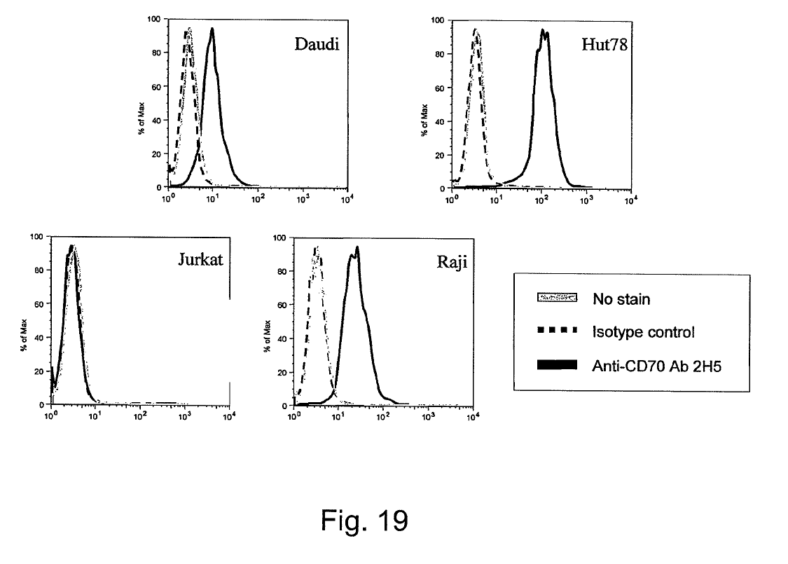

The lymphoma cell lines Daudi (ATCC Accession No. CCL-213), HuT 78 (ATCC Accession No. TIB-161) and Raji (ATCC Accession No. CCL-86) were each tested for antibody binding. Binding of the HuMAb 2H5 anti-CD70 human monoclonal antibody was assessed by incubating 1×10⁵ cells with 2H5 at a concentration of 1 μg/ml. The cells were washed and binding was detected with a FITC-labeled anti-human IgG Ab. The Jurkat cell line, which does not express CD70 on the cell surface, was used as a negative control. Flow cytometric analyses were performed using a FACSCalibur flow cytometry (Becton Dickinson, San Jose, Calif.). The results are shown in FIG. 19. The anti-CD70 monoclonal antibody 2H5 bound to the lymphoma cell lines Daudi, HuT 78 and Raji, as measured by the mean fluorescent intensity (MFI) of staining.

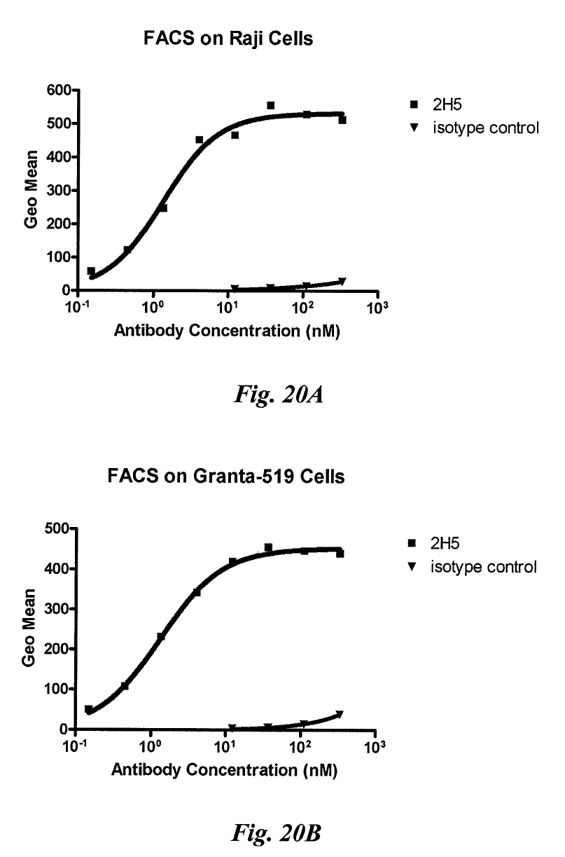

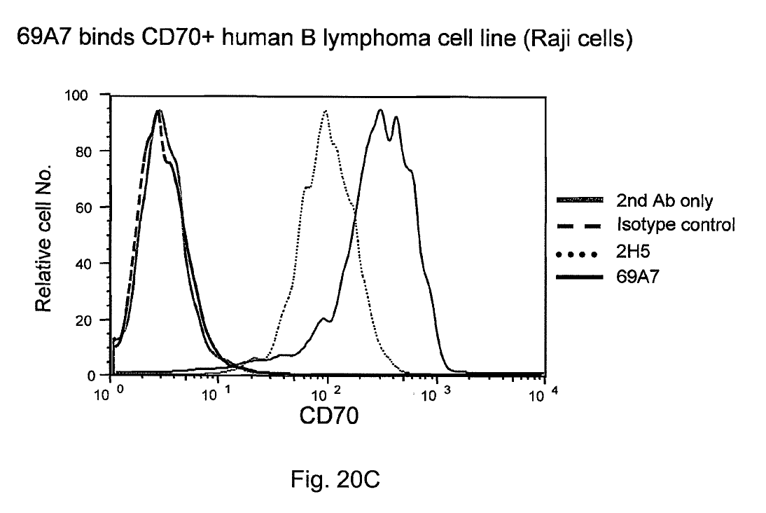

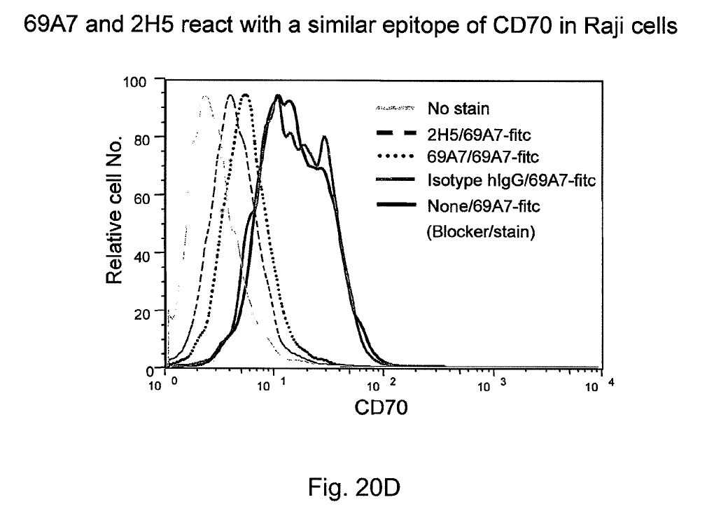

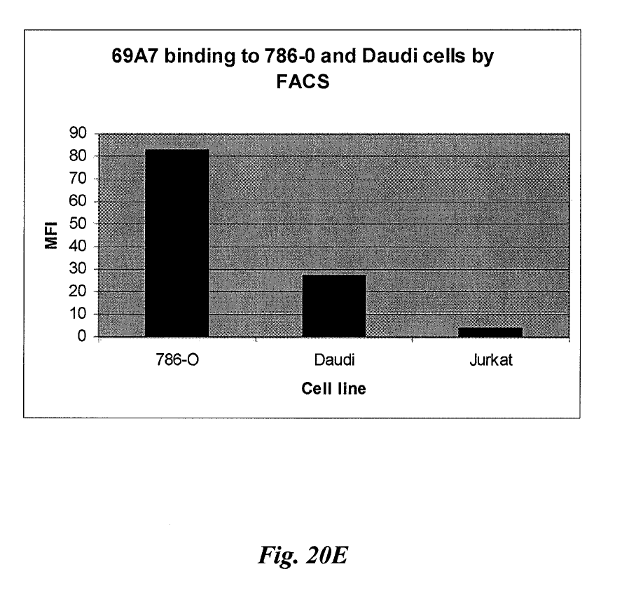

The lymphoma cell lines Raji and Granta 519 (DSMZ Accession No. 342) were tested for binding of the HuMAb anti-CD70 human monoclonal antibody 2H5 at varying concentrations. Binding of the anti-CD70 human monoclonal antibodies was assessed by incubating 5×10⁵ cells with antibody at a starting concentration of 50 μg/ml and serially diluting the antibody at a 1:3 dilution. An isotype control antibody was used as a negative control. The cells were washed and binding was detected with a PE-labeled anti-human IgG Ab. Flow cytometric analyses were performed using a FACSCalibur flow cytometry (Becton Dickinson, San Jose, Calif.). The results are shown in FIGS. 20A (Raji) and 20B (Granta 519). The anti-CD70 monoclonal antibody 2H5 bound to the lymphoma cell lines Raji and Granta 519 in a concentration dependent manner, as measured by the mean fluorescent intensity (MFI) of staining. The EC₅₀ values for the anti-CD70 antibody were 1.332 nM for the Raji cells and 1.330 nM for the Granta 519 cells.

Binding of the HuMAbs 2H5 and 69A7 anti-CD70 human monoclonal antibodies to the Raji lymphoma cell line was assessed by incubating 2×10⁵ cells with HuMAb at a concentration of 10 μg/ml. The cells were washed and binding was detected with a FITC-labeled anti-human IgG Ab. An isotype control antibody and secondary antibody alone were used as negative control. Flow cytometric analyses were performed using a FACSCalibur flow cytometry (Becton Dickinson, San Jose, Calif.). The results are shown in FIG. 20C. Both anti-CD70 monoclonal antibodies bound to the Raji lymphoma cell line, as measured by the mean fluorescent intensity (MFI) of staining.

A competition FACS assay was carried out to elucidate the binding specificity of 69A7 against 2H5. Raji cells were incubated with either naked 69A7, 2H5, an isotype control antibody or no antibody at a concentration of 10 μg/ml. After wash, the cells were incubated with FITC-conjugated 69A7 at a concentration of 10 μg/ml. The cells were washed and binding was detected with a FITC-labeled anti-human IgG Ab. Flow cytometric analyses were performed using a FACSCalibur flow cytometry (Becton Dickinson, San Jose, Calif.). The results are shown in FIG. 20D. Both the anti-CD70 antibody 69A7 and 2H5 blocked binding of FITC-labeled 69A7, indicating that both 2H5 and 69A7 share a similar binding epitope.

The Daudi lymphoma cell line and 786-O renal carcinoma cell were further tested for antibody binding. Binding of the HuMAb 69A7 anti-CD70 human monoclonal antibody was assessed by incubating 2×10⁵ cells with 69A7 at a concentration of 1 μg/ml. The cells were washed and binding was detected with a FITC-labeled anti-human IgG Ab. The Jurkat cell line, which does not express CD70 on the cell surface, was used as a negative control. Flow cytometric analyses were performed using a FACSCalibur flow cytometry (Becton Dickinson, San Jose, Calif.). The results are shown in FIG. 20E. The anti-CD70 monoclonal antibody 69A7 bound to the Daudi lymphoma cell line and 786-O renal carcinoma cell line, as measured by the mean fluorescent intensity (MFI) of staining.

These data demonstrate that the anti-CD70 HuMAbs bind to lymphoma cell lines.

### Example 6

### Scatchard Analysis of Binding Affinity of Anti-CD70 Monoclonal Antibodies

The binding affinity of the 2H5, 8B5, 10B4 and 18E7 monoclonal antibodies was tested for binding affinity to a CD70 transfected CHO cell line using a Scatchard analysis.

CHO cells were transfected with full length CD70 using standard techniques and grown in RPMI media containing 10% fetal bovine serum (FBS). The cells were trypsinized and washed once in Tris based binding buffer (24 mM Tris pH 7.2, 137 mM NaCl, 2.7 mM KCl, 2 mM Glucose, 1 mM CaCl₂, 1 mM MgCl₂, 0.1% BSA) and the cells were adjusted to 2×10⁶ cells/ml in binding buffer. Millipore plates (MAFB NOB) were coated with 1% nonfat dry milk in water and stored a 4° C. overnight. The plates were washed three times with 0.2 ml of binding buffer. Fifty microliters of buffer alone was added to the maximum binding wells (total binding). Twenty-five microliters of buffer alone was added to the control wells (non-specific binding). Varying concentration of ¹²⁵I-anti-CD70 antibody was added to all wells in a volume of 25 μl. Varying concentrations of unlabeled antibody at 100 fold excess was added in a volume of 25 μl to control wells and 25 μl of CD70 transfected CHO cells (2×10⁶ cells/ml) in binding buffer were added to all wells. The plates were incubated for 2 hours at 200 RPM on a shaker at 4° C. At the completion of the incubation the Millipore plates were washed three times with 0.2 ml of cold wash buffer (24 mM Tris pH 7.2, 500 mM NaCl, 2.7 mM KCl, 2 mM Glucose, 1 mM CaCl₂, 1 mM MgCl₂, 0.1% BSA.). The filters were removed and counted in a gamma counter. Evaluation of equilibrium binding was performed using single site binding parameters with the Prism software (San Diego, Calif.).

Using the above scatchard binding assay, the K_{D} of the antibody for CD70 transfected CHO cells was approximately 2.1 nM for 2H5, 5.1 nM or 8B5, 1.6 nM for 10B4 and 1.5 nM for 18E7.

### Example 7

### Internalization of Anti-CD70 Monoclonal Antibody

Anti-CD70 HuMAbs were tested for the ability to internalize into CD70-expressing renal carcinoma cells using a Hum-Zap internalization assay. The Hum-Zap assay tests for internalization of a primary human antibody through binding of a secondary antibody with affinity for human IgG conjugated to the cytotoxin saporin.

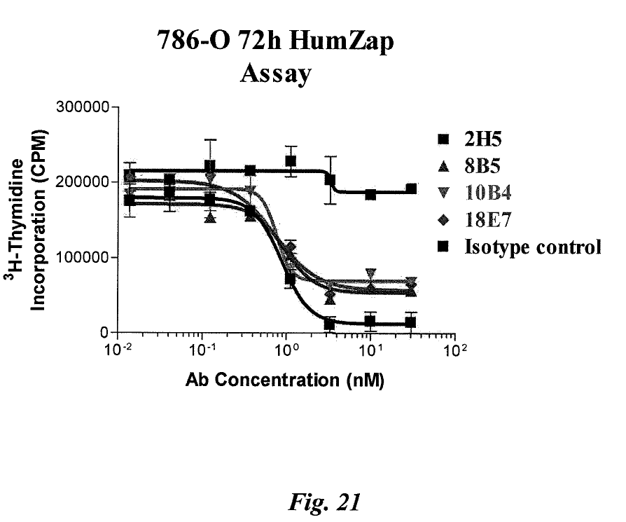

The CD70-expressing renal carcinoma cancer cell line 786-O was seeded at 1.25×10⁴ cells/well in 100 μl wells overnight. The anti-CD70 HuMAb antibodies 2H5, 8B5, 10B4 or 18E7 were added to the wells at a starting concentration of 30 nM and titrated down at 1:3 serial dilutions. An isotype control antibody that is non-specific for CD70 was used as a negative control. The Hum-Zap (Advanced Targeting Systems, San Diego, Calif., IT-22-25) was added at a concentration of 11 nM and plates were allowed to incubate for 72 hours. The plates were then pulsed with 1.0 μCi of ³H-thymidine for 24 hours, harvested and read in a Top Count Scintillation Counter (Packard Instruments, Meriden, Conn.). The results are shown in FIG. 21. The anti-CD70 antibodies 2H5, 8B5, 10B4 and 18E7 showed an antibody concentration dependent decrease in ³H-thymidine incorporation in CD70-expressing 786-O renal carcinoma cancer cells. The EC₅₀ value for the anti-CD70 antibody 2H5 was 0.9 nM. This data demonstrates that the anti-CD70 antibodies 2H5, 8B5, 10B4 and 18E7 internalize into a renal carcinoma cancer cell line.

### Example 8

### Assessment of Cell Killing of a Cytotoxin-Conjugated Anti-CD70 Antibody on Renal Cell Carcinoma Cell Lines

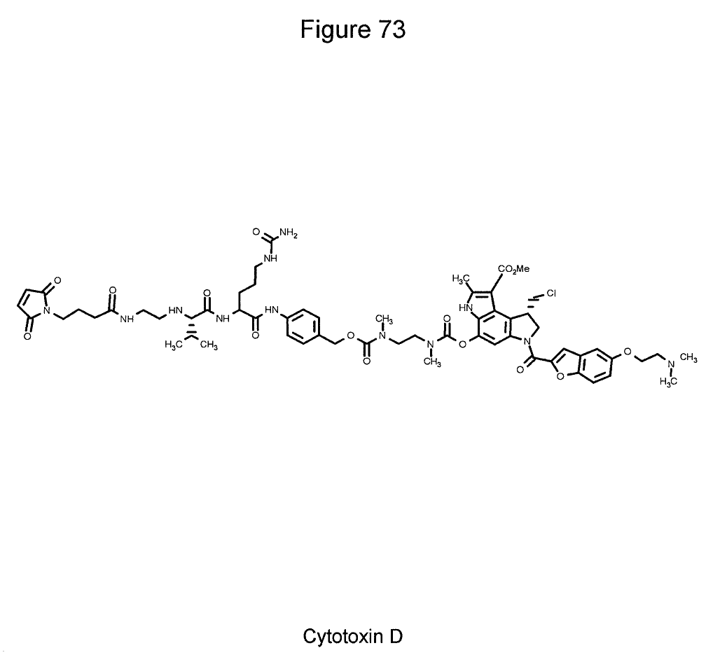

In this example, anti-CD70 monoclonal antibodies conjugated to cytotoxin D (FIG. 73) were tested for the ability to kill CD70+ renal cell carcinoma cell lines in a cell proliferation assay. Cytotoxin D is a prodrug requiring esterase activation.

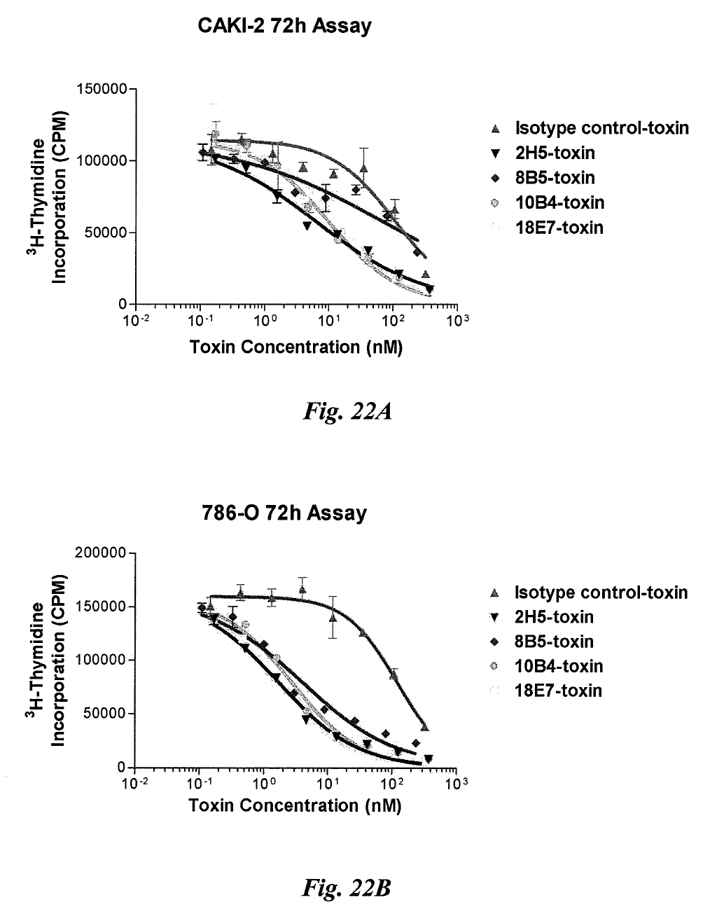

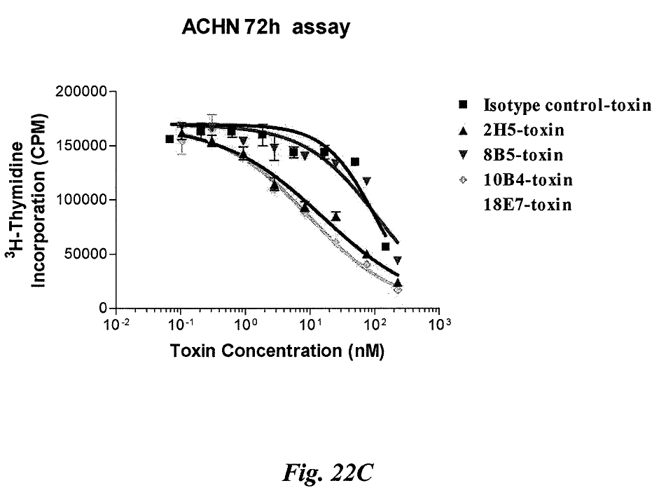

The anti-CD70 HuMAb antibodies 2H5, 8B5, 10B4 or 18E7 were conjugated to cytotoxin D via a linker, such as a peptidyl, hydrazone or disulfide linker. The CD70-expressing renal carcinoma cancer cell lines ACHN and Caki-2 were seeded at 2.5×10⁴ cells/wells and the CD70-expressing renal carcinoma cancer cell line 786-O was seeded at 1.25×10⁴ cells/wells in 100 μl wells for 3 hours. The anti-CD70 antibody-cytotoxin conjugate was added to the wells at a starting concentration of 30 nM and titrated down at 1:3 serial dilutions. An isotype control antibody that is non-specific for CD70 was used as a negative control. Plates were allowed to incubate for 69 hours. The plates were then pulsed with 1.0 μCi of ³H-thymidine for 24 hours, harvested and read in a Top Count Scintillation Counter (Packard Instruments, Meriden, Conn.). The results are shown in FIGS. 22A (Caki-2), 22B (786-O) and 22C (ACHN). The anti-CD70 antibodies 2H5, 8B5, 10B4 and 18E7 showed an antibody-cytotoxin concentration dependent decrease in ³H-thymidine incorporation in CD70-expressing Caki-2, 786-O and ACHN renal carcinoma cancer cells. The EC₅₀ values for the anti-CD70 antibodies ranged from 6 nM to 76 nM in the CAKI-2 cells, 1.6 nM to 3.9 nM in the 786-O cells and 9 nM to 108 nM in the ACHN cells. This data demonstrates that the anti-CD70 antibodies 2H5, 8B5, 10B4 and 18E7 are cytotoxic to renal carcinoma cancer cells when conjugated to a cytotoxin.

### Example 9

### Assessment of ADCC Activity of Anti-CD70 Antibody

In this example, anti-CD70 monoclonal antibodies were tested for the ability to kill CD70+ cell lines in the presence of effector cells via antibody dependent cellular cytotoxicity (ADCC) in a fluorescence cytotoxicity assay.

Human effector cells were prepared from whole blood as follows. Human peripheral blood mononuclear cells were purified from heparinized whole blood by standard Ficoll-paque separation. The cells were resuspended in RPMI1640 media containing 10% FBS and 200 U/ml of human IL-2 and incubated overnight at 37° C. The following day, the cells were collected and washed four times in culture media and resuspended at 2×10⁷ cells/ml. Target CD70+ cells were incubated with BATDA reagent (Perkin Elmer, Wellesley, Mass.) at 2.5 μl BATDA per 1×10⁶ target cells/ml for 20 minutes at 37° C. The target cells were washed four times, spun down and brought to a final volume of 1×10⁵ cells/ml.

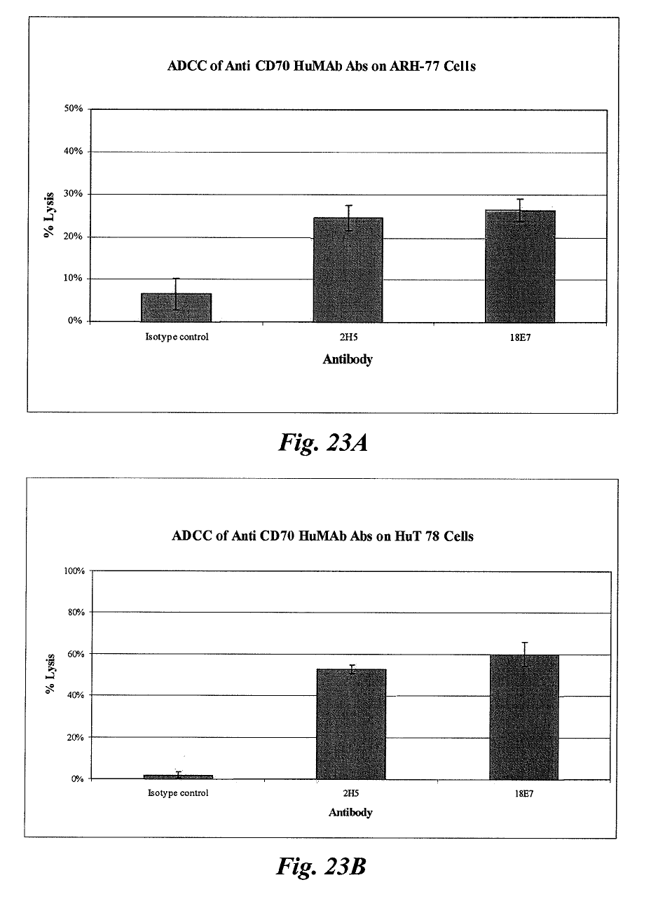

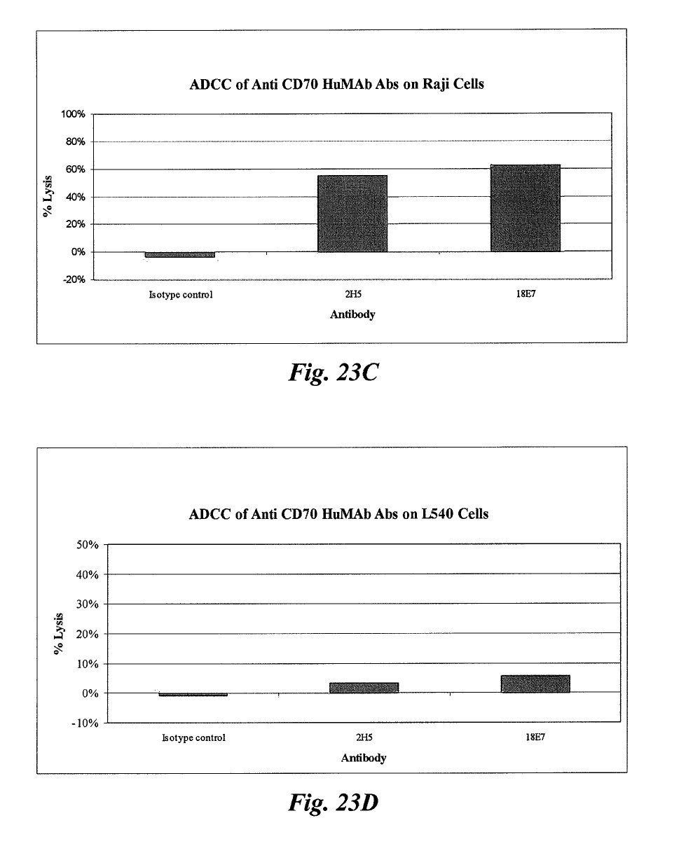

The CD70+ cell lines ARH-77 (human B lymphoblast leukemia; ATCC Accession No. CRL-1621), HuT 78 (human cutaneous lymphocyte lymphoma; ATCC Accession No. TIB-161), Raji (human B lymphocyte Burkitt's lymphoma; ATCC Accession No. CCL-86) and a negative control cell line L540 (human Hodgkin's lymphoma; DSMZ Deposit No. ACC 72) were tested for antibody specific ADCC to the human anti-CD70 monoclonal antibodies using the Delfia fluorescence emission analysis as follows. Each target cell line (100 μl of labeled target cells) was incubated with 50 μl of effector cells and 50 μl of antibody. A target to effector ratio of 1:50 was used throughout the experiments. In all studies, a human IgG1 isotype control was used as a negative control. Following a 2000 rpm pulse spin and one hour incubation at 37° C., the supernatants were collected, quick spun again and 20 μl of supernatant was transferred to a flat bottom plate, to which 180 μl of Eu solution (Perkin Elmer, Wellesley, Mass.) was added and read in a RubyStar reader (BMG Labtech). The % lysis was calculated as follows: (sample release−spontaneous release*100)/(maximum release−spontaneous release), where the spontaneous release is the fluorescence from wells which only contain target cells and maximum release is the fluorescence from wells containing target cells and have been treated with 2% Triton-X. Cell cytotoxicity % lysis for the ARH-77, HuT 78, Raji and L-540 cell lines are shown in FIGS. 23A-D, respectively. Each of the CD70+expressing cell lines ARH-77, HuT 78 and Raji showed antibody mediated cytotoxicity with the HuMAb anti-CD70 antibodies 2H5 and 18E7, while the negative control cell line L-540 did not have appreciable cell cytotoxicity in the presence of anti-CD70 antibodies. This data demonstrates that HuMAb anti-CD70 antibodies show specific cytotoxicity to CD70+ expressing cells.

### Example 10

### Assessment of Cell Killing of a Cytotoxin-Conjugated anti-CD70 Antibody on Human Lymphoma Cell Lines

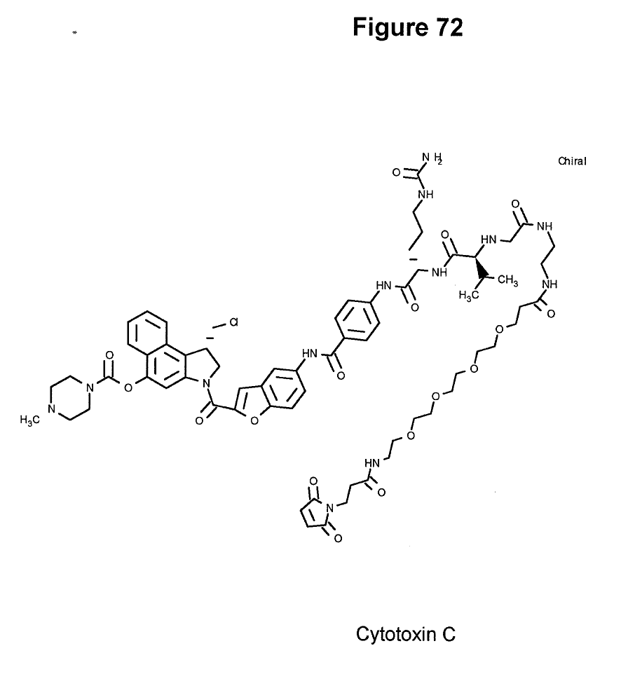

In this example, anti-CD70 monoclonal antibody 2H5 conjugated to cytotoxin C (FIG. 72) was tested for the ability to kill CD70+ human lymphoma cell lines in a cell proliferation assay. Cytotoxin C is a prodrug requiring esterase activation.

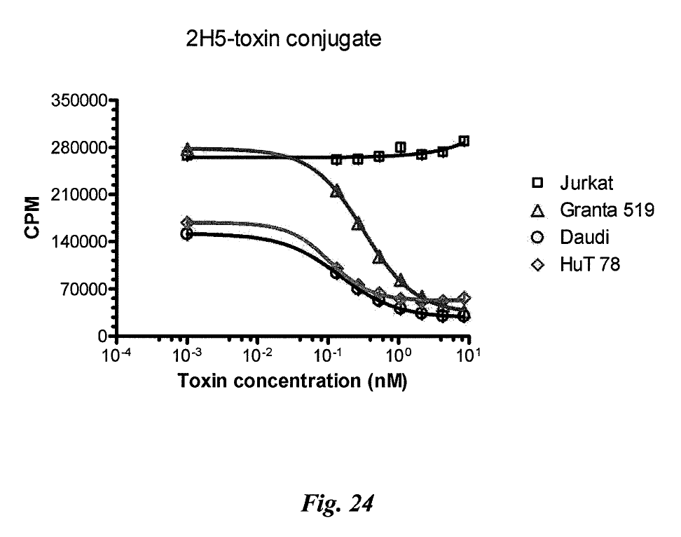

The anti-CD70 HuMAb antibody 2H5 was conjugated to cytotoxin C via a linker such as a peptidyl, hydrazone or disulfide linker. Examples of cytotoxin compounds that may be conjugated to the antibodies of the current disclosure are described in the concurrently filed application with U.S. Ser. No. 60/720,499, filed on Sep. 26, 2005, and PCT Publication No. WO 07/038658, filed on Sep. 26, 2006, the contents of which are hereby incorporated herein by reference. The CD70-expressing human lymphoma cancer cell lines Daudi, HuT 78, Granta 519 and Raji were seeded at 10⁵ cells/well in 100 μl wells for 3 hours. The anti-CD70 antibody-cytotoxin conjugate was added to the wells at a starting concentration of 30 nM and titrated down at 1:2 serial dilutions. The HuMAb antibody 2H5-cytotoxin conjugate was also tested on Jurkat cells a negative control cell line that does not express CD70 on the cell surface. Plates were allowed to incubate for 72 hours. The plates were then pulsed with 0.5 μCi of ³H-thymidine for 8 hours before termination of the culture, harvested and read in a Top Count Scintillation Counter (Packard Instruments). FIG. 24 showed the effects of the 2H5-conjugate on the Daudi, HuT 78, Granta 519 and Jurkat cells. The anti-CD70 antibody 2H5 showed an antibody-cytotoxin concentration dependent decrease in ³H-thymidine incorporation in CD70-expressing Daudi, HuT 78 and Granta 519 B-cell lymphoma cancer cells, but not in the Jurkat cells.

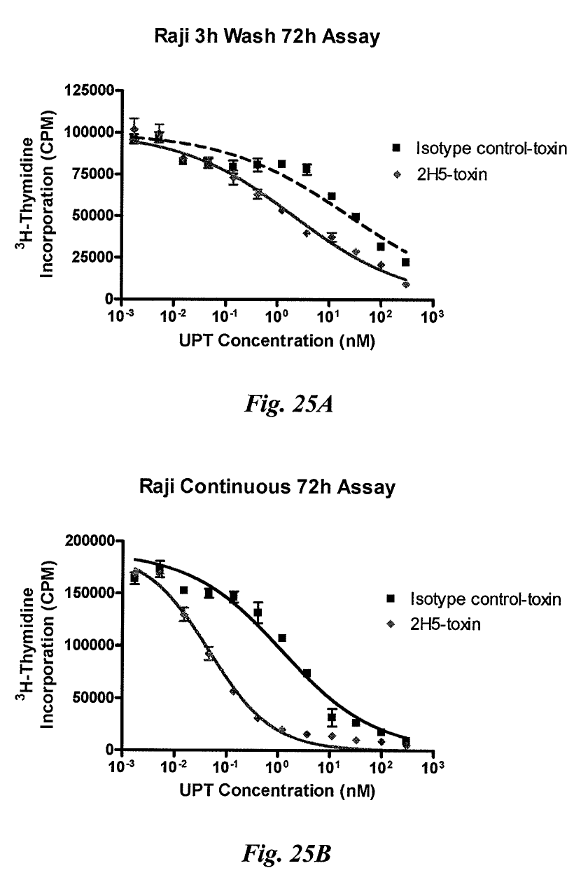

In a separate assay, the CD70-expressing human lymphoma cancer cell line Raji was seeded at 10⁴ cells/well in 100 μl wells for 3 hours. An anti-CD70 antibody-cytotoxin conjugate was added to the wells at a starting concentration of 30 nM and titrated down at 1:3 serial dilutions. A cytotoxin-conjugate isotype control antibody was used as a control. Plates were allowed to incubate for 72 hours with either a wash at 3 hours or a continuous wash. The plates were then pulsed with 0.5 μCi of ³H-thymidine for 8 hours before termination of the culture, harvested and read in a Top Count Scintillation Counter (Packard Instruments). FIGS. 25A and 25B showed an antibody-cytotoxin concentration dependent decrease in ³H-thymidine incorporation on Raji cells with a 3 hour wash or with a continuous wash, respectively.

This data demonstrates that anti-CD70 antibodies conjugated to a cytotoxin show specific cytotoxicity to human lymphoma cancer cells.

### Example 11

### Treatment of in vivo Tumor Xenograft Model Using Naked and Cytotoxin-Conjugated Anti-CD70 Antibodies

Mice implanted with a renal cell carcinoma tumor were treated in vivo with cytotoxin-conjugated anti-CD70 antibodies to examine the in vivo effect of the antibodies on tumor growth.

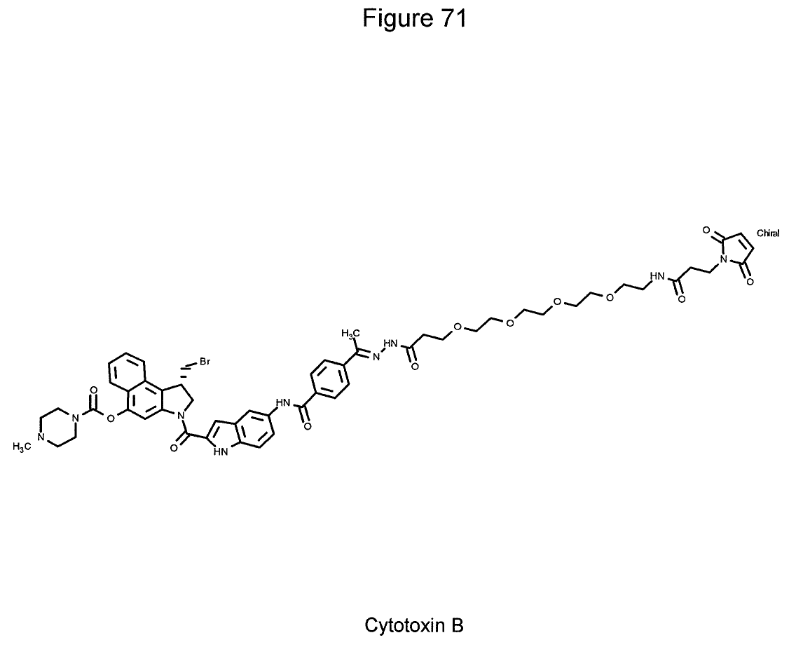

A-498 (ATCC Accession No. HTB-44) and ACHN (ATCC Accession No. CRL-1611) cells were expanded in vitro using standard laboratory procedures. Male Ncr athymic nude mice (Taconic, Hudson, N.Y.) between 6-8 weeks of age were implanted subcutaneously in the right flank with 7.5 ×10⁶ ACHN or A-498 cells in 0.2 ml of PBS/Matrigel (1:1) per mouse. Mice were weighed and measured for tumors three dimensionally using an electronic caliper twice weekly after implantation. Tumor volumes were calculated as height×width×length. Mice with ACHN tumors averaging 270 mm³ or A498 tumors averaging 110 mm³ were randomized into treatment groups. The mice were dosed intraperitoneally with PBS vehicle, cytotoxin-conjugated isotype control antibody or cytotoxin-conjugated anti-CD70 HuMAb 2H5 on Day 0. Examples of cytotoxin compounds that may be conjugated to the antibodies of the current disclosure are described in U.S. Provisional Application Ser. No 60/720,499 and PCT Publication No. WO 07/038658, filed on Sep. 26, 2006, the contents of which are hereby incorporated herein by reference. The mice in the A-498 sample group were tested with three different cytotoxin compounds (cytotoxin A (N1), cytotoxin B (FIG. 71), and cytotoxin C (FIG. 72)). Mice were monitored for tumor growth for 60 days post dosing. Mice were euthanized when the tumors reached tumor end point (2000 mm³).

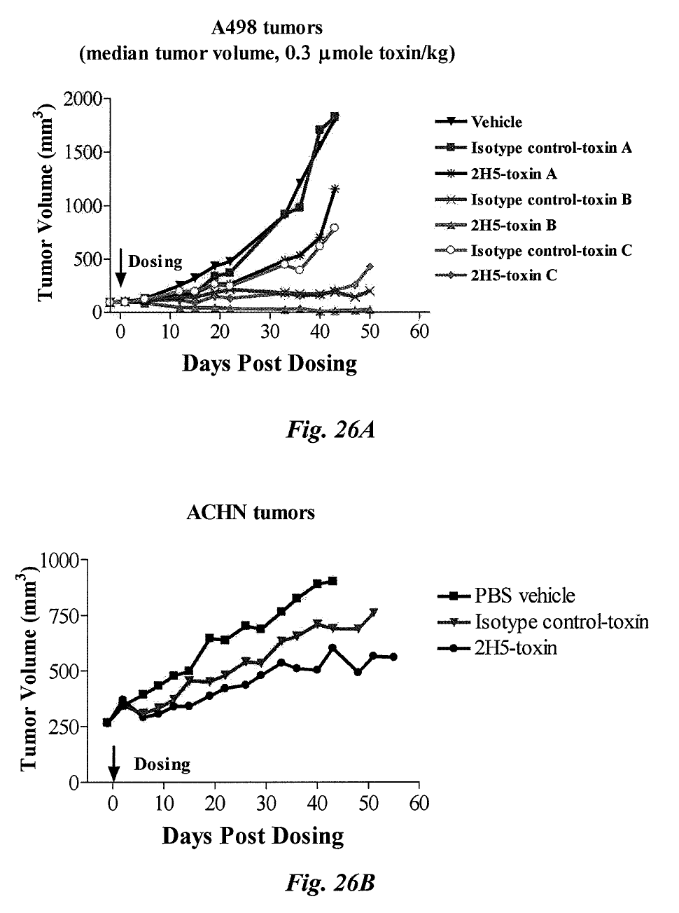

The results are shown in FIG. 26A (A-498 tumors) and 26B (ACHN tumors). The anti-CD70 antibody 2H5 conjugated to a cytotoxin extended the mean time to reach the tumor end point volume (2000 mm³) and slowed tumor growth progression. Thus, treatment with an anti-CD70 antibody-cytotoxin conjugate had a direct in vivo inhibitory effect on tumor growth.

### Example 12

### Immunohistochemistry with 2H5

The ability of the anti-CD70 HuMAb 2H5 to recognize CD70 by immunohistochemistry was examined using clinical biopsies from clear cell renal cell carcinoma (ccRCC), lymphoma and glioblastoma patients.

For immunohistochemistry, 5μm frozen sections were used (Ardais Inc, USA). After drying for 30 minutes, sections were fixed with acetone (at room temperature for 10 minutes) and air-dried for 5 minutes. Slides were rinsed in PBS and then pre-incubated with 10% normal goat serum in PBS for 20 min and subsequently incubated with 10 μg/ml fitcylated 2H5 in PBS with 10% normal goat serum for 30 min at room temperature. Next, slides were washed three times with PBS and incubated for 30 min with mouse anti-FITC 10 μg/ml DAKO) at room temperature. Slides were washed again with PBS and incubated with Goat anti-mouse HRP conjugate (DAKO) for 30 minutes at room temperature. Slides were washed again 3× with PBS. Diaminobenzidine (Sigma) was used as substrate, resulting in brown staining. After washing with distilled water, slides were counter-stained with hematoxyllin for 1 min. Subsequently, slides were washed for 10 secs in running distilled water and mounted in glycergel (DAKO) Clinical biopsy immunohistochemical staining displayed positive staining in the Non-Hodgkin's s Lymphoma, plasmacytoma, ccRcc and glioblastoma sections. Only malignant cells were positive in each case, adjacent normal tissue was not stained.

### Example 13

### Production of Defucosylated HuMAbs

Antibodies with reduced amounts of fucosyl residues have been demonstrated to increase the ADCC ability of the antibody. In this example, the 2H5 HuMAb that is lacking in fucosyl residues has been produced.

The CHO cell line Ms704-PF, which lacks the fucosyltransferase gene, FUT 8 (Biowa, Inc., Princeton, N.J.) was electroporated with a vector which expresses the heavy and light chains of antibody 2H5. Drug-resistant clones were selected by growth in Ex-Cell 325-PF CHO media (JRH Biosciences, Lenexa, Kans.) with 6 mM L-glutamine and 500 μg/ml G418 (Invitrogen, Carlsbad, Calif.). Clones were screened for IgG expression by standard ELISA assay. Two separate clones were produced, B8A6 and B8C11, which had production rates ranging from 1.0 to 3.8 picograms per cell per day.

### Example 14

### Assessment of ADCC Activity of Defucosylated anti-CD70

In this example, a defucosylated and non-defucosylated anti-CD70 monoclonal antibody was tested for the ability to kill CD70+cells in the presence of effector cells via antibody dependent cellular cytotoxicity (ADCC) in a fluorescence cytotoxicity assay

Human Anti-CD70 monoclonal antibody 2H5 was defucosylated as described above. Human effector cells were prepared from whole blood as follows. Human peripheral blood mononuclear cells were purified from heparinized whole blood by standard Ficoll-paque separation. The cells were resuspended in RPMI1640 media containing 10% FBS (culture media) and 200 U/ml of human IL-2 and incubated overnight at 37° C. The following day, the cells were collected and washed once in culture media and resuspended at 2×10⁷ cells/ml. Target CD70+cells were incubated with BATDA reagent (Perkin Elmer, Wellesley, Mass.) at 2.5 μl BATDA per 1×10⁶ target cells/mL in culture media supplemented with 2.5 mM probenecid (assay media) for 20 minutes at 37° C. The target cells were washed four times in PBS with 20 mM HEPES and 2.5 mM probenecid, spun down and brought to a final volume of 1×10⁵ cells/ml in assay media.

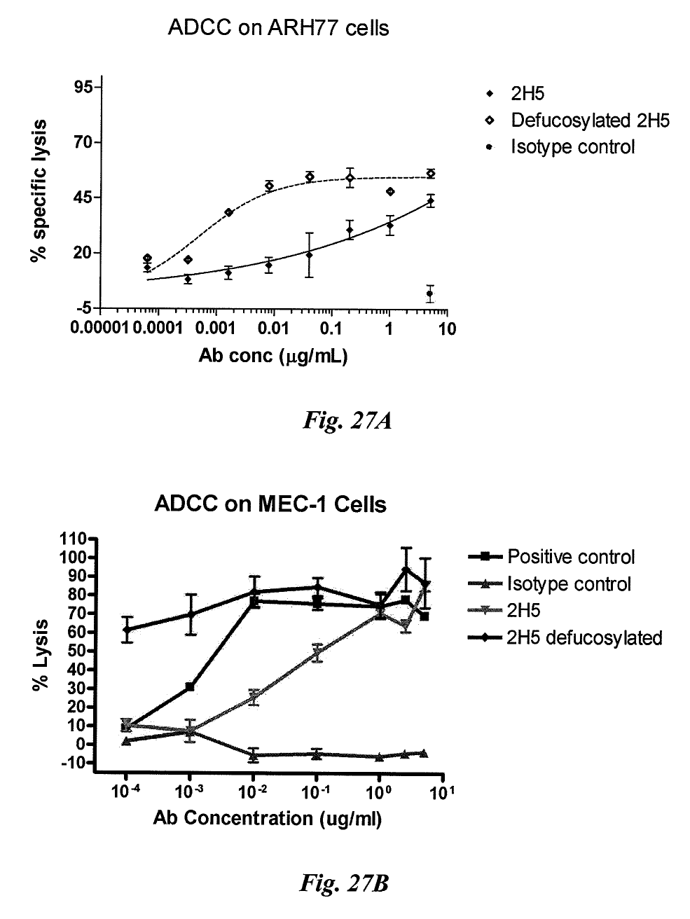

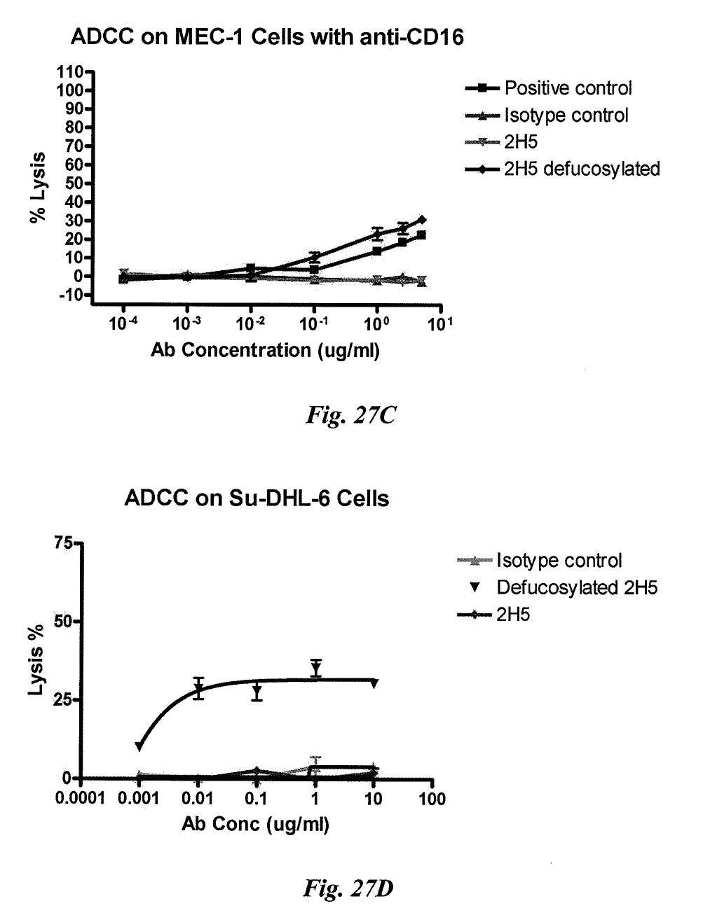

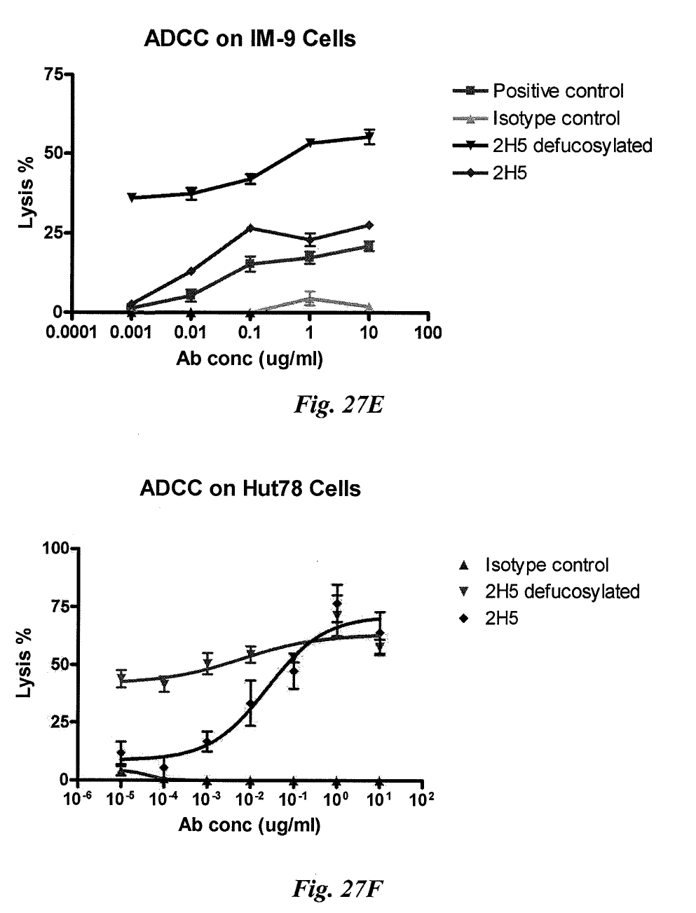

The CD70+ cell lines ARH-77 (human B lymphoblast leukemia; ATCC Accession No. CRL-1621), MEC-1 (human chronic B cell leukemia; DSMZ Accession No. ACC 497), SU-DHL-6 (human B cell lymphoma, DSMZ Accession No. Acc572), IM-9 (human B lymphoblast; ATCC Accession No. CCL-159) and HuT 78 (human cutaneous lymphocyte lymphoma; ATCC Accession No. TIB-161), were tested for antibody specific ADCC to the defucosylated and non-defucosylated human anti-CD70 monoclonal antibody 2H5 using the Delfia fluorescence emission analysis as follows. The target cell line ARH77 (100 μl of labeled target cells) was incubated with 50 μl of effector cells and 50 μl of either 2H5 or defucosylated 2H5 antibody. A target to effector ratio of 1:50 was used throughout the experiments. A human IgG1 isotype control was used as a negative control. Following a 2100 rpm pulse spin and one hour incubation at 37° C., the supernatants were collected, quick spun again and 20 μl of supernatant was transferred to a flat bottom plate, to which 180 μl of Eu solution (Perkin Elmer, Wellesley, Mass.) was added and read in a Fusion Alpha TRF plate reader (Perkin Elmer). The % lysis was calculated as follows: (sample release−spontaneous release*100), (maximum release−spontaneous release), where the spontaneous release is the fluorescence from wells which only contain target cells and maximum release is the fluorescence from wells containing target cells and have been treated with 3% Lysol Cell cytotoxicity % specific lysis for the ARH-77 cell line is shown in FIGS. 27A-F. The CD70+ expressing cell lines ARH-77, MEC-1, SU-DHL-6, IM-9 and HuT 78 showed antibody mediated cytotoxicity with the HuMAb anti-CD70 antibody 2H5 and an increased percentage of specific lysis associated with the defucosylated form of the anti-CD70 antibody 2H5. In addition, anti-CD 16 antibody was shown to block the ADCC effect in the MEG-1 cell line. This data demonstrates that defucosylated HuMAb anti-CD70 antibodies show increased specific cytotoxicity to CD70+ expressing cells.

### Example 15

### Assessment of ADCC Activity of Anti-CD70 Antibody Using a ⁵¹Cr-Release Assay

In this example, an anti-CD70 monoclonal antibody was tested for the ability to kill CD70+ Raji B lymphocyte cells in the presence of effector cells via antibody dependent cellular cytotoxicity (ADCC) in a ⁵¹Cr-release assay.

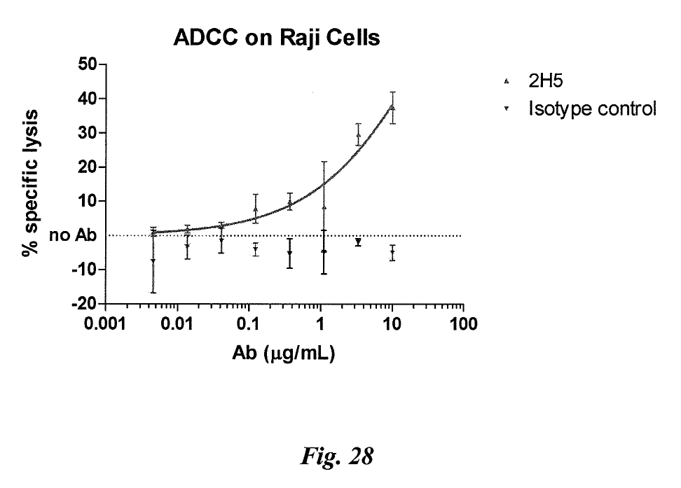

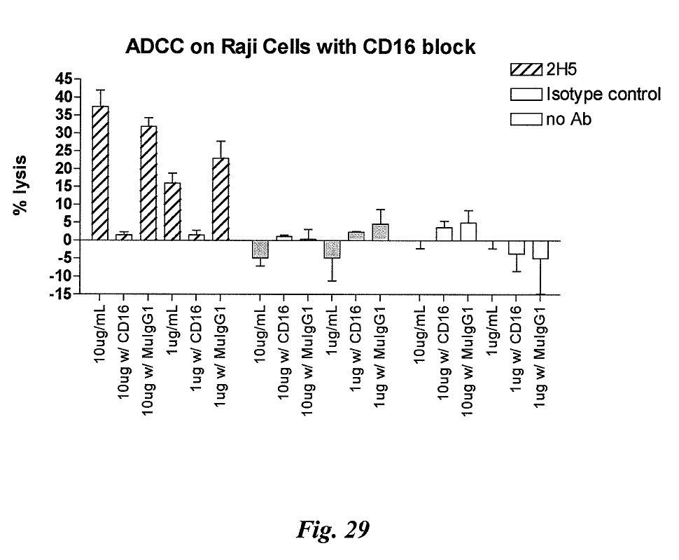

Human peripheral blood mononuclear cells (effector cells) were purified from heparinized whole blood by standard Ficoll-paque separation. The cells were resuspended at 2×10⁶/mL in RPMI1640 media containing 10% FBS and 200 U/ml of human IL-2 and incubated overnight at 37° C. The following day, the cells were collected and washed once in culture media and resuspended at 2×10⁷ cells/ml. Two million target Raji cells (human B lymphocyte Burkitt's lymphoma; ATCC Accession No. CCL-86) were incubated with 200 μCi ⁵¹Cr in 1 ml total volume for 1 hour at 37° C. The target cells were washed once, resuspended in 1 ml of media, and incubated at 37° C. for an additional 30 minutes. After the final incubation, the target cells were washed once and brought to a final volume of 1×10⁵ cells/ml. For the final ADCC assay, 100 μl of labeled Raji cells were incubated with 50 μl of effector cells and 50 μl of antibody. A target to effector ratio of 1:100 was used throughout the experiments. In all studies, human IgG1 isotype control was used as a negative control. In some studies, the PBMC culture was separated equally into tubes containing either 20 μg/mL of an anti-human CD16 antibody, an irrelevant mouse IgG1 antibody, or no antibody prior to adding PBMC to the assay plate. Following a 15 minute incubation at 27° C., the blood cells were used as described above without washing. Following a 4 hour incubation at 37° C., the supernatants were collected and counted on a Cobra II auto-gamma Counter (Packard Instruments) with a reading window of 240-400 keV. The counts per minute were plotted as a function of antibody concentration and the data was analyzed by non-linear regression, sigmoidal dose response (variable slope) using Prism software (San Diego, Calif.). The percent lysis was determined by the following equation: % Lysis=(Sample CPM-no antibody CPM)/TritonX CPM-No antibody CPM)×100. An antibody titration curve for cell cytotoxicity % specific lysis for the Raji cell line is shown in FIG. 28. This data demonstrates that anti-CD70 antibodies have an ADCC effect on the Raji cell line. The EC₅₀ value for the anti-CD70 antibody against Raji cells was 36 nM. A graph of cytotoxicity on Raji cells in the presence of an anti-CD16 antibody is shown in FIG. 29. This data demonstrates that the ADCC effect of anti-CD70 antibodies on Raji cells is dependent upon CD 16.

### Example 16

### Assessment of ADCC Activity of Anti-CD70 Antibody on Activated T Cells

In this example, a defucosylated and non-defucosylated anti-CD70 monoclonal antibody was tested for the ability to kill activated T cells in the presence of effector cells via antibody dependent cellular cytotoxicity (ADCC) in a fluorescence cytotoxicity assay.

Human Anti-CD70 monoclonal antibody 2H5 was defucosylated as described above. Human effector cells were prepared as described above. Human spleen T cells were positively selected with anti-CD3 coated magnetic beads (Purity>90%). The cells were stimulated with anti-CD3 and anti-CD28 coated beads and 25 ng/ml IL-2 in Iscove's media+10% heat inactivated FCS for 6 days. Cells were collected and assayed for viability by propidium iodide incorporation (60% viable) and live cells were gated and analyzed for CD70 expression (˜65% CD70+ on live cells) prior to inclusion in ADCC assays.

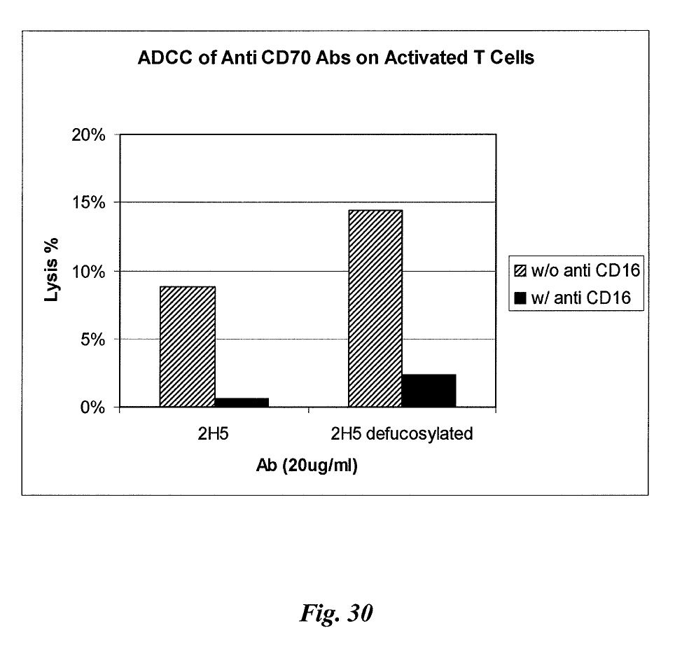

The activated T cells were tested for antibody specific ADCC to the defucosylated and non-defucosylated human anti-CD70 monoclonal antibody 2H5 using the Delfia fluorescence emission analysis as follows. The target activated T cells (100 μl of labeled target cells) was incubated with 50 μl of effector cells and 50 μl of either 2H5 or defucosylated 2H5 antibody. A target to effector ratio of 1:50 was used throughout the experiments. A human IgG1 isotype control was used as a negative control. Following a 2100 rpm pulse spin and one hour incubation at 37° C., the supernatants were collected, quick spun again and 20 μl of supernatant was transferred to a flat bottom plate, to which 180 μl of Eu solution (Perkin Elmer, Wellesley, Mass.) was added and read in a Fusion Alpha TRF plate reader (Perkin Elmer). The % lysis was calculated as follows: (sample release−spontaneous release*100)/(maximum release−spontaneous release), where the spontaneous release is the fluorescence from wells which only contain target cells and maximum release is the fluorescence from wells containing target cells and have been treated with 3% Lysol. Cell cytotoxicity % specific lysis for the activated T cells is shown in FIG. 30. The activated T cells showed antibody mediated cytotoxicity with the HuMAb anti-CD70 antibody 2H5 and an increased percentage of specific lysis associated with the defucosylated form of the anti-CD70 antibody 2H5. The antibody mediated cytotoxicity was blocked by the addition of anti-CD16 antibody in both the defucosylated and non-defucosylated forms of anti-CD70 antibody. The control IgG had no effect on cytotoxicity. This data demonstrates that defucosylated HuMAb anti-CD70 antibodies show increased specific cytotoxicity to activated T cells.

### Example 17

### Blocking Assay for Receptor-Ligand CD70-CD27 Binding

In this example, anti-CD70 monoclonal antibodies were tested for their ability to block the interaction of CD70 with the ligand CD27 using a blocking assay.

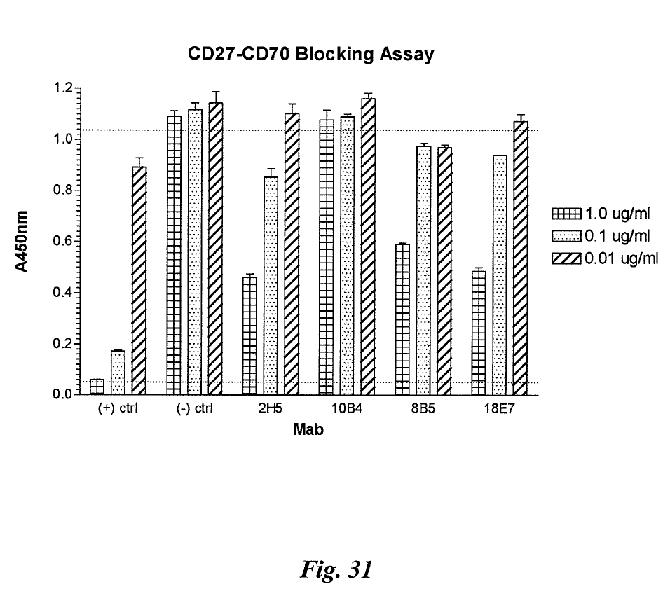

Wells were coated overnight with 100 μl/well of an anti-IgG antibody (Fc-sp.) at 2 μg/ml at 4° C. The wells were blocked with 200 μl/well 1% BSA/PBS for 1 hour at room temperature. To each well was added 100 μl/well of CD27-Fc-his at 0.16 μg/ml for 1 hour at 37° C. while shaking. Each well was washed 5 times with 200 μl/well PBS/Tween 20 (0.05% (v:v)). Anti-CD70 antibody was diluted in 10% NHS+1% BSA/PBS and mixed with CD70-myc-his at 0.05 μg/ml, incubated for 1 hour at room temperature and washed 5 times with 200 μl/well PBS/Tween 20 (0.05% (v:v)). A known antibody that blocks CD70/CD27 interaction was used as a positive control and an isotype control antibody was used as a negative control. The mixture of CD70 and anti-CD70 antibody was blocked with an anti-Fc antibody and 100 μl/well CD70-myc-his+ antibody was added to the wells containing CD27-Fc-his. The mixture was incubated for 1 hour shaking at 37° C. To the mixture was added 100 μl/well of anti-myc-HRP (1:1000 diluted in 10% NHS+1% BSA/PBS) and incubated for 1 hour while shaking at 37° C. The signal was detected by adding 100 μl TMB substrate, incubated for 5-10 min at RT, then 75 μl 0.25 M H2SO4 was added and the results were read at A450 nm. The results are shown in FIG. 31. This data demonstrates that some anti-CD70 antibodies, including 2H5, 8B5, and 18E7, block binding of CD70 to CD27, while other antibodies do not affect the interaction between CD70 and CD27.

### Example 18

### Treatment of in vivo Tumor Xenograft Model Using Naked Anti-CD70 Antibodies

Mice implanted with a lymphoma tumor were treated in vivo with naked anti-CD70 antibodies to examine the in vivo effect of the antibodies on tumor growth.

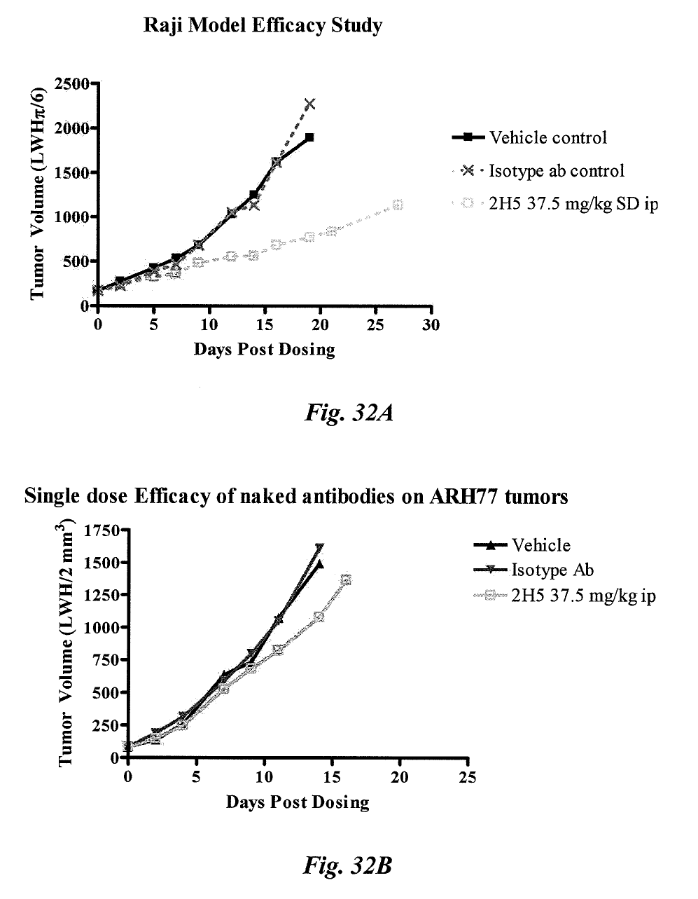

ARH-77 (human B lymphoblast leukemia; ATCC Accession No. CRL-1621) and Raji (human B lymphocyte Burkitt's lymphoma; ATCC Accession No. CCL-86) cells were expanded in vitro using standard laboratory procedures. Male Ncr athymic nude mice (Taconic, Hudson, N.Y.) between 6-8 weeks of age were implanted subcutaneously in the right flank with 5×10⁶ ARH-77 or Raji cells in 0.2 ml of PBS/Matrigel (1:1) per mouse. Mice were weighed and measured for tumors three dimensionally using an electronic caliper twice weekly after implantation. Tumor volumes were calculated as height×width×length/2. Mice with ARH-77 tumors averaging 80 mm³ or Raji tumors averaging 170 mm³ were randomized into treatment groups. The mice were dosed intraperitoneally with PBS vehicle, isotype control antibody or naked anti-CD70 HuMAb 2H5 on Day 0. Mice were euthanized when the tumors reached tumor end point (2000 mm³). The results are shown in FIGS. 32A (Raji tumors) and 32B (ARH-77 tumors). The naked anti-CD70 antibody 2H5 extended the mean time to reaching the tumor end point volume (2000 mm³) and slowed tumor growth progression. Thus, treatment with an anti-CD70 antibody alone has a direct in vivo inhibitory effect on tumor growth.

### Example 19

### Treatment of in vivo Lymphoma Tumor Xenograft Model Using Cytotoxin-Conjugated Anti-CD70 Antibodies

Mice implanted with a lymphoma tumor were treated in vivo with cytotoxin-conjugated anti-CD70 antibodies to examine the in vivo effect of the antibodies on tumor growth.

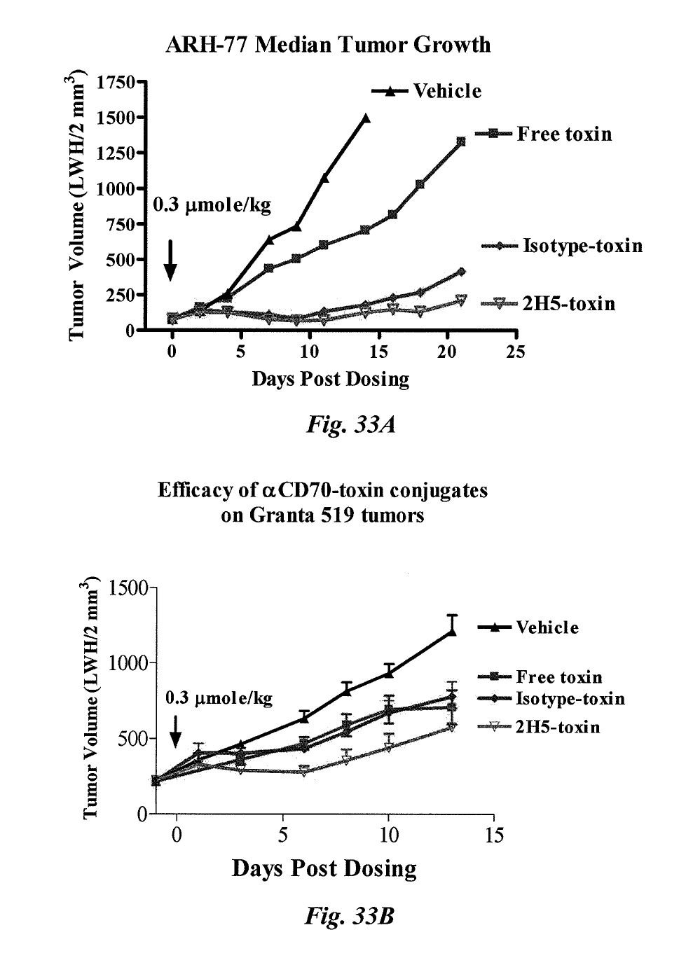

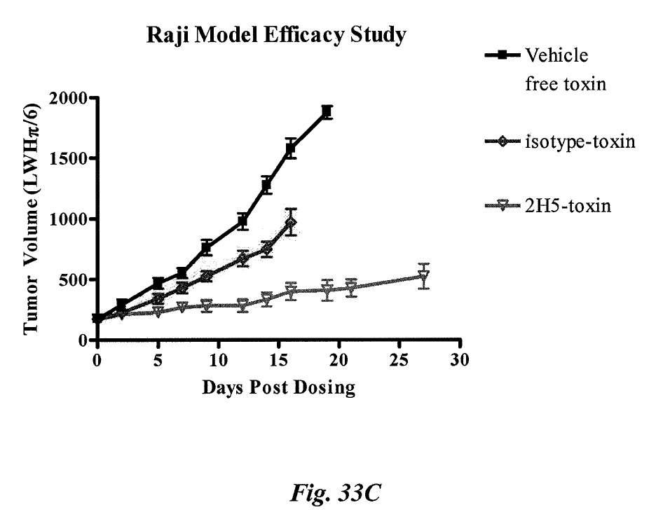

ARH-77 (human B lymphoblast leukemia; ATCC Accession No. CRL-1621), Granta 519 (DSMZ Accession No. 342) and Raji (human B lymphocyte Burkitt's lymphoma; ATCC Accession No. CCL-86) cells were expanded in vitro using standard laboratory procedures. Male Ncr athymic nude mice (Taconic, Hudson, N.Y.) between 6-8 weeks of age were implanted subcutaneously in the right flank with 5×10⁶ ARH-77, 10×10⁶ Granta 519 or 5×10⁶ Raji cells in 0.2 ml of PBS/Matrigel (1:1) per mouse. Mice were weighed and measured for tumors three dimensionally using an electronic caliper twice weekly after implantation. Tumor volumes were calculated as height×width×length/2. Mice with tumors averaging 80 mm³ (ARH-77), 220 mm³ (Granta 519), or 170 mm³ (Raji), were randomized into treatment groups. The mice were dosed intraperitoneally with PBS vehicle, cytotoxin-conjugated isotype control antibody or cytotoxin-conjugated anti-CD70 HuMAb 2H5 on Day 0. The conjugate used in this experiment was the free toxin released by cleavage of the linker in N1. Examples of cytotoxin compounds that may be conjugated to the antibodies of the current disclosure are described in U.S. Provisional Application Ser. No. 60/720,499, filed on Sep. 26, 2005 and PCT Publication No. WO 07/038658, filed on Sep. 26, 2006, the contents of which are hereby incorporated herein by reference. Mice were euthanized when the tumors reached tumor end point (2000 mm³). The results are shown in FIGS. 33A (ARH-77), 33B (Granta 519) and 33C (Raji tumors). The anti-CD70 antibody 2H5 conjugated to the cytotoxin extended the mean time to reaching the tumor end point volume (2000 mm³) and slowed tumor growth progression. Thus, treatment with an anti-CD70 antibody-cytotoxin conjugate has a direct in vivo inhibitory effect on lymphoma tumor growth.

### Example 20

### Cross-Reactivity of Anti-CD70 Antibody with Rhesus B Lymphoma Cells

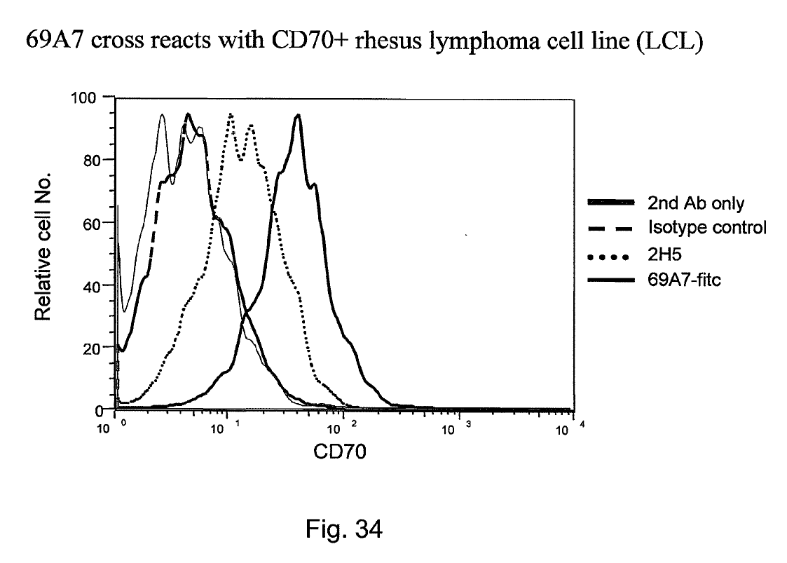

FACS analysis was also employed to access the ability of the anti-CD70 antibody 69A7 cross reacting with the monkey rhesus CD70+B lymphoma cell line, LCL8664 (ATCC#: CRL-1805). Binding of the HuMAb 69A7 anti-CD70 human monoclonal antibody was assessed by incubating 1×10⁵ cells with 69A7 at a concentration of 1 μg/ml. The cells were washed and binding was detected with a FITC-labeled anti-human IgG Ab. An isotype control antibody was used as a negative control. Flow cytometric analyses were performed using a FACSCalibur flow cytometry (Becton Dickinson, San Jose, Calif.). The results are shown in FIG. 34. The result demonstrated that the anti-CD70 antibody 69A7 cross-reacts with monkey CD70+B lymphoma cells.

### Example 21

### Internalization of Anti-CD70 Antibody Upon Binding to 786-O Renal Carcinoma Cells

The 786-O human renal cancer cell line was used to test the internalization of HuMab anti-CD70 antibodies 69A7 and 2H5 upon binding to the cells using immuno-fluorescence staining. 786-O cells (1×10⁴ cells per 100 μl per well in a 96-well plate) were harvested from a tissue culture flask by treatment with 0.25% Trypsin/EDTA, then incubated with each of the HuMab anti-CD70 antibodies at 5 μg/ml in FACS buffer (PBS+5% FBS, media) for 30 minutes on ice. A human IgG1 isotype control was used as a negative control. Following 2 washes with media, the cells were re-suspended in the media (100 μl per well) and then incubated with goat anti-human secondary antibody conjugated with PE (Jackson ImmunoResearch Lab) at 1:100 dilution on ice for 30 minutes. The cells were either immediately imaged for morphology and immunofluorescence intensity under a fluorescent microscope (Nikon) at 0 min or incubated at 37° C. for various times. Fluorescence was observed in the cells stained with HuMab anti-CD70 antibodies, but not in the control antibody. Similar results were also obtained with FITC-direct conjugated HuMab anti-CD70 antibodies in the assays. The results showed the appearance of the fluorescence on the cell surface membrane with both anti-CD70 HuMabs at 0 min. Following a 30 min incubation, the membrane fluorescence intensity significantly decreased while the internal fluorescence increased. At the 120 min timepoint, membrane fluorescence was not apparent, but instead appeared to be present in intracellular compartments. The data demonstrates that HuMab anti-CD70 antibodies can be specifically internalized upon binding to CD70-expressing endogenous tumor cells.

### Example 22

### HuMAb Anti-CD70 Blocks the Binding of a Known Mouse Anti-CD70 Antibody

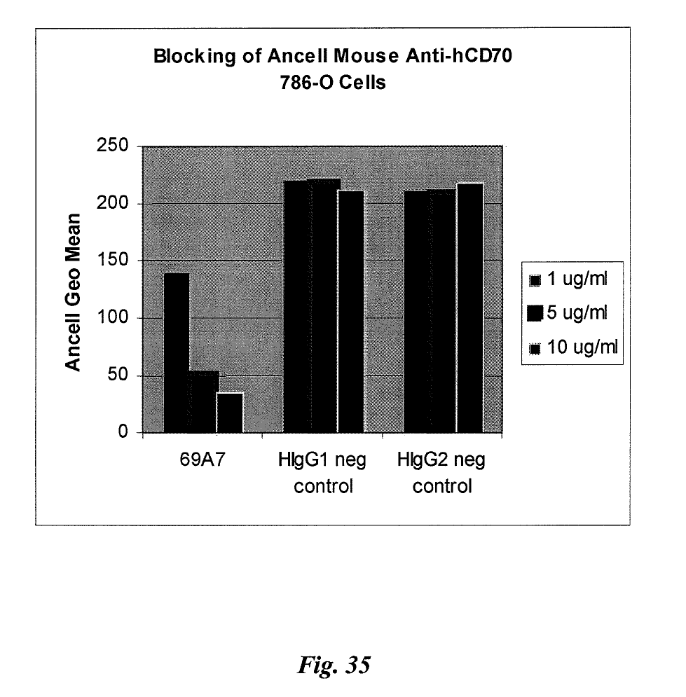

In this experiment, the HuMAb anti-CD70 antibody 69A7 was tested for its ability to block binding of a known mouse anti-CD70 antibody to CD70+renal carcinoma 786-O cells. 786-O cells were incubated with the mouse anti-CD70 antibody BU-69 (Ancell, Bayport, Minn.) at 1 μg/ml and the HuMAb 69A7 at 1, 5 or 10 μg/ml for 20 minutes on ice. IgG1 and IgG2 isotype control antibodies were used as negative controls. The cells were washed twice and binding was detected with a FITC-labeled anti-human IgG Ab. Flow cytometric analyses were performed using a FACSCalibur flow cytometry (Becton Dickinson, San Jose, Calif.). The results are shown in FIG. 35. The anti-CD70 HuMAb 69A7 blocks binding of a mouse anti-CD70 antibody in a concentration dependent manner.

### Example 23

### HuMAb Anti-CD70 Inhibits Inflammatory Response

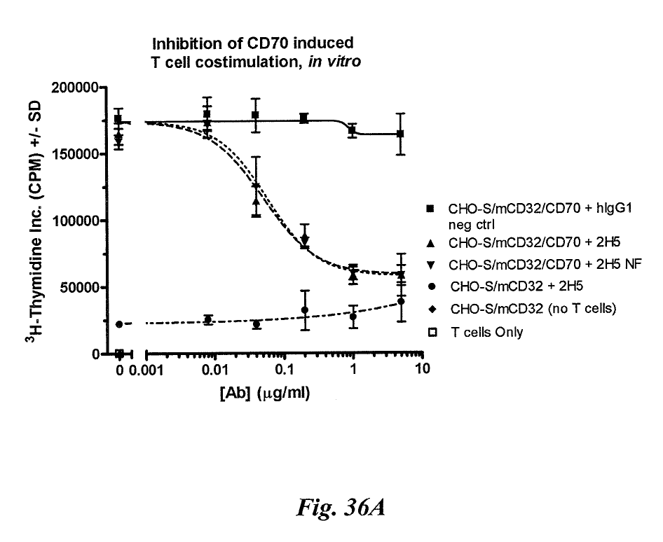

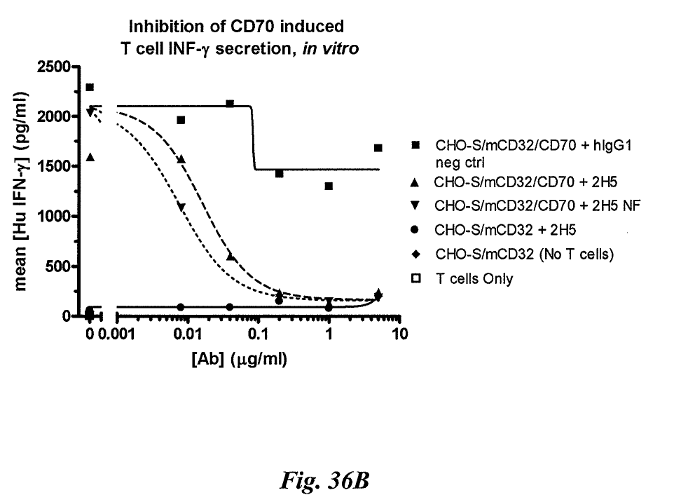

In this experiment, the HuMAb anti-CD70 antibody 2H5 was tested for inhibition of inflammatory responses. CHO-S cells stably transfected with mouse CD32 (CHO-S/mCD32 cells) were transiently transfected with a full length human CD70 construct (CHO-S/mCD32/CD70 cells). Surface expression was confirmed by flow cytometry using 2A5 and PE conjugated anti-human IgG secondary Ab (data not shown). RosetteSep® Human T Cell Enrichment Kit (Cat# 15061; StemCell Technologies Inc) purified human peripheral blood CD3⁺ T cells were stimulated in vitro at 1×10⁶/well with 1×10⁵ CHO-S/mCD32 or CHO-S/mCD32/CD70 cells/well, 1μg/ml anti-hCD3 (clone OKT3; BD Bioscience) and serial dilutions of either the HuMAb 2H5 or non-fucosylated 2H5 (2H5 NF) in triplicate wells of a 96 well plate. After 3 days supernatant aliquots were collected and interferon-gamma (INF-γ) secretion was measured by a quantitative ELISA kit (BD Biosciences). The plates were pulsed with 1 μCi/ml of ³H-thymidine, incubated for 8 hours, cells were harvested and ³H-thymidine incorporation was read on a Trilux® 1450 Microbeta Counter (Wallac, Inc.). An IgG1 isotype control antibody was used as a negative control. The results are shown in FIGS. 36A-B. Both 2H5 and 2H5 NF completely inhibited CD70 co-stimulated proliferation in a dose dependent manner (FIG. 36A). Data also show 2H5 inhibition is specific to CD70 costimulation as 2H5 had no effect on anti-CD3⁺ CHO-S/mCD32 mediated proliferation. Both 2H5 and 2H5 NF completely inhibited CD70 co-stimulated INF-γ secretion in a dose dependent manner as well (FIG. 36B). Data also show 2H5 inhibition is specific to CD70 costimulation as 2H5 had no effect on anti-CD3⁺ CHO-S/mCD32 mediated INF-γ secretion. Together data show 2H5 and 2H5 NF functionally block CD70 human T cell costimulation.

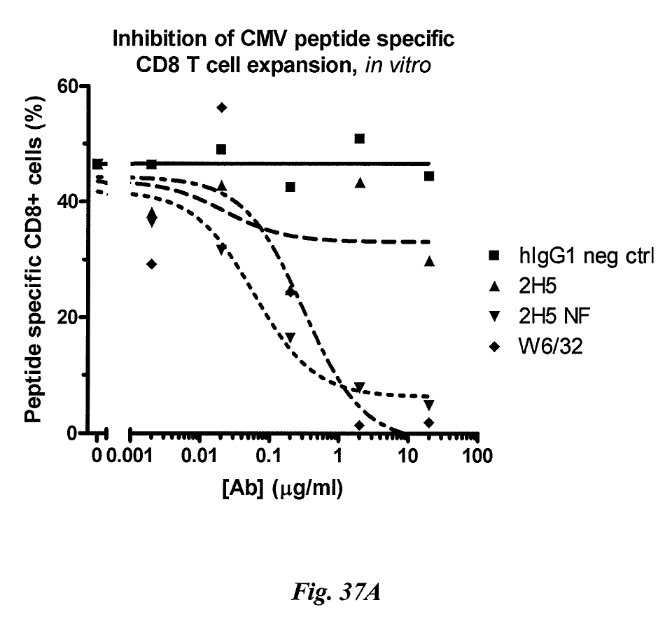

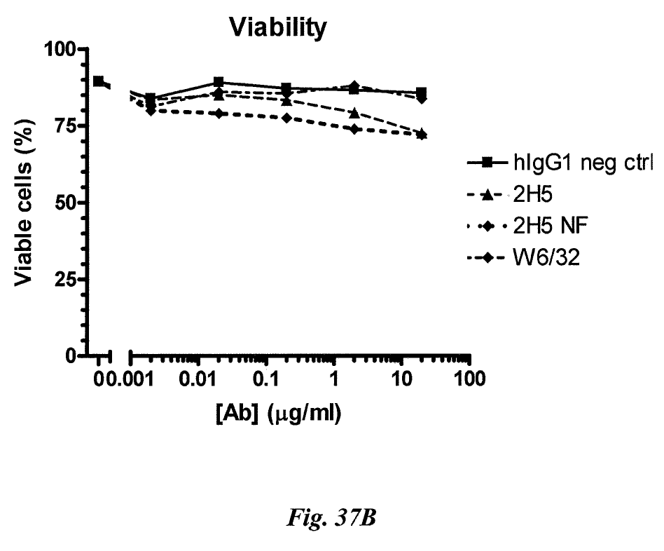

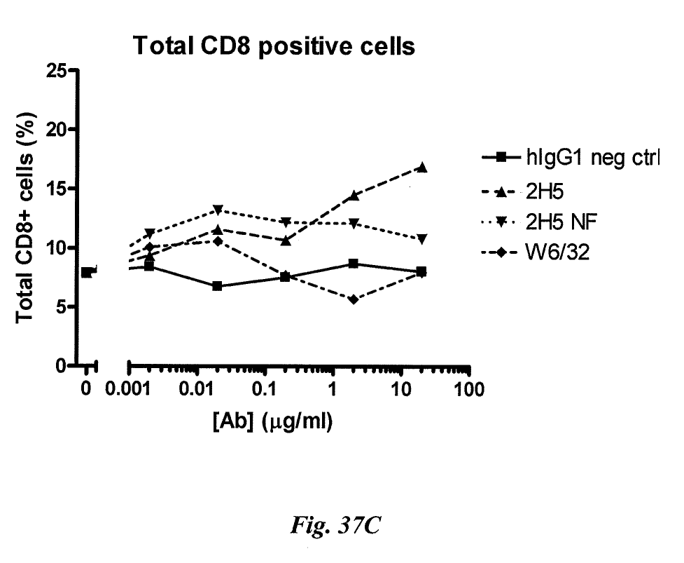

Human MHC class I haplotype B*3501+ peripheral blood mononuclear cells (PBMC) pre-screened for cytomegalovirus (CMV) specific T cell responses (Astarte, Inc) were cultured in the presence of 25 ng/ml of B*3501 binding CMV peptide IPSINVHHY (SEQ ID NO:90) (ProImmune, Oxford, UK) and serial dilutions of the HuMAb 2H5 for 11 days. Cultures were analyzed by flow cytometry for CD8+ T cells by PE conjugated anti-CD8 staining (clone RPA-T8, BD Biosciences), for peptide specific CD8+ T cells by APC labeled peptide-MHC Class I pentameric oligomer staining (F114-4B; ProImmunc) and for viability by lack of propidium iodide staining. An isotype control antibody was used as a negative control. The results are shown in FIG. 37A-C. 2H5 partially inhibited peptide specific CD8+ T cell expansion and 2H5 NF and positive control anti-MHC Class I Ab (clone W6/32; BD Bioscience) completely inhibited peptide specific. CD8+ T cell expansion (FIG. 37A). There was no significant reduction of total cell viability observed (FIG. 37B). There was no significant reduction of total CD8+ cell numbers was observed (FIG. 37C). Together, data show 2H5 and 2H5 NF effects were specific to peptide stimulated CD8+ T cells. Data is representative of one additional experiment performed with the same donor.

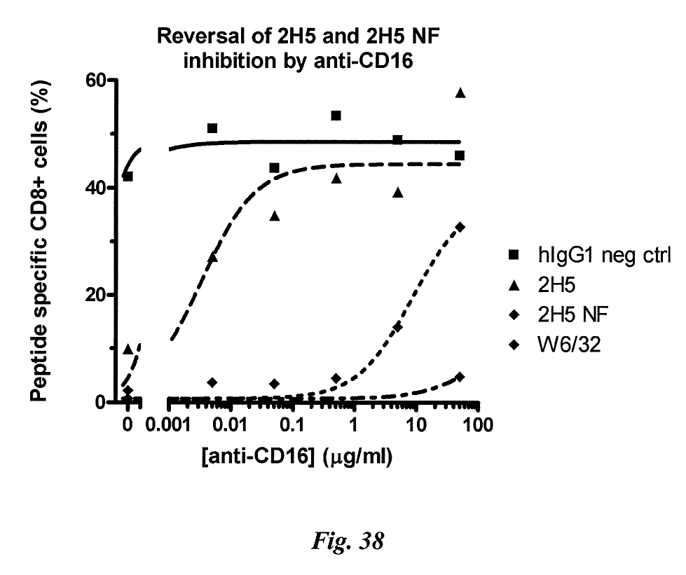

Human MHC class I haplotype B*3501+PBMC pre-screened for cytomegalovirus (CMV) specific T cell responses (Astarte, Inc) were cultured in presence of 25 ng/ml of B*3501 binding CMV peptide IPSINVHHY (ProImmune) (SEQ ID NO:90) and 20 μgs/ml of the HuMAb 2H5 in the presence or absence of serial dilutions of an anti-human CD16 (FcRγIII) functional blocking Ab (clone 3G8; BD Biosciences) for 11 days and were then analyzed by flow cytometry for peptide specific CD8+ cell numbers as described above. The results are shown in FIG. 38. Dose dependent reversal of 2H5 and 2H5 NF mediated inhibition of peptide specific CD8+ T cell expansion by anti-CD 16 shows 2H5 and 2H5 NF inhibition is mediated through interaction of 2H5 and 2H5 NF with CD16+ effector cells. Approximately 1000-fold more 3G8 was required to reverse 2H5 NF mediated inhibition compared to 2H5. There was no inhibition of peptide specific CD8+ T cell expansion by the negative isotype control irrespective of 3G8 concentration and little to no effect of 3G8 on inhibition of peptide specific CD8+ T cell expansion by a functional blocking positive control W6/32.

### Example 24

### Treatment of in vivo Renal Carcinoma Tumor Xenograft Model Using Cytotoxin-Conjugated Anti-CD70 Antibodies

Mice implanted with a renal carcinoma tumor were treated in vivo with cytotoxin conjugated anti-CD70 antibodies to examine the in vivo effect of the antibodies on tumor growth. In this example, anti-CD70 antibody 2H5 was conjugated to N2. N2 is a prodrug requiring esterase activation

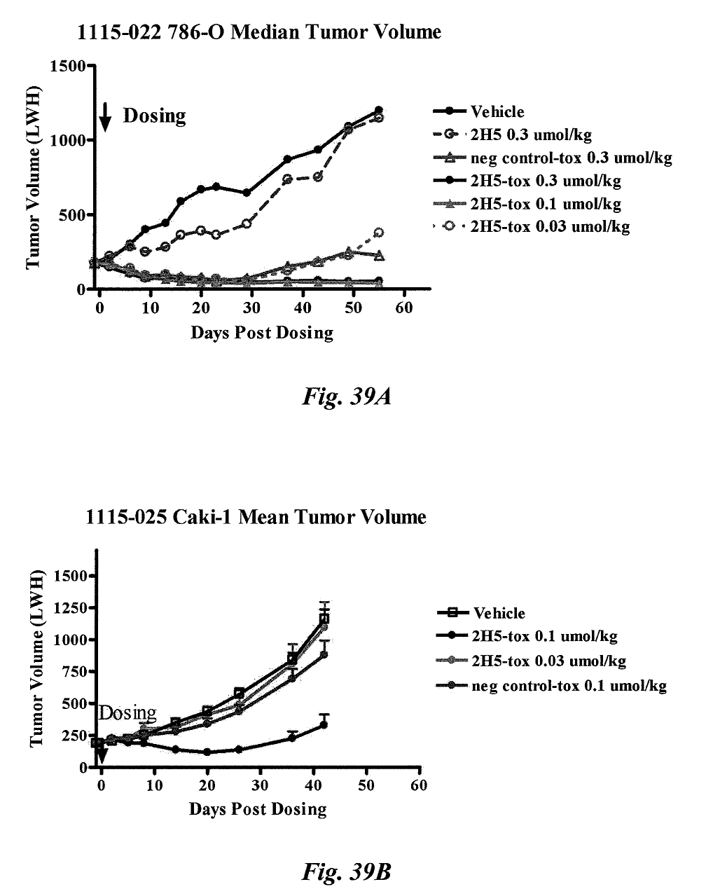

786-O (ATCC Accession No. CRL-1932) and Caki-1 (ATCC Accession No. HTB-46) cells were expanded in vitro using standard laboratory procedures. Male CB17.SCID mice (Taconic, Hudson, N.Y.) between 6-8 weeks of age were implanted subcutaneously in the right flank with 2.5 million 786-O or Caki-1 cells in 0.2 ml of PBS/Matrigel (1:1) per mouse. Mice were weighed and measured for tumors three dimensionally using an electronic caliper twice weekly after implantation. Tumor volumes were calculated as height×width×length. Mice with tumors averaging 200 mm³ were randomized into treatment groups. The mice were dosed intraperitoneally with PBS vehicle, cytotoxin-conjugated isotype control antibody or cytotoxin-conjugated anti-CD70 HuMAb 2H5 on Day 0. Examples of cytotoxin compounds that may be conjugated to the antibodies of the current disclosure are described in U.S. Provisional Application Ser. No. 60/720,499, filed on Sep. 26, 2005 and PCT Publication No. WO 07/038658, filed on Sep. 26, 2006, the contents of which are hereby incorporated herein by reference. Mice were euthanized when the tumors reached a tumor volume end point (2000 mm³). The results are shown in FIG. 39A (786-O) and FIG. 39B (Caki-1). The anti-CD70 antibody 2H5 conjugated to N2 extended the mean time to reaching the tumor end point volume (2000 mm³) and slowed tumor growth progression. There was a less than 10% body weight change in the treated animals. Thus, treatment with an anti-CD70 antibody-cytotoxin conjugate has a direct in vivo inhibitory effect on lymphoma tumor growth.

### Example 25

### Treatment of in vivo Renal Cell Carcinoma Xenograft Model Using Anti-CD70 Immunoconjugates

Mice implanted with a renal carcinoma tumor were treated in vivo with cytotoxin-conjugated anti-CD70 antibodies to examine the in vivo effect of the antibodies on tumor growth.

Immunoconjugates of complex N1 or N2 linked to thiolated anti-CD70 2H5 antibody were prepared as described previously (see, e.g., U.S. Pat. Appl. Pub. Nos. 2006/0024317; and PCT Appl. No. PCT/US2006/37793). NOD-SCID mice were implanted subcutaneously with 2.5×10⁶ 786-O cells. Tumor formation was monitored until the mean tumor volume was measured (using precision calipers) to be about 80 mm³. Groups of eight tumor-bearing mice were treated with a single dose of one of: (a) a vehicle control, (b) immunoconjugate anti-CD70-N1, or (c) immunoconjugate anti-CD70O-N2 Immunoconjugates anti-CD70-N1 and anti-CD70-N2 were administered to the mice intraperitoneally (i.p.) at a dose of 0.3 μmol/kg of N1 equivalents and 0.1 μmol/kg of N2 equivalents, respectively. The anti-CD70-N1 group received a second treatment at the same dose on day 21 after the first dose. Tumor growth was monitored by measurement with precision calipers over the 62 day course of the experiment.

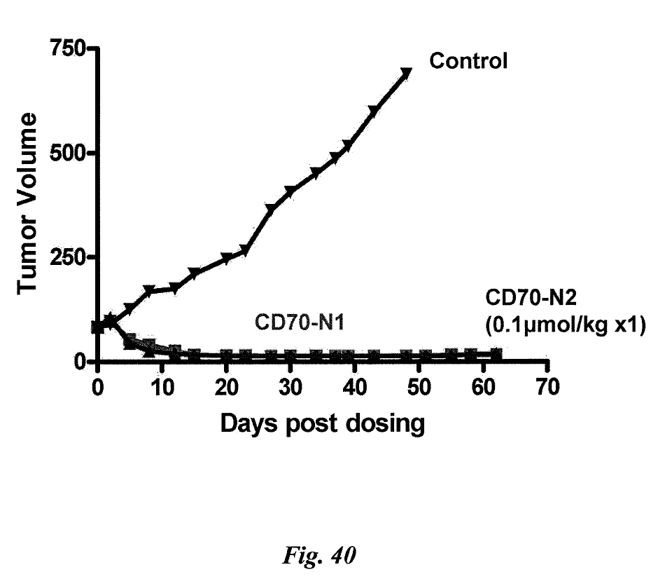

As is evident in FIG. 40, a single dose treatment with immunoconjugate anti-CD70-N1 or anti-CD70-N2 resulted in tumor-free mice within 15 days (and remained tumor-free up to 62 days) as compared to the mice having substantial tumor growth when treated with only the vehicle control.

### Example 26

### Treatment of in vivo Renal Cell Carcinoma Xenograft Model Using Immunoconjugate Anti-CD70-N2

Mice implanted with a renal carcinoma tumor were treated in vivo with cytotoxin-conjugated anti-CD70 antibodies to examine the in vivo effect of the antibodies on tumor growth.

An immunoconjugate of complex N2 linked to thiolated anti-CD70 2H5 antibody was prepared as described in Example 25. SCID mice were implanted subcutaneously with 2.5×10⁶ 786-O cells in 0.1 ml PBS and 0.1 ml matrigel per mouse. Tumor formation was monitored until the mean tumor volume was measured (using precision calipers) to be about 105 mm³. Groups of eight tumor-bearing mice were treated with a single dose of one of: (a) a vehicle control, (b) isotype control, (c) anti-CD70 antibody 2H5 alone, or (d) immunoconjugate anti-CD70-N2. Immunoconjugates anti-CD70-N2 and isotype control-N2 (IgG-N2) were administered to the mice i.p. at a dose of 0.1 μmol/kg of N2 equivalents. Anti-CD70 antibody was administered at 10 mg/kg (i.e., the equivalent protein dose to the N2 equivalents used for the immunoconjugate CD70-N2). Tumor growth was monitored by measurement with precision calipers over the 62 day course of the experiment.

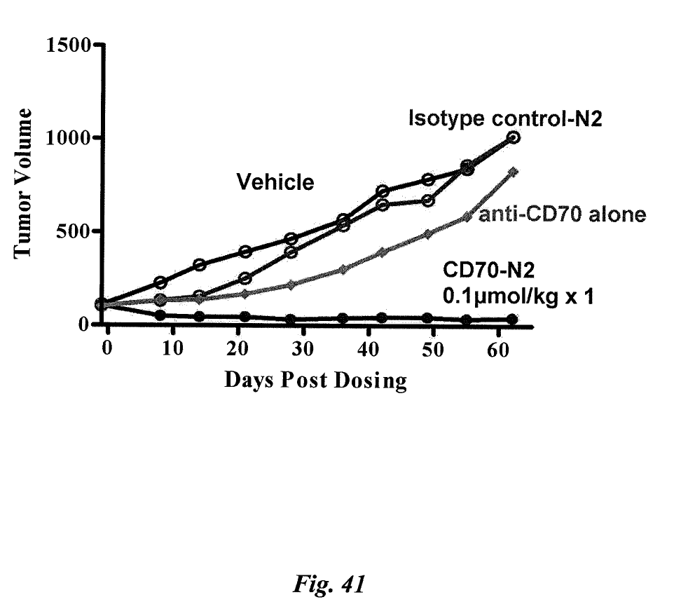

As is evident in FIG. 41, a single, low dose treatment with immunoconjugate anti-CD70-N2 resulted in mice with minimally detectable tumors within 10 days (and remained that way for up to 62 days) as compared to the mice having substantial tumor growth when treated with only the controls or anti-CD70 antibody alone.

### Example 27

### Dose Response of in vivo Renal Cell Carcinoma Xenograft to Immunoconjugate Anti-CD70-N2

Mice implanted with a renal carcinoma tumor were treated in vivo with cytotoxin-conjugated anti-CD70 antibodies to examine the in vivo effect of the antibodies on tumor growth.

An immunoconjugate of complex N2 linked to thiolated anti-CD70 2H5 antibody was prepared as described in Example 25. SCID mice were implanted subcutaneously with 2.5×10⁶ 786-O cells in 0.1 ml PBS and 0.1 ml matrigel per mouse. Tumor formation was monitored until the mean tumor volume was measured (using precision calipers) to be about 280 mm³. Groups of eight tumor-bearing mice were treated with either (a) a vehicle control or (b) immunoconjugate anti-CD70-N2. Immunoconjugate anti-CD70-N2 was administered to each group of mice i.p. at one of the following doses: 0.03 μmol/kg, 0.01 μmol/kg, or 0.005 μmol/kg of N2 equivalents. Tumor growth was monitored by measurement with precision calipers over the course of the experiment.

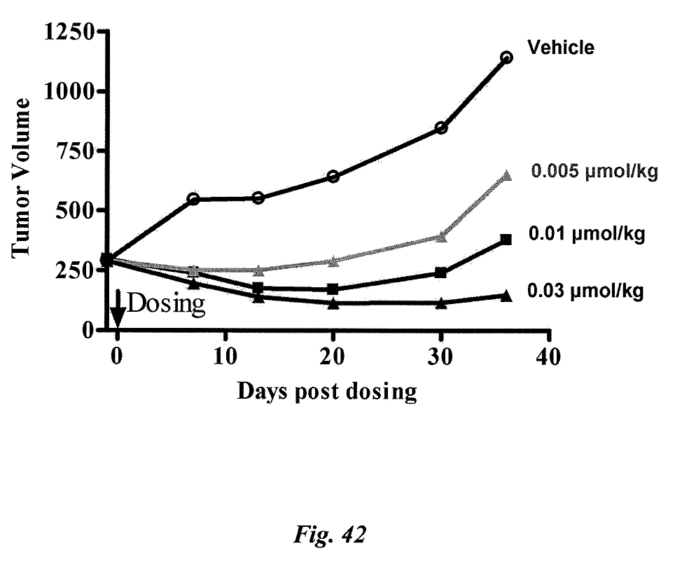

As is evident in FIG. 42, a surprisingly low dose of immunoconjugate anti-CD70-N2 resulted in tumor volume being reduced, and the tumor volume reduction occurred in a dose-dependent manner.

### Example 28

### Effectiveness of Immunoconjugate Anti-CD70-N2 in vivo on Another Renal Cell Carcinoma Xenograft Model

Mice implanted with a renal carcinoma tumor were treated in vivo with cytotoxin-conjugated anti-CD70 antibodies to examine the in vivo effect of the antibodies on tumor growth.

An immunoconjugate of complex N2 linked to thiolated anti-CD70 21-15 antibody was prepared as described in Example 25. SCID mice were implanted subcutaneously with 2.5×10⁶ Caki-1 cells in 0.1 ml PBS and 0.1 ml matrigel per mouse. Tumor formation was monitored until the mean tumor volume was measured (using precision calipers) to be about 105 mm³. Groups of eight tumor-bearing mice were treated with a single dose of one of: (a) a vehicle control, (b) isotype control, (c) anti-CD70 antibody 2H5 alone, or (d) immunoconjugate anti-CD70-N2. Immunoconjugates anti-CD70-N2 and isotype control-N2 were administered to the mice i.p. at a dose of 0.3 μmol/kg of N2 equivalents. Anti-CD70 antibody was administered at 11.5 mg/kg (i.e., the equivalent protein dose to the N2 equivalents used for the immunoconjugate CD70-N2). Tumor growth was monitored by measurement with precision calipers over the 62 day course of the experiment.

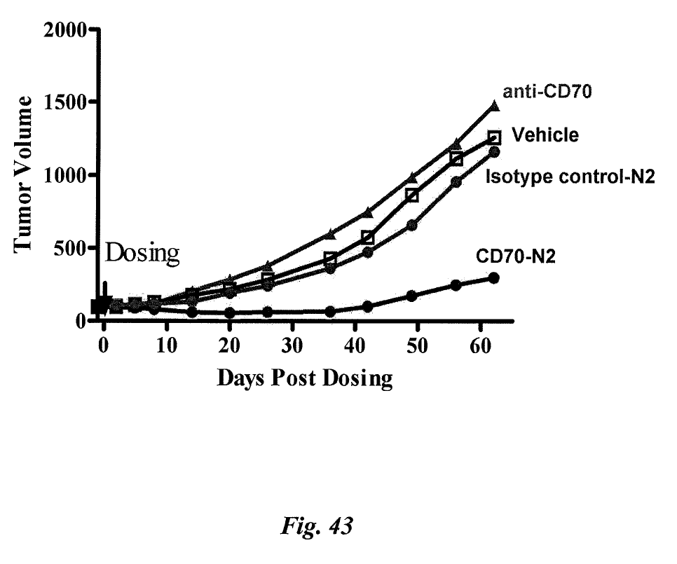

As is evident in FIG. 43, a single dose treatment with immunoconjugate anti-CD70-N2 resulted in mice with minimally detectable tumors for up to about 40 days as compared to the mice having substantial tumor growth when treated with only the controls or anti-CD70 antibody alone. Thus, anti-CD70 immunoconjugates are effective against multiple renal cancer models.

### Example 29

### Effectiveness of Immunoconjugate Anti-CD70-N2 in vivo in Lymphoma Model

Mice implanted with a lymphoma tumor were treated in vivo with cytotoxin-conjugated anti-CD70 antibodies to examine the in vivo effect of the antibodies on tumor growth.

An immunoconjugate of complex N2 linked to thiolated anti-CD70 2H5 antibody was prepared as described in Example 25. SCID mice were implanted subcutaneously with 1.0×10⁷ Raji cells in 0.1 ml PBS and 0.1 ml matrigel per mouse. Tumor formation was monitored until the mean tumor volume was measured (using precision calipers) to be about 50 mm³. Groups of eight tumor-bearing mice were treated with a single dose of one of: (a) a vehicle control, (b) isotype control, or (c) immunoconjugate anti-CD70-N2. Immunoconjugate anti-CD70-N2 was administered to the mice i.p. at a dose of 0.3 μmol/kg of N2 equivalents. Tumor growth was monitored by measurement with precision calipers over the 60 day course of the experiment.

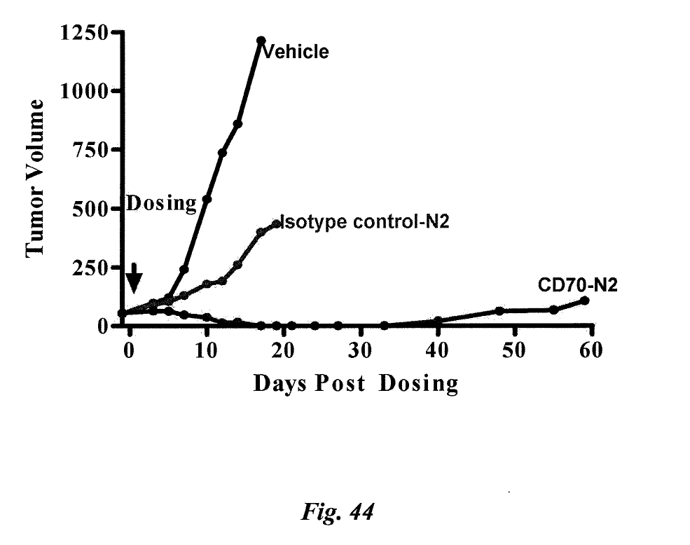

As is evident in FIG. 44, a single dose treatment with immunoconjugate anti-CD70-N2 resulted in mice with minimally detectable tumors for up to about 40 days as compared to the mice having substantial tumor growth when treated with only the controls or anti-CD70 antibody alone. Thus, anti-CD70 immunoconjugates are also effective against lymphoma.

### Example 30

### Safety Study of Immunoconjugate Anti-CD70-N2

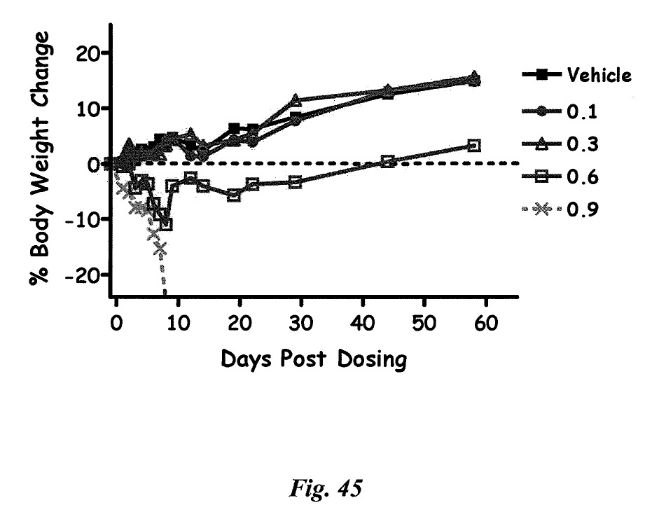

BALB/c mice were treated with immunoconjugate anti-CD70-N2 i.p. at one of the following doses: 0.1 μmol/kg, 0.3 μmol/kg, 0.6 μmol/kg, or 0.9 μmol/kg of N2 equivalents. The weight of the mice was measured on a daily basis for the first 12 days and periodically thereafter up to 60 days post dosing. Mice were euthanized when body weight loss exceeded 20% of the starting body weight. Data plotted in FIG. 45 is the mean body weight for each group.

As is evident in FIG. 45, the anti-CD70-N2 immunoconjugate was well tolerated and safe when administered at a dose below 0.9 μmol/kg of N2 equivalents. Thus, the doses at which immunoconjugate anti-CD70-N2 has shown efficacy (ranging from about 0.005-0.3 μmol/kg of N2 equivalents) will have a good safety profile.

### Example 31

### Further Safety Study of Immunoconjugate Anti-CD70-N2

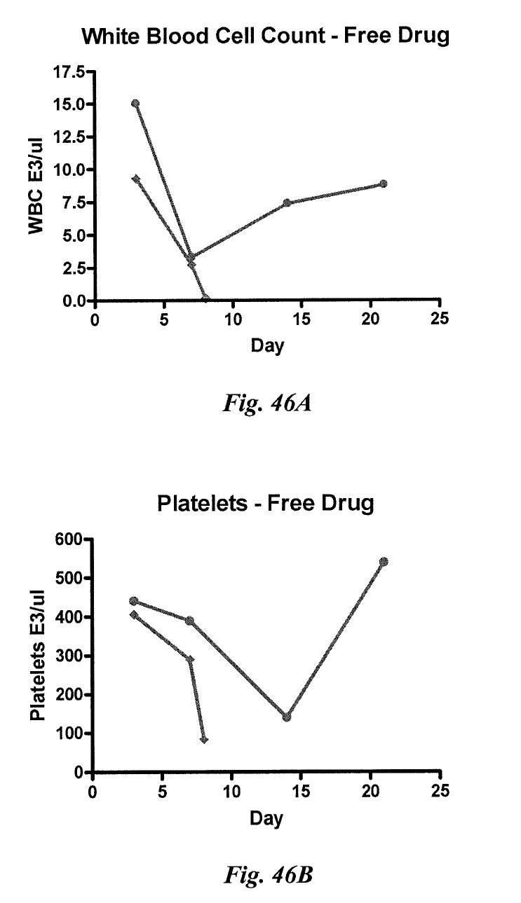

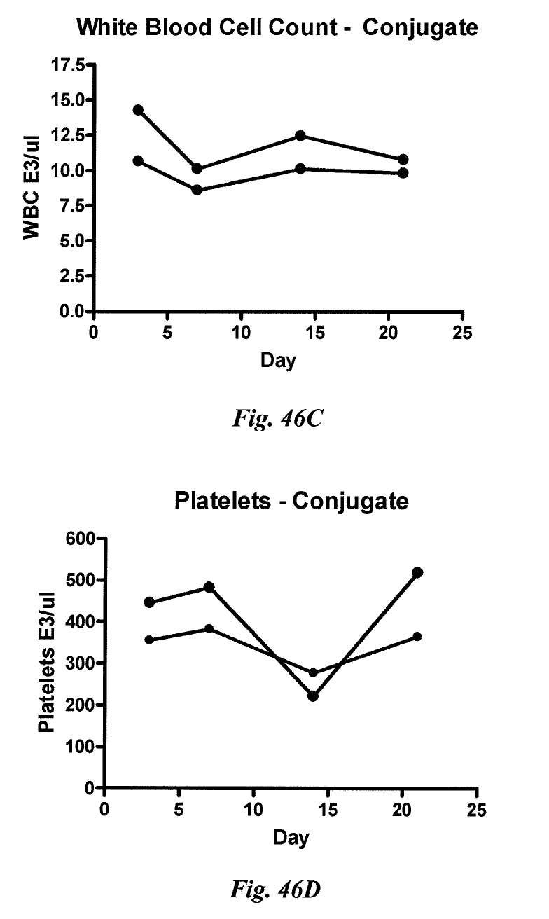

A further safety study of immunoconjugate anti-CD70-N2 was carried out in male beagles. The immunoconjugate was compared to drug alone. Immunoconjugate anti-CD70-N2 at 0.18 μmol/kg of N2 equivalents and N2 drug alone (without the linker in the N2 structure) at 0.15 μmol/kg were dosed intravenously in two beagle dogs each. The dogs were monitored hourly for 4 hours post dosing, and clinical observation was done twice daily for 28 days. Body weights were measured daily until 8 days post dosing and weekly afterwards. Standard hematology, coagulation and clinical chemistry were performed twice during the predose phase and on days 3, 7, 14 and 28 post-dosing. The results are shown in FIG. 46A-D. One dog in the free drug group was euthanized at day 8 post-dosing due to clinical signs of toxicity. As shown in FIG. 46A-D, the anti-CD70-N2 immunoconjugate was well tolerated by the treated dogs.

### Example 32

### Anti-CD70 Antibody Mediated ADCC of Activated Human B Cells

In this study, a HuMAb anti-CD70 antibody and the nonfucosylated form were tested for their ability to mediate ADCC effects on human B cells. Frozen human spleen cells were thawed and B cells were negatively purified by magnetic beads. Purified B cells were cultured at 2×10⁶/ml in RPMI+10% FBS supplemented with NEAA, sodium pyruvate, β-ME and penicillin/streptomycin. B cells were activated by 10 μg/ml of LPS and 5 μg/ml anti-CD40 for 3 days. The cells were harvested, washed and an aliquot was stained with biotin conjugated nonfucosylated 2H5 (2H5 NF-bio)+streptavidin-APC. Human peripheral blood mononuclear effector cells were purified from heparinized whole blood by standard Ficoll-Paque separation and cultured overnight in of 50 U/ml IL-2. The activated B cells were labeled with 100 μCi of Na₂ ⁵¹CrO₄ (Perkin Elmer, Wellesley, Mass.) per 1×10⁶ cells for 1 hour. Effector cells were added to labeled target cells at a ratio of 1:100 in the presence of serial dilutions of 2H5 and 2H5 NF (non-fucosylated). In addition, the test articles were assayed at 10 μg/ml in the presence of 20 μg/ml murine anti-CD 16 antibody 3G8 or mouse isotype control antibody. Following 4 incubation for 4 hours at 37° C., cells were centrifuged and the supernate was read on the Cobra II auto-gamma counter (Perkin Elmer) with a reading window of 240-400 KeV. The percent specific lysis was calculated as: (experimental release−spontaneous release)/(maximal release−spontaneous release)×100 where: (i) target cells with no effector cells and no antibody control for spontaneous release and (ii) target and effector cells in presence of 3% Lysol detergent control for maximal release. Percent specific lysis was plotted against antibody concentration and the data was analyzed by non-linear regression, sigmoidal dose response (variable slope) using GraphPad Prism™ 3.0 software (San Diego, Calif.).

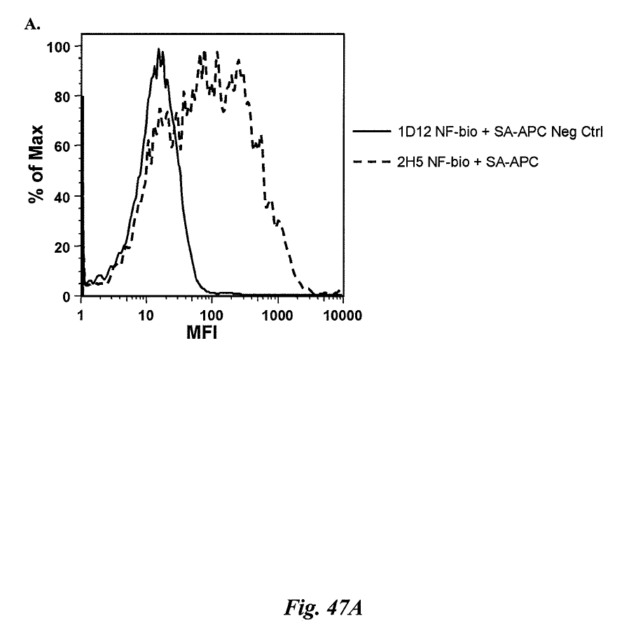

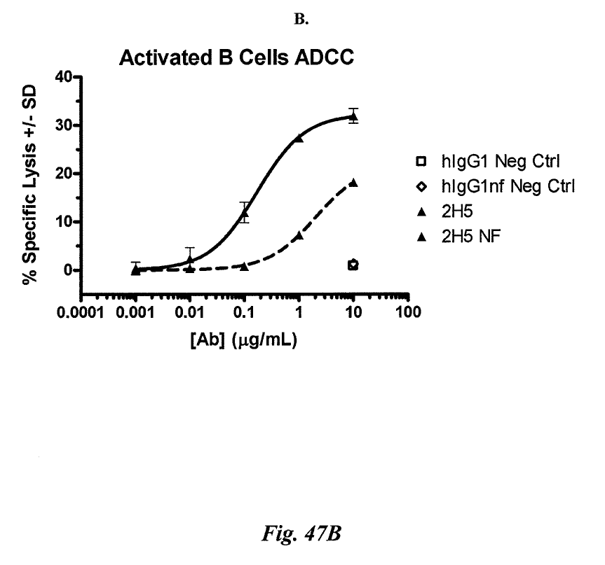

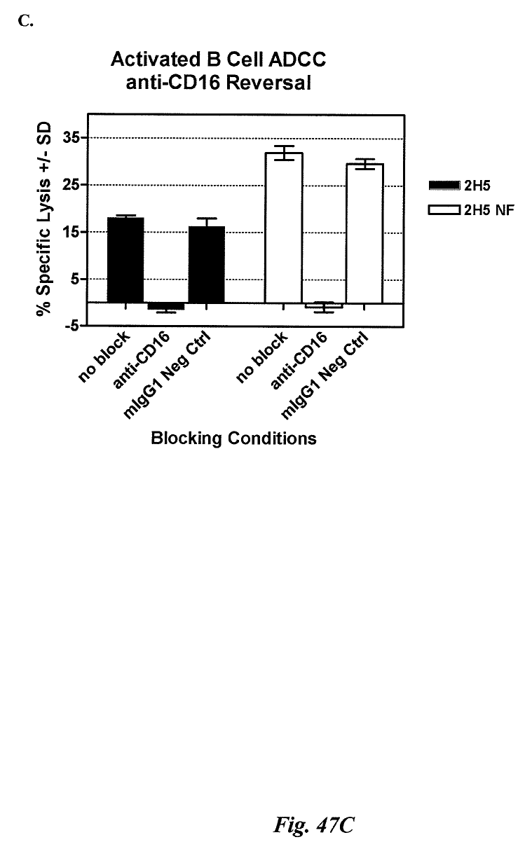

The data is shown in FIG. 47. 2H5 NF binds to ˜60% of the activated B cells. Both 2H5 NF and 2H5 induced lysis of activated human B cells, but 2H5 NF was approximately 10-fold more potent and more efficacious than 2H5. The anti-CD 16 reversal of Ab induced lysis confirms that the mechanism of action of the Ab mediated lysis was NK cell mediated ADCC. Thus, both 2H5 and 2H5 NF mediate ADCC of human activated B cells.

### Example 33

### Anti-CD70 Antibody Inhibition of CMV Ag Stimulated Human CD4+ T Cell Expansion, in vitro

This study demonstrates the capability of anti-CD70 antibodies to mediate lysis of Ag activated, CD70+ human T cells (cells which are key contributors to the inflammatory process in autoimmune and inflammatory disease) via ADCC by effector cells naturally present in stimulated human PBMC cultures.

CMV positive pre-screened donors were cultured in AIM-V media supplemented with 10% heat-inactivated FCS at 1×10⁶ cells/ml on 24-well culture plates and stimulated with 5.0 μg/ml of CMV lysate in the presence of 2 μg/ml of biotinylated 2H5, 2H5 NF or hIgG1nf control Abs. Cells were harvested on day 9 and the number of viable cells/ml in each culture was determined by counting an aliquot using a hemocytometer and trypan blue exclusion. The cells were washed in staining buffer and blocked with 5% human serum. Biotinylated 2H5, 2H5 NF, or hIgG1nf were added to an equal volume of cells at 20 μg/ml final concentration. Cells were incubated for 30 minutes, washed and stained with anti-CD4-FITC and PE-conjugated streptavidin. Cells were again incubated for 30 minutes, washed twice and then fixed and permeabilized using BD Cytofix/Cytoperm kit. The cells were washed twice in perm/wash buffer and intracellular stained with anti-INFγ-APC (BD Clone B27). Cells were incubated for 30 minutes, washed and resuspended in staining buffer. Cells were analyzed by flow cytometry for CD70 surface and INFγ intracellular expression by gating on live CD4+ cells. The number of CD4+/CD70+ and CD4+/INFγ+ cells/ml in each condition were calculated by multiplying the percent CD70+ or INFγ+ cells in the CD4 gate by the percent of total CD4+ cells times the total number of viable cells/ml ((%CD70+ or INFγ+)×(%CD4+)×(total viable cells/ml)).

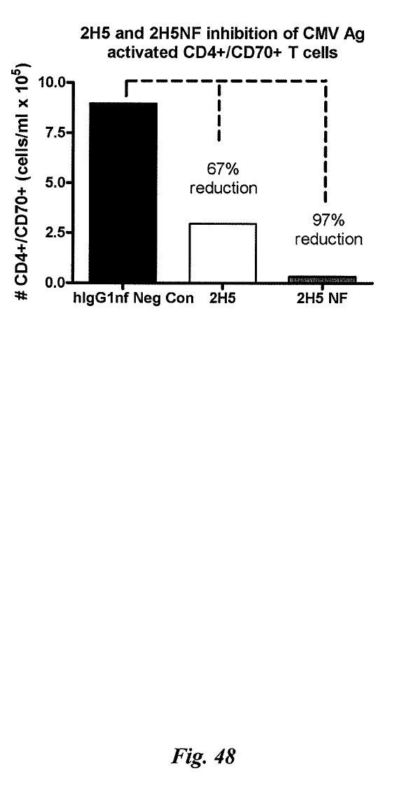

The data is shown in FIG. 48. 2H5 and 2H5 NF at 2 μg/ml depleted 67% and 97% of CMV activated CD70+/CD4+ cells on day 9, respectively. Both antibodies were effective, but 2H5 NF was more potent than 2H5 for mediating ADCC of Ag activated CD4+/CD70+ T cells by CD 16+ effector cells that are present in normal human blood.

### Example 34

### Relative Binding Characteristic of Human CD70 Antibodies 1F4, 1F4 NF and 2H5 NF Binding to CD70+ Renal Carcinoma Cell Line 786-0

This study investigated the binding characteristics of anti-CD70 antibodies to natively expressing CD70+ human cancer cell line 786-0 cells. Human renal cell adenocarcinoma cell line 786-0 were grown to confluence, harvested with trypsin, washed in staining buffer and incubated with 1F4, 1F4 NF, 2H5 NF, hIgG1-NF or hIgG4 at final concentrations of 30, 10, 3, 1, 0.4, 0.1, 0.04 and 0.01 ug/ml. The cells were incubated for 30 minutes on ice, washed twice in staining buffer and stained with Goat F(ab)′2-anti-human-IgG(Fc)-PE conjugate for 30 minutes. The cells were washed and resuspended in staining buffer for analysis by flow cytometry.

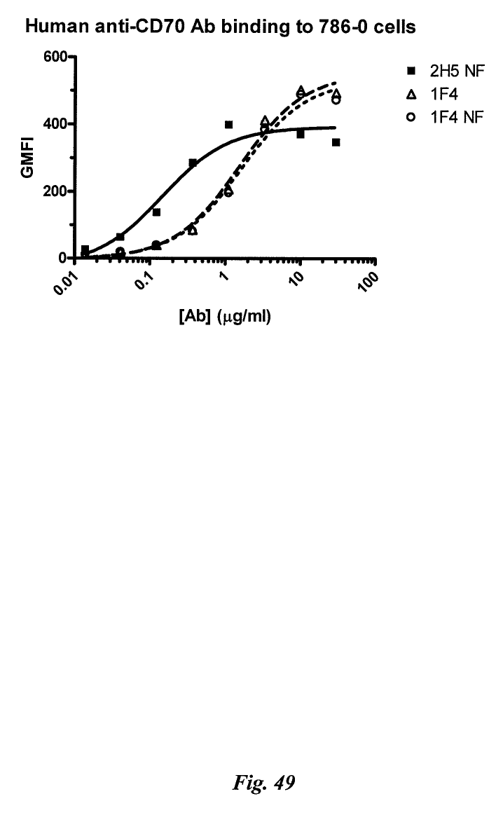

The data is shown in FIG. 49. 2H5 NF binds at lower concentration than 1F4 and 1F4 NF. 2H5 NF has superior binding affinity for native cell surface expressed CD70 than 1F4 and 1F4 NF. 1F4 and 1F4 NF bind equally well to the 786-0 cell showing no affect of the specific binding characteristics due to the NF isotype.

### Example 35

### Relative Capability of 1F4 and 1F4 NF to Mediate ADCC on the CD70+Lymphoma Cell Line ARH77

In this study, fucosylated and non-fucosylated (nf) anti-CD70 antibodies were tested for their relative capability to mediate ADCC on the CD70+ lymphoma cell line ARH77. Human peripheral blood mononuclear effector cells were purified from heparinized whole blood by standard Ficoll-Paque separation and cultured overnight in the presence of 50 U/ml IL-2. The ARH77 cells were labeled with 100 μCi of Na₂ ⁵¹CrO₄ (Perkin Elmer, Wellesley, Mass.) per 1×10⁶ cells for 1 hour. Effector cells were added to labeled target cells at a ratio of 1:100 in the presence of serial dilutions of 2H5 and 2H5nf. In addition, the test articles were assayed at 5 μg/ml. Following incubation for 4 hours at 37° C., cells were centrifuged and the supernate was read on the Cobra II auto-gamma counter (Perkin Elmer) with a reading window of 240-400 KeV. The percent specific lysis was calculated as: (experimental release−spontaneous release)/(maximal release−spontaneous release)×100 where: (i) target cells with no effector cells and no antibody control for spontaneous release and (ii) target and effector cells in presence of 3% Lysol detergent control for maximal release.

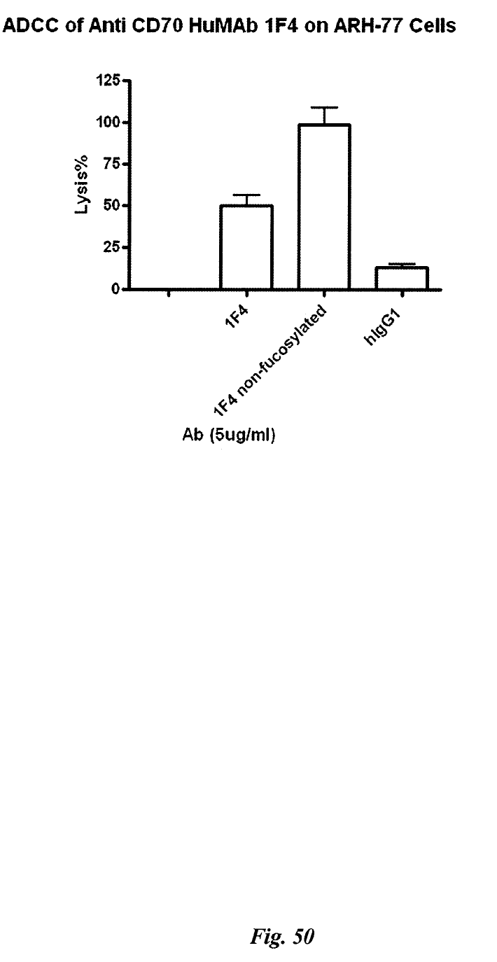

The data is shown in FIG. 50. Both 1F4 and 1F4 NF mediate ADCC on CD70+ ARH77 cells, and 1F4 NF is a more potent mediator of ADCC than 1F4.

### Example 36

### Tumor Growth Inhibition in vivo by Anti-CD70-Cytotoxin E

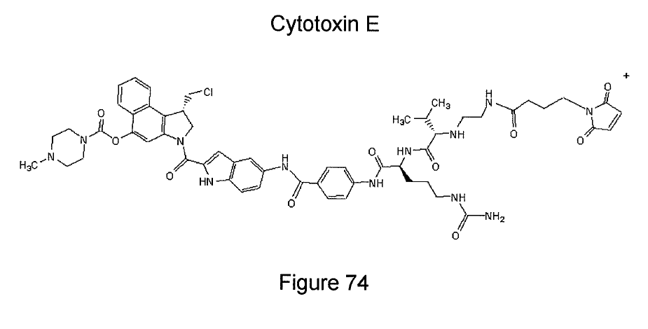

In order to demonstrate the broad utility of anti-CD70-cytotoxin E conjugate as a targeted therapeutic against different tumor cells, three renal cell cancer xenograft models and two lymphoma models in SCID mice were used to test the efficacy of the anti-CD70-cytotoxin E conjugate in vivo. A cytotoxin conjugate of the CD70 antibody 2H5 is referred to herein as anti-CD70-cytotoxin E, which is comprised of a recombinant 2H5 anti-CD70 antibody linked to cytotoxin E (FIG. 74), described further in U.S. Application Ser. No. 60/882,461, filed Dec. 28, 2006, the entire content of which is specifically incorporated herein by reference. Cytotoxin E is in prodrug form, and requires not only release from the antibody for activity but also cleavage of a 4′ carbamate group to release the active moiety.

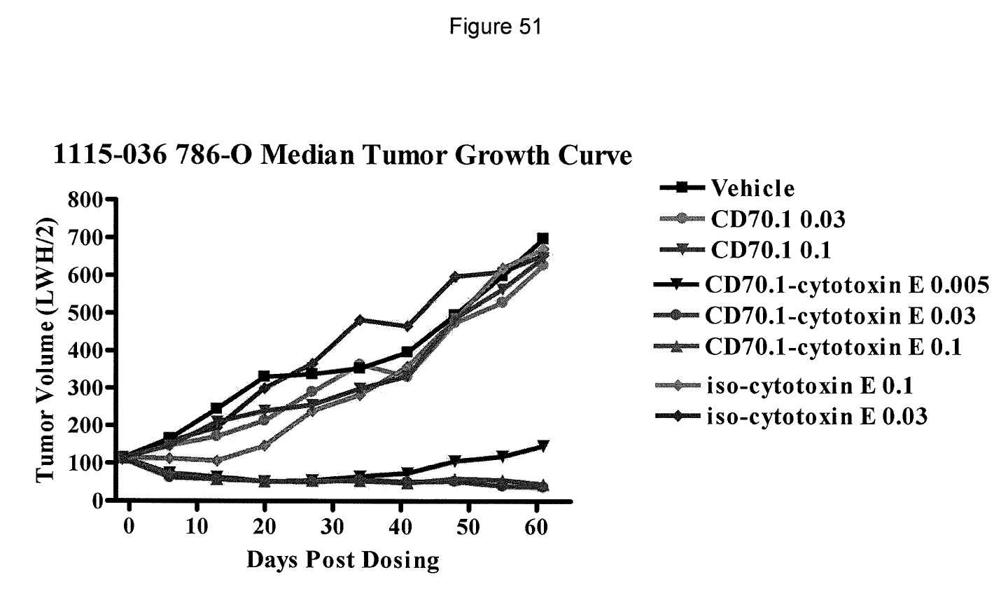

To demonstrate the activity of anti-CD70-cytotoxin E on 786-O cell xenografts, 2.5 million 786-O cells in 0.1 ml PBS and 0.1 ml Matrigel™ per mouse were implanted subcutaneously into SCID mice, and when tumors reached an average size of 110 mm³, groups of 8 mice were treated by ip injection of a single dose of either anti-CD70-cytotoxin E at 0.005, 0.03 or 0.1 μmol/kg body weight. In addition, control groups were injected with either vehicle alone, anti-CD70 antibody alone (at doses equivalent to those used for anti-CD70-cytotoxin E at 0.03 and 0.1 μmol/kg), or an isotype control antibody linked to cytotoxin E at doses of 0.03 and 0.1 μmol/kg. Tumor volumes (LWH/2) and weights of mice were recorded throughout the course of the study, which was allowed to proceed for 61 days post dosing. The results are shown in FIG. 51. In this particular mouse xenograft model, which is immunocompomised, and at the stated dosage, treatment with the naked CD70 antibody did not show an effect on tumor volume (i.e., did not inhibit tumor growth). The isotype control also had little effect on the growth of the tumors. In contrast, the anti-CD70-cytotoxin E conjugate clearly showed dose-dependent anti-tumor efficacy. The therapeutic effect of the specific conjugate appears to be maximal even at 0.03 μmol/kg.

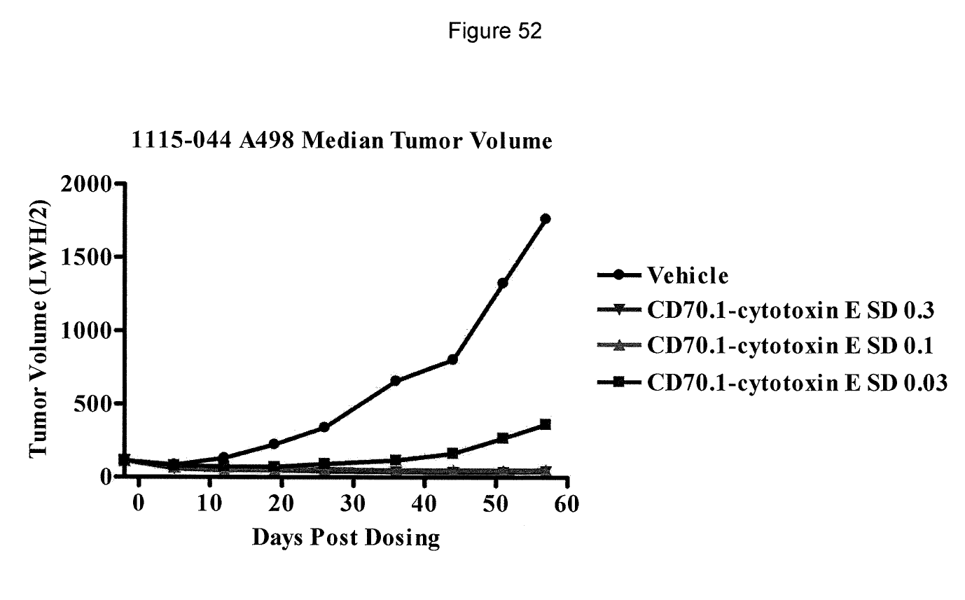

The activity of anti-CD70-cytotoxin E was next demonstrated in SCID mice bearing A498 tumor xenografts. A498 cells (5 million in 0.1 ml PBS and 0.1 ml Matrigel™/mouse) were implanted subcutaneously into SCID mice, and when tumors reached an average size of 110 mm³, groups of 8 mice were treated by ip injection of a single dose of either anti-CD70-cytotoxin Eat 0.03, 0.1 or 0.3 μmol/kg body weight. In addition, a control group was injected with vehicle alone. Tumor volumes (LWH/2) and weights of mice were recorded throughout the course of the study, which was allowed to proceed for approximately 60 days post dosing. The results are shown in FIG. 52. The results indicate that the anti-CD70-cytotoxin E conjugate is efficacious in the treatment of renal cancer in this model, and that therapy is dose-dependent.

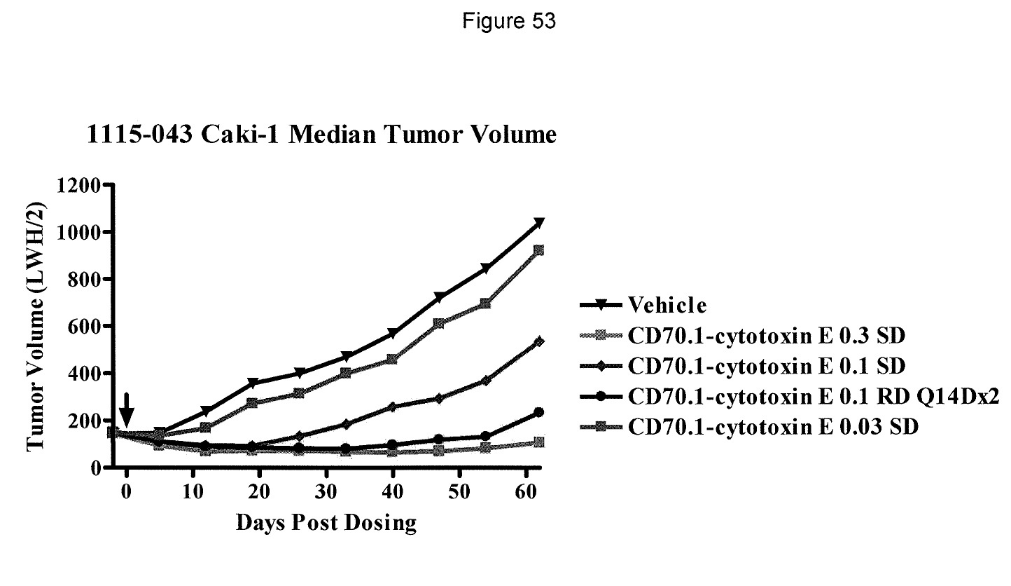

The activity of anti-CD70-cytotoxin E was next demonstrated in SCID mice bearing Caki-1 tumor xenografts. Caki-1 cells (2.5 million in 0.1 ml PBS and 0.1 ml Matrigel™/mouse) were implanted subcutaneously into SCID mice, and when tumors reached an average size of 150 mm³, groups of 8 mice were treated by ip injection of a single dose of either anti-CD70-cytotoxin Eat 0.03, 0.1 or 0.3 μmol/kg body weight. An extra group was also used to study the effect of a repeat dose therapy by dosing with two doses of anti-CD70-cytotoxin E conjugate at 0.1 μmol/kg, separated by 14 days. In addition, a control group was injected with vehicle alone. Tumor volumes (LWH/2) and weights of mice were recorded throughout the course of the study, which was allowed to proceed for 62 days post dosing. The results are shown in FIG. 53. The results indicate that the anti-CD70-cytotoxin E conjugate is efficacious in the treatment of renal cancer in mice bearing caki-1 tumors, and that therapy is dose-dependent.

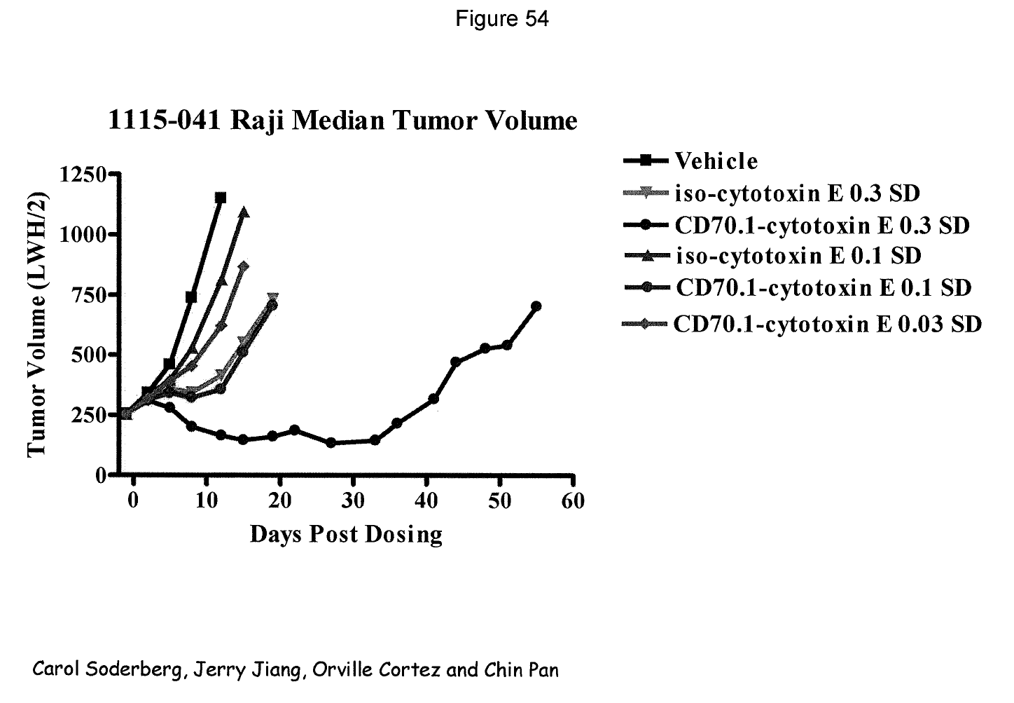

To demonstrate the activity of anti-CD70-cytotoxin E in a model of lymphoma, a therapy study was carried out in SCID mice bearing subcutaneous Raji xenografts. Raji cells (10 million in 0.1 ml PBS and 0.1 ml Matrigel™/mouse) were implanted subcutaneously into SCID mice, and when tumors reached an average size of 250 mm³, groups of 8 mice were treated by ip injection of a single dose of anti-CD70-cytotoxin E at 0.03, 0.1 or 0.3 μmol/kg body weight. In addition, control group were injected with vehicle alone, or isotype control antibody linked to cytotoxin E at 0.1 or 0.3 μmol/kg body weight. Tumor volumes (LWH/2) and weights of mice were recorded throughout the course of the study, which was allowed to proceed for approximately 60 days post dosing. The results are shown in FIG. 54. The results indicate that the anti-CD70-cytotoxin E conjugate is also efficacious in the treatment of lymphoma in this model, and that therapy is dose-dependent.

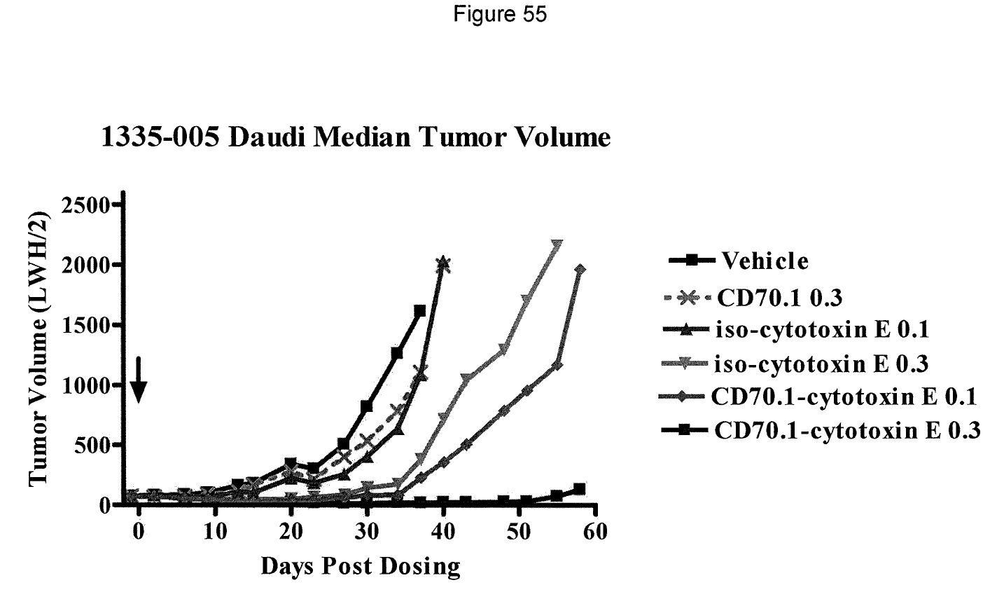

A second lymphoma model was carried out using Daudi xenografts. Daudi cells (10 million in 0.1 ml PBS and 0.1 ml Matrigel™/mouse) were implanted subcutaneously into SCID mice, and when tumors reached an average size of 70 mm³, groups of 8 mice were treated by ip injection of a single dose of either anti-CD70-cytotoxin E at 0.1 or 0.3 μmol/kg body weight. In addition, control groups were injected with vehicle alone, anti-CD70 antibody alone, or isotype control antibody cytotoxin E conjugate at 0.1 or 0.3 μmol/kg body weight. Tumor volumes (LWH/2) and weights of mice were recorded throughout the course of the study, which was allowed to proceed for approximately 60 days post dosing. The results are shown in FIG. 55. In this particular mouse xenograft model, which is immunocompomised, and at the stated dosage, treatment with the naked CD70 antibody did not show an effect on tumor volume (i.e., did not inhibit tumor growth). In contrast, the anti-CD70-cytotoxin E conjugate is efficacious against lymphoma in this model, and that therapy is dose-dependent.

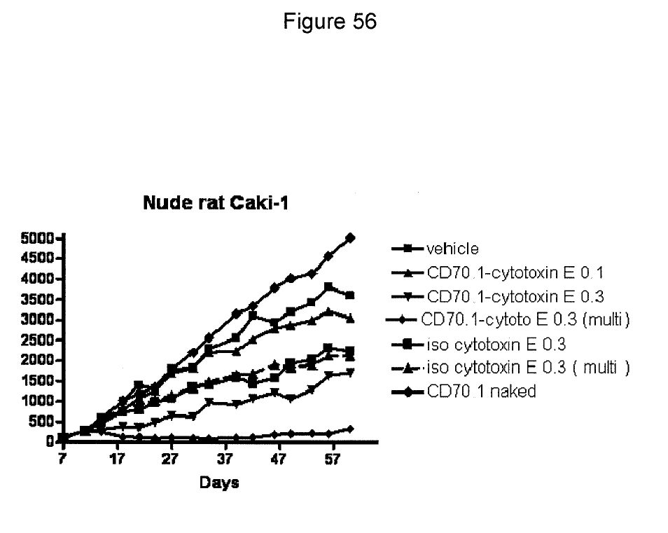

In order to demonstrate that efficacy could be observed in multiple species, a xenograft model in the nude rat was tested. In this model whole-body γ-irradiated nude rats were implanted subcutaneously with Caki-1 cells (10 million in 0.2 ml RPMI-1640/rat) and when tumors reached an average size of 100 mm³, groups of rats were treated by ip injection of a single dose of either anti-CD70-cytotoxin E at 0.1 or 0.3μmol/kg body weight. Alternatively multi-dose therapy was carried out in which rats received 3 doses of 0.3 μmol/kg body weight, on days 8, 15 and 22. In addition, control groups were injected with vehicle alone, anti-CD70 antibody alone, or isotype control antibody cytotoxin E conjugate at 0.3 μmol/kg body weight as a single dose or in the same multi-dose regime. Tumor volumes (LW²/2) and weights of rats were recorded throughout the course of the study. The results are shown in FIG. 56. In this particular mouse xenograft model, which is immunocompomised, and at the stated dosage, treatment with the naked CD70 antibody did not show an effect on tumor volume (i.e., did not inhibit tumor growth). In contrast, the anti-CD70-cytotoxin E conjugate showed a marked anti-tumor effect. Efficacy is increased with multi-dose therapy, without significant effect on the body weight of the animals. The isotype control conjugate showed far less effect on tumor growth even with the repeat dosing regime.

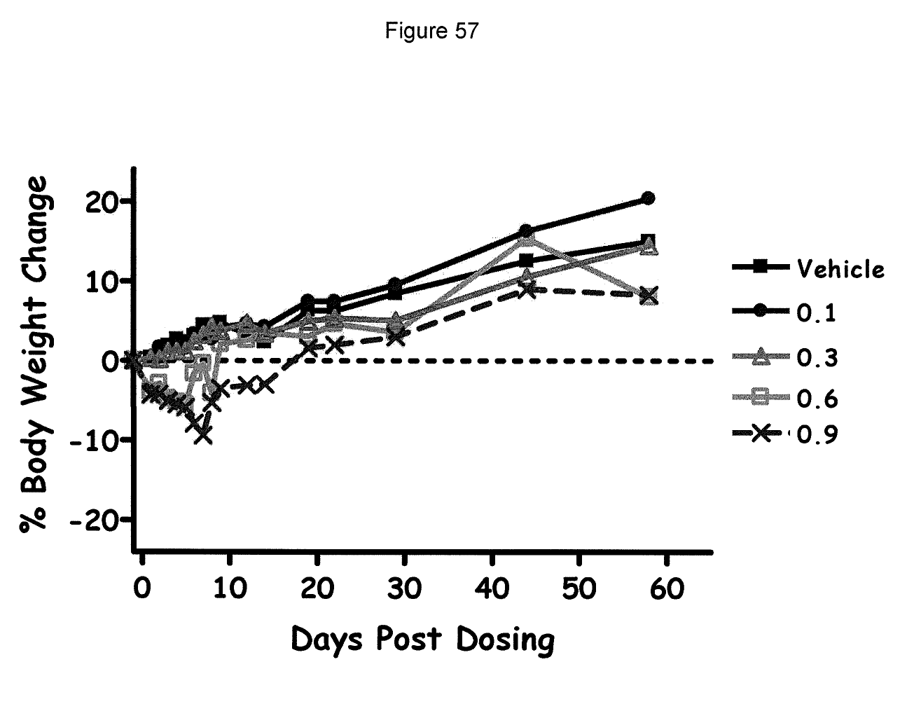

Safety of anti-CD70 conjugates was tested in three different animal species. Groups of 5 normal balb/c mice were dosed (ip) with anti-CD70-cytotoxin E at doses of 0.1, 0.3, 0.6 and 0.9 μmol/kg body weight and the body weight of the animals monitored over 60 days compared to animals injected with vehicle alone. Over the course of the study, control animals gained 10-20% in body weight. Mice dosed with anti-CD70-cytotoxin E showed that the conjugate was generally well tolerated with little effect on body weight at the lower doses. There was a dose-dependent increase in apparent toxicity, with the high doses causing a transient decrease in body weight of the animals before recovery. Nevertheless the conjugate is well tolerated at doses in excess of those required for efficacy in xenograft models. The results are shown in FIG. 57.

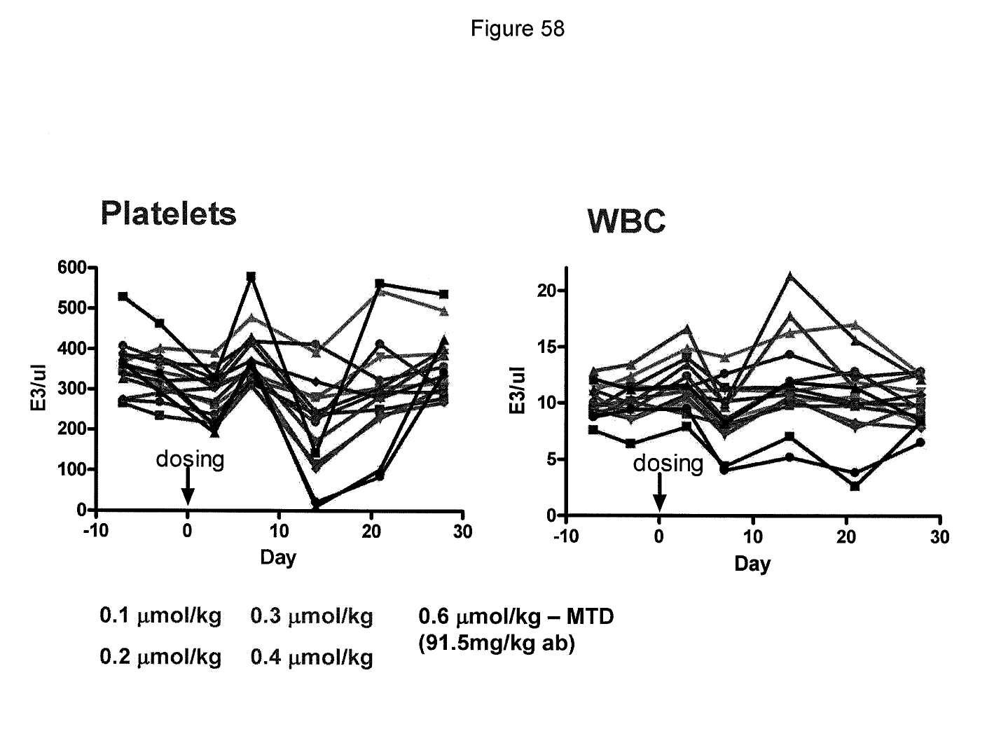

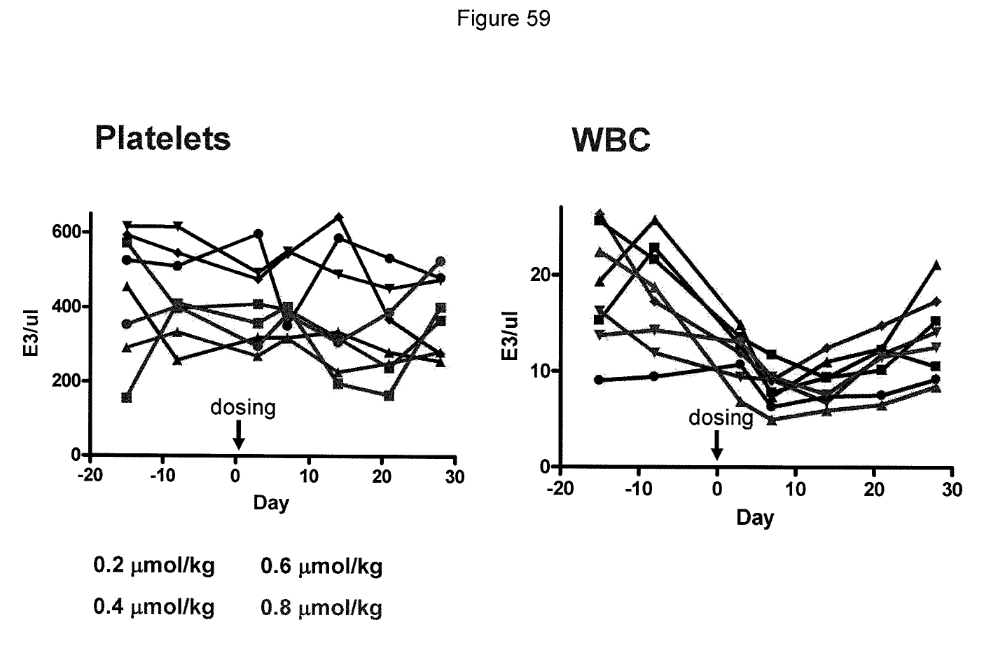

Toxicity was also tested in both dogs and monkeys. Groups of three dogs were dosed at 0.1, 0.2, 0.3, 0.4 and 0.6 μmol/kg body weight, and groups of two monkeys were dosed 0.2, 0.4, 0.6 and 0.8 μmol/kg body weight. Particular attention was paid to the total white blood cell count and the platelet count in each study as these are believed to be particularly sensitive indicators of toxicity for the anti-CD70 antibody-cytotoxin E conjugate. In dogs no significant changes in cell counts were observed until a dose of 0.6 μmol/kg body weight was reached. At this dose a transient drop in platelet count occurred, and white blood cell counts were also diminished. In monkeys, little change in these parameters were observed at any dose. Both studies support that the toxic dose of the anti-CD70 conjugate in animals is significantly higher than the efficacious dose in xenograft models. The results are shown in FIGS. 58 (results for dogs) and 59 (results for monkeys).

### Example 37

### Tumor Growth Inhibition in vivo by Anti-CD70-Cytotoxin F

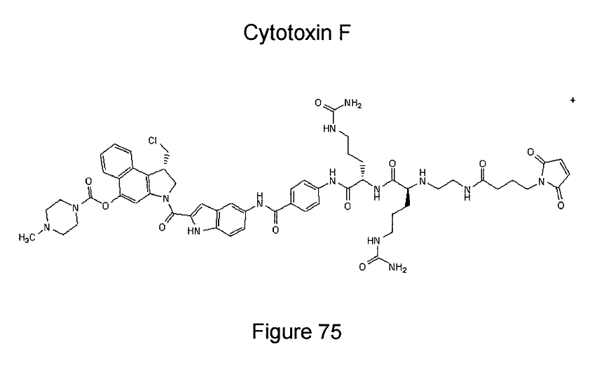

In this example, the efficacy of anti-CD70-cytotoxin F is demonstrated in two xenograft models of kidney cancer and one of lymphoma. A cytotoxin conjugate of the CD70 antibody 2H5 is referred to herein as CD70-cytotoxin F, which is comprised of a recombinant 2H5 anti-CD70 antibody linked to cytotoxin F (FIG. 75). Cytotoxin F is a prodrug requiring esterase activation.

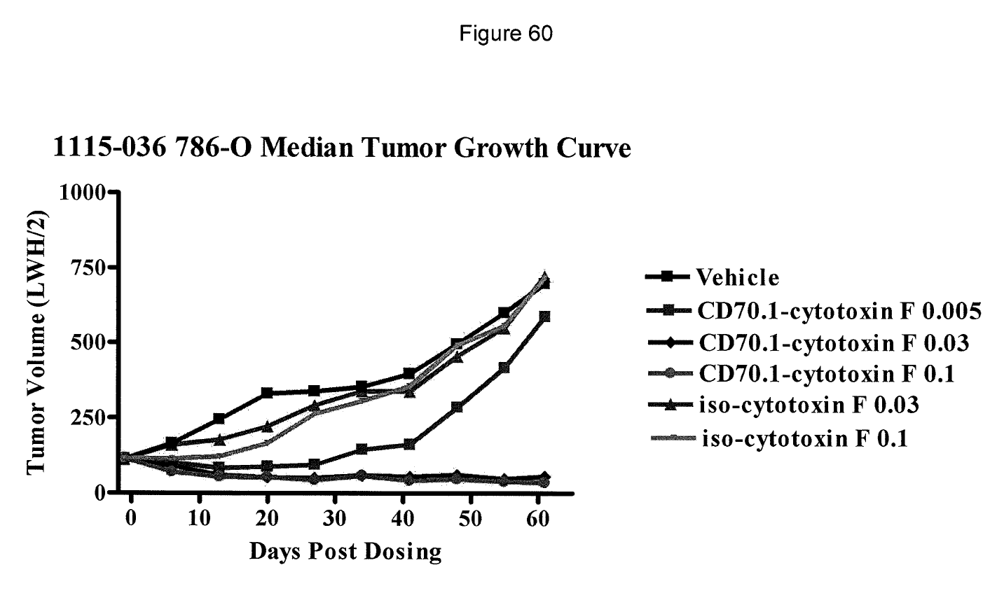

To demonstrate the activity of anti-CD70-cytotoxin F on 786-O cell xenografts, 2.5 million 786-O cells in 0.1 ml PBS and 0.1 ml Matrigel™ per mouse were implanted subcutaneously into SCID mice, and when tumors reached an average size of 110 mm³, groups of 8 mice were treated by ip injection of a single dose of either anti-CD70-cytotoxin F at 0.005, 0.03 or 0.1 μmol/kg body weight. In addition, control groups were injected with either vehicle alone, or an isotype control antibody linked to cytotoxin F at doses of 0.03 and 0.1 μmol/kg. Tumor volumes (LWH/2) and weights of mice were recorded throughout the course of the study, which was allowed to proceed for 62 days post dosing. The results are shown in FIG. 60. In this particular mouse xenograft model, which is immunocompomised, and at the stated dosage, treatment with the naked CD70 antibody did not show an effect on tumor volume (i.e., did not inhibit tumor growth). The isotype control conjugate also had little effect on the growth of the tumors in this experiment, whereas anti-CD70-cytotoxin F-treated mice clearly showed dose-dependent anti-tumor efficacy. The therapeutic effect of the specific conjugate appeared to be maximal even at 0.03 μmol/kg.

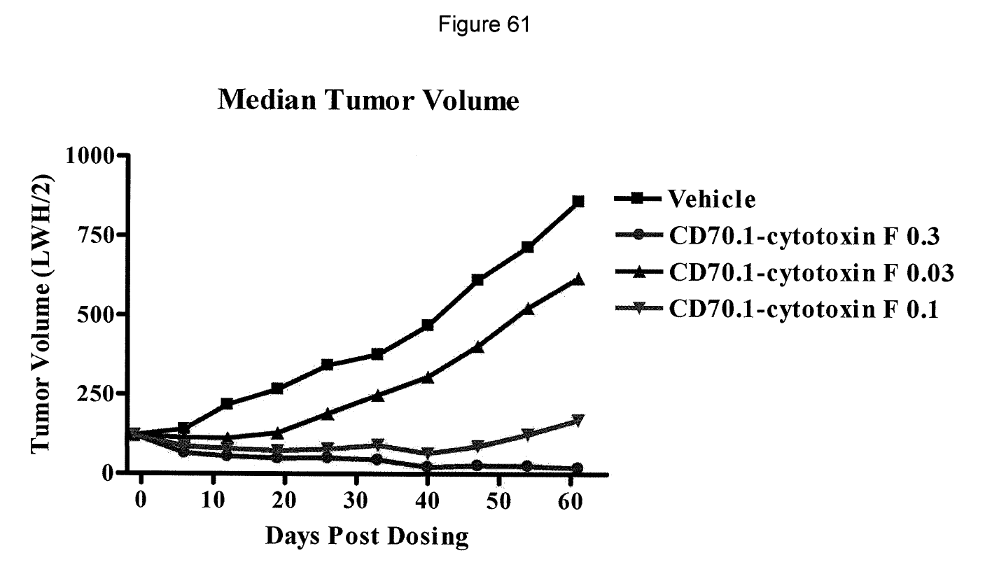

The activity of anti-CD70-cytotoxin F was next demonstrated in SCID mice bearing Caki-1 tumor xenografts. Caki-1 cells (2.5 million in 0.1 ml PBS and 0.1 ml Matrigel™/mouse) were implanted subcutaneously into SCID mice, and when tumors reached an average size of 120 mm³, groups of 8 mice were treated by ip injection of a single dose of either anti-CD70-cytotoxin F at 0.03, 0.1 or 0.3 μmol/kg body weight. In addition, a control group was injected with vehicle alone. Tumor volumes and weights of mice were recorded throughout the course of the study, which was allowed to proceed for 62 days post dosing. The results are shown in FIG. 61. The results indicate that the anti-CD70-cytotoxin F conjugate is efficacious in mice bearing caki-1 tumors, and that therapy is dose-dependent.

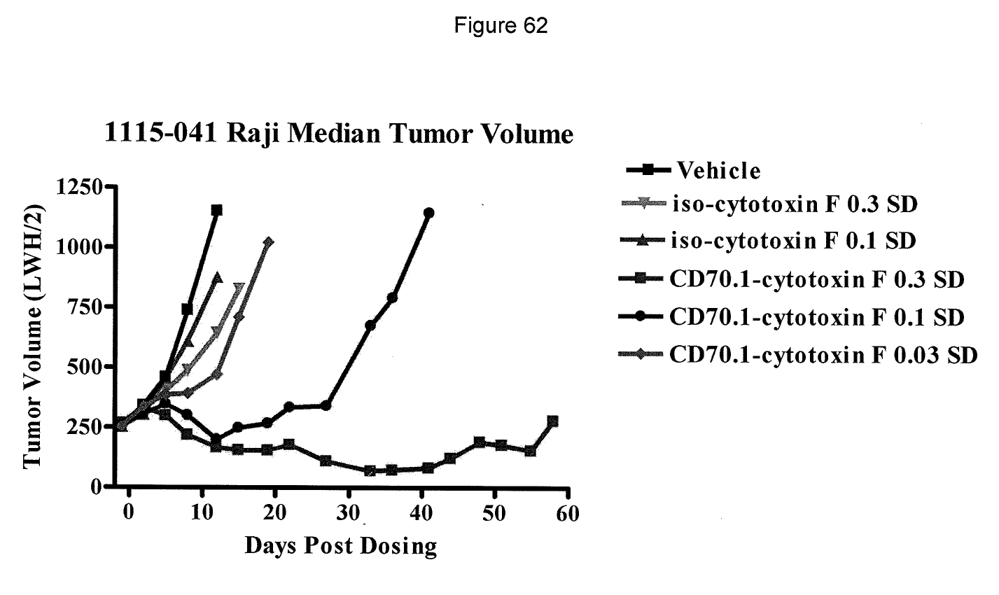

To demonstrate the activity of anti-CD70-cytotoxin F in a model of lymphoma, a therapy study was carried out in SCID mice bearing subcutaneous Raji xenografts. Raji cells (10 million in 0.1 ml PBS and 0.1 ml Matrigel™/mouse) were implanted subcutaneously into SCID mice, and when tumors reached an average size of 250 mm³, groups of 8 mice were treated by ip injection of a single dose of either anti-CD70-cytotoxin F at 0.03, 0.1 or 0.3 μmol/kg body weight. In addition, control group were injected with vehicle alone, or isotype control antibody linked to cytotoxin F at 0.1 or 0.3 μmol/kg body weight. Tumor volumes (LWH/2) and weights of mice were recorded throughout the course of the study, which was allowed to proceed for approximately 60 days post dosing. The results are shown in FIG. 62. The results indicate that the anti-CD70-cytotoxin F conjugate is also efficacious against lymphoma, and that therapy is dose-dependent.

### Example 38

### Tumor Growth Inhibition in vivo by Anti-CD70-Cytotoxin G

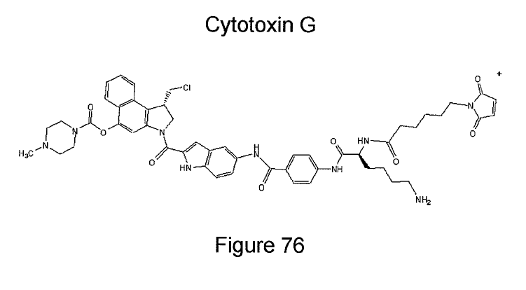

In this example, the efficacy of anti-CD70-cytotoxin G is demonstrated in two xenograft models of renal cancer. A cytotoxin conjugate of the CD70 antibody 2H5 is referred to herein as CD70-cytotoxin G, which is comprised of a recombinant 2H5 anti-CD70 antibody linked to cytotoxin G (FIG. 76). Cytotoxin G is a prodrug requiring esterase activation.

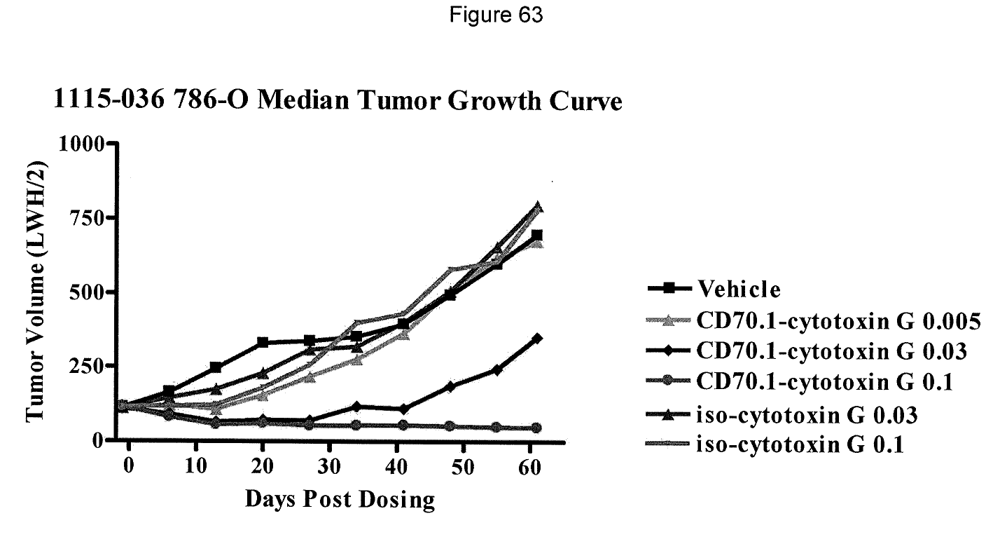

To demonstrate the activity of anti-CD70-cytotoxin G on 786-O cell xenografts, 2.5 million 786-O cells in 0.1 ml PBS and 0.1 ml Matrigel™ per mouse were implanted subcutaneously into SCID mice, and when tumors reached an average size of 110 mm³, groups of 8 mice were treated by ip injection of a single dose of either anti-CD70-cytotoxin G at 0.005, 0.03 or 0.1 μmol/kg body weight. In addition, control groups were injected with either vehicle alone, or an isotype control antibody linked to cytotoxin G at doses of 0.03 and 0.1 μmol/kg. Tumor volumes (LWH/2) and weights of mice were recorded throughout the course of the study, which was allowed to proceed for 61 days post dosing. The results are shown in FIG. 63. The results indicate that the anti-CD70 antibody alone or the isotype control conjugates has little effect on the growth of the tumors in this experiment, whereas the anti-CD70-cytotoxin G treated mice clearly shows dose-dependent anti-tumor efficacy.

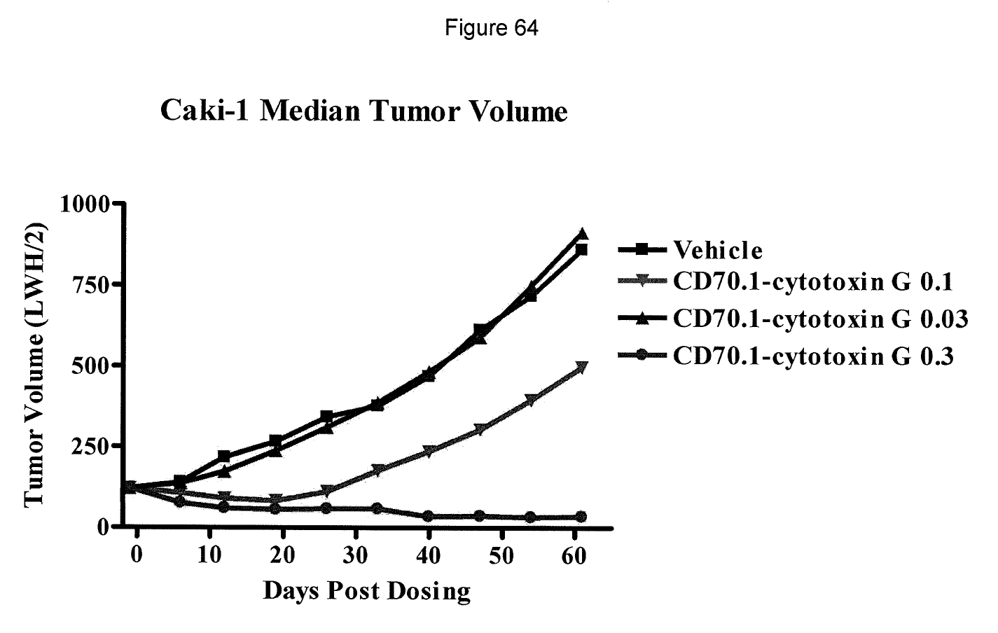

The activity of anti-CD70-cytotoxin G was next demonstrated in SCID mice bearing Caki-1 tumor xenografts. Caki-1 cells (2.5 million in 0.1 ml PBS and 0.1 ml Matrigel™/mouse) were implanted subcutaneously into SCID mice, and when tumors reached an average size of 120 mm³, groups of 8 mice were treated by ip injection of a single dose of either anti-CD70-cytotoxin G at 0.03, 0.1 or 0.3 μmol/kg body weight. In addition, a control group was injected with vehicle alone. Tumor volumes (LWH/2) and weights of mice were recorded throughout the course of the study, which was allowed to proceed for 61 days post dosing. The results are shown in FIG. 64. The results indicate that the anti-CD70-cytotoxin G conjugate is efficacious against renal cancer in mice bearing caki-1 tumors, and that therapy is dose-dependent.

### Example 39

### Tumor Growth Inhibition in vivo by Anti-CD70-Cytotoxin H

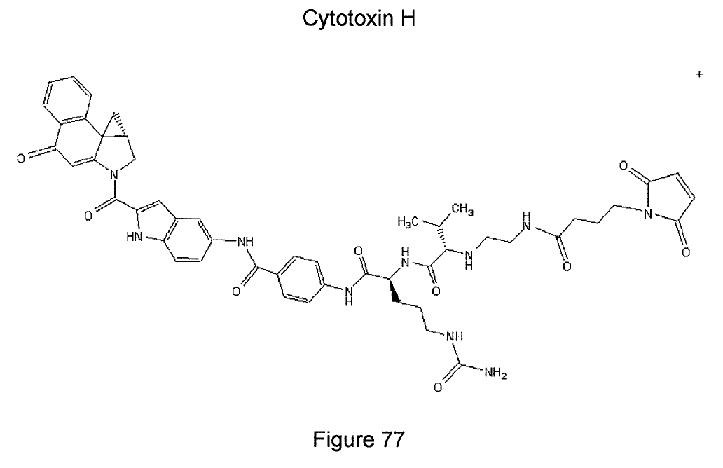

In this example, the efficacy of anti-CD70-cytotoxin H in two xenograft models of renal cancer is demonstrated. A cytotoxin conjugate of the CD70 antibody 2H5 is referred to herein as CD70-cytotoxin H, which is comprised of a recombinant 2H5 anti-CD70 antibody linked to cytotoxin H (FIG. 77).

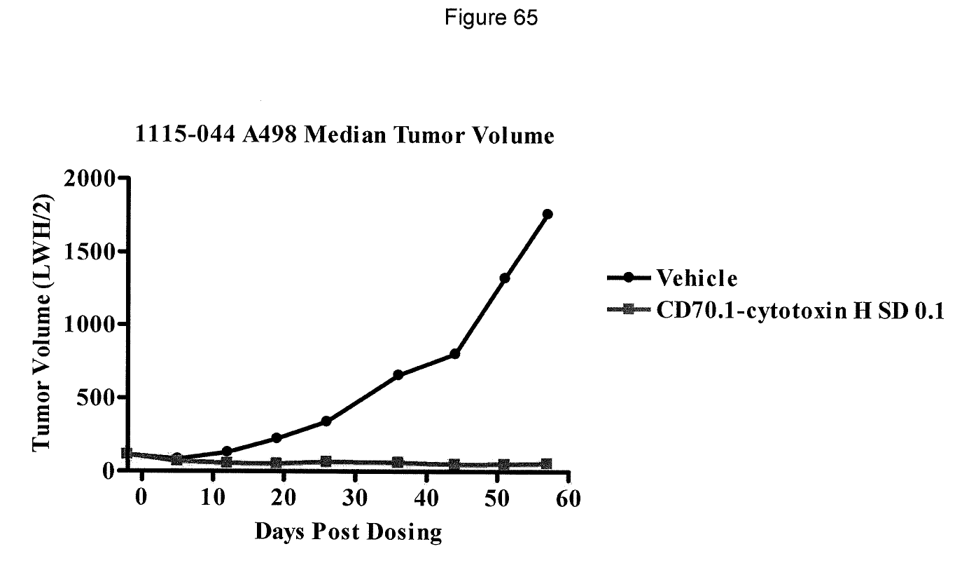

The activity of anti-CD70-cytotoxin H was demonstrated in SCID mice bearing A498 tumor xenografts. A498 cells (5 million in 0.1 ml PBS and 0.1 ml Matrigel™/mouse) were implanted subcutaneously into SCID mice, and when tumors reached an average size of 110 mm³, groups of 8 mice were treated by ip injection of a single dose of either anti-CD70-cytotoxin H at 0.1 μmol/kg body weight. In addition, a control group was injected with vehicle alone. Tumor volumes (LWH/2) and weights of mice were recorded throughout the course of the study, which was allowed to proceed for approximately 60 days post dosing. These results are shown in FIG. 65. The results indicate that the anti-CD70-cytotoxin H conjugate is efficacious against renal cancer.

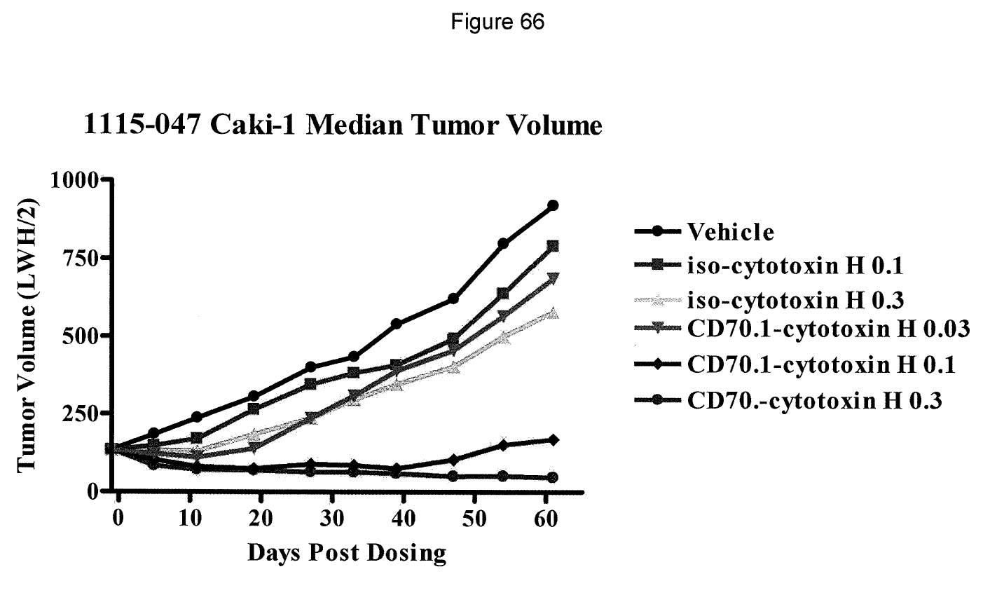

To demonstrate the activity of anti-CD70-cytotoxin H on Caki-1 cell xenografts, 2.5 million Caki-1 cells in 0.1 ml PBS and 0.1 ml Matrigel™ per mouse were implanted subcutaneously into SCID mice, and when tumors reached an average size of 130 mm³, groups of 8 mice were treated by ip injection of a single dose of either anti-CD70-cytotoxin H at 0.03, 0.1 or 0.3 μmol/kg body weight. In addition, control groups were injected with either vehicle alone, or an isotype control antibody linked to cytotoxin H at doses of 0.1 and 0.3 μmol/kg. Tumor volumes (LWH/2) and weights of mice were recorded throughout the course of the study, which was allowed to proceed for 61 days post dosing. The results are shown in FIG. 66. In this particular mouse xenograft model, which is immunocompomised, and at the stated dosage, treatment with the naked CD70 antibody did not show an effect on tumor volume (i.e., did not inhibit tumor growth). The isotype control conjugates also have little effect on the growth of the tumors in this experiment. In contrast, anti-CD70-cytotoxin H conjugate clearly shows dose-dependent antitumor efficacy.

### Example 40

### Tumor Growth Inhibition in vivo by Anti-CD70-Cytotoxin I

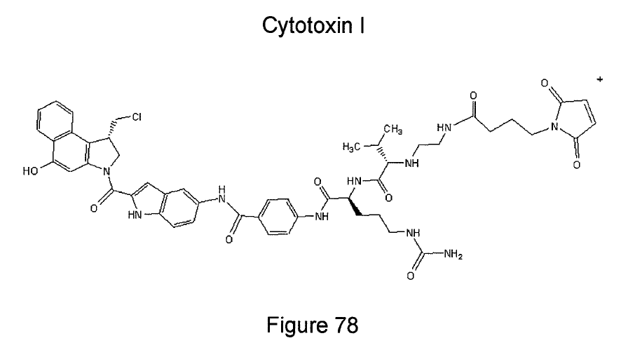

In this example, the efficacy of anti-CD70-cytotoxin I has been demonstrated in two xenograft models of kidney cancer, 786-O cells in SCID mice, and Caki-1 cells in nude rats. A cytotoxin conjugate of the CD70 antibody 2H5 is referred to herein as CD70-cytotoxin I, which is comprised of a recombinant 2H5 anti-CD70 antibody linked to cytotoxin I (FIG. 78).

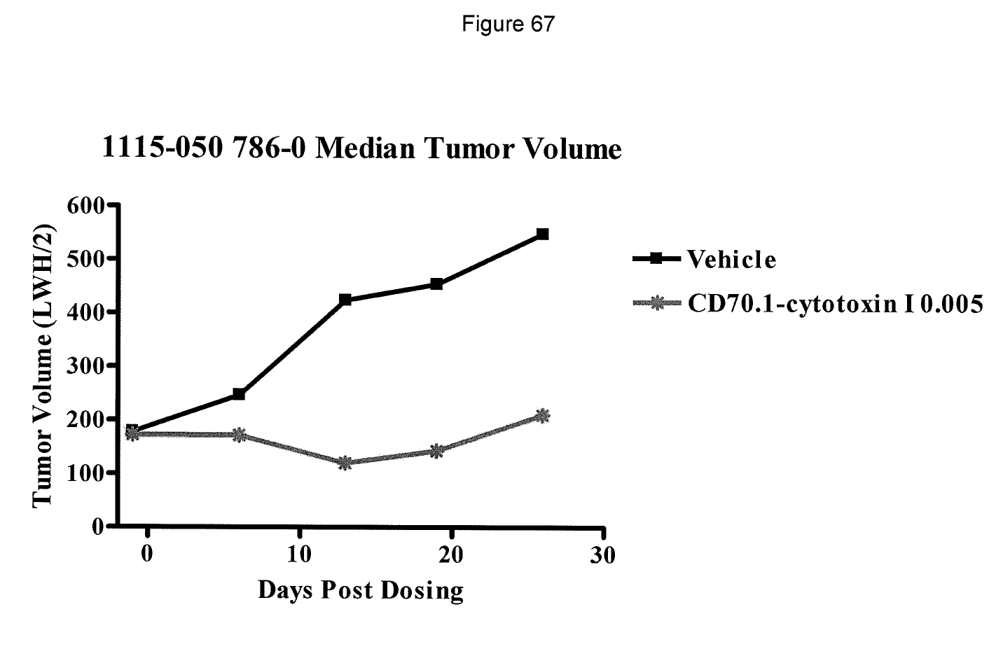

The activity of anti-CD70-cytotoxin I was demonstrated in SCID mice bearing 786-O tumor xenografts. 786-O cells (2.5 million in 0.1 ml PBS and 0.1 ml Matrigel™/mouse) were implanted subcutaneously into SCID mice, and when tumors reached an average size of 170 mm³, groups of 6 mice were treated by ip injection of a single dose of anti-CD70-cytotoxin I at 0.005 μmol/kg body weight. In addition, a control group was injected with vehicle alone. Tumor volumes (LWH/2) and weights of mice were recorded throughout the course of the study. The results are shown in FIG. 67. These results demonstrate that the anti-CD70-cytotoxin I conjugate is efficacious against renal cancer, even at a low dose.

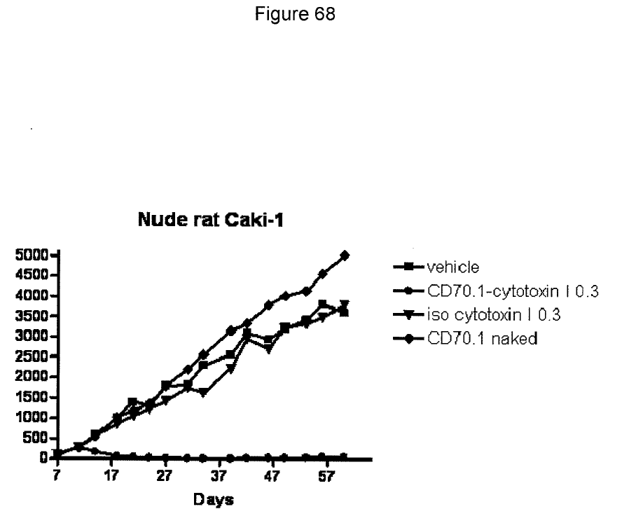

In order to demonstrate that efficacy could be observed in multiple species, a xenograft model in the nude rat was tested. In this model nude rats were implanted subcutaneously with Caki-1 cells (10 million in 0.2 ml RPMI-1640/rat) and when tumors reached an average size of 100 mm³, groups of rats were treated by ip injection of a single dose of either anti-CD70-cytotoxin I at 0.3 μmol/kg body weight. In addition, control groups were injected with vehicle alone, anti-CD70 antibody alone, or isotype control antibody cytotoxin I conjugate at 0.3 μmol/kg body weight as a single dose. Tumor volumes (LW²/2) and weights of rats were recorded throughout the course of the study. The results are shown in FIG. 68. The results show that the CD70 antibody alone has little effect on tumor growth, and the isotype control conjugate shows no effect on tumor growth. However, the anti-CD70-cytotoxin I conjugate shows a marked anti-tumor effect. Tumor regression was achieved. Therefore, the anti-CD70-cytotoxin I conjugate shows an anti-tumor effect in multiple species.

### Example 41

### Tumor Growth Inhibition in vivo by Anti-CD70-Cytotoxin J

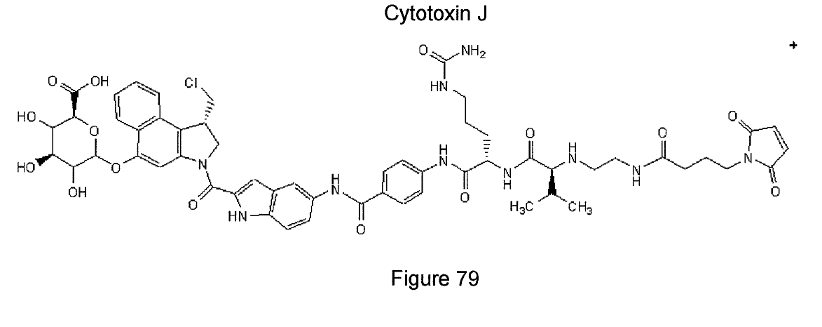

In this example, the efficacy of anti-CD70-cytotoxin J has been demonstrated in a xenograft models of kidney cancer, 786-O cells in SCID mice. A cytotoxin conjugate of the CD70 antibody 2H5 is referred to herein as CD70-cytotoxin J, which is comprised of a recombinant 2H5 anti-CD70 antibody linked to cytotoxin J (FIG. 79). Cytotoxin J is a prodrug requiring cleavage by glucuronidase for activation.

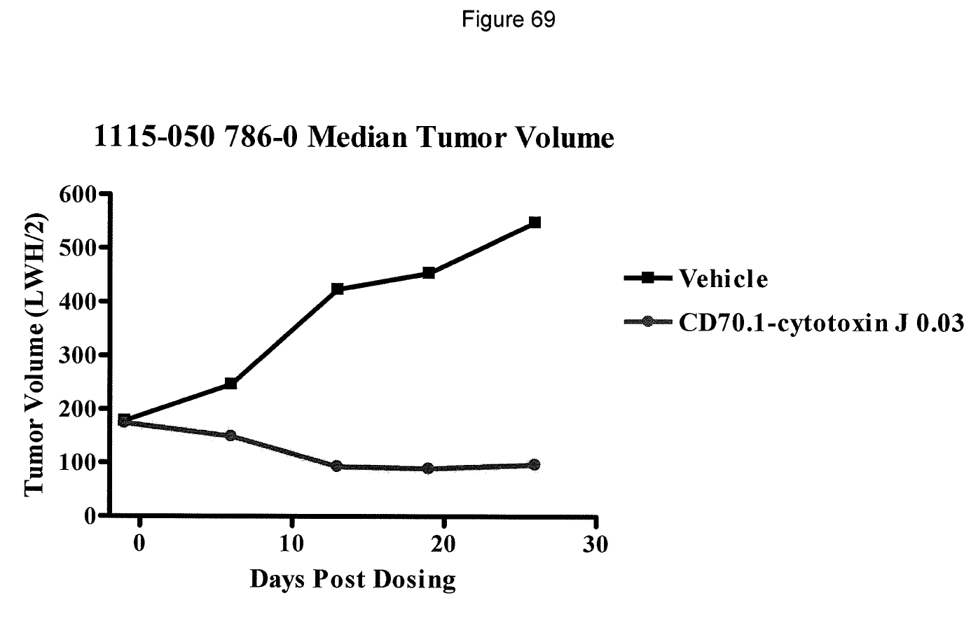

The activity of anti-CD70-cytotoxin J was demonstrated in SCID mice bearing 786-O tumor xenografts. 786-O cells (2.5 million in 0.1 ml PBS and 0.1 ml Matrigel™/mouse) were implanted subcutaneously into SCID mice, and when tumors reached an average size of 170 mm³, groups of 6 mice were treated by ip injection of a single dose of anti-CD70-cytotoxin J at 0.03 μmol/kg body weight. In addition, a control group was injected with vehicle alone. Tumor volumes (LWH/2) and weights of mice were recorded throughout the course of the study. The results are shown in FIG. 69. The results demonstrate that the anti-CD70-cytotoxin J conjugate is efficacious against renal cancer in this model.

### Example 42

### Functional Blocking of CD70 Costimulated T Cell Proliferation by Anti-CD70 Antibodies

This example describes the analysis and characterization of the functional blocking of CD70 costimulated T cell proliferation by anti-CD70 antibodies 1F4 IgG1, 1F4 IgG4, 2H5, 2H5 F(ab′)₂ and 2H5 Fab.

Human CD3⁺ T cells were isolated from cryopreserved PBMC using MACS CD3 Microbeads and then cultured at 2×10⁶ cells/ml in RPMI-1640 complete media+10% heat inactivated FCS in the presence of Mitomycin C treated CHO cells stably transfected with both mouse CD32 and human CD70. Cells were stimulated with I anti-CD3 (clone OKT3) for 3 days, 1 μCi/well of ³H-Thymidine was added for 6 hours and the cells were harvested. Proliferation was measured as CPM incorporated by scintillation counting.

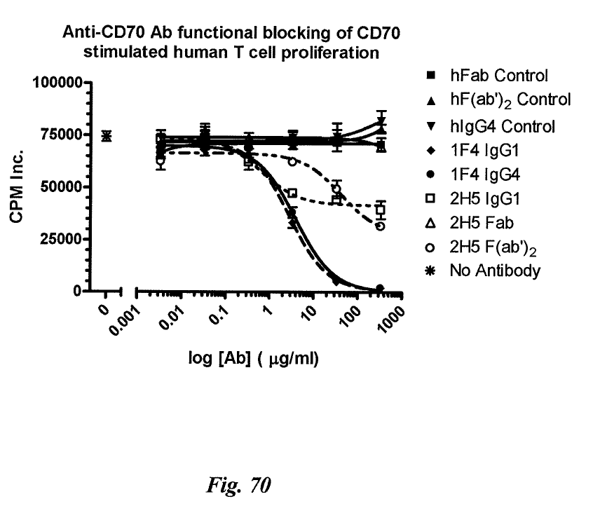

The data show that 1F4 and 2H5 antibodies can block CD70-mediated CD27 signaling induced proliferation by human anti-CD3 stimulated T-cells in a dose dependent manner. The data also show that functional blocking by 2H5 atypically requires IgG1 Fc region mediated cell surface CD70 multimerization to affect blocking activity whereas 1F4 typically does not. See FIG. 70. That unusual characteristic of the epitope bound by 2H5 is demonstrated by the reduced functional blocking efficacy of 2H5 F(ab′)₂ and the complete lack of functional blocking activity of 2H5 Fab relative to 2H5 IgG1. In contrast, the equivalent functional blocking activity of 1F4 IgG4 relative to 1F4 IgG1 demonstrates that the epitope bound by 1F4 typically does not require IgG1 Fc region mediated CD70 multimerization to affect blocking activity, as is typically observed with Abs having functional blocking activity.

Therefore, these data show that 2H5 binds an epitope that has unusual and possibly unique properties with respect to functional blocking of CD70-mediated human T-cell activation. In addition, the epitope bound by 2H5 may also contribute favorably to the quality and potency of 2H5 IgG1 or 2H5 NF mediated ADCC, internalization, affinity, etc.

The ability of antibodies 1F4 and 2H5 to block CD70-mediated CD27 signaling induced proliferation by human anti-CD3 stimulated T-cells is relevant for the treatment of any inflammation indication where CD70 function has a role in disease progression.

### LIST OF SEQUENCE IDENTIFIERS

| SEQ ID NO: | SEQUENCE |
|---|---|
| 1 | VH a.a. 2H5 |
| 2 | VH a.a. 10B4 |
| 3 | VH a.a. 8B5 |
| 4 | VH a.a. 18E7 |
| 5 | VH a.a. 69A7 |
| 6 | VH a.a. 1F4 |
| 7 | VK a.a. 2H5 |
| 8 | VK a.a. 10B4 |
| 9 | VK a.a. 8B5 |
| 10 | VK a.a. 18E7 |
| 11 | VK a.a. 69A7 and 69A7Y |
| 12 | VK a.a. 1F4 |
| 13 | VH CDR1 a.a. 2H5 |
| 14 | VH CDR1 a.a. 10B4 |
| 15 | VH CDR1 a.a. 8B5 |
| 16 | VH CDR1 a.a. 18E7 |
| 17 | VH CDR1 a.a. 69A7 and 69A7Y |
| 18 | VH CDR1 a.a. 1F4 |
| 19 | VH CDR2 a.a. 2H5 |
| 20 | VH CDR2 a.a. 10B4 |
| 21 | VH CDR2 a.a. 8B5 |
| 22 | VH CDR2 a.a. 18E7 |
| 23 | VH CDR2 a.a. 69A7 and 69A7Y |
| 24 | VH CDR2 a.a. 1F4 |
| 25 | VH CDR3 a.a. 2H5 |
| 26 | VH CDR3 a.a. 10B4 |
| 27 | VH CDR3 a.a. 8B5 |
| 28 | VH CDR3 a.a. 18E7 |
| 29 | VH CDR3 a.a. 69A7 |
| 30 | VH CDR3 a.a. 1F4 |
| 31 | VK CDR1 a.a. 2H5 |
| 32 | VK CDR1 a.a. 10B4 |
| 33 | VK CDR1 a.a. 8B5 |
| 34 | VK CDR1 a.a. 18E7 |
| 35 | VK CDR1 a.a. 69A7 and 69A7Y |
| 36 | VK CDR1 a.a. 1F4 |
| 37 | VK CDR2 a.a. 2H5 |
| 38 | VK CDR2 a.a. 10B4 |
| 39 | VK CDR2 a.a. 8B5 |
| 40 | VK CDR2 a.a. 18E7 |
| 41 | VK CDR2 a.a. 69A7 and 69A7Y |
| 42 | VK CDR2 a.a. 1F4 |
| 43 | VK CDR3 a.a. 2H5 |
| 44 | VK CDR3 a.a. 10B4 |
| 45 | VK CDR3 a.a. 8B5 |
| 46 | VK CDR3 a.a. 18E7 |
| 47 | VK CDR3 a.a. 69A7 and 69A7Y |
| 48 | VK CDR3 a.a. 1F4 |
| 49 | VH n.t. 2H5 |
| 50 | VH n.t. 10B4 |
| 51 | VH n.t. 8B5 |
| 52 | VH n.t. 18E7 |
| 53 | VH n.t. 69A7 |
| 54 | VH n.t. 1F4 |
| 55 | VK n.t. 2H5 |
| 56 | VK n.t. 10B4 |
| 57 | VK n.t. 8B5 |
| 58 | VK n.t. 18E7 |
| 59 | VK n.t. 69A7 and 69A7Y |
| 60 | VK n.t. 1F4 |
| 61 | VH 3-30.3 germline a.a. |
| 62 | VH 3-33 germline a.a. |
| 63 | VH 4-61 germline a.a. |
| 64 | VH 3-23 germline a.a. |
| 65 | VK L6 germline a.a. |
| 66 | VK L18 germline a.a. |
| 67 | VK L15 germline a.a. |
| 68 | VK A27 germline a.a. |
| 69 | JH 4b germline a.a. |
| 70 | JK 4 germline a.a. |
| 71 | JK 3 germline a.a. |
| 72 | JK 2 germline a.a. |
| 73 | VH a.a. 69A7Y |
| 74 | VH n.t. 69A7Y |
| 75 | VH CDR3 a.a. 69A7Y |
| 76 | human CD70 (P32970) |
| 77 | peptide linker |
| 78 | peptide linker |
| 79 | peptide linker |
| 80 | peptide linker |
| 81 | peptide linker |
| 82 | peptide linker |
| 83 | peptide linker |
| 84 | peptide linker |
| 85 | peptide linker |
| 86 | peptide linker |
| 87 | peptide linker |
| 88 | peptide linker |
| 89 | peptide linker |
| 90 | cytomegalovirus peptide |
| 91 | peptide linker |
| 92 | peptide linker |

1. An antibody-partner molecule conjugate comprising an isolated human monoclonal antibody, or an antigen-binding portion thereof, wherein the antibody binds human CD70 and exhibits at least one, two, three, four, five, or all six of the following properties:
(a) binds to human CD70 with a K_{D} of 1×10⁻⁷ M or less; and
(b) binds to a renal cell carcinoma tumor cell line;
(c) binds to a lymphoma cell line;
(d) is internalized by CD70-expressing cells;
(e) exhibits antibody dependent cellular cytotoxicity (ADCC) against CD70-expressing cells; and
(f) inhibits growth of CD70-expressing cells in vivo when conjugated to a cytotoxin, and a partner molecule, wherein the partner molecule is a therapeutic agent.

2.-9. (canceled)

10. The antibody-partner molecule conjugate of claim 1, wherein the monoclonal antibody, or antigen binding portion thereof, binds an epitope on human CD70 recognized by a reference antibody, wherein the reference antibody comprises:
(a) a heavy chain variable region comprising the amino acid sequence of SEQ ID NO:1 and a light chain variable region comprising the amino acid sequence of SEQ ID NO:7;
(b) a heavy chain variable region comprising the amino acid sequence of SEQ ID NO:2 and a light chain variable region comprising the amino acid sequence of SEQ ID NO:8;
(c) a heavy chain variable region comprising the amino acid sequence of SEQ ID NO:3 and a light chain variable region comprising the amino acid sequence of SEQ ID NO:9;
(d) a heavy chain variable region comprising the amino acid sequence of SEQ ID NO:4 and a light chain variable region comprising the amino acid sequence of SEQ ID NO:10;
(e) a heavy chain variable region comprising the amino acid sequence of SEQ ID NO:5 and a light chain variable region comprising the amino acid sequence of SEQ ID NO:11;
(f) a heavy chain variable region comprising the amino acid sequence of SEQ ID NO:73 and a light chain variable region comprising the amino acid sequence of SEQ ID NO:11; or (g) a heavy chain variable region comprising the amino acid sequence of SEQ ID NO:6 and a light chain variable region comprising the amino acid sequence of SEQ ID NO:12, and a partner molecule, wherein the partner molecule is a therapeutic agent.

11.-17. (canceled)

18. An antibody-partner molecule conjugate comprising an isolated monoclonal antibody, or an antigen-binding portion thereof, comprising a heavy chain variable region that is the product of or derived from a human V_{H} 3-30.3 gene, human V_{H} 3-33 gene, human V_{H} 4-61 gene, or human V_{H} 3-23 gene, and a light chain variable region that is the product of or derived from a human V_{K} L6 human V_{K} L15 gene, human V_{K}L6gene, or human V_{K} A27 gene, wherein the antibody specifically binds CD70, and a partner molecule, wherein the partner molecule is a therapeutic agent.

19.-20. (canceled)

21. The antibody-partner molecule conjugate of claim 1, wherein the antibody or antigen-binding portion thereof, comprises:
(a) a heavy chain variable region CDR1 comprising SEQ ID NO:13;
(b) a heavy chain variable region CDR2 comprising SEQ ID NO:19;
(c) a heavy chain variable region CDR3 comprising SEQ ID NO:25;
(d) a light chain variable region CDR1 comprising SEQ ID NO:31;
(e) a light chain variable region CDR2 comprising SEQ ID NO:37; and
(f) a light chain variable region CDR3 comprising SEQ ID NO:43, or
(a) a heavy chain variable region CDR1 comprising SEQ ID NO:14;
(b) a heavy chain variable region CDR2 comprising SEQ ID NO:20;
(c) a heavy chain variable region CDR3 comprising SEQ ID NO:26;
[d) a light chain variable region CDR1 comprising SEQ ID NO:32;
(e) a light chain variable region CDR2 comprising SEQ ID NO:38; and
(f) a light chain variable region CDR3 comprising SEQ ID NO:44, or
(a) a heavy chain variable region CDR1 comprising SEQ ID NO:15;
(b) a heavy chain variable region CDR2 comprising SEQ ID NO:21;
(c) a heavy chain variable region CDR3 comprising SEQ ID NO:27;
(d) a light chain variable region CDR1 comprising SEQ ID NO:33;
(e) a light chain variable region CDR2 comprising SEQ ID NO:39; and
(f) a light chain variable region CDR3 comprising SEQ ID NO:45; or
(a) a heavy chain variable region CDR1 comprising SEQ ID NO:16;
(b) a heavy chain variable region CDR2 comprising SEQ ID NO:22;
(c) a heavy chain variable region CDR3 comprising SEQ ID NO:28;
(d) a light chain variable region CDR1 comprising SEQ ID NO:34;
(e) a light chain variable region CDR2 comprising SEQ ID NO:40; and
(f) a light chain variable region CDR3 comprising SEQ ID NO:46; or
(a) a heavy chain variable region CDR1 comprising SEQ ID NO:17;
(b) a heavy chain variable region CDR2 comprising SEQ ID NO:23;
(c) a heavy chain variable region CDR3 comprising SEQ ID NO:29;
(d) a light chain variable region CDR1 comprising SEQ ID NO:35;
(e) a light chain variable region CDR2 comprising SEQ ID NO:41; and
(f) a light chain variable region CDR3 comprising SEQ ID NO:47 or
(a) a heavy chain variable region CDR1 comprising SEQ ID NO:17;
(b) a heavy chain variable region CDR2 comprising SEQ ID NO:23;
(c) a heavy chain variable region CDR3 comprising SEQ ID NO:75;
(d) a light chain variable region CDR1 comprising SEQ ID NO:35;
(e) a light chain variable region CDR2 comprising SEQ ID NO:41; and
(f) a light chain variable region CDR3 comprising SEQ ID NO:47 or
(a) a heavy chain variable region CDR1 comprising SEQ ID NO:18;
(b) a heavy chain variable region CDR2 comprising SEQ ID NO:24;
(c) a heavy chain variable region CDR3 comprising SEQ ID NO:30;
(d) a light chain variable region CDR1 comprising SEQ ID NO:36;
(e) a light chain variable region CDR2 comprising SEQ ID NO:42; and
(f) a light chain variable region CDR3 comprising SEQ ID NO:48.

22.-27. (canceled)

28. An The antibody-partner molecule conjugate of claim 1, wherein the isolated monoclonal antibody, or antigen binding portion thereof, comprises:
(a) a heavy chain variable region comprising an amino acid sequence selected from the group consisting of SEQ ID NOs: 1-6, and 73; and
(b) a light chain variable region comprising an amino acid sequence selected from the group consisting of SEQ ID NOs: 7-12;
wherein the antibody specifically binds a human CD70 protein, and a partner molecule, wherein the partner molecule is a therapeutic agent.

29. The antibody-partner molecule conjugate of claim 28, wherein the antibody or antigen-binding portion thereof, comprises:
(a) a heavy chain variable region comprising the amino acid sequence of SEQ ID NO: 1; and
(b) a light chain variable region comprising the amino acid sequence of SEQ ID NO: 7.

30. The antibody-partner molecule conjugate of claim 28, wherein the antibody or antigen-binding portion thereof, comprises:
(a) a heavy chain variable region comprising the amino acid sequence of SEQ ID NO: 2; and
(b) a light chain variable region comprising the amino acid sequence of SEQ ID NO: 8.

31. The antibody-partner molecule conjugate of claim 28, wherein the antibody or antigen-binding portion thereof, comprises:
(a) a heavy chain variable region comprising the amino acid sequence of SEQ ID NO: 3; and
(b) a light chain variable region comprising the amino acid sequence of SEQ ID NO: 9.

32. The antibody-partner molecule conjugate of claim 28, wherein the antibody or antigen-binding portion thereof, comprises:
(a) a heavy chain variable region comprising the amino acid sequence of SEQ ID NO: 4; and
(b) a light chain variable region comprising the amino acid sequence of SEQ ID NO: 10.

33. The antibody-partner molecule conjugate of claim 28, wherein the antibody or antigen-binding portion thereof, comprises:
(a) a heavy chain variable region comprising the amino acid sequence of SEQ ID NO: 5; and
(b) a light chain variable region comprising the amino acid sequence of SEQ ID NO: 11.

34. The antibody-partner molecule conjugate of claim 28, wherein the antibody or antigen-binding portion thereof, comprises:
(a) a heavy chain variable region comprising the amino acid sequence of SEQ ID NO: 73; and
(b) a light chain variable region comprising the amino acid sequence of SEQ ID NO: 11.

35. The antibody-partner molecule conjugate of claim 28, wherein the antibody or antigen-binding portion thereof, comprises:
(a) a heavy chain variable region comprising the amino acid sequence of SEQ ID NO: 6; and
(b) a light chain variable region comprising the amino acid sequence of SEQ ID NO: 12.

36.-37. (canceled)

38. The antibody-partner molecule conjugate of claim 1, wherein the therapeutic agent is a cytotoxin or a radioactive isotope.

39.-41. (canceled)

42. A method of inhibiting growth of a CD70-expressing tumor cell comprising contacting the CD70-expressing tumor cell with the antibody-partner molecule conjugate of claim 1 such that growth of the CD70-expressing tumor cell is inhibited.

43. The method of claim 42, wherein the CD70-expressing tumor cell is a renal tumor cell or a lymphoma cell.

44. The method of claim 42, wherein the CD70-expressing tumor cell is from a cancer selected from the group consisting of renal cell carcinoma or lymphoma.

45. A method of treating cancer in a subject comprising administering to the subject an antibody-partner molecule of claim 1 such that the cancer is treated in the subject.

46. The method of claim 45, wherein the cancer is a renal cell carcinoma or lymphoma.

47. The method of claim 45, wherein the cancer is selected from the group consisting of: renal cell carcinomas (RCC), clear cell RCC, glioblastoma, non-Hodgkin's lymphoma (NHL), acute lymphocytic leukemia (ALL), chronic lymphocytic leukemia (CLL), Burkitt's lymphoma, anaplastic large-cell lymphomas (ALCL), multiple myeloma, cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), entroblastic/centrocytic (cb/cc) follicular lymphomas cancers, diffuse large cell lymphomas of B cell lineage, angioimmunoblastic lymphadenopathy (AILD)-like T cell lymphoma, HIV associated body cavity based lymphomas, embryonal carcinomas, undifferentiated carcinomas of the rhino-pharynx, Schmincke's tumor, Castleman's disease, Kaposi's Sarcoma, multiple myeloma, Waldenstrom's macroglobulinemia and B-cell lymphomas.

48. A method of treating or preventing an autoimmune disease in a subject comprising administering to the subject an antibody-partner molecule of claim 1 whereby the autoimmune disease is treated or prevented in the subject.

49. A method of treating or preventing inflammation in a subject comprising administering to the subject an antibody-partner molecule of claim 1 such that the inflammation is treated or prevented in the subject.

50. A method of treating a viral infection in a subject comprising administering to the subject an antibody-partner molecule of claim 1 such that the viral infection is treated in the subject.

51. The antibody-partner molecule conjugate of claim 1, wherein the partner molecule is conjugated to the antibody by a chemical linker.

52. The antibody-partner molecule conjugate of claim 51, wherein the chemical linker is selected from the group consisting of peptidyl linkers, hydrazine linkers, and disulfide linkers.

53.-55. (canceled)

56. The antibody-partner molecule conjugate of claim 1, wherein the antibody, or antigen binding portion thereof, is nonfucosylated.

57. An isolated monoclonal antibody, or an antigen-binding portion thereof, comprising: a heavy chain variable region comprising the amino acid sequence of SEQ ID NO: 6 and a light chain variable region comprising the amino acid sequence of SEQ ID NO: 12.

58. An isolated monoclonal antibody, or an antigen binding portion thereof, which binds an epitope on the human CD70 protein recognized by an antibody comprising: a heavy chain variable region comprising the amino acid sequence of SEQ ID NO: 6 and a light chain variable region comprising the amino acid sequence of SEQ ID NO: 12.

59.-63. (canceled)


SEQUENCE LISTING (92 sequences)

SEQ ID NO: 1 - protein, 118 aa, Homo sapiens
QVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTVSS

SEQ ID NO: 2 - protein, 119 aa, Homo sapiens
QIQLVESGGGVVQPGRSLRLSCAASGFTFGYYAMHWVRQAPGKGLEWVAVISYDGSIKYYADSVKGRFTISRDNSKNTLYLQMNSLRAEDTAVYYCAREGPYSNYLDYWGQGTLVTVSS

SEQ ID NO: 3 - protein, 118 aa, Homo sapiens
QVQLVESGGGVVQPGRSLRLSCATSGFTFSDYGMHWVRQAPGKGLEWVAVIWYDGSNKYYADSVKGRFTISRDNSKKTLSLQMNSLRAEDTAVYYCARDSIMVRGDYWGQGTLVTVSS

SEQ ID NO: 4 - protein, 118 aa, Homo sapiens
QVQLVESGGGVVQPGRSLRLSCAASGFTFSDHGMHWVRQAPGKGLEWVAVIWYDGSNKYYADSVKGRFTISRDNSKNTLYLQMNSLRAEDTAVYYCARDSIMVRGDYWGQGTLVTVSS

SEQ ID NO: 5 - protein, 122 aa, Homo sapiens
QVQLQESGPGLVKPSETLSLTCTVSGGSVSSDYYYWSWIRQPPGKGLEWLGYIYYSGSTNYNPSLKSRVTISVDTSKNQFSLKLRSVTTADTAVYYCARGDGDYGGNCFDYWGQGTLVTVSS

SEQ ID NO: 6 - protein, 120 aa, Homo sapiens
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSS

SEQ ID NO: 7 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIK

SEQ ID NO: 8 - protein, 107 aa, Homo sapiens
AIQLTQSPSSLSASVGDRVTITCRASQGISSALAWYQQKPGKAPKFLIYDASSLESGVPSRFSGSGSGTDFTLTISSLQPEDFATYYCQQFNSYPFTFGPGTKVDIK

SEQ ID NO: 9 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 10 - protein, 107 aa, Homo sapiens; identical to SEQ ID NO: 9

SEQ ID NO: 11 - protein, 107 aa, Homo sapiens
EIVLTQSPATLSLSPGERATLSCRASQSVSSYLAWYQQKPGQAPRLLIFDASNRATGIPARFSGSGSGTDFTLTISSLEPEDFAVYYCQQRSNWPLTFGGGTKVEIK

SEQ ID NO: 12 - protein, 108 aa, Homo sapiens
EIVLTQSPGTLSLSPGERATLSCRASQSISSSYLAWYQQKPGQAPRLLIYGASSRATGIPDRFSGSGSGTDFTLTISRLEPEDFAVYYCQQYGSSPYTFGQGTKLEIK

SEQ ID NO: 13 - protein, 5 aa, Homo sapiens
SYIMH

SEQ ID NO: 14 - protein, 5 aa, Homo sapiens
YYAMH

SEQ ID NO: 15 - protein, 5 aa, Homo sapiens
DYGMH

SEQ ID NO: 16 - protein, 5 aa, Homo sapiens
DHGMH

SEQ ID NO: 17 - protein, 7 aa, Homo sapiens
SDYYYWS

SEQ ID NO: 18 - protein, 5 aa, Homo sapiens
IYAMS

SEQ ID NO: 19 - protein, 17 aa, Homo sapiens
VISYDGRNKYYADSVKG

SEQ ID NO: 20 - protein, 17 aa, Homo sapiens
VISYDGSIKYYADSVKG

SEQ ID NO: 21 - protein, 17 aa, Homo sapiens
VIWYDGSNKYYADSVKG

SEQ ID NO: 22 - protein, 17 aa, Homo sapiens
VIWYDGSNKYYADSVKG

SEQ ID NO: 23 - protein, 16 aa, Homo sapiens
YIYYSGSTNYNPSLKS

SEQ ID NO: 24 - protein, 17 aa, Homo sapiens
AISDSGGRTYFADSVRG

SEQ ID NO: 25 - protein, 9 aa, Homo sapiens
DTDGYDFDY

SEQ ID NO: 26 - protein, 10 aa, Homo sapiens
EGPYSNYLDY

SEQ ID NO: 27 - protein, 9 aa, Homo sapiens
DSIMVRGDY

SEQ ID NO: 28 - protein, 9 aa, Homo sapiens
DSIMVRGDY

SEQ ID NO: 29 - protein, 12 aa, Homo sapiens
GDGDYGGNCFDY

SEQ ID NO: 30 - protein, 11 aa, Homo sapiens
VDYSNYLFFDY

SEQ ID NO: 31 - protein, 11 aa, Homo sapiens
RASQSVSSYLA

SEQ ID NO: 32 - protein, 11 aa, Homo sapiens
RASQGISSALA

SEQ ID NO: 33 - protein, 11 aa, Homo sapiens
RASQGISSWLA

SEQ ID NO: 34 - protein, 11 aa, Homo sapiens
RASQGISSWLA

SEQ ID NO: 35 - protein, 11 aa, Homo sapiens
RASQSVSSYLA

SEQ ID NO: 36 - protein, 12 aa, Homo sapiens
RASQSISSSYLA

SEQ ID NO: 37 - protein, 7 aa, Homo sapiens
DASNRAT

SEQ ID NO: 38 - protein, 7 aa, Homo sapiens
DASSLES

SEQ ID NO: 39 - protein, 7 aa, Homo sapiens
AASSLQS

SEQ ID NO: 40 - protein, 7 aa, Homo sapiens
AASSLQS

SEQ ID NO: 41 - protein, 7 aa, Homo sapiens
DASNRAT

SEQ ID NO: 42 - protein, 7 aa, Homo sapiens
GASSRAT

SEQ ID NO: 43 - protein, 9 aa, Homo sapiens
QQRTNWPLT

SEQ ID NO: 44 - protein, 9 aa, Homo sapiens
QQFNSYPFT

SEQ ID NO: 45 - protein, 9 aa, Homo sapiens
QQYNSYPLT

SEQ ID NO: 46 - protein, 9 aa, Homo sapiens
QQYNSYPLT

SEQ ID NO: 47 - protein, 9 aa, Homo sapiens
QQRSNWPLT

SEQ ID NO: 48 - protein, 9 aa, Homo sapiens
QQYGSSPYT

SEQ ID NO: 49 - DNA, 354 bp, Homo sapiens
caggtgcagctggtggagtctgggggaggcgtggtccagcctgggaggtccctgagactctcctgtgcagcctctggatttaccttcagtagctatattatgcactgggtccgccaggctccaggcaaggggctggagtgggtggcagttatatcatatgatggaagaaacaaatactacgcagactccgtgaagggccgattcaccatctccagagacaattccaagaacacgctgtatctgcaaatgaacagcctgagagctgaggacacggctgtgtattactgtgcgagagatacggatggctacgattttgactactggggccagggaaccctggtcaccgtctcctca

SEQ ID NO: 50 - DNA, 357 bp, Homo sapiens
caaatacaactggtggagtctgggggaggcgtggtccagcctgggaggtccctgagactctcctgtgcagcctctggattcaccttcggttactatgctatgcactgggtccgccaggctccaggcaaggggctggagtgggtggcagttatatcatatgatggaagcattaaatactacgcagactccgtgaagggccgattcaccatctccagagacaattccaagaacacgctgtatctgcaaatgaacagcctgagagctgaggacacggctgtgtattactgtgcgagagagggcccttacagtaactaccttgactactggggccagggaaccctggtcaccgtctcctca

SEQ ID NO: 51 - DNA, 354 bp, Homo sapiens
caggtgcagctggtggagtctgggggaggcgtggtccagcctgggaggtccctgagactctcctgtgcgacgtctggattcaccttcagtgactatggcatgcactgggtccgccaggctccaggcaaggggctggagtgggtggcagttatatggtatgatggaagtaataaatactatgcagactccgtgaagggccgattcaccatctccagagacaattccaagaaaacgctgtctctgcaaatgaacagcctgagagccgaggacacggctgtgtattactgtgcgagagattctattatggttcggggggactactggggccagggaaccctggtcaccgtctcctca

SEQ ID NO: 52 - DNA, 354 bp, Homo sapiens
caggtgcagctggtggagtctgggggaggcgtggtccagcctgggaggtccctgagactctcctgtgcagcgtctggattcaccttcagcgaccatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagattctattatggttcggggggactactggggccagggaaccctggtcaccgtctcctca

SEQ ID NO: 53 - DNA, 366 bp, Homo sapiens
caggtgcagctgcaggagtcgggcccaggactggtgaagccttcggagaccctgtccctcacctgcactgtctctggtggctccgtcagcagtgattattactactggagctggatccggcagcccccagggaagggactggagtggcttgggtatatctattacagtgggagcaccaactacaacccctccctcaagagtcgagtcaccatatcagtagacacgtccaagaaccagttctccctgaagctgaggtctgtgaccactgcggacacggccgtgtattactgtgcgagaggggatggggactacggtggtaactgttttgactactggggccagggaaccctggtcaccgtctcctca

SEQ ID NO: 54 - DNA, 360 bp, Homo sapiens
gaggtgcagctgttggagtctgggggaggcttggtacagcctggggggtccctgagactctcctgtgcagcctctggattcacctttagcatctatgccatgagctgggtccgccaggctccagggaaggggctggagtgggtctcagctattagtgatagtggtggtcgcacatacttcgcagactccgtgaggggccggttcaccatctccagagacaattccaagaacacgctgtctctgcaaatgaacagcctgagagccgaggacacggccgtatattactgtgcgaaggtcgactacagtaactacctattctttgactactggggccagggaaccctggtcaccgtctcctca

SEQ ID NO: 55 - DNA, 321 bp, Homo sapiens
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtaccaactggccgctcactttcggcggagggaccaaggtggagatcaaa

SEQ ID NO: 56 - DNA, 321 bp, Homo sapiens
gccatccagttgacccagtctccatcctccctgtctgcatctgtaggagacagagtcaccatcacttgccgggcaagtcagggcattagcagtgctttagcctggtatcagcagaaaccagggaaagctcctaagttcttgatctatgatgcctccagtttggaaagtggggtcccatcaaggttcagcggcagtggatctgggacagatttcactctcaccatcagcagcctgcagcctgaagattttgcaacttattactgtcaacagtttaatagttacccattcactttcggccctgggaccaaagtggatatcaaa

SEQ ID NO: 57 - DNA, 321 bp, Homo sapiens
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgctcactttcggcggagggaccaaggtggagatcaaa

SEQ ID NO: 58 - DNA, 321 bp, Homo sapiens; identical to SEQ ID NO: 57

SEQ ID NO: 59 - DNA, 321 bp, Homo sapiens
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctttgatgcatccaacagggccactggcatcccagccaggttcagtggcagtgggtctgggacagacttcactctcaccatcagcagcctagagcctgaagattttgcagtttattactgtcagcaacgtagcaactggccgctcactttcggcggagggaccaaggtggagatcaaa

SEQ ID NO: 60 - DNA, 324 bp, Homo sapiens
gaaattgtgttgacgcagtctccaggcaccctgtctttgtctccaggggaaagagccaccctctcctgcagggccagtcagagtattagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtacacttttggccaggggaccaagctggagatcaaa

SEQ ID NO: 61 - protein, 98 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAR

SEQ ID NO: 62 - protein, 98 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 63 - protein, 99 aa, Homo sapiens
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAR

SEQ ID NO: 64 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 65 - protein, 94 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNW

SEQ ID NO: 66 - protein, 95 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYP

SEQ ID NO: 67 - protein, 95 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYP

SEQ ID NO: 68 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 69 - protein, 15 aa, Homo sapiens
YFDYWGQGTLVTVSS

SEQ ID NO: 70 - protein, 12 aa, Homo sapiens
LTFGGGTKVEIK

SEQ ID NO: 71 - protein, 12 aa, Homo sapiens
FTFGPGTKVDIK

SEQ ID NO: 72 - protein, 12 aa, Homo sapiens
YTFGQGTKLEIK

SEQ ID NO: 73 - protein, 122 aa, Homo sapiens
QVQLQESGPGLVKPSETLSLTCTVSGGSVSSDYYYWSWIRQPPGKGLEWLGYIYYSGSTNYNPSLKSRVTISVDTSKNQFSLKLRSVTTADTAVYYCARGDGDYGGNYFDYWGQGTLVTVSS

SEQ ID NO: 74 - DNA, 366 bp, Homo sapiens
caggtgcagctgcaggagtcgggcccaggactggtgaagccttcggagaccctgtccctcacctgcactgtctctggtggctccgtcagcagtgattattactactggagctggatccggcagcccccagggaagggactggagtggcttgggtatatctattacagtgggagcaccaactacaacccctccctcaagagtcgagtcaccatatcagtagacacgtccaagaaccagttctccctgaagctgaggtctgtgaccactgcggacacggccgtgtattactgtgcgagaggggatggggactacggtggtaactattttgactactggggccagggaaccctggtcaccgtctcctca

SEQ ID NO: 75 - protein, 12 aa, Homo sapiens
GDGDYGGNYFDY

SEQ ID NO: 76 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 77 - protein, 4 aa, Artificial
  Peptide linker
ALAL

SEQ ID NO: 78 - protein, 4 aa, Artificial
  Peptide linker
ALAL

SEQ ID NO: 79 - protein, 4 aa, Artificial
  Peptide linker
GFLG

SEQ ID NO: 80 - protein, 4 aa, Artificial
  Peptide linker
PRFK

SEQ ID NO: 81 - protein, 4 aa, Artificial
  Peptide linker
TRLR

SEQ ID NO: 82 - protein, 4 aa, Artificial
  Peptide linker
SKGR

SEQ ID NO: 83 - protein, 4 aa, Artificial
  Peptide linker
PNDK

SEQ ID NO: 84 - protein, 6 aa, Artificial
  Peptide linker
PVGLIG

SEQ ID NO: 85 - protein, 5 aa, Artificial
  Peptide linker
GPLGV

SEQ ID NO: 86 - protein, 8 aa, Artificial
  Peptide linker
GPLGIAGQ

SEQ ID NO: 87 - protein, 4 aa, Artificial
  Peptide linker
PLGL

SEQ ID NO: 88 - protein, 8 aa, Artificial
  Peptide linker
GPLGMLSQ

SEQ ID NO: 89 - protein, 8 aa, Artificial
  Peptide linker
GPLGLWAQ

SEQ ID NO: 90 - protein, 9 aa, Cytomegalovirus
IPSINVHHY

SEQ ID NO: 91 - protein, 4 aa, Artificial
  Peptide linker
LLGL

SEQ ID NO: 92 - protein, 4 aa, Artificial
  Peptide linker
ALAL